SEQUENCE LISTING (258 sequences)

SEQ ID NO: 1 - DNA, 14 bp, Artificial Sequence
  Synthetic polynucleotide
atgttgtttaaaat

SEQ ID NO: 2 - DNA, 63 bp, Artificial Sequence
  Synthetic polynucleotide
cgcgtactcgagatggatccggggaatgagaattcagccacagaggctgccgcgatcatagac

SEQ ID NO: 3 - DNA, 31 bp, Artificial Sequence
  Synthetic polynucleotide
ccggaacccgggtcagggatctggctcaaag

SEQ ID NO: 4 - DNA, 28 bp, Artificial Sequence
  Synthetic polynucleotide
gtccccgctcctgtgcttggccccttcc

SEQ ID NO: 5 - DNA, 28 bp, Artificial Sequence
  Synthetic polynucleotide
ggaaggggccaagcacaggagcggggac

SEQ ID NO: 6 - DNA, 43 bp, Artificial Sequence
  Synthetic polynucleotide
gcaaagccccccgccgcagagccgcagccatggatagcagcag

SEQ ID NO: 7 - DNA, 43 bp, Artificial Sequence
  Synthetic polynucleotide
ctgctgctatccatggctgcggctctgcggcggggggctttgc

SEQ ID NO: 8 - DNA, 35 bp, Artificial Sequence
  Synthetic polynucleotide
gtccacgaagcagcgcaaatgccagcagtgtcagc

SEQ ID NO: 9 - DNA, 35 bp, Artificial Sequence
  Synthetic polynucleotide
gctgacactgctggcatttgcgctgcttcgtggac

SEQ ID NO: 10 - DNA, 23 bp, Artificial Sequence
  Synthetic polynucleotide
cgatcgataattttcttcattcg

SEQ ID NO: 11 - DNA, 23 bp, Artificial Sequence
  Synthetic polynucleotide
gcgttttattttcactgatggag

SEQ ID NO: 12 - DNA, 20 bp, Artificial Sequence
  Synthetic polynucleotide
atggatcaggaagtgaacgg

SEQ ID NO: 13 - DNA, 20 bp, Artificial Sequence
  Synthetic polynucleotide
cttctttcatgtgccccatt

SEQ ID NO: 14 - DNA, 20 bp, Artificial Sequence
  Synthetic polynucleotide
tatccgtctcatcctcccag

SEQ ID NO: 15 - DNA, 20 bp, Artificial Sequence
  Synthetic polynucleotide
cacaaaaagtgcctgagcaa

SEQ ID NO: 16 - DNA, 20 bp, Artificial Sequence
  Synthetic polynucleotide
aattccccactgcccttact

SEQ ID NO: 17 - DNA, 20 bp, Artificial Sequence
  Synthetic polynucleotide
agcccatggcaaaacattag

SEQ ID NO: 18 - DNA, 23 bp, Artificial Sequence
  Synthetic polynucleotide
tctagaaaattccgggaaaaact

SEQ ID NO: 19 - DNA, 23 bp, Artificial Sequence
  Synthetic polynucleotide
ggtgtgctggaaatagacttttg

SEQ ID NO: 20 - DNA, 20 bp, Artificial Sequence
  Synthetic polynucleotide
gggtccaactgtcatcgtct

SEQ ID NO: 21 - DNA, 20 bp, Artificial Sequence
  Synthetic polynucleotide
ctggcaccaagtcaaaatca

SEQ ID NO: 22 - DNA, 25 bp, Artificial Sequence
  Synthetic polynucleotide
ggctgatctcaaccagtattacaca

SEQ ID NO: 23 - DNA, 23 bp, Artificial Sequence
  Synthetic polynucleotide
gaacaaaagagcacatcccaaac

SEQ ID NO: 24 - DNA, 20 bp, Artificial Sequence
  Synthetic polynucleotide
cggccgtaatgcaggaatat

SEQ ID NO: 25 - DNA, 20 bp, Artificial Sequence
  Synthetic polynucleotide
aagaagcgatgctccaaacg

SEQ ID NO: 26 - DNA, 20 bp, Artificial Sequence
  Synthetic polynucleotide
cggagaggaaatgccagttg

SEQ ID NO: 27 - DNA, 22 bp, Artificial Sequence
  Synthetic polynucleotide
acggtctctttcctccaatctg

SEQ ID NO: 28 - DNA, 22 bp, Artificial Sequence
  Synthetic polynucleotide
gtcgtaccaccaggatcacaaa

SEQ ID NO: 29 - DNA, 24 bp, Artificial Sequence
  Synthetic polynucleotide
tgctgcagctcatcattaactttt

SEQ ID NO: 30 - DNA, 22 bp, Artificial Sequence
  Synthetic polynucleotide
caacatattggcgtcacattcg

SEQ ID NO: 31 - DNA, 18 bp, Artificial Sequence
  Synthetic polynucleotide
tgcccgttcgaccagagt

SEQ ID NO: 32 - DNA, 21 bp, Artificial Sequence
  Synthetic polynucleotide
cgagaaatcaactccggaaca

SEQ ID NO: 33 - DNA, 20 bp, Artificial Sequence
  Synthetic polynucleotide
aagtgtgtcgcgaagagcaa

SEQ ID NO: 34 - DNA, 22 bp, Artificial Sequence
  Synthetic polynucleotide
tttccaagtcgagtacgctcaa

SEQ ID NO: 35 - DNA, 20 bp, Artificial Sequence
  Synthetic polynucleotide
ttgccacgaattgcaatcat

SEQ ID NO: 36 - DNA, 21 bp, Artificial Sequence
  Synthetic polynucleotide
tcagcactggaaccactctca

SEQ ID NO: 37 - DNA, 19 bp, Artificial Sequence
  Synthetic polynucleotide
tcggttccgacgacaactc

SEQ ID NO: 38 - DNA, 24 bp, Artificial Sequence
  Synthetic polynucleotide
attttggagcgtgattttaaggtt

SEQ ID NO: 39 - DNA, 17 bp, Artificial Sequence
  Synthetic polynucleotide
ggcgcgttggacaagct

SEQ ID NO: 40 - DNA, 18 bp, Artificial Sequence
  Synthetic polynucleotide
gctccacgcgatttcgtt

SEQ ID NO: 41 - DNA, 21 bp, Artificial Sequence
  Synthetic polynucleotide
gcgccagaagttttcacaaac

SEQ ID NO: 42 - DNA, 20 bp, Artificial Sequence
  Synthetic polynucleotide
gggcaacagccgtacttgtt

SEQ ID NO: 43 - DNA, 23 bp, Artificial Sequence
  Synthetic polynucleotide
cgatccataatggcatagcagaa

SEQ ID NO: 44 - DNA, 21 bp, Artificial Sequence
  Synthetic polynucleotide
ctgcaatttgtgccacatcac

SEQ ID NO: 45 - DNA, 20 bp, Artificial Sequence
  Synthetic polynucleotide
tcacgtccattggtggaaga

SEQ ID NO: 46 - DNA, 21 bp, Artificial Sequence
  Synthetic polynucleotide
gatatgagcgtccggaactca

SEQ ID NO: 47 - DNA, 21 bp, Artificial Sequence
  Synthetic polynucleotide
acggaacgaatccttcatcaa

SEQ ID NO: 48 - DNA, 20 bp, Artificial Sequence
  Synthetic polynucleotide
ctctacgccaaacgttgcaa

SEQ ID NO: 49 - DNA, 19 bp, Artificial Sequence
  Synthetic polynucleotide
actccggcgacaacaagtg

SEQ ID NO: 50 - DNA, 23 bp, Artificial Sequence
  Synthetic polynucleotide
tggaaacatcaaggtgcttatgg

SEQ ID NO: 51 - DNA, 19 bp, Artificial Sequence
  Synthetic polynucleotide
ctcatgagagcgggatcga

SEQ ID NO: 52 - DNA, 18 bp, Artificial Sequence
  Synthetic polynucleotide
cctgacgccgctagcaaa

SEQ ID NO: 53 - DNA, 20 bp, Artificial Sequence
  Synthetic polynucleotide
gcaacttcagacggcatctg

SEQ ID NO: 54 - DNA, 20 bp, Artificial Sequence
  Synthetic polynucleotide
tggagaaggaccacgaatgg

SEQ ID NO: 55 - DNA, 20 bp, Artificial Sequence
  Synthetic polynucleotide
gatgggctgttttcctttgc

SEQ ID NO: 56 - DNA, 21 bp, Artificial Sequence
  Synthetic polynucleotide
gtcaagttgtccgacggattc

SEQ ID NO: 57 - DNA, 21 bp, Artificial Sequence
  Synthetic polynucleotide
tggcaaacattccagcttctg

SEQ ID NO: 58 - DNA, 27 bp, Artificial Sequence
  Synthetic polynucleotide
gaagatgaatgtcaacaaggatgtaaa

SEQ ID NO: 59 - DNA, 20 bp, Artificial Sequence
  Synthetic polynucleotide
tgcattgtgctccgttgaag

SEQ ID NO: 60 - DNA, 22 bp, Artificial Sequence
  Synthetic polynucleotide
ccgaagaatccgatgagaaaac

SEQ ID NO: 61 - DNA, 19 bp, Artificial Sequence
  Synthetic polynucleotide
cactttttgctgcgcatca

SEQ ID NO: 62 - DNA, 21 bp, Artificial Sequence
  Synthetic polynucleotide
cggaaagccaaagacaatcac

SEQ ID NO: 63 - DNA, 23 bp, Artificial Sequence
  Synthetic polynucleotide
gagatattcatcgttcgccaact

SEQ ID NO: 64 - DNA, 25 bp, Artificial Sequence
  Synthetic polynucleotide
tgacattgttgaacagatggagatc

SEQ ID NO: 65 - DNA, 16 bp, Artificial Sequence
  Synthetic polynucleotide
tgcggctgcgtttgaa

SEQ ID NO: 66 - DNA, 23 bp, Artificial Sequence
  Synthetic polynucleotide
atacattgtggcgaaggctattg

SEQ ID NO: 67 - DNA, 21 bp, Artificial Sequence
  Synthetic polynucleotide
tgtaccgaggtccatcacgaa

SEQ ID NO: 68 - DNA, 22 bp, Artificial Sequence
  Synthetic polynucleotide
ggagagcacaacatgcgtatga

SEQ ID NO: 69 - DNA, 20 bp, Artificial Sequence
  Synthetic polynucleotide
cagcgagacgttcgaaagtg

SEQ ID NO: 70 - DNA, 25 bp, Artificial Sequence
  Synthetic polynucleotide
aatattggaaaagcacctgaaatgt

SEQ ID NO: 71 - DNA, 25 bp, Artificial Sequence
  Synthetic polynucleotide
tttgatgtggaagtgaagtcattgt

SEQ ID NO: 72 - DNA, 20 bp, Artificial Sequence
  Synthetic polynucleotide
aacttggcgaaggcaaagac

SEQ ID NO: 73 - DNA, 19 bp, Artificial Sequence
  Synthetic polynucleotide
aagcaaacgccgaattggt

SEQ ID NO: 74 - DNA, 20 bp, Artificial Sequence
  Synthetic polynucleotide
tcattcagttggccgctctt

SEQ ID NO: 75 - DNA, 22 bp, Artificial Sequence
  Synthetic polynucleotide
tgtggcgatagatagcgatcaa

SEQ ID NO: 76 - DNA, 22 bp, Artificial Sequence
  Synthetic polynucleotide
tcaggaagttcacgatgatgga

SEQ ID NO: 77 - DNA, 20 bp, Artificial Sequence
  Synthetic polynucleotide
tctgaaggcacatgctcgaa

SEQ ID NO: 78 - DNA, 22 bp, Artificial Sequence
  Synthetic polynucleotide
gggtgcagccaagagttttaga

SEQ ID NO: 79 - DNA, 22 bp, Artificial Sequence
  Synthetic polynucleotide
ggagttgacatcgttcctccat

SEQ ID NO: 80 - DNA, 27 bp, Artificial Sequence
  Synthetic polynucleotide
agtactatgatttgtgtcgacaattcg

SEQ ID NO: 81 - DNA, 19 bp, Artificial Sequence
  Synthetic polynucleotide
ggagccgagttggttggaa

SEQ ID NO: 82 - DNA, 21 bp, Artificial Sequence
  Synthetic polynucleotide
acgttttcgctatgccacagt

SEQ ID NO: 83 - DNA, 17 bp, Artificial Sequence
  Synthetic polynucleotide
tggatcgaccgcttcga

SEQ ID NO: 84 - DNA, 23 bp, Artificial Sequence
  Synthetic polynucleotide
caaaggagccaaaagatcttgtc

SEQ ID NO: 85 - DNA, 23 bp, Artificial Sequence
  Synthetic polynucleotide
cactgagaaccttcgtcaactca

SEQ ID NO: 86 - DNA, 22 bp, Artificial Sequence
  Synthetic polynucleotide
ttgaagttttgacatcctcgaa

SEQ ID NO: 87 - DNA, 26 bp, Artificial Sequence
  Synthetic polynucleotide
ttattagtgtcttctcgtgaacctcg

SEQ ID NO: 88 - DNA, 21 bp, Artificial Sequence
  Synthetic polynucleotide
ctgctggacaggaagattacg

SEQ ID NO: 89 - DNA, 21 bp, Artificial Sequence
  Synthetic polynucleotide
ctcggacattctcgaatgaag

SEQ ID NO: 90 - DNA, 21 bp, Artificial Sequence
  Synthetic polynucleotide
gttcccgtgttcatcactcat

SEQ ID NO: 91 - DNA, 21 bp, Artificial Sequence
  Synthetic polynucleotide
acaccgtcgagaagctgtaga

SEQ ID NO: 92 - DNA, 21 bp, Artificial Sequence
  Synthetic polynucleotide
gtcgcttcaaatcagttcagc

SEQ ID NO: 93 - DNA, 22 bp, Artificial Sequence
  Synthetic polynucleotide
gttcttgtcaagtgatccgaca

SEQ ID NO: 94 - DNA, 19 bp, Artificial Sequence
  Synthetic polynucleotide
cacattccatgcccaagta

SEQ ID NO: 95 - DNA, 19 bp, Artificial Sequence
  Synthetic polynucleotide
gcgcttagactgtgacatg

SEQ ID NO: 96 - DNA, 20 bp, Artificial Sequence
  Synthetic polynucleotide
aaggataagggcgacagcaa

SEQ ID NO: 97 - DNA, 19 bp, Artificial Sequence
  Synthetic polynucleotide
agaagccgatatgtggacc

SEQ ID NO: 98 - DNA, 21 bp, Artificial Sequence
  Synthetic polynucleotide
cacttaggctttgtagcaaga

SEQ ID NO: 99 - DNA, 21 bp, Artificial Sequence
  Synthetic polynucleotide
gcgtgttcatatctactcttt

SEQ ID NO: 100 - DNA, 21 bp, Artificial Sequence
  Synthetic polynucleotide
tgatagtgacatgatacaaac

SEQ ID NO: 101 - DNA, 18 bp, Artificial Sequence
  Synthetic polynucleotide
ctggcacccagcacaatg

SEQ ID NO: 102 - DNA, 21 bp, Artificial Sequence
  Synthetic polynucleotide
ccgatccacacggagtacttg

SEQ ID NO: 103 - DNA, 20 bp, Artificial Sequence
  Synthetic polynucleotide
gcaccgtcaaggctgagaac

SEQ ID NO: 104 - DNA, 21 bp, Artificial Sequence
  Synthetic polynucleotide
ggatctcgctcctggaagatg

SEQ ID NO: 105 - DNA, 21 bp, Artificial Sequence
  Synthetic polynucleotide
aacatgtatggcttccggaaa

SEQ ID NO: 106 - DNA, 22 bp, Artificial Sequence
  Synthetic polynucleotide
tgtcgtctctctctggcttgac

SEQ ID NO: 107 - DNA, 19 bp, Artificial Sequence
  Synthetic polynucleotide
tcatgtcaacctatggcag

SEQ ID NO: 108 - DNA, 18 bp, Artificial Sequence
  Synthetic polynucleotide
catggtgcttaccgtgtg

SEQ ID NO: 109 - DNA, 18 bp, Artificial Sequence
  Synthetic polynucleotide
tgccggctggaagaactc

SEQ ID NO: 110 - DNA, 20 bp, Artificial Sequence
  Synthetic polynucleotide
ccgcatgaatcgactatgca

SEQ ID NO: 111 - DNA, 24 bp, Artificial Sequence
  Synthetic polynucleotide
tggtccgtactgtaccctacttca

SEQ ID NO: 112 - DNA, 20 bp, Artificial Sequence
  Synthetic polynucleotide
ggcggatcgagttcttccat

SEQ ID NO: 113 - DNA, 26 bp, Artificial Sequence
  Synthetic polynucleotide
gcctgtgtctcgtcttgtatctctaa

SEQ ID NO: 114 - DNA, 20 bp, Artificial Sequence
  Synthetic polynucleotide
tccggccatatcgttgtgtt

SEQ ID NO: 115 - DNA, 23 bp, Artificial Sequence
  Synthetic polynucleotide
tgaatatgctttggctgctgtag

SEQ ID NO: 116 - DNA, 23 bp, Artificial Sequence
  Synthetic polynucleotide
ccacaccatttgcagctttaatt

SEQ ID NO: 117 - DNA, 20 bp, Artificial Sequence
  Synthetic polynucleotide
gaatgacggtgcgcaactct

SEQ ID NO: 118 - DNA, 21 bp, Artificial Sequence
  Synthetic polynucleotide
cagccccaataaccgtatgaa

SEQ ID NO: 119 - DNA, 27 bp, Artificial Sequence
  Synthetic polynucleotide
actatattttctccagaaggtcgctta

SEQ ID NO: 120 - DNA, 20 bp, Artificial Sequence
  Synthetic polynucleotide
caaacaggtgcctgcatgtc

SEQ ID NO: 121 - DNA, 22 bp, Artificial Sequence
  Synthetic polynucleotide
ccagagtggagacacagaacca

SEQ ID NO: 122 - DNA, 23 bp, Artificial Sequence
  Synthetic polynucleotide
gggtcacactctccactgtcatt

SEQ ID NO: 123 - DNA, 17 bp, Artificial Sequence
  Synthetic polynucleotide
cccgagggtcggctcta

SEQ ID NO: 124 - DNA, 22 bp, Artificial Sequence
  Synthetic polynucleotide
gatgtaaggccaccctggttaa

SEQ ID NO: 125 - DNA, 21 bp, Artificial Sequence
  Synthetic polynucleotide
gccagtctgaagcagcgttat

SEQ ID NO: 126 - DNA, 20 bp, Artificial Sequence
  Synthetic polynucleotide
tgagggcagagatgccaaac

SEQ ID NO: 127 - DNA, 24 bp, Artificial Sequence
  Synthetic polynucleotide
aggcgcttctttccatactatgtt

SEQ ID NO: 128 - DNA, 20 bp, Artificial Sequence
  Synthetic polynucleotide
gccccctttccttcttcatc

SEQ ID NO: 129 - DNA, 24 bp, Artificial Sequence
  Synthetic polynucleotide
gatgcgaaatggatatgaattgtc

SEQ ID NO: 130 - DNA, 20 bp, Artificial Sequence
  Synthetic polynucleotide
aggtttcggcgtgtgaagtt

SEQ ID NO: 131 - DNA, 17 bp, Artificial Sequence
  Synthetic polynucleotide
ctgtggaccgggatgca

SEQ ID NO: 132 - DNA, 24 bp, Artificial Sequence
  Synthetic polynucleotide
gattttgtccttctcgatgatgtg

SEQ ID NO: 133 - DNA, 24 bp, Artificial Sequence
  Synthetic polynucleotide
acatgcttggtgtagcctatgaag

SEQ ID NO: 134 - DNA, 21 bp, Artificial Sequence
  Synthetic polynucleotide
agaggctgagccaagtatgca

SEQ ID NO: 135 - DNA, 21 bp, Artificial Sequence
  Synthetic polynucleotide
cagaagagccaggaatcgaaa

SEQ ID NO: 136 - DNA, 19 bp, Artificial Sequence
  Synthetic polynucleotide
tccatggcggaacttgaag

SEQ ID NO: 137 - DNA, 23 bp, Artificial Sequence
  Synthetic polynucleotide
caaggaagagtgtctgcaattca

SEQ ID NO: 138 - DNA, 16 bp, Artificial Sequence
  Synthetic polynucleotide
ccgctccatggccaaa

SEQ ID NO: 139 - DNA, 19 bp, Artificial Sequence
  Synthetic polynucleotide
cagccaatcggatgctgaa

SEQ ID NO: 140 - DNA, 20 bp, Artificial Sequence
  Synthetic polynucleotide
agggctgcaccaatgtaacc

SEQ ID NO: 141 - DNA, 18 bp, Artificial Sequence
  Synthetic polynucleotide
agaccaggccgcattgac

SEQ ID NO: 142 - DNA, 20 bp, Artificial Sequence
  Synthetic polynucleotide
tgcgcttcttcgtcttttca

SEQ ID NO: 143 - DNA, 21 bp, Artificial Sequence
  Synthetic polynucleotide
ttggctgcagataagcagaca

SEQ ID NO: 144 - DNA, 24 bp, Artificial Sequence
  Synthetic polynucleotide
tctttgtacacctggcaacctgta

SEQ ID NO: 145 - DNA, 19 bp, Artificial Sequence
  Synthetic polynucleotide
cacacggctgctggacagt

SEQ ID NO: 146 - DNA, 22 bp, Artificial Sequence
  Synthetic polynucleotide
cttggagctcatgggtgactct

SEQ ID NO: 147 - DNA, 19 bp, Artificial Sequence
  Synthetic polynucleotide
ggcccggccagataagatt

SEQ ID NO: 148 - DNA, 24 bp, Artificial Sequence
  Synthetic polynucleotide
caacattccactctcctgacagat

SEQ ID NO: 149 - DNA, 18 bp, Artificial Sequence
  Synthetic polynucleotide
cgagaacggcgagtccat

SEQ ID NO: 150 - DNA, 22 bp, Artificial Sequence
  Synthetic polynucleotide
ttctgcatgaccttggctactg

SEQ ID NO: 151 - DNA, 24 bp, Artificial Sequence
  Synthetic polynucleotide
ccagagttatttcagcgtgtagga

SEQ ID NO: 152 - DNA, 24 bp, Artificial Sequence
  Synthetic polynucleotide
cgtacctggtggtccatataacaa

SEQ ID NO: 153 - DNA, 19 bp, Artificial Sequence
  Synthetic polynucleotide
gcttctgtgcctggatcca

SEQ ID NO: 154 - DNA, 24 bp, Artificial Sequence
  Synthetic polynucleotide
tccatcgagtcactgtctttctct

SEQ ID NO: 155 - DNA, 21 bp, Artificial Sequence
  Synthetic polynucleotide
gcgtgagtgcctcaagtatcg

SEQ ID NO: 156 - DNA, 23 bp, Artificial Sequence
  Synthetic polynucleotide
ccagatgcctgacatactcatga

SEQ ID NO: 157 - DNA, 20 bp, Artificial Sequence
  Synthetic polynucleotide
gagcaggccaacaacaatga

SEQ ID NO: 158 - DNA, 21 bp, Artificial Sequence
  Synthetic polynucleotide
gctttgattcgccctgtgtag

SEQ ID NO: 159 - DNA, 24 bp, Artificial Sequence
  Synthetic polynucleotide
cactatggtgtgtgtggacaacag

SEQ ID NO: 160 - DNA, 19 bp, Artificial Sequence
  Synthetic polynucleotide
tgcagcctggtgggtaaga

SEQ ID NO: 161 - DNA, 22 bp, Artificial Sequence
  Synthetic polynucleotide
tgacaaagagggagctctgaca

SEQ ID NO: 162 - DNA, 19 bp, Artificial Sequence
  Synthetic polynucleotide
gacccagggccacagtttt

SEQ ID NO: 163 - DNA, 23 bp, Artificial Sequence
  Synthetic polynucleotide
gaagctgaggaagttggagttga

SEQ ID NO: 164 - DNA, 18 bp, Artificial Sequence
  Synthetic polynucleotide
tgcccaccgtcgtgtctt

SEQ ID NO: 165 - DNA, 20 bp, Artificial Sequence
  Synthetic polynucleotide
gccatctactgcccccctaa

SEQ ID NO: 166 - DNA, 23 bp, Artificial Sequence
  Synthetic polynucleotide
atggataatacccgactgcatgt

SEQ ID NO: 167 - DNA, 23 bp, Artificial Sequence
  Synthetic polynucleotide
gactcgtactgcttccgatatgg

SEQ ID NO: 168 - DNA, 27 bp, Artificial Sequence
  Synthetic polynucleotide
tcatagcacatcttcactactgcaact

SEQ ID NO: 169 - DNA, 18 bp, Artificial Sequence
  Synthetic polynucleotide
gggtccaagcggcttttc

SEQ ID NO: 170 - DNA, 22 bp, Artificial Sequence
  Synthetic polynucleotide
gctcctcatcctggtctgtctt

SEQ ID NO: 171 - DNA, 23 bp, Artificial Sequence
  Synthetic polynucleotide
tgaacagctggcaataaagaatg

SEQ ID NO: 172 - DNA, 24 bp, Artificial Sequence
  Synthetic polynucleotide
agtacatccacatgttcctccaaa

SEQ ID NO: 173 - DNA, 23 bp, Artificial Sequence
  Synthetic polynucleotide
ggaggaagctgacaacaatgaaa

SEQ ID NO: 174 - DNA, 17 bp, Artificial Sequence
  Synthetic polynucleotide
ggcctgcacgagggttt

SEQ ID NO: 175 - DNA, 25 bp, Artificial Sequence
  Synthetic polynucleotide
aaatctaagaggtggcagaaaaaca

SEQ ID NO: 176 - DNA, 20 bp, Artificial Sequence
  Synthetic polynucleotide
gccttctgcgtcacaccatt

SEQ ID NO: 177 - DNA, 16 bp, Artificial Sequence
  Synthetic polynucleotide
tgcgagcgctgcacat

SEQ ID NO: 178 - DNA, 21 bp, Artificial Sequence
  Synthetic polynucleotide
tcatgagcgtcttggttttcc

SEQ ID NO: 179 - DNA, 21 bp, Artificial Sequence
  Synthetic polynucleotide
cgccgctacaagttccttaaa

SEQ ID NO: 180 - DNA, 19 bp, Artificial Sequence
  Synthetic polynucleotide
ggatctgctcgtcgaaggg

SEQ ID NO: 181 - DNA, 21 bp, Artificial Sequence
  Synthetic polynucleotide
ctggtgccaacaattgaagct

SEQ ID NO: 182 - DNA, 21 bp, Artificial Sequence
  Synthetic polynucleotide
aggcacacaggcgcttatatg

SEQ ID NO: 183 - DNA, 27 bp, Artificial Sequence
  Synthetic polynucleotide
gtactaatggcaagaaaatcgaagttt

SEQ ID NO: 184 - DNA, 20 bp, Artificial Sequence
  Synthetic polynucleotide
gccgttgttcagggtaagca

SEQ ID NO: 185 - DNA, 20 bp, Artificial Sequence
  Synthetic polynucleotide
cctgcaggcggaaatagaac

SEQ ID NO: 186 - DNA, 23 bp, Artificial Sequence
  Synthetic polynucleotide
gcacacatagccatcacataagg

SEQ ID NO: 187 - DNA, 19 bp, Artificial Sequence
  Synthetic polynucleotide
catttttcctgcgcgtcat

SEQ ID NO: 188 - DNA, 18 bp, Artificial Sequence
  Synthetic polynucleotide
gcgacatccctgcgttct

SEQ ID NO: 189 - DNA, 19 bp, Artificial Sequence
  Synthetic polynucleotide
ctcggcagccaagtgaaaa

SEQ ID NO: 190 - DNA, 21 bp, Artificial Sequence
  Synthetic polynucleotide
ggtccgtgtacaccactcgat

SEQ ID NO: 191 - DNA, 25 bp, Artificial Sequence
  Synthetic polynucleotide
cataccaagcgtgtcatcaataaac

SEQ ID NO: 192 - DNA, 17 bp, Artificial Sequence
  Synthetic polynucleotide
tgcgcccatctgttgct

SEQ ID NO: 193 - DNA, 23 bp, Artificial Sequence
  Synthetic polynucleotide
acgaggagttttcagcaacaaat

SEQ ID NO: 194 - DNA, 21 bp, Artificial Sequence
  Synthetic polynucleotide
ttggcacttgcatggagtttt

SEQ ID NO: 195 - DNA, 21 bp, Artificial Sequence
  Synthetic polynucleotide
ctccgcaaacacaagacgaac

SEQ ID NO: 196 - DNA, 20 bp, Artificial Sequence
  Synthetic polynucleotide
cacatgggccgtgtagagtc

SEQ ID NO: 197 - DNA, 21 bp, Artificial Sequence
  Synthetic polynucleotide
gagggagcggctgacattatt

SEQ ID NO: 198 - DNA, 24 bp, Artificial Sequence
  Synthetic polynucleotide
accagggtttactggagtcatttc

SEQ ID NO: 199 - DNA, 17 bp, Artificial Sequence
  Synthetic polynucleotide
agcgcgtgccttcatca

SEQ ID NO: 200 - DNA, 24 bp, Artificial Sequence
  Synthetic polynucleotide
gtggtagttgtggtgtgacagttg

SEQ ID NO: 201 - DNA, 20 bp, Artificial Sequence
  Synthetic polynucleotide
tccgtgtcccagacgttctc

SEQ ID NO: 202 - DNA, 20 bp, Artificial Sequence
  Synthetic polynucleotide
gagaggacagggtggatgga

SEQ ID NO: 203 - DNA, 22 bp, Artificial Sequence
  Synthetic polynucleotide
tgaggttgctcatcggtttaaa

SEQ ID NO: 204 - DNA, 21 bp, Artificial Sequence
  Synthetic polynucleotide
gcaatcaacaccaaggctttg

SEQ ID NO: 205 - DNA, 20 bp, Artificial Sequence
  Synthetic polynucleotide
tgaagggcaatcacaacagg

SEQ ID NO: 206 - DNA, 20 bp, Artificial Sequence
  Synthetic polynucleotide
tgaccccaacatgacctctg

SEQ ID NO: 207 - DNA, 20 bp, Artificial Sequence
  Synthetic polynucleotide
ggccaagttctgggaagtca

SEQ ID NO: 208 - DNA, 19 bp, Artificial Sequence
  Synthetic polynucleotide
cgagtcgcccacgtagttg

SEQ ID NO: 209 - DNA, 27 bp, Artificial Sequence
  Synthetic polynucleotide
aaagaagctcaacataaagaccagact

SEQ ID NO: 210 - DNA, 22 bp, Artificial Sequence
  Synthetic polynucleotide
gtggagaaggaggcagattagc

SEQ ID NO: 211 - DNA, 16 bp, Artificial Sequence
  Synthetic polynucleotide
tgggcatcggcattgc

SEQ ID NO: 212 - DNA, 26 bp, Artificial Sequence
  Synthetic polynucleotide
acagcacaagatcatactgaagatca

SEQ ID NO: 213 - DNA, 17 bp, Artificial Sequence
  Synthetic polynucleotide
caggggacgcaccaagg

SEQ ID NO: 214 - DNA, 17 bp, Artificial Sequence
  Synthetic polynucleotide
cagcacgcgggtcatgg

SEQ ID NO: 215 - DNA, 27 bp, Artificial Sequence
  Synthetic polynucleotide
cctctactacaagctgcacaatgttaa

SEQ ID NO: 216 - DNA, 20 bp, Artificial Sequence
  Synthetic polynucleotide
ccgagtctggatcccttcct

SEQ ID NO: 217 - DNA, 26 bp, Artificial Sequence
  Synthetic polynucleotide
ttcacaatattaacagacccctgact

SEQ ID NO: 218 - DNA, 25 bp, Artificial Sequence
  Synthetic polynucleotide
gatttgctagacatttttcggtttc

SEQ ID NO: 219 - DNA, 18 bp, Artificial Sequence
  Synthetic polynucleotide
gaagcagcagcggaaacg

SEQ ID NO: 220 - DNA, 20 bp, Artificial Sequence
  Synthetic polynucleotide
ggcaagggatgagcttcaga

SEQ ID NO: 221 - DNA, 20 bp, Artificial Sequence
  Synthetic polynucleotide
cggcaggagggaaaggttac

SEQ ID NO: 222 - DNA, 23 bp, Artificial Sequence
  Synthetic polynucleotide
tcccaaactccgatagtccatag

SEQ ID NO: 223 - DNA, 19 bp, Artificial Sequence
  Synthetic polynucleotide
cgccgccaagacatatgag

SEQ ID NO: 224 - DNA, 22 bp, Artificial Sequence
  Synthetic polynucleotide
gcttccgattggtcttgtgttt

SEQ ID NO: 225 - DNA, 23 bp, Artificial Sequence
  Synthetic polynucleotide
tgagaagaaggtccttggagaga

SEQ ID NO: 226 - DNA, 22 bp, Artificial Sequence
  Synthetic polynucleotide
cgccaccgtatagttgatcttg

SEQ ID NO: 227 - DNA, 24 bp, Artificial Sequence
  Synthetic polynucleotide
gcaagtgacaagaaaaaacatgct

SEQ ID NO: 228 - DNA, 21 bp, Artificial Sequence
  Synthetic polynucleotide
agcaggtcggtgaactctttg

SEQ ID NO: 229 - DNA, 25 bp, Artificial Sequence
  Synthetic polynucleotide
gcctgctgagatcaaagactacagt

SEQ ID NO: 230 - DNA, 23 bp, Artificial Sequence
  Synthetic polynucleotide
gtggctgtgagaatcttgttcct

SEQ ID NO: 231 - DNA, 20 bp, Artificial Sequence
  Synthetic polynucleotide
ccacctccgtatcccacaga

SEQ ID NO: 232 - DNA, 22 bp, Artificial Sequence
  Synthetic polynucleotide
gacatgatgaacagcccaacct

SEQ ID NO: 233 - DNA, 25 bp, Artificial Sequence
  Synthetic polynucleotide
gaagtccgtactttgaccaattctc

SEQ ID NO: 234 - DNA, 22 bp, Artificial Sequence
  Synthetic polynucleotide
ccactcacaaccagacgatagc

SEQ ID NO: 235 - DNA, 23 bp, Artificial Sequence
  Synthetic polynucleotide
aggatgaaagacatggagttgga

SEQ ID NO: 236 - DNA, 22 bp, Artificial Sequence
  Synthetic polynucleotide
cattgatgtggtcacggatctg

SEQ ID NO: 237 - DNA, 22 bp, Artificial Sequence
  Synthetic polynucleotide
tgagagccgagattgacaatgt

SEQ ID NO: 238 - DNA, 17 bp, Artificial Sequence
  Synthetic polynucleotide
tccgcaatggcgttctg

SEQ ID NO: 239 - DNA, 17 bp, Artificial Sequence
  Synthetic polynucleotide
acccgacctccggatga

SEQ ID NO: 240 - DNA, 19 bp, Artificial Sequence
  Synthetic polynucleotide
attggctgcttggctcaga

SEQ ID NO: 241 - DNA, 22 bp, Artificial Sequence
  Synthetic polynucleotide
tctgcctcttccaaacttttcc

SEQ ID NO: 242 - DNA, 23 bp, Artificial Sequence
  Synthetic polynucleotide
aaccagagggagtgaatccagat

SEQ ID NO: 243 - DNA, 23 bp, Artificial Sequence
  Synthetic polynucleotide
ggtgacaagttcaagctcaacaa

SEQ ID NO: 244 - DNA, 22 bp, Artificial Sequence
  Synthetic polynucleotide
catctgtccttttccccaagaa

SEQ ID NO: 245 - DNA, 20 bp, Artificial Sequence
  Synthetic polynucleotide
ctctcctccgtcttcctgtg

SEQ ID NO: 246 - DNA, 20 bp, Artificial Sequence
  Synthetic polynucleotide
gtgacctgatgcagcaaaga

SEQ ID NO: 247 - DNA, 20 bp, Artificial Sequence
  Synthetic polynucleotide
aagaggaaggccaagtcgag

SEQ ID NO: 248 - DNA, 20 bp, Artificial Sequence
  Synthetic polynucleotide
cacacgtctcggtcatggta

SEQ ID NO: 249 - DNA, 25 bp, Artificial Sequence
  Synthetic polynucleotide
cttccaggagtgaaacacaaatctt

SEQ ID NO: 250 - DNA, 20 bp, Artificial Sequence
  Synthetic polynucleotide
gccactttgcccaactcaaa

SEQ ID NO: 251 - DNA, 23 bp, Artificial Sequence
  Synthetic polynucleotide
agcgactacgccaagtactatgc

SEQ ID NO: 252 - DNA, 21 bp, Artificial Sequence
  Synthetic polynucleotide
catcatgaagggtggaaatgg

SEQ ID NO: 253 - DNA, 22 bp, Artificial Sequence
  Synthetic polynucleotide
tctcctcccagaacgtcacagt

SEQ ID NO: 254 - DNA, 18 bp, Artificial Sequence
  Synthetic polynucleotide
tgagccagcaggctgatg

SEQ ID NO: 255 - DNA, 21 bp, Artificial Sequence
  Synthetic polynucleotide
caccatcggaaatgaacgttt

SEQ ID NO: 256 - DNA, 20 bp, Artificial Sequence
  Synthetic polynucleotide
gactccatcccgatgaagga

SEQ ID NO: 257 - DNA, 21 bp, Artificial Sequence
  Synthetic polynucleotide
ccgacaccagatccagtccta

SEQ ID NO: 258 - DNA, 21 bp, Artificial Sequence
  Synthetic polynucleotide
gggaatcgttagtgcccttca

(19) United States
(12) Patent Application Publication
(10) Pub. No.: US 2013/0065834 A1
(43) Pub. Date: Mar. 14, 2013

(54) MODULATORS OF PROTEASOME ACTIVITY

(71) Applicants: David Vilchez, San Diego, CA (US); Andrew Dillin, San Diego, CA (US)

(72) Inventors: David Vilchez, San Diego, CA (US); Andrew Dillin, San Diego, CA (US)

(73) Assignee: SALK INSTITUTE FOR BIOLOGICAL STUDIES, La Jolla, CA (US)

(21) Appl. No.: 13/558,307

(22) Filed: Jul. 25, 2012

Related U.S. Application Data
(60) Provisional application No. 61/511,460, filed on Jul. 25, 2011.

(51) Int. Cl. A61K 38/06; C12N 15/85; A61P 25/00; A61P 25/28; A61P 25/16; C12N 5/071; A61K 31/4015

(52) U.S. Cl. 514/17.8; 435/375; 435/455; 435/377; 514/21.9; 514/17.7; 514/425

(57) ABSTRACT

Methods of modulating proteasome activity, increasing life span and neurogenesis are provided herein.

### CROSS-REFERENCES TO RELATED APPLICATIONS

This application claims the benefit of U.S. Provisional Application No. 61/511,460 filed Jul. 25, 2011, which is hereby incorporated in its entirety and for all purposes.

### STATEMENT AS TO RIGHTS TO INVENTIONS MADE UNDER FEDERALLY SPONSORED RESEARCH AND DEVELOPMENT

The invention was made with government support under P01 AG031097 and RCI AG036024 awarded by the National Institutes of Health. The Government has certain rights in the invention.

### BACKGROUND OF THE INVENTION

An organism's choice to protect its germ cell lineages from damage often comes at a considerable cost: limited metabolic resources become partitioned away from maintenance of the soma, leaving the aging somatic tissues to navigate survival amid a crowded pool of damaged and poorly functioning proteins. Historically, experimental paradigms that limit reproductive investment result in lifespan extension. In nature, food sources are largely unpredictable and insufficient. The constant pressures that limited energetic resources place on an organism have long been theorized to cause a significant life-history trade-off: the absolute need for repairing and preventing damage to the germline—and for ensuring elimination of damage in progeny—necessarily dominates resource allocation strategies, while conversely little or no evolutionary pressure will be placed on the maintenance of the soma [Kirkwood, T. B. Evolution of ageing. Nature 270, 301-304 (1977)]. Thus, aging, post-reproductive organisms that escape predation witness the gradual deterioration of their own somatic tissues. In support of such theories, modulations of reproduction that eliminate germ cells provide effective mechanisms for extending lifespan [Kenyon, C. A pathway that links reproductive status to lifespan in Caenorhabditis elegans. Ann N Y Acad Sci 1204, 156-162 (2010); Partridge, L., Gems, D. & Withers, D. J. Sex and death: what is the connection? Cell 120, 461-472 (2005)], phenotypes that may be caused by heightened resource availability within the post-mitotic soma. Likewise, it has been proposed that animals undergoing dietary restriction adopt a strategy in which resources are re-allocated towards somatic maintenance, extending lifespan and prolonging reproduction until conditions for survival become more favorable [Shanley, D. P. & Kirkwood, T. B. Calorie restriction and aging: a life-history analysis. Evolution; international journal of organic evolution 54, 740-750 (2000)].

When proliferating germline cells of C. elegans are removed, worms live up to 60% longer than normal and appear resistant to a variety of environmental stressors [Arantes-Oliveira, N., Apfeld, J., Dillin, A. & Kenyon, C. Regulation of life-span by germ-line stem cells in Caenorhabditis elegans. Science 295, 502-505 (2002); Hsin, H. & Kenyon, C. Signals from the reproductive system regulate the lifespan of C. elegans. Nature 399, 362-366 (1999); Wang, M. C., O'Rourke, E. J. & Ruvkun, G. Fat metabolism links germline stem cells and longevity in C. elegans. Science 322, 957-960 (2008)]. While germline ablation affords an obvious protection, the downstream effectors of such protection remain somewhat ambiguous. The reallocation of resources to the soma seems directed through a specific, genetically defined stress-responsive pathway. Germline removal extends lifespan by triggering an active signaling network, involving the nuclear localization and activation of DAF-16, a forkhead transcription factor (FOXO) [Lin, K., Hsin, H., Libina, N. & Kenyon, C. Regulation of the Caenorhabditis elegans longevity protein DAF-16 by insulin/IGF-1 and germline signaling. Nat Genet. 28, 139-145 (2001)] and the major downstream effector of the daf-2/insulin/insulin-like growth factor (IGF) signaling (IIS) pathway. However, while worms with an ablated germline exhibit a daf-16 dependent extension in lifespan, longevity caused by germline ablation functions in a synergistic manner with mutations in the IIS receptor, daf-2 [Hsin, H. & Kenyon, C. Signals from the reproductive system regulate the lifespan of C. elegans. Nature 399, 362-366 (1999)]. Additionally, in germline ablated animals but not daf-2 mutant worms, activities of kri-1, daf-9 and the nuclear hormone receptor daf-12 are also required for the constitutive nuclear localization of daf-16 [Berman, J. R. & Kenyon, C. Germ-cell loss extends C. elegans life span through regulation of DAF-16 by kri-1 and lipophilic-hormone signaling. Cell 124, 1055-1068 (2006); Gerisch, B., Weitzel, C., Kober-Eisermann, C., Rottiers, V. & Antebi, A. A hormonal signaling pathway influencing C. elegans metabolism, reproductive development, and life span. Dev Cell 1, 841-851 (2001)].

Importantly, post-mitotic somatic cells also hold an especial distinction for their susceptibility to age-onset protein aggregation diseases. As the somatic cell ages, the accumulation of damaged proteins represent a particular challenge to the aging cell, especially as they aggregate in inclusions and aggresomes capable of overwhelming the cellular machinery required for their degradation [Bence, N. F., Sampat, R. M. & Kopito, R. R. Impairment of the ubiquitin-proteasome system by protein aggregation. Science 292, 1552-1555 (2001); Bennett, E. J., Bence, N. F., Jayakumar, R. & Kopito, R. R. Global impairment of the ubiquitin-proteasome system by nuclear or cytoplasmic protein aggregates precedes inclusion body formation. Molecular cell 17, 351-365 (2005)]. These effects are likely compounded by age-related dysregulation of chaperones, a downregulation of degradation machinery itself, and a continually accelerating loss in general cellular homeostasis. As such, a rapid decline in the capacity of the cell to protect its proteome has been highly correlated with multiple age-related disorders [Powers, E. T., Morimoto, R. I., Dillin, A., Kelly, J. W. & Balch, W. E. Biological and chemical approaches to diseases of proteostasis deficiency. Annual review of biochemistry 78, 959-991 (2009)]. This conversely suggests that the long-lived somatic cells, such as those found in a germline-ablated animal, might exhibit a heightened capacity for clearing damaged proteins, and that this proteostatic capacity might contribute to the increased longevity in these mutants. Ad of today little is known how alterations of the protein homeostasis machinery can impact the aging process. In the case of stem cells, genome stability is a central function required for stem cell survival however, proteome stability might also play a central role in stem cell identity and function. Therefore, a firm understanding of how organisms in general and more specifically stem cells maintain protein homeostasis is of central importance.

Provided herein are solutions to this and other problems in the art.

### BRIEF SUMMARY OF THE INVENTION

In one aspect, a method of modulating a proteasome activity in a cell is provided. The method includes modulating an rpn-6.1 protein activity or an rpn-6.1 protein level in the cell thereby modulating the proteasome activity.

In another aspect, a method of increasing cell survival of a cell, which suffers from proteotoxic stress is provided. The method includes increasing an rpn-6.1 protein activity or an rpn-6.1 protein level in the cell and thereby increasing cell survival of the cell, which suffers from proteotoxic stress.

In another aspect, a method of treating a protein-misfolding disease in a subject in need thereof is provided. The method includes administering to the subject a therapeutically effective amount of an rpn-6.1 modulator.

In another aspect, a method of increasing neurogenesis in a cell is provided. The method includes increasing a Foxo4 protein activity or a Foxo4 protein level in the cell.

In another aspect, a method of preparing an induced pluripotent stem cell is provided. The method includes modulating a Foxo4 protein activity or a Foxo4 protein level in a non-pluripotent cell thereby forming a modulated non-pluripotent cell. The modulated non-pluripotent cell is allowed to divide and thereby forms the induced pluripotent stem cell.

### BRIEF DESCRIPTION OF THE DRAWINGS

FIG. 1. Germline-lacking animals have increased proteasome activity. FIG. 1a, Chymotrypsin-like activity of the proteasome monitored by Z-GGL-AMC digestion in day 7 adult worm extract containing equal amounts of total protein. Proteasome activity (relative slope to control strain fer-15(b26);fem-1(hc17)) represents the mean±s.e.m. (n=50, *P=1.83*10⁻¹⁷). FIG. 1b, Caspase-like (Z-LLE-AMC) proteasome activity (relative slope) represents the mean±s.e.m. (n=8, *P<0.00001). FIG. 1c, Trypsin-like (Ac-RLR-AMC) proteasome activity (relative slope) represents the mean±s.e.m. (n=7, *P<0.00001). FIG. 1d, Representative polyubiquitinylated protein immunoblot. α-tubulin loading control.

FIG. 2. DAF-16 is required for proteasome activity in glp-1(e2141) mutant. FIG. 2a, Chymotrypsin-like proteasome activity (relative slope to fer-15(b26);fem-1(hc17)) represents the mean±s.e.m. (n=11, *P<0.00005). FIG. 2b, Caspase-like proteasome activity (relative slope to fer-15(b26);fem-1(hc17)) represents the mean±s.e.m. (n=6, *P<0.0001). FIG. 2c, Trypsin-like proteasome activity (relative slope to fer-15(b26);fem-1(hc17)) represents the mean±s.e.m. (n=6, *P<0.005). FIG. 2d, glp-1(e2141) worms fed daf-16 RNAi bacteria have decreased in chymotrypsin-like proteasome activity (P=8.5*10⁻⁸ vector RNAi-glp-1 mutant versus daf-16 RNAi-glp-1 mutant, n=16). daf-16 RNAi knock-down does not affect proteasome activity infer-15;fem-1 worms (P=0.79 vector RNAi-fer-15;fem-1 vs daf-16 RNAi-fer-15;fem-1, n=9). FIG. 2e, Chymotrypsin-like proteasome activity in glp-1(e2141) worms fed daf-16, daf-12, daf-9 or kri-1 RNAi bacteria ((n=4), vector RNAi vs daf-16 RNAi (P=4.17*10⁻⁵), vector RNAi vs daf-12 RNAi (P<0.01), vector RNAi vs daf-9 RNAi (P<0.05), vector RNAi vs kri-1 RNAi (P<0.01)). FIG. 2f, Chymotrypsin-like proteasome activity (relative slope to vector RNAi-glp-1 mutant) represents the mean±s.e.m. (n=8, vector RNAi vs daf-16 RNAi (P<0.0001), vector RNAi vs hsf-1 RNAi (P=0.74)). FIG. 2g, skn-1 RNAi does not affect chymotrypsin-like proteasome activity of glp-1(e2141) worms (vector RNAi vs daf-16 RNAi (P<0.00001), vector RNAi vs hsf-1 RNAi (P=0.46)). Chymotrypsin-like proteasome activity (relative slope to vector RNAi-glp-1 mutant) represents the mean±s.e.m. (n=9, vector RNAi vs daf-16 RNAi (P<0.00001), vector RNAi vs hsf-1 RNAi (P=0.46)). All activities measured at day 5 of adulthood. All RNAi treatment initiated day 1 of adulthood. All statistical comparisons were made by Student's t-test for unpaired samples.

FIG. 3. DAF-16 is necessary for increased expression of rpn-6.1 in glp-1 mutants. FIG. 3a, Data represent the mean±s.e.m. of the relative expression levels to fer-15(b26);fem-1(hc17) (n=11, P=2.09*10⁻⁶). FIG. 3b, Western blot analysis of RPN-2, rpn-6.1, RPN-8, RPT-6, alpha 6 and alpha 1+2+3+5+6+7. β-actin loading control. FIG. 3c glp-1 mutants fed rpn-6.1 RNAi bacteria starting day 1 of adulthood have decreased chymotrypsin-like proteasome activity (P<0.05). Proteasome activity (relative slope to fer-15(b26);fem-1(hc17)) represents the mean±s.e.m. (n=6). FIG. 3d, Increased chymotrypsin-like proteasome activity in rpn-6.1 overexpressing N2 worms (rpn-6.1, GFP OE), in day 1 adult worm extract (P<0.001). Proteasome activity (relative slope to GFP OE worms) represents the mean±s.e.m. (n=9). FIG. 3e, daf-16;glp-1 double mutants have decreased rpn-6.1 mRNA (P<0.001). Graph represents the mean±s.e.m (n=6). FIG. 3f, Representative images of RFP expressed under control of rpn-6.1 promoter. rpn-6.1 is expressed in the pharynx and posterior intestine in N2 worms. Increased rpn-6.1 expression in glp-1(e2141) animals relative to N2. daf-16;glp-1 mutant worms have decreased rpn-6.1 expression compared to glp-1 mutants. DIC, differential interference contrast microscopy. Scale bar represents 100 μm. g, Quantification of RFP signal intensity (mean±s.e.m. (n=5)). glp-1(e2141) worms have increased rpn-6.1 expression compared to N2 (P<0.01) and daf-16(mu86);glp-1(e2141) double mutants (P<0.05). All statistical comparisons were made by Student's t-test for unpaired samples.

FIG. 4. rpn-6.1 is a determinant of stress resistance and viability. FIG. 4a, rpn-6.1 overexpressing (OE) worms live longer than controls under oxidative stress (log rank, P<0.0001, GFP OE: mean=2.97±0.10, n=43/60; rpn-6.1, GFP OE: mean=4.89±0.17, n=40/59). FIG. 4b, rpn-6.1 OE worms live longer than controls under heat stress conditions (34° C.) (log rank, P<0.0001, GFP OE: mean=16.20±0.47, n=60/60; rpn-6.1, GFP OE: mean=29.83±0.99, n=60/60). FIG. 4c, Ultraviolet stress assay on rpn-6.1 OE worms (log rank, P=0.06, GFP OE: mean=6.01±0.27, n=59/60, rpn-6.1, GFP OE: mean=6.57±0.19, n=58/59). FIG. 4d, rpn-6.1 OE does not affect lifespan at 20° C. (log rank, P=0.15, GFP OE: mean=16.33±0.34, n=90/100; rpn-6.1, GFP OE: mean=17.05±0.34, n=95/100). rpn-6.1 OE extends lifespan at 25° C. (log rank, P<0.0001, GFP OE: mean=10.19±0.32, n=109/120; rpn-6.1, GFP OE: mean=12.73±0.29, n=113/120). FIG. 4e, daf-16 RNAi (starting day 1 of adulthood) blocked lifespan extension induced by rpn-6.1 OE (rpn-6.1, GFP OE+vector RNAi versus rpn-6.1, GFP OE+daf-16 RNAi, log rank, P<0.0001). GFP OE+vector RNAi: mean=11.36±0.45, n=86/100; GFP OE+daf-16 RNAi: mean=10.59±0.36, n=77/100; rpn-6.1, GFP OE+vector RNAi: mean=13.27±0.37, n=87/100; rpn-6.1, GFP OE+daf-16 RNAi: mean=10.59±0.28, n=88/100. FIG. 4f, hsf-1 RNAi treated-rpn-6.1 OE worms were long-lived compared to controls (log rank, P<0.0001). RNAi initiated day 1 of adulthood. GFP OE+hsf-1 RNAi: mean=7.74±0.09, n=93/99; rpn-6.1, GFP OE+hsf-1 RNAi: mean=9.92±0.13, n=94/100.

FIG. 5. rpn-6.1 protects from polyglutamine aggregation. FIG. 5a, rpn-6.1 improves motility in polyQ67 worms. Bar graphs represent average (±s.e.m.) thrashing over a 30 second period on day 1 (P<0.0001, GFP OE; Q67 (n=40), rpn-6.1, GFP OE; Q67 (n=41)), day 3 (P<0.0001, GFP OE; Q67 (n=40), rpn-6.1, GFP OE; Q67 (n=44)) and day 5 (P<0.05, GFP OE; Q67 (n=31), rpn-6.1, GFP OE; Q67 (n=39)) of adulthood. FIG. 5 b, Loss of rpn-6.1 worsens the motility defects of polyQ67 worms. Bar graphs represent average (±s.e.m.) thrashing over a 30 second period on day 1 (P=0.15, Q67+vector RNAi (n=58), Q67+rpn-6.1 RNAi (n=30)), day 3 (P<0.0001, Q67+vector RNAi (n=44), Q67+rpn-6.1 RNAi (n=51)) and day 5 (P<0.0001, Q67+vector RNAi (n=41), Q67+rpn-6.1 RNAi (n=47)) of adulthood. All statistical comparisons were made by Student's t-test for unpaired samples. FIG. 5c, Filter trap analysis indicates rpn-6.1 OE results in reduced polyQ aggregates (detected by anti-GFP antibody). Right panel, SDS-PAGE analysis with antibodies to GFP, RPN-6 and α-tubulin loading control.

FIG. 6. Analysis of proteasome activity in different long-lived worms. Germline-lacking glp-1(e2141) mutant shows a 6-fold increase in chymotrypsin-like proteasome activity compared to control strain fer-15(b26);fem-1(hc17) (P=1.83*10-17). eat-2 mutant worms also show an increased proteasome activity compared to the control strain (P<0.05), although to a lesser extent than glp-1 mutants. In contrast, daf-2 mutant (P=0.92) and cco-1 RNAi-treated (P=0.52) animals do not show increased proteasome activity when compared to control strain. Proteasome activity (relative slope to control strain fer-15(b26);fem-1(hc17)) represents the mean±s.e.m. (fer-15(b26);fem-1(hc17) (n=40), eat-2(ad1116);fer-15(b26);fem-1(hc17) (n=17), fer-15(b26);fem-1(hc17)+cco-1 RNAi (n=6), daf-2(mu150);fer-15(b26);fem-1(hc17) (n=20); glp-1(e2141) (n=40)). All statistical comparisons were made by Student's t-test for unpaired samples.

FIG. 7. Analysis of proteasome activity in glp-1 mutants at different adulthood stages. FIG. 7a, glp-1(e2141) animals have increased chymotrypsin-like proteasome activity at all adulthood ages analyzed. Wild-type (N2) and fer-15(b26);fem-1(hc17) strains displayed similar proteasome activity, as measured fluorometrically by digestion of the peptide Z-GGL-AMC. FIG. 7b, Similar results to FIG. 7a were obtained when an alternative fluorogenic substrate of the chymotrypsin-like activity of the proteasome, Suc-Leu-Leu-Val-Tyr-AMC was monitored fluorometrically, in 4 day adult worm lysate samples containing equal amounts of total protein. Proteasome activity (relative slope to control strain fer-15(b26);fem-1(hc17)) represents the mean±s.e.m. (n=5). glp-1(e2141) animals have increased chymotrypsin-like proteasome activity (P=4.52*10⁻⁵). FIG. 7c, The differences in proteasome activity between glp-1 and fer-15(b26);fem-1(hc17) strains were similar whether Applicants compared samples containing equal amounts of total protein or equal number of worms (mean±s.e.m. (n=4), P<0.05)). All statistical comparisons were made by Student's t-test for unpaired samples.

FIG. 8. Proteasome inhibitors block degradation of the proteasome substrate. Worm lysates were incubated with proteasome inhibitors 20 min prior to adding Z-GGL-AMC fluorogenic substrate. All proteasome inhibitors tested blocked chymotrypsin-like proteasome activity in both glp-1(e2141) mutant (FIG. 8a) and fer-15(b26);fem-1(hc17) lysates (FIG. 8b). Proteasome activity (relative slope to DMSO-treated worm lysate) represents the mean±s.e.m. (n=3). glp-1(e2141): DMSO vs MG-132 (P<0.005), DMSO vs PI-I (P<0.005), DMSO vs lactacystin (P<0.001). fer-15(b26);fem-1(hc17): DMSO vs MG-132 (P<0.005), DMSO vs PI-I (P<0.05), DMSO vs lactacystin (P<0.001). Statistical comparisons were made by Student's t-test for unpaired samples.

FIG. 9. glp-1 mutants degrade more rapidly UbG76V-Dendra2 proteasome reporter than control strain. Representative fluorescence of worms expressing UbG76V-Dendra2 (top) and control Dendra2 (bottom) in body-wall muscle, imaged before and after photoconversion (fer-15(b26);fem-1(hc17);unc-54::UbG76V-Dendra2 (n=7 worms), glp-1 (e2141); unc-54::UbG76V-Dendra2 (n=10), fer-15 (b26);fem-1(hc17); unc-54::Dendra2 (n=8), glp-1(e2141);unc-54::Dendra2 (n=7)). Graphs show average percentage of fluorescence relative to intensity at the point of photoconversion (Oh after conversion), *P<0.005. All statistical comparisons were made by Student's t-test for unpaired samples.

FIG. 10. Sterile control strain has a similar lifespan to wild-type. glp-1 (e2141) animals live significantly longer than wild-type (N2) and fer-15(b26);fem-1(hc17) worms (log rank, P<0.0001, in both cases). This lifespan extension is not a result of sterility, as there is no significant difference between N2 and the sterile fer-15(b26);fem-1(hc17) strain (log rank, P=0.70). fer-15(b26);fem-1(hc17): mean=19.43±0.44, n=93/100; glp-1(e2141): mean=22.98±0.89, n=93/113; N2: mean=19.21±0.49, n=70/100. Graph is representative of 2 independent experiments.

FIG. 11. Proteasome activity in worms treated with FUdR. Worms were grown at 25° C. and treated with 5-fluoro-2′ deoxyuridine (FUdR) at 100 μg ml⁻¹ for the first three days of adulthood to induce sterility in N2 worms. Under FUdR treatment, the wild-type strain had a significant lower chymotrypsin-like proteasome activity compared to glp-1 worms (P<0.005). Applicants could not detect significant differences between wild-type and fer-15(b26);fem-1(hc17) worms (P=0.30). Proteasome activity in day 3 adults (relative slope to control strain N2) represents the mean±s.e.m. (n=3). Statistical comparisons were made by Student's t-test for unpaired samples.

FIG. 12. glp-1(e2141) worms grown at permissive temperature (15° C.) do not have increased proteasome activity. Digestion of Z-GGL-AMC, monitored fluorometrically, in extracts of day 3 adult worms grown at 15° C. Chymotrypsin-like proteasome activity (relative slope to control strain N2) represents the mean±s.e.m. (N2 (n=6), fer-15(b26);fem-1(hc17) (n=6), glp-1(e2141) (n=6), daf-16(mgDf47);glp-1(e2141) (n=3), daf-16 (mu86);glp-1(e2141) (n=3)). No significant differences were observed among the different strains (P=0.57, One-way analysis of variance (ANOVA), F=0.74, df=4).

FIG. 13. Germline-lacking animals have increased proteasomal activity when down shifted to permissive temperature. Digestion of a fluorogenic substrate of the chymotrypsin-like activity of the proteasome (Z-GGL-AMC), in day 4 adult worm extract containing equal amounts of total protein. Proteasome activity (relative slope to control strain fer-15(b26);fem-1(hc17) 20° C.) represents the mean±s.e.m. (n=4). glp-1(e2141) animals maintain increased chymotrypsin-like proteasome activity at 20° C. (P<0.005).

FIG. 14. daf-16 knock-down does not affect proteasome activity in wild-type worms. N2 worms fed daf-16 RNAi bacteria from hatching did not show a decrease in chymotrypsin-like proteasome activity, as monitored fluorometrically by digestion of the peptide Z-GGL-AMC at day 1 ((n=4), P=0.27)). Proteasome activity (relative slope to vector RNAi) represents the mean±s.e.m. Statistical comparisons were made by Student's t-test for unpaired samples.

FIG. 15. Regulation of proteasome activity by daf-12, daf-9 and kri-1 in control strains. FIG. 15a, Chymotrypsin-like proteasome activity in (b26);fem-1(hc17) worms fed daf-16, daf-12, daf-9 or kri-1 RNAi bacteria starting from day 1 of adulthood ((n=4), vector RNAi vs daf-16 RNAi (P=0.19), vector RNAi vs daf-12 RNAi (P=0.07), vector RNAi vs daf-9 RNAi (P<0.05), vector RNAi vs kri-1 RNAi (P=0.10)). FIG. 15b, Chymotrypsin-like proteasome activity in daf-16(mgDf47);glp-1(e2141) worms fed daf-16, daf-12, daf-9 or kri-1 RNAi bacteria starting from day 1 of adulthood ((n=4), vector RNAi vs daf-16 RNAi (P=0.37), vector RNAi vs daf-12 RNAi (P=0.70), vector RNAi vs daf-9 RNAi (P=0.45), vector RNAi vs kri-1 RNAi (P<0.15)). Chymotrypsin-like proteasome activity was measured fluorometrically by digestion of the peptide Z-GGL-AMC at day 5 of adulthood. Statistical comparisons were made by Student's t-test for unpaired samples.

FIG. 16. skn-1 is required for long-lifespan of glp-1(e2141) mutant. skn-1 is necessary for longevity phenotype of glp-1 mutant (glp-1(e2141)+vector RNAi vs glp-1(e2141)+skn-1 RNAi, log rank, P<0.0001). N2+vector RNAi: mean=19.96±0.50, n=76/101; N2±skn-1 RNAi: mean=16.36±0.28, n=91/100; glp-1(e2141)+vector RNAi: mean=21.33±0.53, n=98/104; glp-1(e2141)+skn-1 RNAi: mean=15.65±0.25, n=93/97.

FIG. 17. Knock-down of either hsf-1 or skn-1 did not further decrease low proteasome activity of daf-16;glp-1 double mutant animals. FIG. 17a, daf-16(mgDf47);glp-1(e2141) worms fed hsf-1 or skn-1 RNAi bacteria starting from day 1 of adulthood do not show a decreased in their chymotrypsin-like proteasome activity (vector RNAi vs hsf-1 RNAi (P=0.81); vector RNAi vs skn-1 RNAi (P=0.36)). Proteasome activity (relative slope to vector RNAi) represents the mean±s.e.m. (n=4). FIG. 17b, daf-16(mu86);glp-1(e2141) double mutants fed hsf-1 or skn-1 RNAi bacteria starting from day 1 of adulthood do not show a decreased in their chymotrypsin-like proteasome activity (vector RNAi vs hsf-1 RNAi (P=0.68); vector RNAi vs skn-1 RNAi (P=0.29)). Proteasome activity (relative slope to vector RNAi) represents the mean±s.e.m. (n=4).

FIG. 18. nhr-80 is not required for increased proteasome activity of glp-1 (e2141) mutant. glp-1(e2141) worms fed nhr-80 RNAi bacteria starting from day 1 of adulthood do not show a decreased in their chymotrypsin-like proteasome activity (glp-1(e2141)+vector RNAi vs glp-1(e2141)+daf-16 RNAi (P<0.005); glp-1(e2141)+vector RNAi vs glp-1(e2141)+nhr-80 RNAi (P=0.25)). Proteasome activity (relative slope to vector RNAi-glp-1 mutant) represents the mean±s.e.m. (glp-1(e2141)+vector RNAi (n=7), glp-1(e2141)+daf-16 RNAi (n=5), glp-1(e2141)+nhr-80 RNAi (n=7)).

FIG. 19. 20S proteasome subunit expression levels in glp-1 mutants. Only one of the 20S proteasome subunits is increased in glp-1(e2141) mutants: pbs-5 (glp-1(e2141) vs fer-15(b26);fem-1(hc17), P<0.005). Statistical comparisons were made by Student's t-test for unpaired samples. Data represent the mean±s.e.m. of the relative expression levels to fer-15(b26);fem-1(hc17) (n=11).

FIG. 20. Impact of the knock-down of 19S non-ATPase subunits on the proteasome activity of glp-1(e2141) mutant. glp-1 worms fed either rpn-1, rpn-2 or rpn-11 RNAi bacteria starting from day 1 of adulthood do not show a decreased in their proteasome activity, which was measured by monitoring fluorographically the digestion of the peptide Z-GGL-AMC (vector RNAi vs rpn-1 RNAi (P=0.41); vector RNAi vs rpn-2 RNAi (P=0.29), vector RNAi vs rpn-11 RNAi (P=0.37)). Graph represents the mean±s.e.m. (vector RNAi (n=8), rpn-1 (n=3), rpn-2 (n=4), rpn-11 RNAi (n=4)). Statistical comparisons were made by Student's t-test for unpaired samples.

FIG. 21. daf-16 is required for increased expression of rpn-6.1. glp-1(e2141) worms fed daf-16 RNAi bacteria starting from day 1 of adulthood show a decrease in the expression of rpn-6.1 at day 5 of adulthood (P<0.001 vector RNAi-glp-1 mutant vs daf-16 RNAi-glp-1 mutant, n=5). In contrast, daf-16 RNAi did not affect rpn-6.1 mRNA levels in fer-15(b26);fem-1(hc17) strain (P=0.57 vector RNAi-fer-15;fem-1 vs daf-16 RNAi-fer-15;fem-1, n=4).

FIG. 22. daf-16 specifically regulates rpn-6.1 levels. Analysis of the 26S proteasome subunit mRNA levels in daf-16(mgDf47);glp-1(e2141) and daf-16(mu86);glp-1(e2141) double mutant strains. rpn-6.1 levels are decreased in daf-16;glp-1 double mutants compared to the glp-1 strain (statistical analysis in Table 4). Graphs represent the mean±s.e.m. of the relative expression levels to fer-15(b26);fem-1(hc17) worms (fer-15(b26);fem-1(hc17) (n=7), glp-1 (e2141) (n=7), daf-16(mgDf47),glp-1(e2141) (n=7), daf-16(mu86);glp-1(e2141) (n=4)).

FIG. 23. Putative DAF-16 binding site within first intron of rpn-6.1. Applicants identified a strong hit (position chrIII:6,961,360-6,961,373, matrix match=0.911) to the TRANSFAC [(Matys, V., et al. TRANSFAC and its module TRANSCompel: transcriptional gene regulation in eukaryotes. Nucleic acids research 34, D108-110 (2006)] matrix N$DAF16_{—}01 [Furuyama, T., Nakazawa, T., Nakano, I. & Mori, N. Identification of the differential distribution patterns of mRNAs and consensus binding sequences for mouse DAF-16 homologues. Biochem J 349, 629-634 (2000) using the program MATCH [Kel, A. E., et al. MATCH: A tool for searching transcription factor binding sites in DNA sequences. Nucleic acids research 31, 3576-3579 (2003)]. This binding site is supported by a DAF-16 binding peak identified through ChIP-Seq of a DAF-16::GFP fusion protein [Celniker, S. E., et al. Unlocking the secrets of the genome. Nature 459, 927-930 (2009)] (modENCODE project: “Identification of Transcription Factor DAF-16::GFP Binding Regions in L4 Young Adult”, position chrIII:6,961,067-6,961,535, score=4.6e-23).

FIG. 24. rpn-6.1 is essential for viability of adult animals. Lifespan of glp-1 (e2141), N2, fer-15(b26);fem-1(hc17), daf-2(e1370), eat-2 (ad1116) and isp-1(qm150) mutant worms fed rpn-6.1 RNAi from day 1 of adulthood. FIG. 24a, glp-1(e2141)+vector RNAi: mean=22.98±0.89, n=93/113; glp-1(e2141)+rpn-6.1 RNAi: mean=13.63±0.23, n=104/104 (log rank, P<0.0001). FIG. 24b, N2+vector RNAi: mean=19.21±0.49, n=70/100; N2+rpn-6.1 RNAi: mean=12.48±0.19, n=91/100 (log rank, P<0.0001). FIG. 24c, fer-15(b26);fem-1(hc17)+vector RNAi: mean=19.43±0.44, n=93/100; fer-15(b26);fem-1(hc17)+rpn-6.1 RNAi: mean=13.16±0.23, n=89/100 (log rank, P<0.0001). FIG. 24d, daf-2(e1370)+vector RNAi: mean=48.19±1.35, n=80/98; daf-2(e1370)+rpn-6.1 RNAi: mean=21.96±0.41, n=106/107 (log rank, P<0.0001). FIG. 24e, eat-2(ad1116)+vector RNAi: mean=20.83±0.93, n=87/110; eat-2(ad1116)+rpn-6.1 RNAi: mean=12.28±0.21, n=96/110 (log rank, P<0.0001). FIG. 24f, isp-1(qm150)+vector RNAi: mean=23.43±1.46, n=56/101; isp-1(qm150)+rpn-6.1 RNAi: mean=12.48±0.25, n=86/102 (log rank, P<0.0001). All graphs are representative of two independent experiments.

FIG. 25. rpn-6.1 regulates lifespan and stress resistance. FIG. 25a, rpn-6.1 OE worms survive longer than controls under oxidative stress conditions (log rank, P<0.0001, GFP OE: mean=2.97±0.10, N2: mean=3.33±0.10, n=48/60; rpn-6.1, GFP OE clone 1: mean=4.89±0.17, n=40/59, rpn-6.1, GFP OE clone 2: mean=4.18±0.28, n=41/52). Animals were grown on plates containing 7.5 mM paraquat. No significant difference was found between N2 and GFP OE worms. FIG. 25b, rpn-6.1 OE worms survive longer than controls under heat stress conditions (34° C.) (log rank, P<0.0001, GFP OE: mean=16.20±0.47, N2: mean=15.91±0.44, n=60/60; rpn-6.1, GFP OE clone 1: mean=29.83±0.99, n=60/60, rpn-6.1, GFP OE clone 2: mean=28.97±1.05, n=60/60). Similar results were obtained with 2 different clones of rpn-6.1 OE worms. No significant difference was found between N2 and GFP OE worms. FIG. 25c, Ultraviolet stress assay on rpn-6.1 OE worms (GFP OE: mean=6.01±0.27, n=59/60; N2: mean=6.10±0.25, n=56/60, rpn-6.1, rpn-6.1, GFP OE: mean=6.57±0.19, n=58/59, GFP OE clone 2: mean=6.72±0.56, n=59/62). FIG. 25d, Overexpression of rpn-6.1 did not increase lifespan of worms at 20° C. (log rank, P=0.15, N2 mean=17.31±0.37, n=106/110; GFP OE: mean=16.61±0.36, n=102/110; rpn-6.1, GFP OE clone 1: mean=16.92±0.30, n=103/110; rpn-6.1, GFP OE clone 2: mean=16.54±0.28, n=109/111). However, rpn-6.1 OE worms were long-lived at 25° C. (log rank, P<0.0001, N2 mean=10.79±0.39, n=104/120; GFP OE: mean=10.19±0.32, n=109/120; rpn-6.1, GFP OE clone 1: mean=12.73±0.29, n=113/120; rpn-6.1, GFP OE clone 2: mean=11.63±0.29, n=107/116). FIG. 25e, Worms were fed daf-16 RNAi bacteria starting from day 1 of adulthood. daf-16 RNAi treatment blocked the lifespan extension induced by rpn-6.1 OE (log rank, P<0.0001). GFP OE+vector RNAi: mean=11.33±0.34, n=93/99; GFP OE+daf-16 RNAi: mean=10.68±0.28, n=86/99; rpn-6.1, GFP OE clone 2+vector RNAi: mean=13.03±0.32, n=92/100; rpn-6.1, GFP OE clone 2+daf-16 RNAi: mean=11.31±0.24, n=83/99. FIG. 25f, Worms fed hsf-1 RNAi bacteria starting from day 1 of adulthood. hsf-1 RNAi treated-rpn-6.1 OE worms were long-lived compared to control strains under the same treatment (log rank, P<0.0001). GFP OE+vector RNAi: mean=11.36±0.45, n=86/100; GFP OE+hsf-1 RNAi: mean=7.73±0.12, n=82/100; rpn-6.1, GFP OE clone 1+vector RNAi: mean=13.27±0.37, n=87/100; rpn-6.1, GFP OE clone 1+hsf-1 RNAi: mean=9.82±0.11, n=93/100.

FIG. 26. rpn-6.1 overexpression reduces motility defects in worms that accumulate polyQ aggregates in neurons. Thrashing rates of day 1 (FIG. 26a), day 3 (FIG. 26b) and day 5 (FIG. 26c) adulthood animals. At day 1, rpn-6.1 OE substantially improves motility of polyQ67 worms (GFP OE vs rpn-6.1, GFP OE (P=0.14); GFP OE; Q40 vs rpn-6.1, GFP OE; Q40 (P=0.53); GFP OE; Q67 vs rpn-6.1,GFP OE; Q67 (P<0.0001)). At day 3, rpn-6.1 OE significantly improves the motility of both polyQ40 and polyQ67 worms (GFP OE vs rpn-6.1,GFP OE (P=0.50); GFP OE; Q40 vs rpn-6.1,GFP OE; Q40 (P<0.05); GFP OE; Q67 vs rpn-6.1,GFP OE; Q67 (P<0.0001)). At day 5, rpn-6.1 OE significantly improves the motility of both polyQ40 and polyQ67 worms (GFP OE vs rpn-6.1,GFP OE (P=0.98); GFP OE; Q40 vs rpn-6.1,GFP OE; Q40 (P<0.05); GFP OE; Q67 vs rpn-6.1,GFP OE; Q67 (P<0.05)). Statistical comparisons were made by Student's t-test for unpaired samples.

FIG. 27. Loss of rpn-6.1 worsens the motility defects of polyQ67 worms. Thrashing rates of day 1 (FIG. 27a), day 3 (FIG. 27b) and day 5 (FIG. 27c) adulthood animals. At day 1, rpn-6.1 RNAi fed worms do not show a decreased in their motility compared to the vector RNAi fed worms (N2: vector RNAi vs rpn-6.1 RNAi (P=0.68); polyQ40: vector RNAi vs rpn-6.1 RNAi (P=0.26); polyQ67: vector RNAi vs rpn-6.1 RNAi (P=0.15)). At day 3, loss of rpn-6.1 significantly reduces the motility of polyQ67 worms, but not of wild-type or polyQ40 worms (N2: vector RNAi vs rpn-6.1 RNAi (P=0.15); polyQ40: vector RNAi vs rpn-6.1 RNAi (P=0.10); polyQ67: vector RNAi vs rpn-6.1 RNAi (P<0.0001)). At day 5, loss of rpn-6.1 dramatically decreases the motility of all the strains analyzed (N2: vector RNAi vs rpn-6.1 RNAi (P=7.8*10⁻¹²); polyQ40: vector RNAi vs rpn-6.1 RNAi (P=3.6*10⁻¹³); polyQ67: vector RNAi vs rpn-6.1 RNAi (P=3.0*10⁻⁸)). Statistical comparisons were made by Student's t-test for unpaired samples.

FIG. 28. Increased proteasome activity in hESCs and iPSCs. FIG. 28a, Chymotrypsin-like proteasome activity measured fluorometrically by digestion of the peptide Z-GGL-AMC in human cell extracts. Proteasome activity (relative slope to H9 hESCs) represents the mean±s.e.m. (H9 hESCs (n=11), NPCs (n=13), neurons (n=10)). Differentiation of hESCs into NPCs and neurons is associated with a decrease in chymotrypsin-like proteasome activity (P<0.00001). FIG. 28b, Representative immunoblot of polyubiquitinylated protein levels. β-actin was used as a loading control. Total protein was visualized by Coomassie staining in a corresponding protein gel. FIG. 28c, Caspase-like proteasome activity measured fluorometrically by digestion of the peptide Z-LLE-AMC, in human cell extracts. Proteasome activity (relative slope to H9 hESCs) represents the mean±s.e.m. (n=5). hESCs display increased caspase-like proteasome activity compared to NPCs and neurons (P<0.001). FIG. 28d, Trypsin-like proteasome activity measured fluorometrically by digestion of the peptide Ac-RLR-AMC, in human cell extracts. Proteasome activity (relative slope to H9 hESCs) represents the mean±s.e.m. (n=5). Differentiation of hESCs into NPCs and neurons is associated with a decrease in trypsin-like proteasome activity (P<0.0001). FIG. 28e, H9 hESCs lose their high proteasome activity in a progressive manner when they differentiate into trophoblasts (H9 hESCs vs 2 days of differentiation into trophoblasts (P<0.001), H9 hESCs vs 5 days of differentiation into trophoblasts (P=7.7*10⁻⁷), H9 hESCs vs 8 days of differentiation into trophoblasts (P<0.0001)). Proteasome activity (relative slope to H9 hESCs) represents the mean±s.e.m. (H9 hESCs (n=6), 2 days of differentiation into trophoblasts (n=6), 5 days of differentiation into trophoblasts (n=6), trophoblasts (n=7)). FIG. 28f, Differentiation of hESCs into fibroblasts is associated with a decrease in the chymotrypsin-like proteasome activity (P<0.001). Proteasome activity (relative slope to H9 hESCs) represents the mean±s.e.m. (n=3). FIG. 28g, Chymotrypsin-like proteasome activity (relative slope to BJ fibroblasts) represents the mean±s.e.m. (BJ fibroblasts (n=10), iPSC line 1 (n=10), iPSC line 2 (n=9), H9 hESCs (n=5)). iPSC lines derived from BJ fibroblast display increased proteasome activity compared to fibroblasts (P<0.0005) and no significant differences compared to H9 hESCs (iPSC line 1 vs H9 hESCs (P=0.11), iPSC line 2 vs H9 hESCs (P=0.29).

FIG. 29. Increased proteasome assembly and activity in hESCs dependent upon PSMD11 expression. FIG. 29a, Chymotrypsin-like proteasome activity measured fluorometrically by digestion of the peptide Z-GGL-AMC. 0.025% SDS was added to cell lysates 5 minutes prior to digestion assay. Proteasome activity (relative slope to H9 hESCs+0.025% SDS) represents the mean±s.e.m. (H9 hESCs (n=6), NPCs (n=5), neurons (n=5), H9 hESCs+0.025% SDS (n=5), NPCs+0.025% SDS (n=4), neurons+0.025% SDS (n=5)). Differentiation of hESCs into NPCs and neurons is associated with a decrease in chymotrypsin-like proteasome activity (P<0.01). No significant differences were found in chymotrypsin-like proteasome activity among the different cells when SDS was added (H9 hESCs+0.025% SDS vs NPCs+0.025% SDS (P=0.25), H9 hESCs+0.025% SDS vs neurons+0.025% SDS (P=0.09)). FIG. 29b, H9 hESCs have increased expression of PSMD11. Graph (relative expression to H9 hESCs) represents the mean±s.e.m. (H9 hESCs (n=10), NPCs (n=6), neurons (n=8)). Differentiation of hESCs into NPCs and neurons is associated with decreased expression of PSMD11 (P<0.00001). Statistical comparisons were made by Student's t-test for unpaired samples. See FIG. 39 for details on the relative mRNA levels of the other 19S proteasome subunits. FIG. 29c, Western blot analysis of cell extracts with antibodies to PSMD11 and PSMD1. β-actin was used as a loading control. See FIG. 40 for details on the levels of the other 26S proteasome subunits. FIG. 29d, Graph (relative expression to H9 hESCs) represents the mean±s.e.m. (n=4). Differentiation of hESCs into trophoblast is associated with down-regulation in PSMD11 expression (P<0.05). Statistical comparisons were made by Student's t-test for unpaired samples. FIG. 29e, Western blot analysis of PSMD11 in trophoblasts. β-actin was used as a loading control. FIG. 29f, Graph (relative expression to H9 hESCs) represents the mean±s.e.m. (n=4). Differentiation of hESCs into fibroblasts is associated with PSMD11 down-regulation (P<0.05). Statistical comparisons were made by Student's t-test for unpaired samples. FIG. 29g, Western blot analysis of PSMD11 in fibroblasts. β-actin was used as a loading control. FIG. 29h, PSMD11 levels increase when somatic cells are reprogrammed to iPSC (P<0.00001). Graph (relative expression to BJ fibroblasts) represents the mean±s.e.m. (BJ fibroblasts (n=10), iPSC line 1 (n=6), iPSC line 2 (n=6)). Statistical comparisons were made by Student's t-test for unpaired samples. FIG. 29i, Western blot analysis of cell homogenates show an up-regulation in the levels of PSMD11 in iPSC lines. β-actin was used as a loading control. FIG. 29j, Knockdown of PSMD11 decreases proteasome activity in H9 hESCs (LV-Non-targeting shRNA vs LV-PSMD11 shRNA 1 (P=0.01), LV-Non-targeting shRNA vs LV-PSMD11 shRNA 2 (P=0.01)). Proteasome activity (relative slope to LV-Non-targeting shRNA) represents the mean±s.e.m. (Non-targeting shRNA (n=10), PSMD11 shRNA 1 (n=8), PSMD11 shRNA 2 (n=6), PSMC2 shRNA 1 (n=6), PSMC2 shRNA 2 (n=3)). Statistical comparisons were made by Student's t-test for unpaired samples. FIG. 29k, H9 hESCs have more assembled proteasome compared to differentiated counterparts. Native gel electrophoresis followed by western blot with alpha 1+2+3+5+6+7 (20S subunit) or PSMD2 (19S subunit) antibodies. FIG. 29l, Ectopic expression of PSMD11 increases 30S assembly and proteasome activity in HEK293T cells. 3.5% native gel electrophoresis followed by proteasome activity assay with chymotrypsin-like activity substrate LLVY-AMC and immunoblotting with PSMD1 (19S subunit) antibody. Extracts were resolved by SDS-PAGE and immunoblotting for analysis of PSMD11 overexpression levels and loading control. FIG. 29m, Ectopic expression of PSMD11 increases proteasome activity in HEK293T cells (P<0.005). Chymotrypsin-like proteasome activity (relative slope to GFP OE HEK293T cells) represents the mean±s.e.m. (GFP OE (n=4), PSMD11 OE (n=5)). FIG. 29n, Loss of PSMD11 decreases 30S assembly and proteasome activity in HEK293T cells. 3.5% native gel electrophoresis followed by proteasome activity assay with chymotrypsin-like activity substrate LLVY-AMC and immunoblotting with PSMD1 (19S subunit) antibody. Extracts were resolved by SDS-PAGE and immunoblotting for analysis of PSMD11 knockdown levels and loading control.

FIG. 30. FOXO4 regulates proteasome activity in hESCs. FIG. 30a, Chymotrypsin-like proteasome activity measured in H9 hESCs transiently infected with lentiviruses to knock down the genes indicated in the figure. Proteasome activity (relative slope to non-infected cells) represents the mean±s.e.m. (n=19). Knockdown of FOXO4 decreases proteasome activity in H9 hESCs (P<0.00001). FIG. 30b, Chymotrypsin-like proteasome activity measured in stable H9 hESCs that express shRNA to the 3′UTR of FOXO4 transcript. Proteasome activity (relative slope to GFP cells) represents the mean±s.e.m. (GFP (n=7), 3′UTR FOXO4 shRNA 1 (n=6), 3′UTR FOXO4 shRNA 2 (n=3), 3′UTR FOXO4 shRNA 3 (n=6)). Knockdown of FOXO4 decreases proteasome activity in H9 hESCs (GFP vs 3′UTR FOXO4 shRNA 1 (P<0.01), GFP vs 3′UTR FOXO4 shRNA 2 (P<0.01), GFP vs 3′UTR FOXO4 shRNA 3 (P=4.5*10⁻⁸)). FIG. 30c, FOXO4 levels are down-regulated when H9 hESCs differentiate into NPCs, neurons ((H9 hESCs (n=6), NPCs (n=4), neurons (n=4)), trophoblasts (H9 hESCs (n=6), trophoblasts (n=6)) and fibroblasts (H9 hESCs (n=4), fibroblasts (n=4)). No significant differences were found between NPCs and neurons (P=0.43). Graphs represent the mean±s.e.m. of the relative expression levels to H9 hESCs. FOXO4 levels are up-regulated when BJ fibroblasts are reprogrammed into iPSCs (graph represents the mean±s.e.m. of the relative expression levels to BJ fibroblasts (BJ fibroblasts (n=8), iPSC line 1 (n=6), iPSC line 2 (n=6), BJ fibroblasts vs iPSC line 1 (P<0.05), BJ fibroblasts vs iPSC line 2). Statistical comparisons were made by Student's t-test for unpaired samples (*(P<0.05), **(P<0.01), ***(P<0.001)). FIG. 30d, Transient overexpression of constitutively active FOXO4 triple alanine mutant up-regulates chymotrypsin-like proteasome activity in H9 hESCs (non-infected cells versus LV-FOXO4 OE cells (P=0.41), non-infected cells vs LV-FOXO4 AAA OE cells (P<0.05)). Proteasome activity (relative slope to non-infected H9 hESCs) represents the mean±s.e.m. (n=7). FIG. 30e, Ectopic expression of FOXO4 AAA partially rescues low chymotrypsin-like proteasome activity in shFOXO4 hESCs (3′UTRFOXO4 shRNA 3 cells vs 3′UTR_{—}3 FOXO4 shRNA+FOXO4 AAA cells (P<0.01). Proteasome activity (relative slope to GFP hESCs) represents the mean±s.e.m. (n=4). FIG. 30f, Knockdown of FOXO4 decreases expression of PSMD11 in H9 hESCs (P<0.001 GFP vs FOXO4 shRNA, P<0.05 GFP vs 3′UTR FOXO4 shRNA 2, P<0.001 GFP vs 3′UTR FOXO4 shRNA 3). Graph represents the mean±s.e.m (LV-GFP (n=15), LV-FOXO4 shRNA (n=19), LV-3′UTR FOXO4 shRNA 2 (n=4), LV-3′UTR FOXO4 shRNA 3 (n=5)). Stable overexpression of FOXO4 AAA mutant increases PSMD11 expression in H9 hESCs (P=0.69 GFP vs FOXO4 OE cells, P<0.01 GFP vs FOXO4 AAA OE cells). Data represent the mean±s.e.m. of the relative expression levels to GFP hESCs (GFP (n=7), FOXO4 OE (n=8), FOXO4 AAA OE (n=7)). FIG. 30g, Western blot analysis of PSMD11 levels. β-actin loading control. FIG. 30h, PSMD11 overexpression rescues low proteasome activity of shFOXO4 H9 hESCs (GFP vs FOXO4 shRNA (P<0.01), GFP vs FOXO4 shRNA+PSMD11 OE (P=0.50)). Proteasome activity (relative slope to GFP H9 hESCs) represents the mean±s.e.m. (n=4). Statistical comparisons were made by Student's t-test for unpaired samples.

FIG. 31. FOXO4 and proteasomal activity are required for hESC function. FIG. 31a, FOXO4 shRNA H9 embryoid bodies (hEBs) were unable to generate rosettes and neural cells (P=2.6*10⁻²³). Graph represents the percentage of hEBs containing NPCs relative to GFP (mean±s.e.m. (n=20)). FIG. 31b, shRNAs to the 3′UTR of FOXO4 transcript block neural differentiation of H9 embryoid bodies (P<0.001). Graph represents the percentage of hEBs containing NPCs relative to GFP (mean±s.e.m. ((GFP (n=7), 3′UTR FOXO4 shRNA 1 (n=9), 3′UTR FOXO4 shRNA 2 (n=3), 3′UTR FOXO4 shRNA 3 (n=8)). FIG. 31c, After culturing in neural differentiation media, FOXO4 shRNA cells showed decreased expression in neural markers compared to GFP cells (*(P<0.05), **(P<0.01), ***(P<0.001)). Graph (relative expression to GFP cells) represents the mean±s.e.m. (n=12). FIG. 31d, After culturing in neural differentiation media, FOXO4 shRNA cells maintain increased expression of pluripotency markers compared to GFP cells (*(P<0.05), **(P<0.01), ***(P<0.001)). Graph (relative expression to GFP cells) represents the mean±s.e.m. (n=7). FIG. 31e, Immunocytochemistry after neural differentiation assay. β-III-tubulin, OCT4 and DAPI staining were used as markers of neurogenesis, pluripotency and nuclei, respectively. Regions of β-III-tubulin positive cells were reduced by approximately 80% in the FOXO4 shRNA cultures compared to the others. Scale bar represents 100 μm. FIG. 31f, Real Time PCR analysis of pluripotency (OCT4, NANOG, SOX2, UTF1, DPPA4, DPPA2, ZFP42 and TERT), trophectodermal (CDX2), ectodermal (PAX6, FGF5), mesodermal (MSX1) and endodermal (AFP, GATA6, GATA4, Albumin) germ layer markers. Proteasome inhibition (62.5 nM MG-132 24 h) in H9 hESCs induces a decrease in pluripotency markers and modified the levels of markers of the distinct germ cell and extraembryonic layers (P-value: *(P<0.05), **(P<0.01), ***(P<0.001). Graph (relative expression to DMSO control H9 hESCs) represents the mean±s.e.m. (DMSO (n=12), MG-132 (n=13). Statistical comparisons were made by Student's t-test for unpaired samples. FIG. 31g, Western blot analysis of cell extracts with antibodies to SOX2, PAX6, FGF5 and MSX1. β-actin loading control.

FIG. 32. Decrease in proteasome activity when NPCs differentiate into neurons. FIG. 32a, Chymotrypsin-like proteasome activity measured fluorometrically by digestion of the peptide Z-GGL-AMC, in H9 NPCs. Proteasome activity (relative slope to H9 NPCs) represents the mean±s.e.m. (H9 NPCs (n=6), 3 days of differentiation (n=5), 1 week (n=5), 2 weeks (n=5), 3 weeks (n=5), 4 weeks (n=7)). After 2 weeks of neuronal differentiation, these cells show a significant decrease in proteasome activity compared to NPCs (NPCs vs 3 days of neural differentiation treatment cells (P=0.20), NPCs vs 1 week (P=0.68), NPCs vs 2 weeks (P<0.01), NPCs vs 3 weeks (P<0.01), NPCs vs 4 weeks (P<0.01)). FIG. 32b, A distinct NPC line, HUES-6 NPCs, also shows a decrease in proteasome activity when they differentiate into neurons. Proteasome activity (relative slope to HUES-6 NPCs) represents the mean±s.e.m. (HUES-6 NPCs (n=6), 3 days (n=5), 1 week (n=5), 2 weeks (n=5), 3 weeks (n=5), 4 weeks (n=7)). After 2 weeks of neuronal differentiation, these cells show a decrease in proteasome activity compared to NPCs (NPCs vs 3 days of neural differentiation treatment cells (P=0.06), NPCs vs 1 week (P=0.22), NPCs vs 2 weeks (P<0.01), NPCs vs 3 weeks (P<0.05), NPCs vs 4 weeks (P<0.01)). Statistical comparisons were made by Student's t-test for unpaired samples.

FIG. 33. Increased proteasome activity in HUES-6 hESCs. Chymotrypsin-like proteasome activity measured fluorometrically by digestion of the peptide Z-GGL-AMC, in HUES-6 hESCs. Proteasome activity (relative slope to HUES-6 hESCs) represents the mean±s.e.m. (hESCs (n=7), NPCs (n=9), neurons (n=9)). Differentiation of HUES-6 cells into NPCs and neurons is associated with a decrease in chymotrypsin-like proteasome activity (P<0.0001). Statistical comparisons were made by Student's t-test for unpaired samples.

FIG. 34. Proteasome inhibitors block digestion of the proteasome substrate peptide Z-GGL-AMC. Proteasome inhibitors were added to cell lysates 20 minutes prior to digestion assay. All proteasome inhibitors tested block chymotrypsin-like proteasome activity in H9 hESCs (FIG. 34a), H9 NPCs (FIGS. 34b) and H9 neuronal lysates (FIG. 34c).

FIG. 35. Proteasome activity is not affected in hESCs when high passages are used. Chymotrypsin-like proteasome activity measured fluorometrically by digestion of the peptide Z-GGL-AMC, in H9 hESCs. Proteasome activity (relative slope to hESCs passage 43-45) represents the mean±s.e.m. (hESCs passage 43-45 (n=8), hESCs passage 83-85 (n=8). No significant differences were found between different passages (P=0.95). Statistical comparisons were made by Student's t-test for unpaired samples.

FIG. 36. Increased proteasome activity in hESCs compared to differentiated and HEK293T cells. Chymotrypsin-like proteasome activity measured fluorometrically by digestion of the peptide Z-GGL-AMC, in human cell extracts. H9 hESCs show increased proteasome activity compared to BJ fibroblasts (FIG. 36a, n=3, P<0.01), cortical astrocytes (FIG. 36b, n=3, P<0.0001), hippocampal astrocytes (FIG. 36c, n=4, P<0.05) and HEK293T cells (FIG. 36d, n=12, P<0.0001). Statistical comparisons were made by Student's t-test for unpaired samples.

FIG. 37. Upper Panel: Bromodeoxyuridine (BrdU) proliferation assay. H9 hESCs do not show significant differences in the percentage of BrdU positive cells compared to NPCs and HEK293T cells (H9 hESCs vs NPCs (P=0.06), H9 hESCs vs HEK293T cells (P=0.06)). The results are represented as averages±s.e.m. of at least 600 cells scored in Applicants' different fields. Lower Panel: H9 hESCs have increased proteasome activity compared to NPCs and HEK293T cells. Proteasome activity (relative slope to H9 hESCs) represents the mean±s.e.m. (H9 hESCs (n=13), H9 NPCs (n=8), BJ Fibroblasts (n=4), HEK293T (n=12). P-value: **(P<0.01), ***(P<0.001). Statistical comparisons were made by Student's t-test for unpaired samples.

FIG. 38. Upper Panel: No differences in total 20S proteasome activity between HUES-6 hESCS and differentiated cells. Chymotrypsin-like proteasome activity measured fluorometrically by digestion of the peptide Z-GGL-AMC. 0.025 SDS % was added to cell lysates 5 minutes prior to digestion assay. Proteasome activity (relative slope to HUES-6 hESCs) represents the mean±s.e.m. (HUES-6 hESCs (n=6), NPCs (n=6), neurons (n=6), HUES-6 hESCs+0.025% SDS (n=6), NPCs+0.025% SDS (n=6), neurons+0.025% SDS (n=4)). Lower Panel: Differentiation of hESCs into NPCs and neurons is associated with a decrease in chymotrypsin-like proteasome activity (P<0.05). No significant differences were found in chymotrypsin-like proteasome activity among the different cells when SDS was added (HUES-6 hESCs+0.025% vs NPCs+0.025% SDS (P=0.30), HUES-6 hESCs+0.025% vs neurons+0.025% SDS (P=0.42))

FIG. 39. 19S proteasome subunit transcript levels. Graphs represent the mean±s.e.m. of the relative expression levels to H9 hESCs (H9 hESCs (n=10), NPCs (n=6), neurons (n=7)).

FIG. 40. 26S proteasome subunit levels in H9 hESCs. Western blot analysis of cell extracts. β-actin was used as a loading control.

FIG. 41. Increased expression of PSMD11 in HUES-6 hESCs. HUES-6 hESCs have up-regulated expression of PSMD11. Graph (relative expression to HUES-6 hESCs) represents the mean±s.e.m. (HUES-6 hESCs (n=6), NPCs (n=6), neurons (n=8)). Differentiation of HUES-6 hESCs into NPCs and neurons is associated with down-regulation in the expression of PSMD11 (HUES-6 hESCs vs NPCs (P<0.01), HUES-6 hESCs vs neurons (P<0.05)). Statistical comparisons were made by Student's t-test for unpaired samples.

FIG. 42. Knockdown efficiencies in FOXO4 shRNA stable H9 and HUES-6 hESCs. FIG. 42a, Western blot analysis of cell extracts with antibodies to FOXO4 and FOXO1a in H9 hESCs. β-actin was used as a loading control. FIG. 42b, Western blot analysis of cell extracts with antibodies to FOXO4 in HUES-6 hESCs. β-actin was used as a loading control.

FIG. 43. Proteasome activity is down-regulated in stable FOXO4 shRNA H9 and HUES-6 hESCs. FIG. 43a, Chymotrypsin-like proteasome activity measured in stable H9 hESCs with a knockdown in the genes indicated. Proteasome activity (relative slope to GFP cells) represents the mean±s.e.m. (n=8). Knockdown of FOXO4 decreases proteasome activity in hESCs (P<0.00001). FIG. 43b, Chymotrypsin-like proteasome activity measured in independent stable lines generated from different clones than the ones shown in FIG. 43a. Proteasome activity (relative slope to GFP cells) represents the mean±s.e.m. (n=4). Knockdown of FOXO4 decreases proteasome activity in hESCs (P<0.005). FIG. 43c, Chymotrypsin-like proteasome activity measured in stable HUES-6 hESCs with a knockdown in the genes indicated. Proteasome activity (relative slope to GFP cells) represents the mean±s.e.m. (GFP (n=8), HSF1 shRNA (n=5), FOXO1 a shRNA (n=6), FOXO3a shRNA (n=3), FOXO4 shRNA (n=4), 3′UTR FOXO4 shRNA 1 (n=6), 3′UTR FOXO4 shRNA (n=4), 3′UTR FOXO4 shRNA (n=4)). Knockdown of FOXO4 decreases proteasome activity in HUES-6 hESCs (GFP vs FOXO4 shRNA (P=9.6*10⁻⁸), GFP vs 3′UTR shRNA 1 FOXO4 (P<0.05), GFP vs 3′UTR_{—}2 FOXO4 shRNA 2 (P<0.0001), GFP vs 3′UTR FOXO4 shRNA 3 (P<0.01)).

FIG. 44. FOXO expression levels in HUES-6 hESCs. Graph represents the mean±s.e.m. of the relative expression levels to HUES-6 hESCs (hESCs (n=9), NPCs (n=6), neurons (n=9)). Statistical comparisons were made by Student's t-test for unpaired samples. (P-value: *(P<0.05), ***(P<0.001).

FIG. 45. FOXO expression levels in both H9 hESCs and iPSCs. FIG. 45a, Graph represents the mean±s.e.m. of the relative expression levels to H9 hESCs (hESCs (n=6), NPCs (n=4), neurons (n=4)). FIG. 45b, Graph represents the mean±s.e.m. of the relative expression levels to H9 hESCs (hESCs (n=6), trophoblasts (n=6). FIG. 45c, Graph represents the mean±s.e.m. of the relative expression levels to H9 hESCs (hESCs (n=4), fibroblasts (n=4). FIG. 45d, Graph represents the mean±s.e.m. of the relative expression levels to BJ fibroblasts (BJ fibroblasts (n=8), iPSC line 1 (n=6), iPSC line 2 (n=6)). Statistical comparisons were made by Student's t-test for unpaired samples. (P-value: *(P<0.05), **(P<0.01), ***(P<0.001).

FIG. 46. FOXO6 expression levels. FIG. 46a, Graph represents the mean±s.e.m. of the relative expression levels to H9 hESCs (H9 hESCs (n=4), NPCs (n=3), neurons (n=3)). FIG. 46b, Graph represents the mean±s.e.m. of the relative expression levels to H9 hESCs (H9 hESCs (n=4), trophoblasts (n=4). FIG. 46c, Graph represents the mean±s.e.m. of the relative expression levels to H9 hESCs (hESCs (n=4), fibroblasts (n=4). FIG. 46d, Graph represents the mean±s.e.m. of the relative expression levels to HUES-6 hESCs (HUES-6 hESCs (n=8), NPCs (n=9), neurons (n=15)). FIG. 46e, Graph represents the mean±s.e.m. of the relative expression levels to BJ fibroblasts (BJ fibroblasts (n=5), iPSC line 1 (n=6), iPSC line 2 (n=3)). FIG. 46e, Graph represents the mean±s.e.m. of the relative expression levels to HUES-6 hESCs (HUES-6 hESCs (n=8), NPCs (n=9), neurons (n=15)). Statistical comparisons were made by Student's t-test for unpaired samples. (P-value: *(P<0.05), **(P<0.01), ***(P<0.001).

FIG. 47. Proteasome activity in FOXO4 knockdown NPCs and neurons. FIG. 47a, Knockdown of FOXO4 decreases chymotrypsin-like proteasome activity in NPCs (P<0.01). Proteasome activity (relative slope to non-infected NPCs) represents the mean±s.e.m. (n=4). FIG. 47b, Knockdown of FOXO4 does not affect chymotrypsin-like proteasome activity in neurons (P=0.77 non-infected cells vs LV-FOXO4 shRNA cells). Proteasome activity (relative slope to non-infected neurons) represents the mean±s.e.m. (n=4). Statistical comparisons were made by Student's t-test for unpaired samples.

FIG. 48. Loss of FOXO4 does not decrease proteasome activity in dividing cells such as fibroblasts or HEK293T cells. FIG. 48a, Chymotrypsin-like proteasome activity measured in BJ fibroblasts infected with lentiviruses to knock down FOXO4. Proteasome activity (relative slope to Luciferase shRNA infected cells) represents the mean±s.e.m. (n=4). Knockdown of FOXO4 does not decrease proteasome activity in BJ fibroblasts (P=0.11). FIG. 48b, FOXO4 knockdown in BJ fibroblasts. Graph (relative expression to Luciferase shRNA infected cells) represents the mean±s.e.m. (n=3, P<0.05). FIG. 48c, Chymotrypsin-like proteasome activity measured in HEK293T cells infected with lentiviruses to knock down FOXO4. Proteasome activity (relative slope to Luciferase shRNA infected cells) represents the mean±s.e.m. (n=4). Knockdown of FOXO4 does not decrease proteasome activity in HEK293T (P=0.13). FIG. 48d, FOXO4 knockdown in HEK293T cells. Graph (relative expression to Luciferase shRNA infected cells) represents the mean±s.e.m. (n=3, P<0.05). Statistical comparisons were made by Student's t-test for unpaired samples.

FIG. 49. Proteasome activity in stable FOXO4 overexpressing H9 hESCs. Stable overexpression (OE) of constitutively active FOXO4 AAA mutant up-regulates chymotrypsin-like proteasome activity in H9 hESCs (GFP vs FOXO4 OE cells (P=0.69), GFP vs FOXO4 AAA OE cells (P<0.01)). Proteasome activity (relative slope to GFP H9 hESCs) represents the mean±s.e.m. (GFP (n=14), FOXO4 OE (n=12), FOXO4 AAA OE (n=10)). Statistical comparisons were made by Student's t-test for unpaired samples.

FIG. 50. PSMD11 expression in FOXO4 knockdown NPCs and neurons. FIG. 50a, Knockdown of FOXO4 decreases the expression of PSMD11 and in NPCs. Graph represents the mean±s.e.m (LV-GFP (n=7), LV-FOXO4 shRNA (n=5)). FIG. 50b, Knockdown of FOXO4 in neurons. Graph represents the mean±s.e.m (LV-GFP (n=5), LV-FOXO4 shRNA (n=3)).

FIG. 51. Impaired neural differentiation of FOXO4 shRNA H9 hESCs. Phase contrast brightfield images of two week old hEBs after one week of laminin adhesion acquired on an Olympus IX51 microscope. Neural rosettes and projections are easily visualized in GFP, HSF-1 shRNA, FOXO1a shRNA and FOXO3a shRNA but are absent in FOXO4 shRNA.

FIG. 52. FOXO4 is essential for neural differentiation. FIG. 52a, Representative immunoblots of NPC (SOX1, Musashi 1) and neuronal (MAP2, β-III-tubulin) markers of cell extracts after the neural differentiation treatment. β-actin was used as a loading control. FIG. 52b, Immunocytochemistry after differentiation into neural cells. β-III-tubulin, OCT4 and DAPI staining were used as markers of neurogenesis, pluripotency and nuclei, respectively. Scale bar represents 100 μm.

FIG. 53. FOXO4 is required for H9 hESCs differentiation into neural cells. After culturing in neural differentiation media, 3′UTR FOXO4 shRNA H9 hESCs have decreased expression of neural markers compared to GFP control cells (P-value: *(P<0.05), ***(P<0.001). Graph (relative expression to GFP H9 hESCs) represents the mean±s.e.m. ((GFP (n=11), 3′UTR FOXO4 shRNA 1 (n=5), 3′UTR FOXO4 shRNA 2 (n=5), 3′UTR FOXO4 shRNA 3 (n=12). Statistical comparisons were made by Student's t-test for unpaired samples.

FIG. 54. FOXO4 is required for HUES-6 hESCs differentiation into neural cells. After culturing in neural differentiation media, FOXO4 shRNA HUES-6 cells maintain increased expression of pluripotency markers (with the exception of SOX2) and have decreased expression in neural markers compared to GFP cells (P-value: *(P<0.05), **(P<0.01), ***(P<0.001). Graph (relative expression to GFP cells) represents the mean±s.e.m. (GFP (n=9), HSF1 shRNA (n=4), FOXO1a shRNA (n=6), FOXO3a shRNA (n=5), FOXO4 shRNA (n=5), 3′UTR FOXO4 shRNA 1 (n=5), 3′UTR FOXO4 shRNA 2 (n=6)). Statistical comparisons were made by Student's t-test for unpaired samples.

FIG. 55. Ectopic expression of FOXO4 AAA ameliorates the failure in neural differentiation of 3′UTR FOXO4 shRNA hESCs. FIG. 55a, Ectopic expression of FOXO4 AAA partially rescues the failure in neural differentiation of FOXO4 shRNA hESCs (3′UTR FOXO4 shRNA 3 cells vs 3′UTR FOXO4 shRNA 3+FOXO4 AAA cells (P<0.05). Graph represents the percentage of NPCs containing hEBs relative to GFP (mean±s.e.m. (n=4)). FIG. 55b, Ectopic expression of FOXO4 AAA increases the levels of neural markers in FOXO4 shRNA hESCs after differentiation (P<0.05). Graph (relative expression to 3′UTR FOXO4 shRNA 3 hESCs) represents the mean±s.e.m. (n=4). FIG. 55c, Representative immunoblots of NPC (SOX, Musashi 1) and neuronal (MAP2) markers of cell extracts after the neural differentiation treatment. β-actin was used as a loading control. FIG. 55d, Graphs represents the percentage of hEBs containing MAP2-positive cells (mean±s.e.m. (n=3 independent experiments)). GFP clone 1 vs FOXO4 shRNA (P<0.0005), GFP clone 1 vs GFP clone 3 (P=0.93), GFP clone 1 vs 3′UTR FOXO4 shRNA 3 (P<0.05), GFP clone 1 vs 3′UTR FOXO4 shRNA 3+FOXO4 AAA (P=0.41), 3′UTR FOXO4 shRNA 3 vs 3′UTR FOXO4 shRNA 3+FOXO4 AAA (P<0.05). FIG. 55e, Representative images of immunocytochemistry after neural differentiation assay. MAP2 and DAPI staining were used as markers of neurogenesis and nuclei, respectively. Scale bar represents 100 μm.

FIG. 56. Ectopic expression of PSMD11 is not sufficient to rescue the failure in neural differentiation of FOXO4 shRNA hESCs. Graph represents the percentage of NPCs containing hEBs relative to GFP (mean±s.e.m. (n=5)). FOXO4 shRNA cells vs FOXO4 shRNA+PSMD11 OE cells (P=0.98). Statistical comparisons were made by Student's t-test for unpaired samples.

FIG. 57. Trophoblast differentiation of stable FOXO4 shRNA H9 hESCs. FIG. 57a, After differentiation into trophoblasts, cells show both a decrease in pluripotency markers and an increase in trophoblasts markers. Data represent the mean±s.e.m. of the relative expression levels to GFP overexpressing (OE) stable H9 cells. (GFP OE stable H9 cells (n=4), GFP OE trophoblasts (n=6)). FIG. 57b, Graphs (relative expression to GFP cells) represent the mean±s.e.m. (n=8). After culturing in trophoblast differentiation media, FOXO4 shRNA cells show increased expression in trophoblast markers compared to GFP cells (CD9 (P<0.05), CGB (P<0.01), GATA2 (P<0.01), GATA3 (P<0.001), GCM (P<0.001), HEY1 (P<0.01), MSX2 (P<0.001), PAEP (P<0.001), TFAP2 (P<0.001)). Statistical comparisons were made by Student's t-test for unpaired samples.

FIG. 58. Keratinocytes differentiation of stable FOXO4 shRNA H9 hESCs. FIG. 58a, After differentiation into keratinocytes, cells show both a decrease in pluripotency markers and an increase in keratinocytes markers. Data represent the mean±s.e.m. of the relative expression levels to GFP overexpressing (OE) stable H9 cells. (GFP OE stable H9 cells (n=4), GFP OE keratinocytes (n=6)). FIG. 58b, Graphs (relative expression to GFP cells) represent the mean±s.e.m. (n=4). After culturing in keratinocytes differentiation media, FOXO4 shRNA cells show increased expression in keratinocyte markers compared to GFP cells (P-value: *(P<0.05), **(P<0.01), ***(P<0.001)). Statistical comparisons were made by Student's t-test for unpaired samples.

FIG. 59. Neural differentiation in PSMD11 shRNA hESCs. FIG. 59a, After culturing in neural differentiation media, PSMD11 shRNA H9 and HUES-6 hESCs have decreased expression of β-III-tubulin compared to Non-targeting shRNA control cells (P-value: *(P<0.05), **(P<0.01). Graph (relative expression to Non-targeting shRNA hESCs) represents the mean±s.e.m. (H9 Non-targeting shRNA (n=9), H9 PSMD11 shRNA 1 (n=5), H9 PSMD11 shRNA 2 (n=9), HUES-6 Non-targeting shRNA (n=4), HUES-6 PSMD11 shRNA 1 (n=5), HUES-6 PSMD11 shRNA 2 (n=4)). Statistical comparisons were made by Student's t-test for unpaired samples. FIG. 59b, After culturing in neural differentiation media, PSMD11 shRNA H9 hESCs have increased expression of OCT4, NANOG and UTF1 compared to Non-targeting shRNA control cells. Graph (relative expression to Non-targeting shRNA hESCs) represents the mean±s.e.m. (H9 Non-targeting shRNA (n=9), H9 PSMD11 shRNA 1 (n=5), H9 PSMD11 shRNA 2 (n=9). Statistical comparisons were made by Student's t-test for unpaired samples (P-value: *(P<0.05), **(P<0.01), ***(P<0.001). FIG. 59c, After culturing in neural differentiation media, PSMD11 HUES-6 hESCs have decreased expression of nestin and increased expression in OCT4, NANOG and UTF1 compared to Non-targeting shRNA control cells (HUES-6 Non-targeting shRNA (n=4), HUES-6 PSMD11 shRNA 1 (n=5), HUES-6 PSMD11 shRNA 2 (n=4)). Statistical comparisons were made by Student's t-test for unpaired samples (P-value: *(P<0.05), **(P<0.01), ***(P<0.001). d, After culturing in neural differentiation media, PSMC2 shRNA hESCs do not show significant differences in the expression of Nestin, β-III-tubulin and MAP2 compared to Non-targeting shRNA control cells. Graph (relative expression to Non-targeting shRNA hESCs) represents the mean±s.e.m. (H9 Non-targeting shRNA (n=9), H9 PSMC2 shRNA 1 (n=7), HUES-6 Non-targeting shRNA (n=4), HUES-6 PSMC2 shRNA 1 (n=4). Statistical comparisons were made by Student's t-test for unpaired samples. FIG. 59e, Decreased levels of PSMD11 does not affect the percentage of NPCs containing hEBs relative to Non-targeting shRNA (mean±s.e.m. (n=14)). Statistical comparisons were made by Student's t-test for unpaired samples.

FIG. 60. 62.5 nM MG-132 induces accumulation of polyubiquitinylated proteins in hESCs. FIG. 60a, MG-132 (62.5 nM for 24 hours) treatment dramatically decreases proteasome activity in hESCs (P<0.001). Proteasome activity (relative slope to DMSO H9 hESCs) represents the mean±s.e.m. (n=3). Statistical comparisons were made by Student's t-test for unpaired samples. FIG. 60b, Proteasome inhibition (62.5 nM MG-132 24 h) in H9 hESCs induces accumulation of polyubiquitinylated proteins. β-actin was used as a loading control.

### DETAILED DESCRIPTION OF THE INVENTION

### Definitions

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

Construction of suitable vectors containing the desired therapeutic gene coding and control sequences may employ standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides may be cleaved, tailored, and re-ligated in the form desired.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, virus, nucleic acid, protein, or vector, indicates that the cell, virus, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec -2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

A “short hairpin RNA” or “small hairpin RNA” is a ribonucleotide sequence forming a hairpin turn which can be used to silence gene expression. After processing by cellular factors the short hairpin RNA interacts with a complementary RNA thereby interfering with the expression of the complementary RNA.

A “dominant negative protein” is a modified form of a wild-type protein that adversely affects the function of the wild-type protein within the same cell. As a modified version of a wild-type protein the dominant negative protein may carry a mutation, a deletion, an insertion, a post-translational modification or combinations thereof. Any additional modifications of a nucleotide or polypeptide sequence known in the art are included. The dominant-negative protein may interact with the same cellular elements as the wild-type protein thereby blocking some or all aspects of its function.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “transfection” or “transfected” are defined by a process of introducing nucleic acid molecules into a cell by non-viral and viral-based methods. The nucleic acid molecules may be gene sequences encoding complete proteins or functional portions thereof.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair .

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The term “reprogramming” refers to the process of dedifferentiating a non-pluripotent cell into a cell exhibiting pluripotent stem cell characteristics.

An “induced pluripotent stem cell” refers to a pluripotent stem cell artificially derived from a non-pluripotent cell. A non-pluripotent cell can be a cell of lesser potency to self-renew and differentiate than a pluripotent stem cell. Cells of lesser potency can be, but are not limited to somatic stem cells, tissue specific progenitor cells, primary or secondary cells. Without limitation, a somatic stem cell can be a hematopoietic stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

The term “treating” means ameliorating, suppressing, eradicating, and/or delaying the onset of the disease being treated.

By “therapeutically effective dose or amount” herein is meant a dose that produces effects for which it is administered. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). The term “therapeutically effective amount,” as used herein, further refers to that amount of the therapeutic agent sufficient to ameliorate a given disorder or symptoms. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

“Proteasome activity” as provided herein is an activity performed by the proteasome. The proteasome is a high molecular weight structure consisting of cellular enzymes and regulatory proteins, which degrade unneeded, damaged or misfolded proteins in a cell. This degradation process is characterized by a sequence of reactions including protein unfolding and peptide hydrolysis (proteolysis). Thus, the proteasome activity as described herein encompasses the steps associated with protein degradation in a cell, which are performed by the proteasome.

A “proteome” is defined as the entire set of proteins expressed by a genome in a cell, tissue or organism. In some embodiments, a proteome is a set of proteins expressed in a given type of cell or organism. In another embodiment, a proteome is a set of proteins expressed in a cell or organism at a given time.

An “rpn-6.1 protein” as referred to herein includes any of the naturally-occurring forms of rpn-6.1, homologs or variants thereof that maintain rpn-6.1 activity (e.g. within at least 50%, 80%, 90% or 100% activity compared to rpn-6.1). In some embodiments, variants have at least 90% amino acid sequence identity across their whole sequence compared to the naturally occurring rpn-6.1 polypeptide. In other embodiments, the rpn-6.1 protein is the protein as identified by the NCBI reference gi: 74964974.

“Foxo4” as referred to herein includes any of the naturally-occurring forms of the forkhead box protein O4, homologs or variants thereof that maintain Foxo4 protein activity (e.g. within at least 50%, 80%, 90% or 100% activity compared to Foxo4). In some embodiments, variants have at least 90% amino acid sequence identity across their whole sequence compared to the naturally occurring Foxo4 polypeptide. In other embodiments, the Foxo4 protein is the protein as identified by the NCBI reference gi: 103472003 and gi: 283436083 (isoforms 1 and 2).

The terms “agonist,” “activator,” “upregulator,” etc. refer to a substance capable of detectably increasing the expression or activity of a given gene or activity. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or more higher than the expression or activity in the absence of the agonist.

An “rpn-6.1 agonist” is a substance that increases the expression or activity of rpn-6.1 in a cell. rpn-6.1 expression can be increased, e.g., by introducing a nucleic acid encoding an rpn-6.1 protein into a cell under conditions permitting expression, or by addition or activation of a positive regulatory factor upstream of rpn-6.1 expression. rpn-6.1 protein activity can be increased, e.g., by transduction of an rpn-6.1 protein into a cell, or addition or activation of a positive regulatory factor upstream of rpn-6.1 activity. In some aspects, the rpn-6.1 agonist is an inhibitor of an agent that represses rpn-6.1 expression or activity.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life or engraftment potential) or therapeutic measures (e.g., comparison of side effects). Controls can be designed for in vitro applications, e.g., testing the activity of various rpn-6.1 agonists. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

“Subject,” “patient,” “individual in need of treatment” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision.

In the context of the present invention, i.e., methods for treating a protein-misfolding disease, a subject in need of treatment can refer to an individual that suffers from a deficiency affecting the proteasome (e.g. a neurodegenerative disease). The deficiency can be due to a genetic defect, injury, or pathogenic infection.

### DETAILED EMBODIMENTS

### I. Modulation of Proteasome Activity and Life Span Extension

In one aspect, a method of modulating a proteasome activity in a cell is provided. The method includes modulating an rpn-6.1 protein activity or an rpn-6.1 protein level in the cell thereby modulating the proteasome activity. Modulating as defined herein includes increasing as well as decreasing the activity or level of an rpn-6.1 protein or its homolog. A homolog of an rpn-6.1 protein refers to a polypeptide having the same biological function and activity as an rpn-6.1 protein, wherein the polypeptide is derived from a different species. In some embodiments, the method includes increasing the rpn-6.1 protein activity or the rpn-6.1 protein level, thereby increasing the proteasome activity. In other embodiments, the method includes decreasing the rpn-6.1 protein activity or the rpn-6.1 protein level, thereby decreasing the proteasome activity. In order to achieve a modulation of the proteasome activity the level or the activity of an rpn-6.1 protein or its homolog may be modulated. The level of an rpn-6.1 protein refers to a quantity of rpn-6.1 proteins present in a cell. Therefore, modulation of the level of an rpn-6.1 protein may be achieved by modulating such quantity of rpn-6.1 proteins. The modulation of a quantity of rpn-6.1 proteins may be performed by modulating the expression of rpn-6.1 proteins by rpn-6.1 encoding nucleic acids. Thus, in some embodiments, the method of modulating the level of an rpn-6.1 protein activity or an rpn-6.1 protein level in a cell includes introducing to the cell a nucleic acid encoding an rpn-6.1 polypeptide. Subsequent expression of the rpn-6.1 encoding nucleic acid will increase the quantity of rpn-6.1 proteins in the cell thereby increasing the rpn-6.1 protein level in the cell. The rpn-6.1 protein activity as described herein refers to the activity of an rpn-6.1 protein as a regulatory subunit of the proteasome. In some embodiments, modulating an rpn-6.1 protein activity includes administering an rpn-6.1 antagonist or agonist to the cell, thereby modulating the proteasome activity. In some embodiments, the agonist increases the rpn-6.1 protein activity thereby increasing the proteasome activity. An agonist as defined herein is an agent capable of increasing an rpn-6.1 protein activity thereby increasing proteasome activity. Examples of an agonist include without limitation, nucleic acids, proteins, peptides, oligosaccharides, polysaccharides, lipids, phospholipids, glycolipids, monomers, polymers, small molecules and organic compounds.

In one aspect, the cell as provided in the methods herein including embodiments thereof forms an organism. In some embodiments, this organism is a mammal. In other embodiments, the mammal is a human. In other embodiments, the organism is a nematode. In some embodiments, the nematode is C. elegans.

In another aspect, a method of increasing cell survival of a cell, which suffers from proteotoxic stress is provided. The method includes increasing an rpn-6.1 protein activity or an rpn-6.1 protein level in the cell thereby increasing cell survival of the cell, which suffers from proteotoxic stress. The term “increasing cell survival” as provided herein refers to extending a cell's or organism's life span. The term “life span” as provided herein is defined as the time a cell or organism is considered to be alive (e.g. capable of maintaining essential cellular or organism functions, respectively). For example, where life span is increased for a cell, the rate of senescence for that cell is typically decreased. Therefore, the time passed from the point a cell or organism is formed until the point it seizes to be alive is considered life span. Another acceptable term for life span known to those of skill in the art is “longevity.”

“Proteotoxic stress” as defined herein refers to a condition of a cell or organism in which the cell is stressed (i.e. cell functions are adversely affected) due to proteotoxic factors. Proteotoxic stress is induced by exogenous or endogenous proteotoxic factors. Examples of exogenous proteotoxic factors causing proteotoxic stress include without limitation starvation, oxygen deprivation (e.g. oxidative stress), UV irradiation and hyperthermia (e.g. heat shock-induced stress). Examples of endogenous proteotoxic factors causing proteotoxic stress include without limitation misfolded proteins or protein aggregation. The exogenous or endogenous proteotoxic factors damage the proteome, thereby transforming the cell or organism into a state of proteotoxic stress. Therefore, the condition of proteotoxic stress is characterized by a defective proteome of a cell or organism. The method provided herein includes increasing an rpn-6.1 protein activity or an rpn-6.1 protein level in a cell, which suffers from proteotoxic stress, thereby increasing cell survival of the cell. In some embodiments, the cell forms an organism. In other embodiments, the organism is a human. In other embodiments, the proteotoxic stress is oxidative stress. In other embodiments, the proteotoxic stress is heat shock-induced stress. In some embodiments, an rpn-6.1 protein level is increased by introducing to the cell a nucleic acid encoding an rpn-6.1 polypeptide. In other embodiments, an rpn-6.1 protein activity is increased by administering an rpn-6.1 agonist to the cell, thereby increasing the activity of the rpn-6.1 protein.

In some embodiments, increasing the rpn-6.1 protein activity or the rpn-6.1 protein level includes increasing the stress tolerance in a cell suffering from proteotoxic stress. Where the defects in the proteome are of such nature that the cell or organism is able to repair such defects, the cell or organism is considered to be in a state of “stress tolerance.” Stress tolerance is characterized by the summary of cellular processes that result in repair of a defective proteome. Examples of stress tolerance processes include without limitation, processes involved in nucleic acid repair, gene transcription, protein translation and protein folding. Therefore, increasing the rpn-6.1 protein activity or the rpn-6.1 protein level includes increasing the stress tolerance in a cell.

In another aspect, a method for treating a protein-misfolding disease in a subject in need thereof is provided. The method includes administering to the subject a therapeutically effective amount of an rpn-6.1 modulator. In some embodiments, the rpn-6.1 modulator increases an rpn-6.1 protein activity or an rpn-6.1 protein level. In other embodiments, the protein misfolding-disease is a neurodegenerative disease. In some embodiments, the neurodegenerative disease is Huntington's disease. In other embodiments, the neurodegenerative disease is Alzheimer's disease. In other embodiments, the neurodegenerative disease is Parkinson's disease.

### II. Methods Pertaining to Stem Cells and Neurogenesis

In another aspect, a method of increasing neurogenesis in a cell is provided. The method includes increasing a Foxo4 protein activity or a Foxo4 protein level in the cell. In some embodiments, increasing the Foxo4 protein activity or the Foxo4 protein level includes increasing a PSMD 11 protein activity or a PSMD 11 protein level. In other embodiments, increasing the Foxo4 protein activity or the Foxo4 protein level includes increasing the proteasome activity of the cell. In another aspect, the cell as provided in the methods herein including embodiments thereof forms an organism. In some embodiments, the organism is a mammal. In other embodiments, the mammal is a human.

“PSMD 11” as referred to herein includes any of the naturally-occurring forms of the PSMD 11 protein, or variants thereof that maintain PSMD 11 activity (e.g. within at least 50%, 80%, 90% or 100% activity compared to PSMD 11). In some embodiments, variants have at least 90% amino acid sequence identity across their whole sequence compared to the naturally occurring PSMD 11 polypeptide. In other embodiments, the PSMD 11 protein is the protein as identified by the NCBI reference gi: 28872725.

In another aspect, a method for preparing an induced pluripotent stem cell is provided. The method includes modulating a Foxo4 protein activity or a Foxo4 protein level in a non-pluripotent cell thereby forming a modulated non-pluripotent cell. The modulated non-pluripotent cell is allowed to divide and thereby forms the induced pluripotent stem cell. In some embodiments, the modulating includes increasing a Foxo4 protein activity or a Foxo4 protein level in the non-pluripotent cell. As described above for rpn-6.1, a level of a protein (e.g. Foxo4, PSMD 11) may be increased e.g., by introducing a nucleic acid encoding a Foxo4 or a PSMD 11 protein into a cell under conditions permitting expression, or by addition or activation of a positive regulatory factor upstream of Foxo4 or PSMD 11 expression. A Foxo4 or PSMD11 protein activity may be increased in a cell or organism by administering a Foxo4 or a PSMD11 agonist to the cell or organism. A Foxo4 or a PSMD 11 protein activity may be increased, e.g., by transduction of a Foxo4 or a PSMD 11 protein into a cell, or addition or activation of a positive regulatory factor upstream of Foxo4 or PSMD 11 activity. In some aspects, a Foxo4 or a PSMD 11 agonist is an inhibitor of an agent that represses Foxo4 or PSMD 11 expression or activity. In other embodiments, the non-pluripotent cell is a primary cell. In other embodiments, the primary cell is a fibroblast. Allowing the modulated non-pluripotent cell to divide and thereby forming the induced pluripotent stem cell may include expansion of the modulated non-pluripotent cell, optional selection for the modulated non-pluripotent cell and identification of induced pluripotent stem cells. Expansion as used herein includes the production of progeny cells by a modulated non-pluripotent cell in containers and under conditions well known in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblast that secrete such cellular growth factors. Examples of cellular growth factors include, but are not limited to, FGF, bFGF2, and EGF.

### EXAMPLES

The following examples are offered to illustrate, but not to limit the claimed invention.

### 1. Example 1

### Rpn-6.1 as a Modulator of Proteasome Activity

Applicants find that the forced re-investment of resources from the germline to the soma in C. elegans results in elevated somatic proteasome activity, clearance of damaged proteins, and increased longevity. This activity is associated with increased expression of rpn-6, a subunit of the 19S proteasome, by the FOXO transcription factor daf-16. Ectopic expression of rpn-6 is sufficient to confer proteotoxic stress resistance and extend lifespan, positing rpn-6 as a candidate to correct deficiencies in age-related protein homeostasis disorders.

Increased Proteasome Activity in glp-1(e2141) Worms

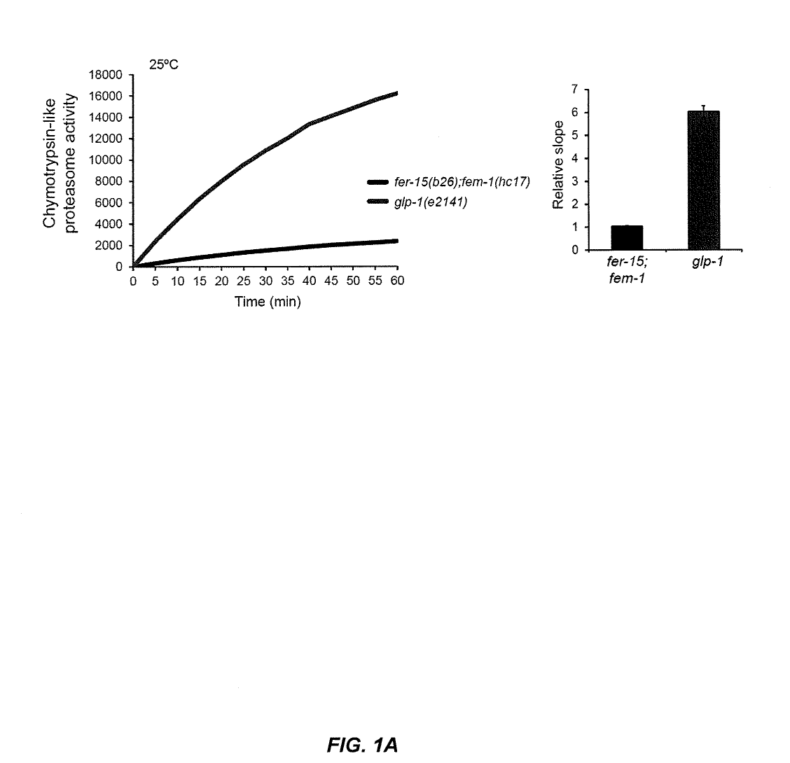

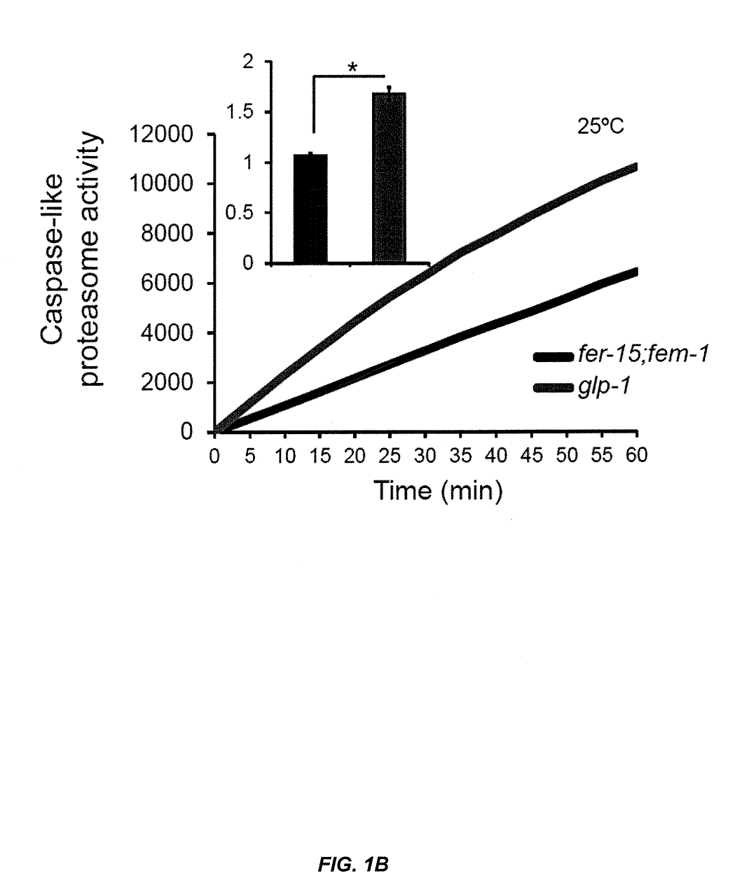

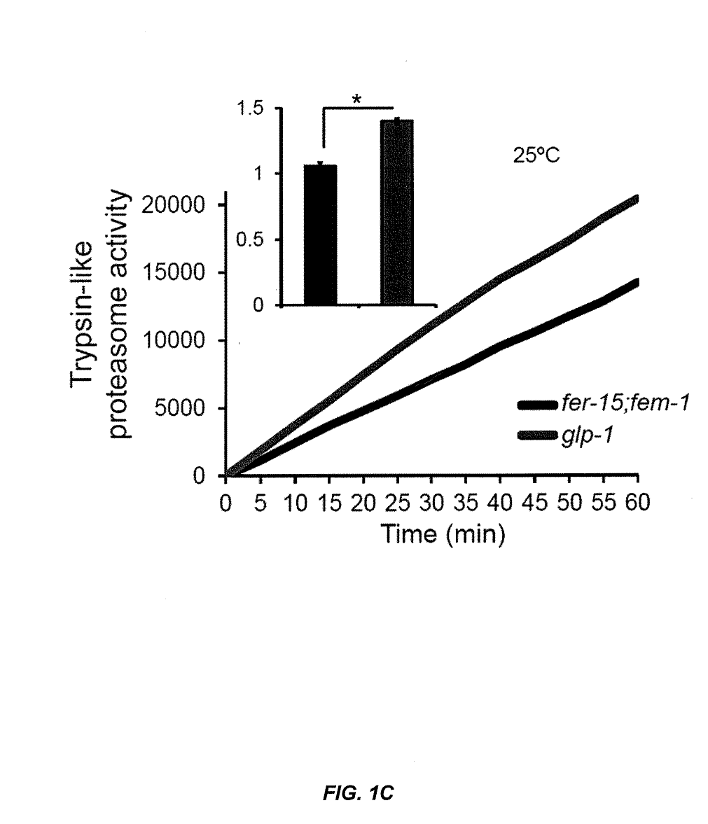

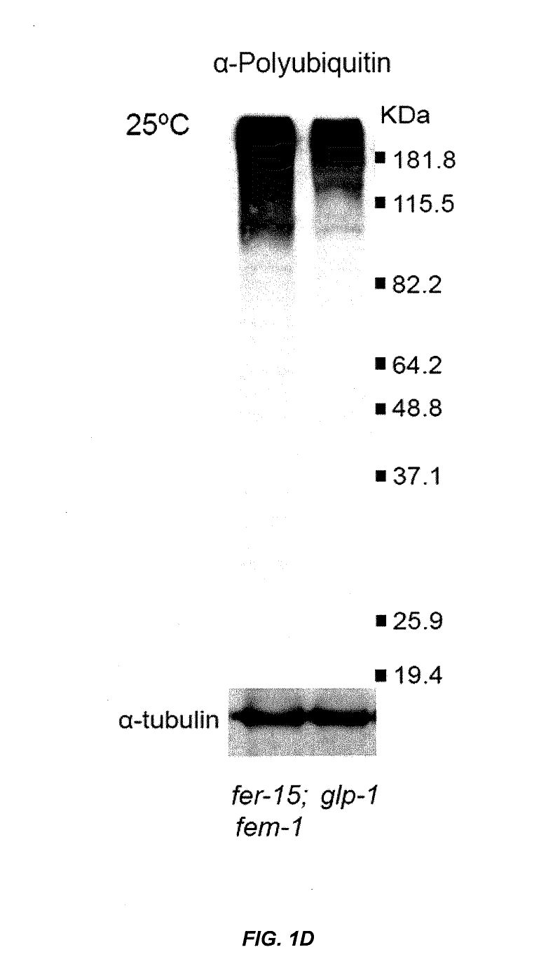

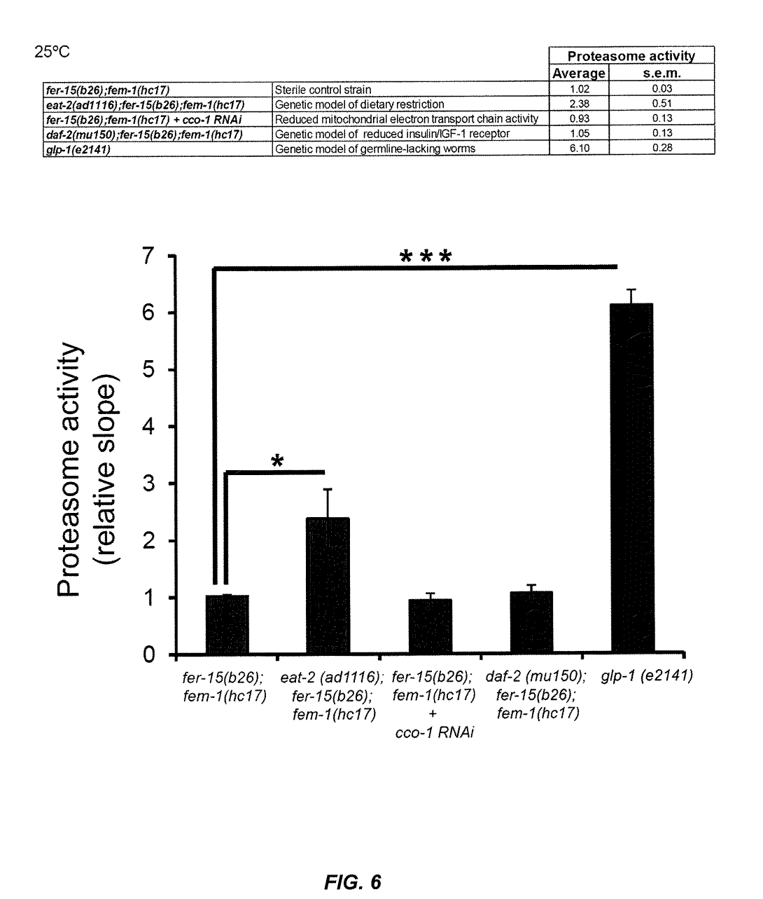

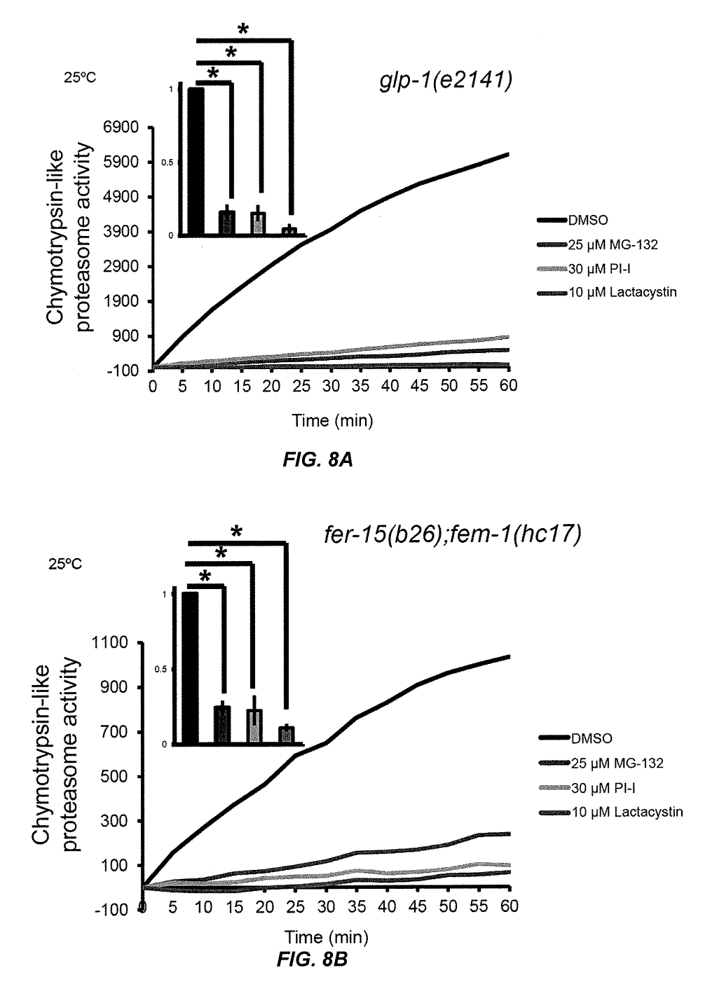

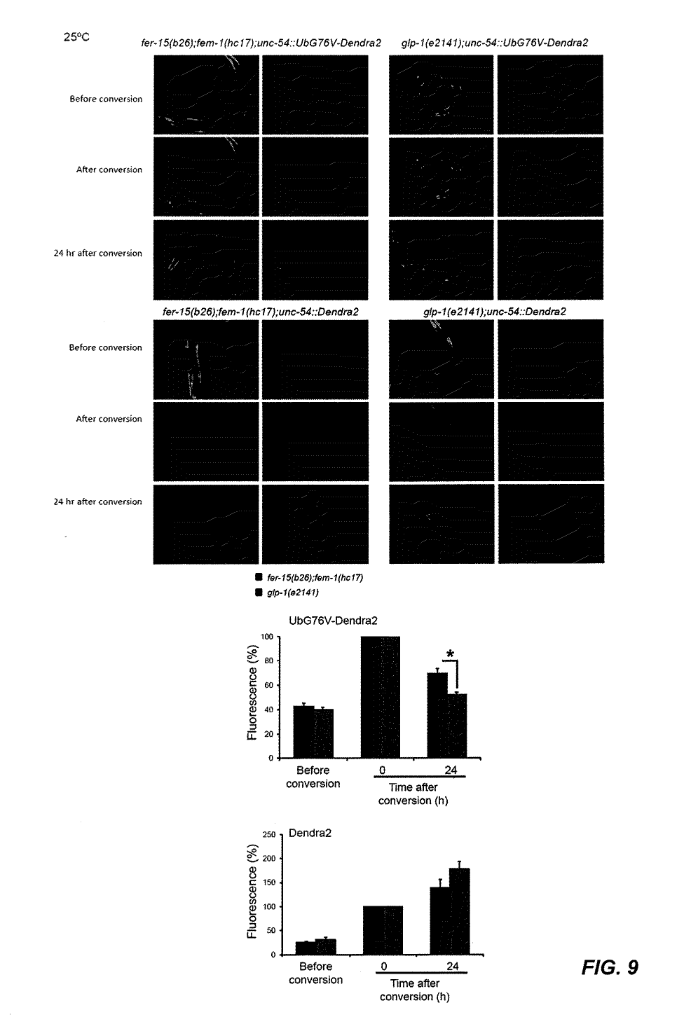

Applicants examined the activity of the 26S/30S proteasome in several long-lived mutants, using a fluorogenic peptide substrate specific for the chymotrypsin-like activity of the proteasome (FIG. 6). Intriguingly, Applicants found that glp-1(e2141) mutant worms, which lack their germline, displayed a dramatic, over 6-fold increase, in the chymotrypsin-like proteasome activity (FIG. 1a and FIG. 6, 7a-c). The proteasome inhibitors MG-132, lactacystin and PI-I blocked activity from extracts in both the glp-1 mutant and the control strain (FIG. 8), indicating that indeed the increased peptidase activity in glp-1(e2141) worms was due to the proteasome. The caspase-like and trypsin-like activities were also increased in glp-1 mutant worms (FIG. 1b, c). Additionally, Applicants found that a C. elegans genetic model of dietary restriction [Lakowski, B. & Hekimi, S. The genetics of caloric restriction in Caenorhabditis elegans. Proceedings of the National Academy of Sciences of the United States of America 95, 13091-13096 (1998)] also induced proteasome activity, although to a lesser extent than the glp-1(e2141) mutation (FIG. 6). In contrast, and rather surprisingly, neither reduced IIS signaling by mutation of daf-2 nor reduced mitochondrial electron transport chain (ETC) activity [Dillin, A., et al. Rates of behavior and aging specified by mitochondrial function during development. Science 298, 2398-2401 (2002)] up-regulated proteasome activity (FIG. 6). Consistent with increased proteasome activity in glp-1(e2141) worms, Applicants observed decreased levels of polyubiquitinylated proteins in these worms (FIG. 1d). To further examine UPS activity in living animals, Applicants used a photoconvertible fluorescent UPS reporter system for live imaging and quantification of protein degradation in C. elegans [Hamer, G., Matilainen, O. & Holmberg, C. I. A photoconvertible reporter of the ubiquitin-proteasome system In vivo. Nat Methods 7, 473-478 (2010)]. This proteasome reporter consists of the photoconvertible fluorescent protein, Dendra2, targeted for proteasomal degradation by fusion to a mutant form of ubiquitin (UbG76V) that cannot be cleaved by ubiquitin hydrolases. Dendra2 can be irreversibly photoconverted from a green to a red fluorescent state, providing quantification of UPS activity independently of protein synthesis. Applicants found that this reporter is degraded more rapidly in glp-1 mutant animals compared to control strains, while Dendra2 lacking the UbG76V signal remained stable (FIG. 9).

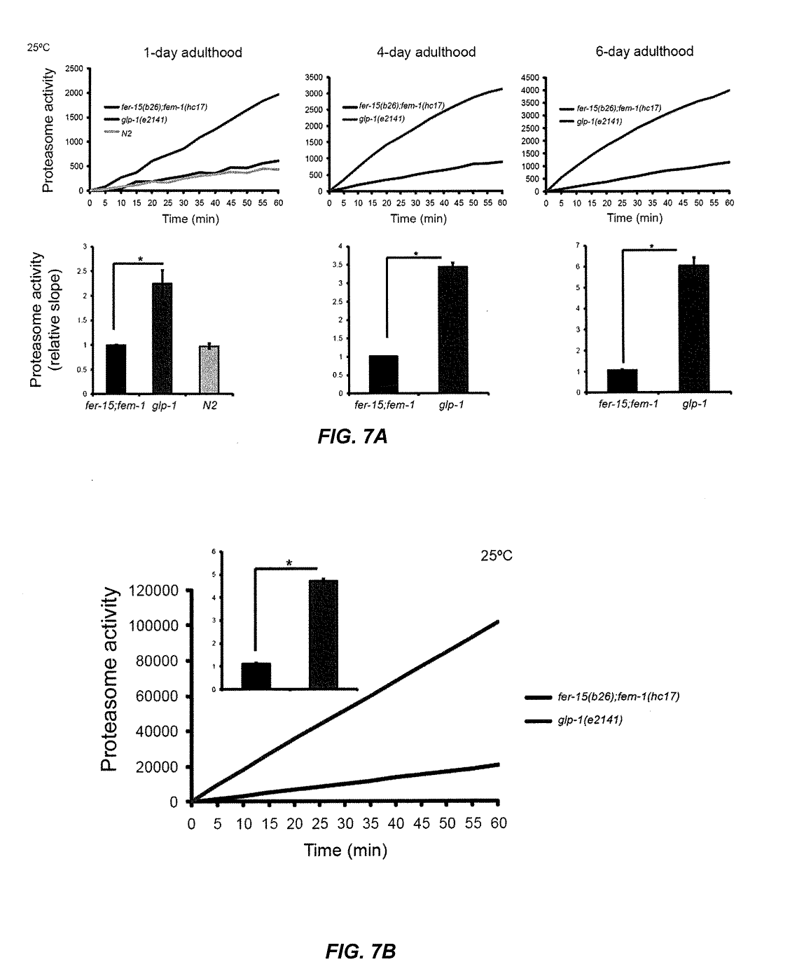

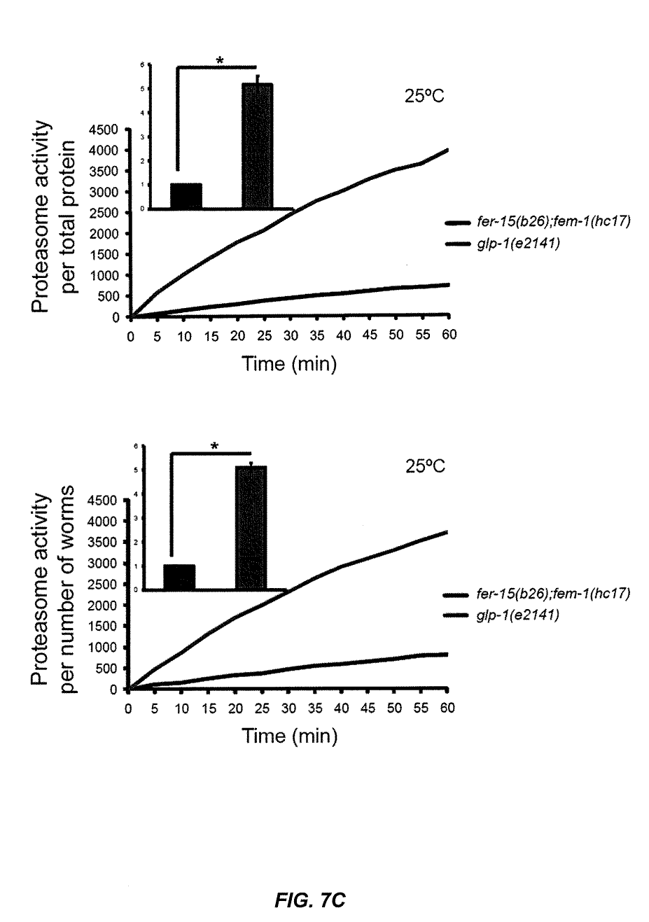

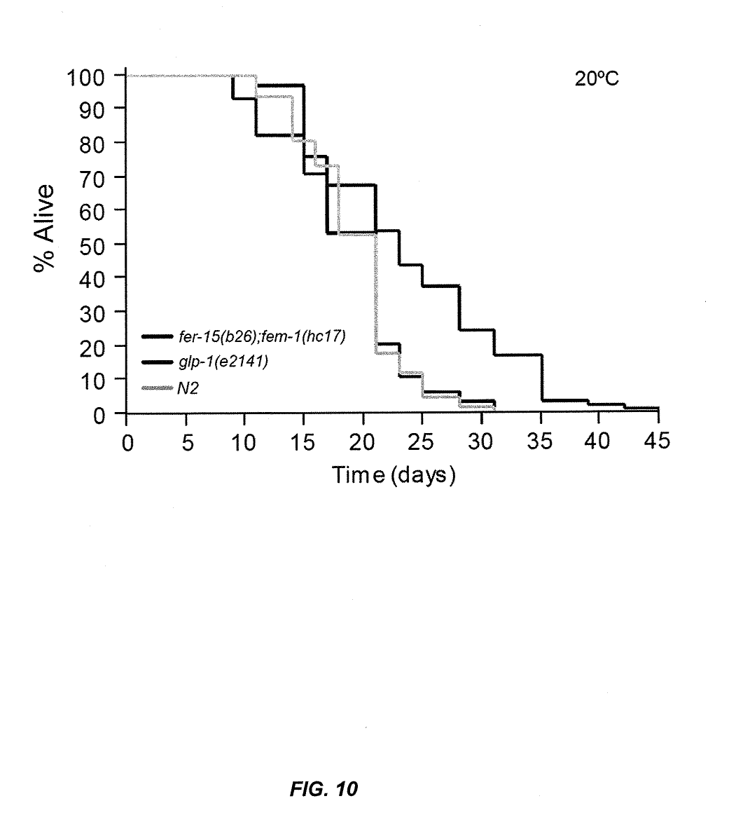

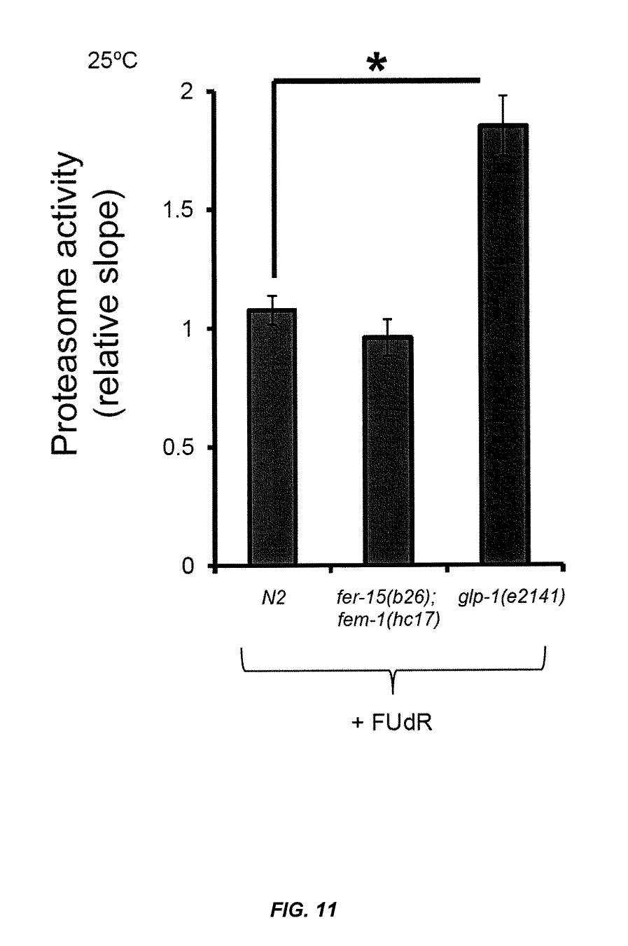

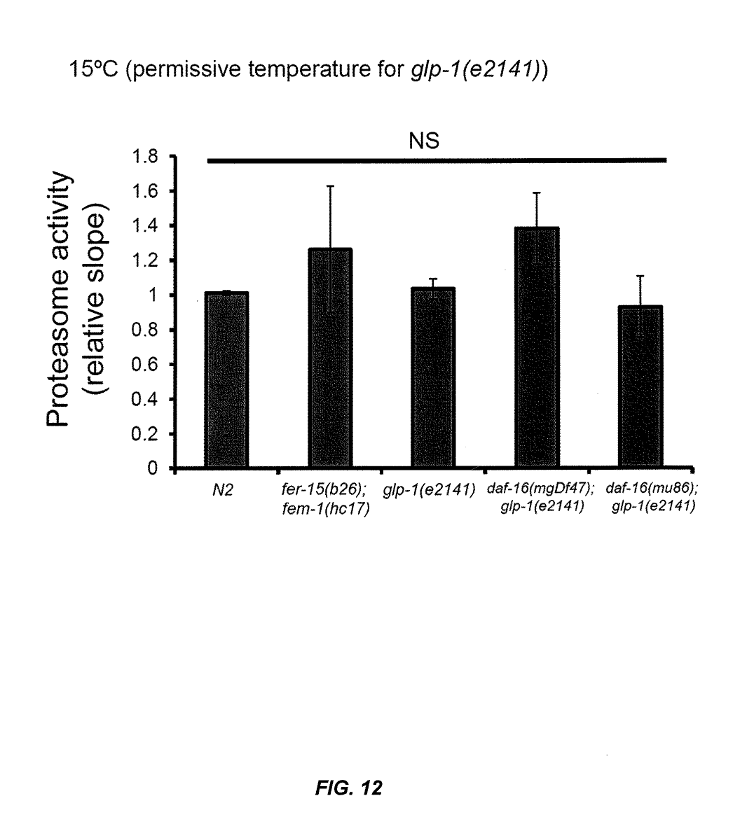

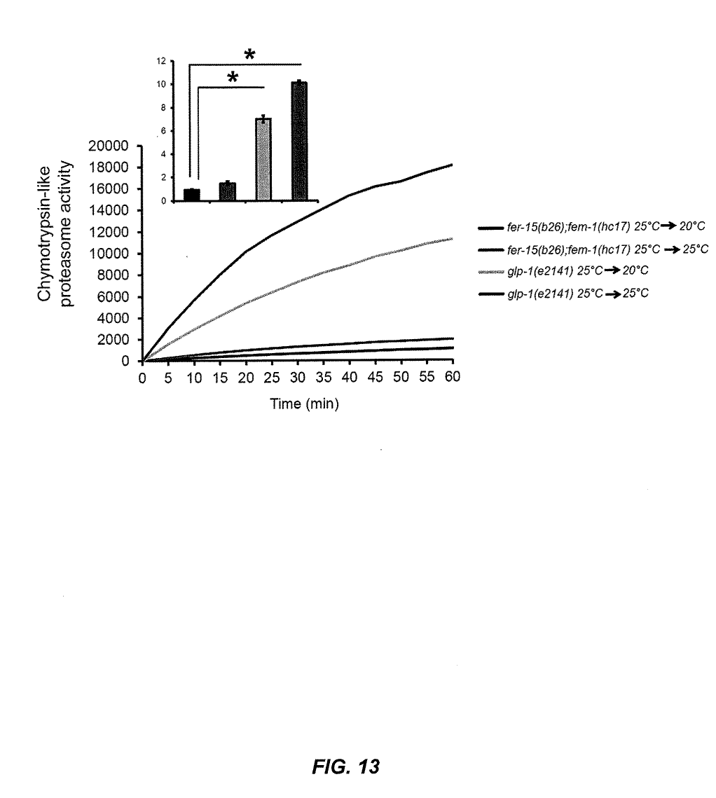

glp-1(e2141) worms differ from other types of reproductive mutants in that their entire germline is missing. Importantly, glp-1 mutants exhibit a significantly increased lifespan in comparison to worms that are also sterile but which still contain a proliferating germline (FIG. 10). Applicants observed that the increased proteasome activity of glp-1 mutants was not due to a benefit of sterility per se because the normal-lived, sterile control fer-15(b26);fem-1(hc17) animals have similar proteasome activity as wild-type animals (FIG. 7a, 11). Furthermore, treatment with 5-fluoro-2′ deoxyuridine (FUdR), a drug used to block progeny production in worms [Mitchell, D. H., Stiles, J. W., Santelli, J. & Sanadi, D. R. Synchronous growth and aging of Caenorhabditis elegans in the presence of fluorodeoxyuridine. Journal of gerontology 34, 28-36 (1979)], did not affect proteasome activity (FIG. 11). The glp-1(e2141) allele is temperature-sensitive for reproduction and longevity [Priess, J. R., Schnabel, H. & Schnabel, R. The glp-1 locus and cellular interactions in early C. elegans embryos. Cell 51, 601-611 (1987)]; these worms are only long-lived when they are shifted to restrictive temperature (25° C.) either during development or in early adulthood [Arantes-Oliveira, N., Apfeld, J., Dillin, A. & Kenyon, C. Regulation of life-span by germ-line stem cells in Caenorhabditis elegans. Science 295, 502-505 (2002)]. Accordingly, proteasome activity is not increased in glp-1(e2141) worms grown continuously at the permissive temperature (FIG. 12). However, resembling the longevity phenotype [Arantes-Oliveira, N., Apfeld, J., Dillin, A. & Kenyon, C. Regulation of life-span by germ-line stem cells in Caenorhabditis elegans. Science 295, 502-505 (2002)], glp-1(e2141) worms maintained high proteasome activity when down shifted to a permissive temperature after germline removal (FIG. 13). These results indicate that different forms of sterility do not have similar effects on proteasome activity, but are specific to loss of the germline.

daf-16 Regulates Proteasome Activity

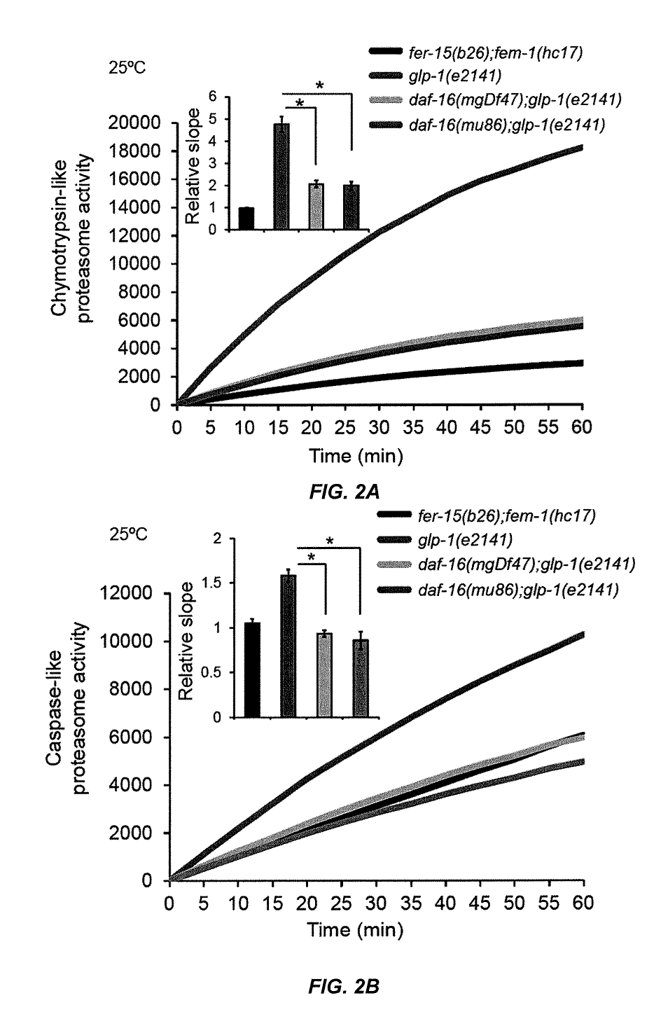

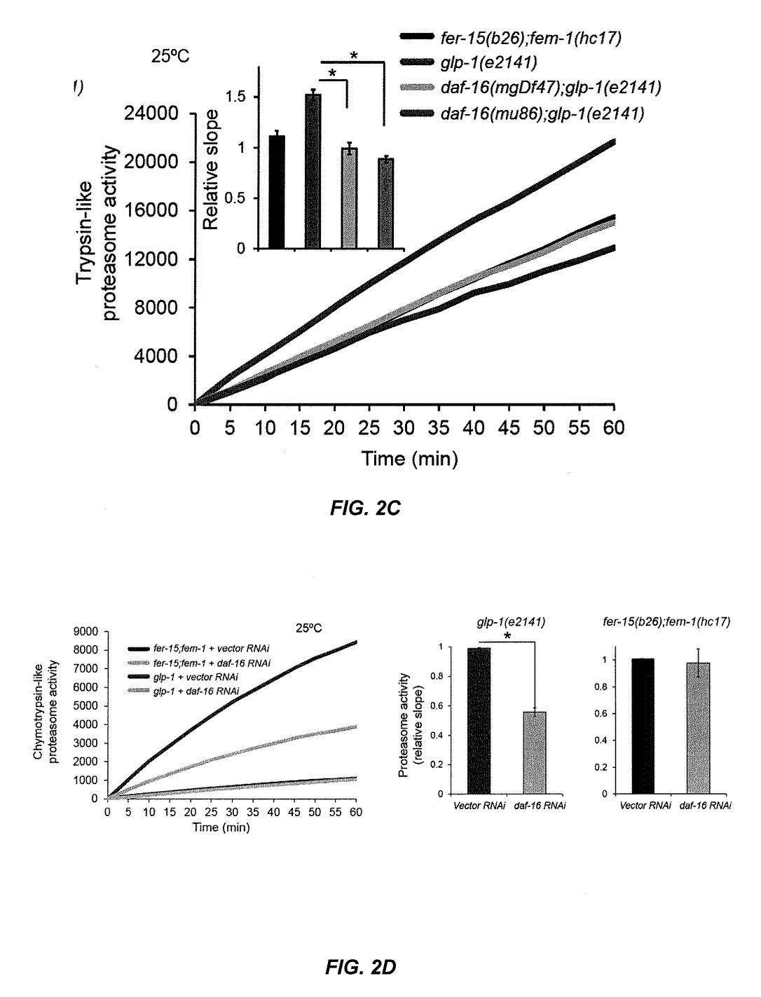

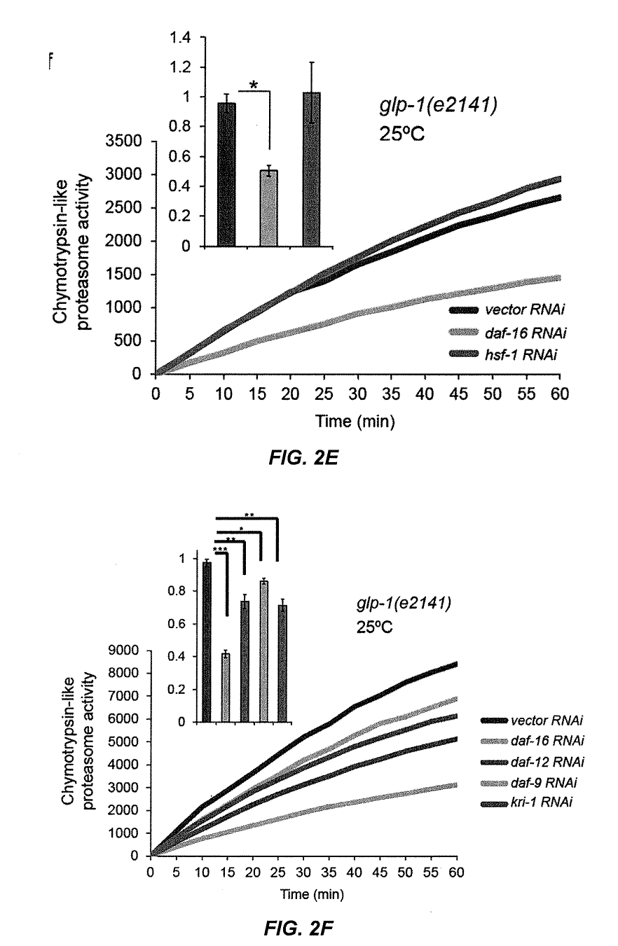

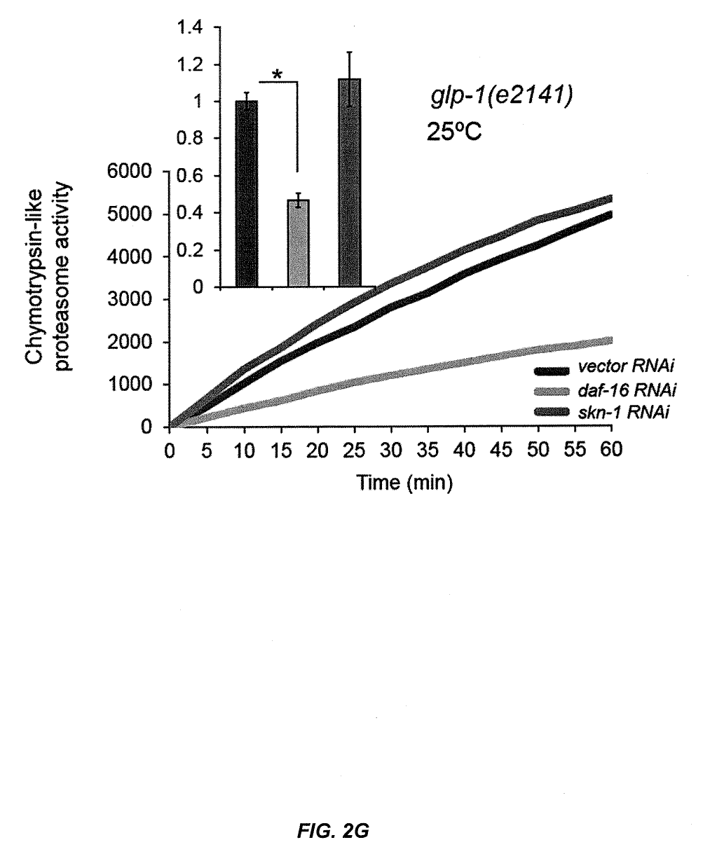

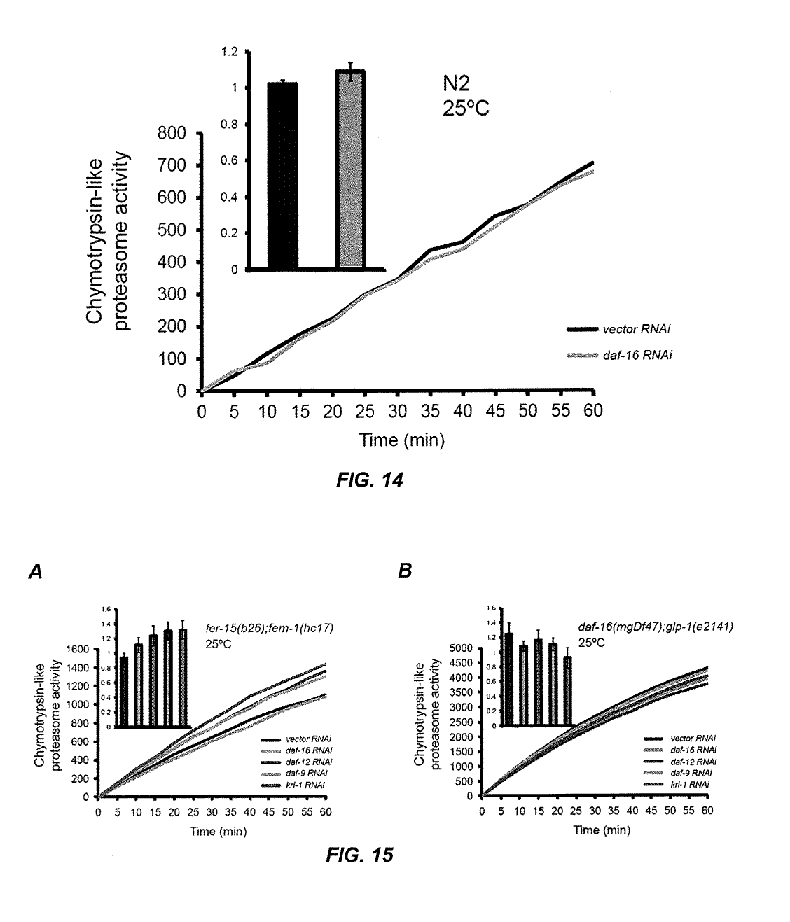

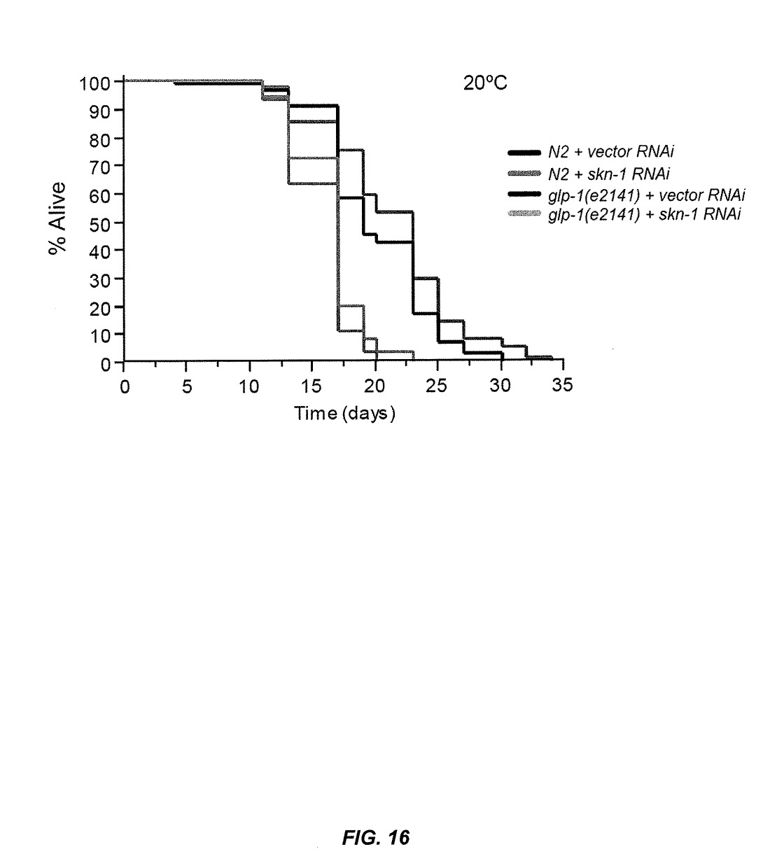

Because DAF-16, the worm FOXO transcription factor, is essential for the increased longevity of glp-1 mutant worms [Arantes-Oliveira, N., Apfeld, J., Dillin, A. & Kenyon, C. Regulation of life-span by germ-line stem cells in Caenorhabditis elegans. Science 295, 502-505 (2002)], Applicants tested whether daf-16 was also required for the increased proteasome activity found in glp-1(e2141) animals. Proteasome activity of glp-1 mutant animals was suppressed to wild-type levels in daf-16;glp-1 double mutant animals (FIG. 2a-c). daf-16 is required during reproductive adulthood to modulate the aging process in worms [Dillin, A., Crawford, D. K. & Kenyon, C. Timing requirements for insulin/IGF-1 signaling in C. elegans. Science 298, 830-834 (2002)]. Accordingly, daf-16 RNAi treatment of glp-1(e2141) animals during adulthood decreased proteasome activity (FIG. 2d). In contrast, daf-16 RNAi did not affect proteasome activity in control strains (FIG. 2d and FIG. 14), where DAF-16 is located in the cytosol and inactive. Loss of daf-16 suppressed longevity and proteasome activity, but not the reproductive phenotype of glp-1(e2141) worms, providing further evidence that increased proteasomal activity could not be separated from the increased longevity mediated by daf-16 in glp-1 mutants. Applicants examined whether other genes required to promote DAF-16/FOXO nuclear localization in the germline longevity pathway, daf-12, daf-9 and kri-1, were also necessary for increased proteasome activity. Accordingly, reduction of any one of these genes in glp-1(e2141) worms resulted in decreased proteasome activity, although not to the extent of daf-16 reduction (FIG. 2e). Furthermore, reduction of either daf-12, daf-9 or kri-1 did not further decrease proteasome activity of the daf-16;glp-1 double mutant animals or the control strain (FIG. 15a, b). In addition to daf-16, glp-1 mediated longevity requires two additional transcription factors, hsf-1 [Hansen, M., Hsu, A. L., Dillin, A. & Kenyon, C. New genes tied to endocrine, metabolic, and dietary regulation of lifespan from a Caenorhabditis elegans genomic RNAi screen. PLoS genetics 1, 119-128 (2005)] and skn-1 (FIG. 16). hsf-1 is required for the regulation of adult lifespan, heat-shock and proteotoxic stress [Cohen, E., Bieschke, J., Perciavalle, R. M., Kelly, J. W. & Dillin, A. Opposing activities protect against age-onset proteotoxicity. Science 313, 1604-1610 (2006); Hsu, A. L., Murphy, C. T. & Kenyon, C. Regulation of aging and age-related disease by DAF-16 and heat-shock factor. Science 300, 1142-1145 (2003)]. skn-1 is the worm orthologue of nrf-2 and plays a central role in oxidative stress responses in worms, flies and mice [Alam, J., et al. Nrf2, a Cap'n'Collar transcription factor, regulates induction of the heme oxygenase-1 gene. The Journal of biological chemistry 274, 26071-26078 (1999); An, J. H. & Blackwell, T. K. SKN-1 links C. elegans mesendodermal specification to a conserved oxidative stress response. Genes Dev 17, 1882-1893 (2003)]. In contrast to daf-16, daf-12, daf-9 and kri-1,

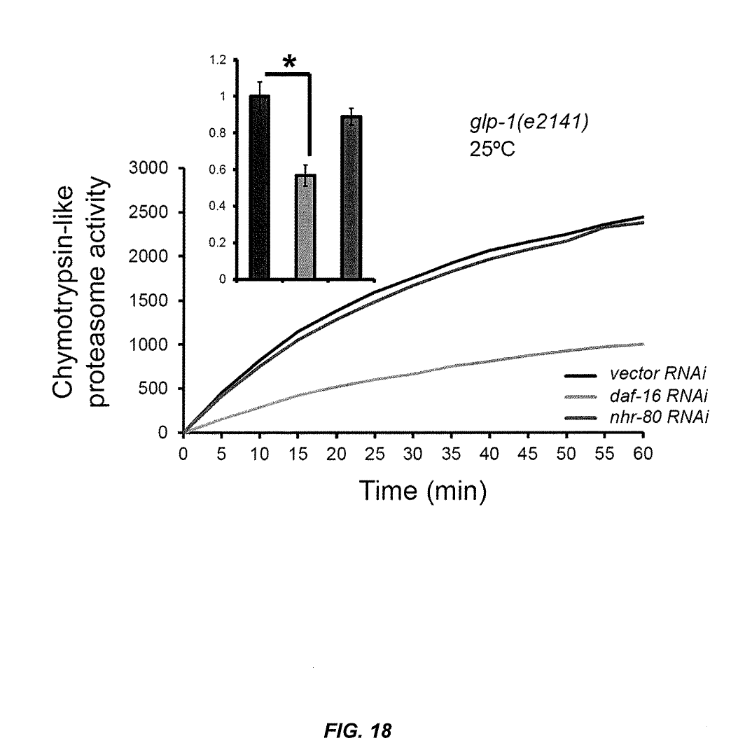

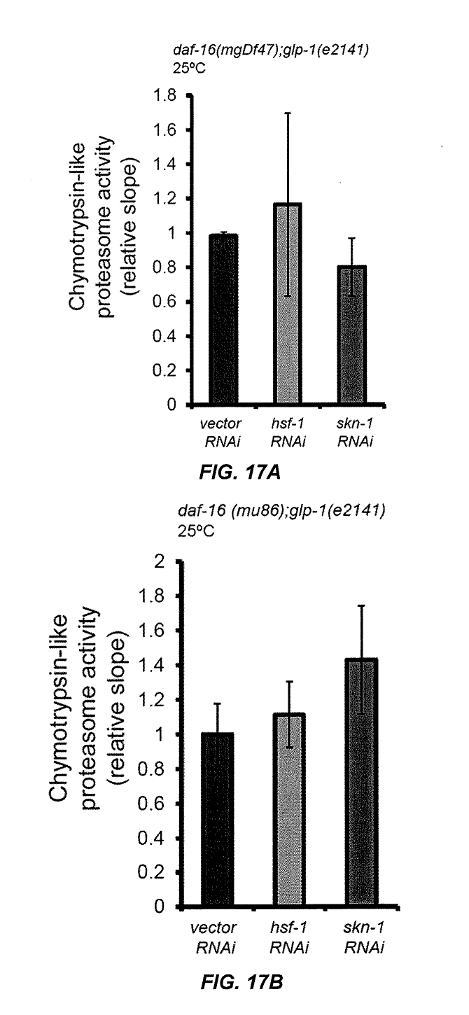

Applicants observed that neither hsf-1 nor skn-1 were required for the increased proteasome activity in glp-1(e2141) animals (FIG. 2f, g). In order to uncover a possible redundancy of either hsf-1 or skn-1 with daf-16, Applicants knocked down these factors in the daf-16;glp-1 double mutant animals. However, in the context of daf-16 loss, neither hsf-1 nor skn-1 further affected proteasome activity in glp-1(e2141) worms (FIG. 17). The nuclear hormone receptor nhr-80 links fatty acid desaturation to lifespan extension through germline ablation in a daf-16 independent manner [Goudeau, J., et al. Fatty acid desaturation links germ cell loss to longevity through NHR-80/HNF4 in C. elegans. PLoS biology 9, e1000599 (2011)]. Consistent with a requirement for daf-16 in proteasome activity, nhr-80 was not required for increased proteasome activity in glp-1 mutant worms (FIG. 18). Taken together, alterations that specifically affect daf-16 activity, but not hsf-1, skn-1 or nhr-80, alter proteasome activity in glp-1 mutants, suggesting that a major output for daf-16 mediated longevity in this mutant is to increase proteasome activity.

daf-16 Regulates rpn-6.1 Levels

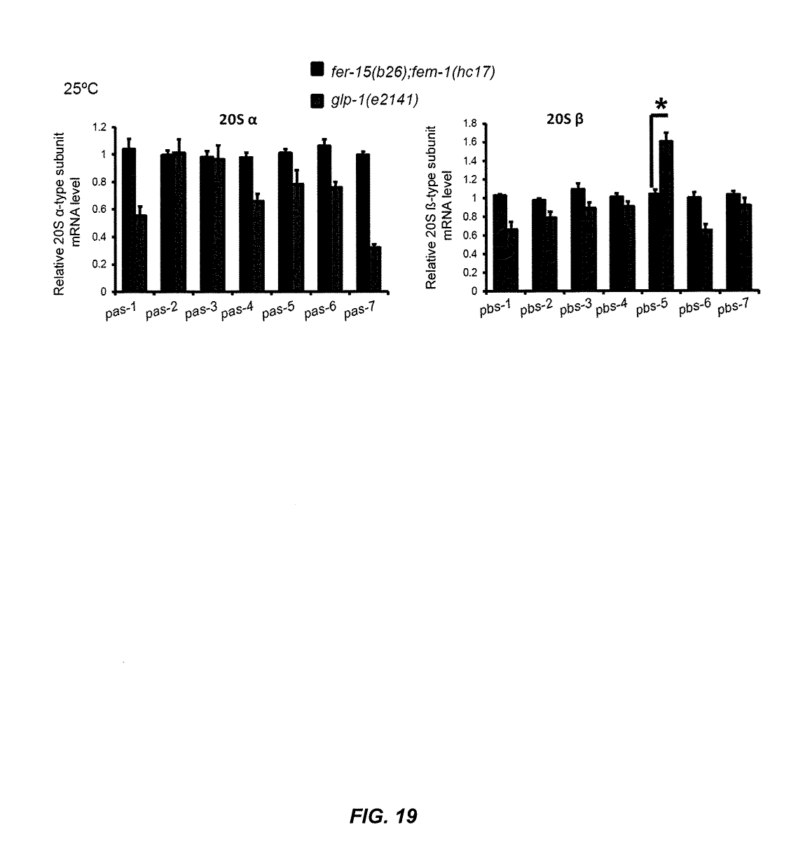

The 26S/30S proteasome consists of a 20S core structure that contains the proteolytic active sites and 19S cap structures that impart regulation on the activity of the holo-complex (26S, single capped and 30S, double capped) [Finley, D. Recognition and processing of ubiquitin-protein conjugates by the proteasome. Annual review of biochemistry 78, 477-513 (2009)]. Although 20S particles can exist in a free form, 20S particles in their most physiological form are inactive, unable to degrade denatured proteins or cleave peptides [Kisselev, A. F. & Goldberg, A. L. Monitoring activity and inhibition of 26S proteasomes with fluorogenic peptide substrates. Methods in enzymology 398, 364-378 (2005)]. The 19S regulatory subunit is responsible for stimulating the 20S proteasome to degrade proteins, since ATPases of the regulatory particle open the 20S core, allowing substrates access to proteolytic active sites [Kohler, A., et al. The axial channel of the proteasome core particle is gated by the Rpt2 ATPase and controls both substrate entry and product release. Molecular cell 7, 1143-1152 (2001)]. Analysis of the mRNA levels of the 20S proteasome subunits revealed that α-subunits were not increased in glp-1 mutants whereas only one of the β-subunits, pbs-5, was moderately increased (FIG. 19 and Table 1). PBS-5 is the 13-type subunit that contains the chymotrypsin-like proteolytic active site [Finley, D. Recognition and processing of ubiquitin-protein conjugates by the proteasome. Annual review of biochemistry 78, 477-513 (2009)].

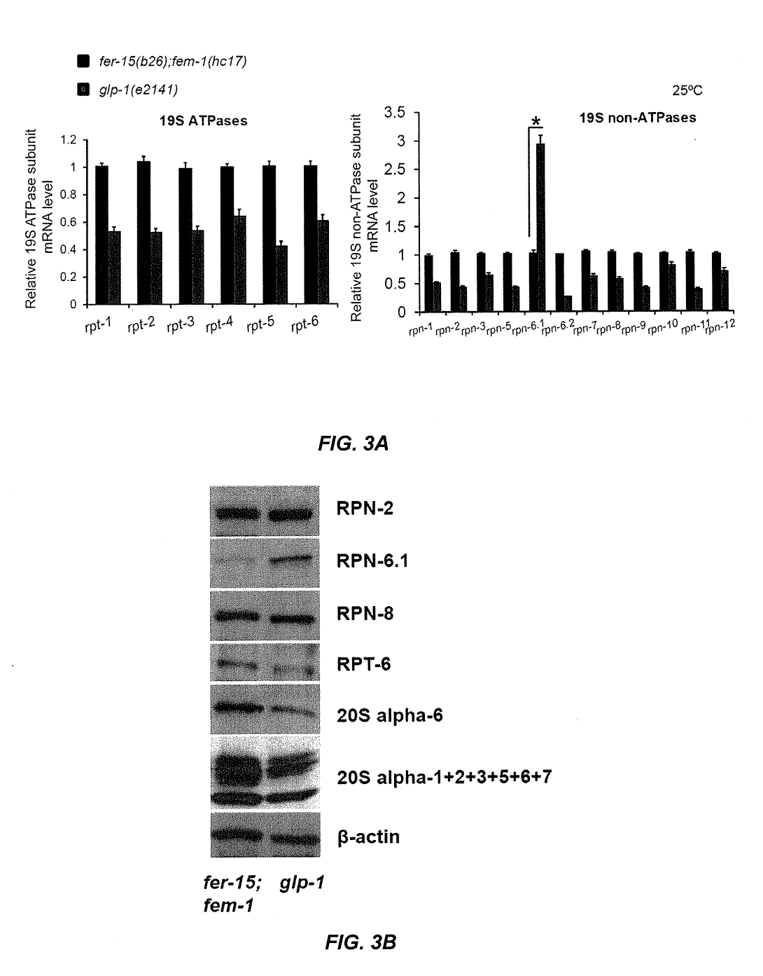

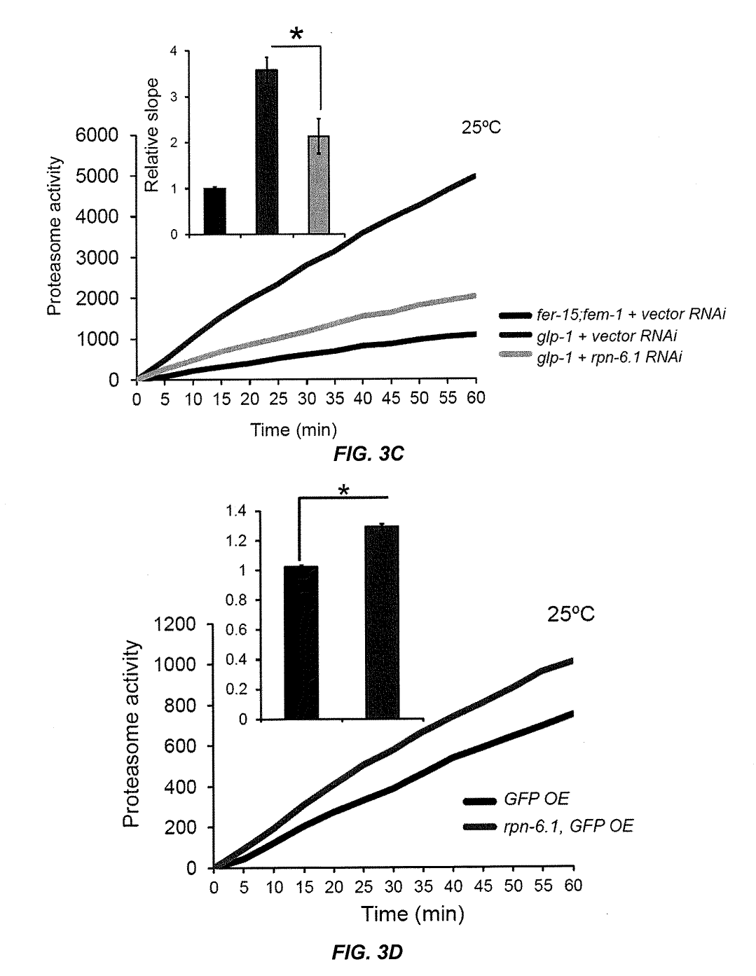

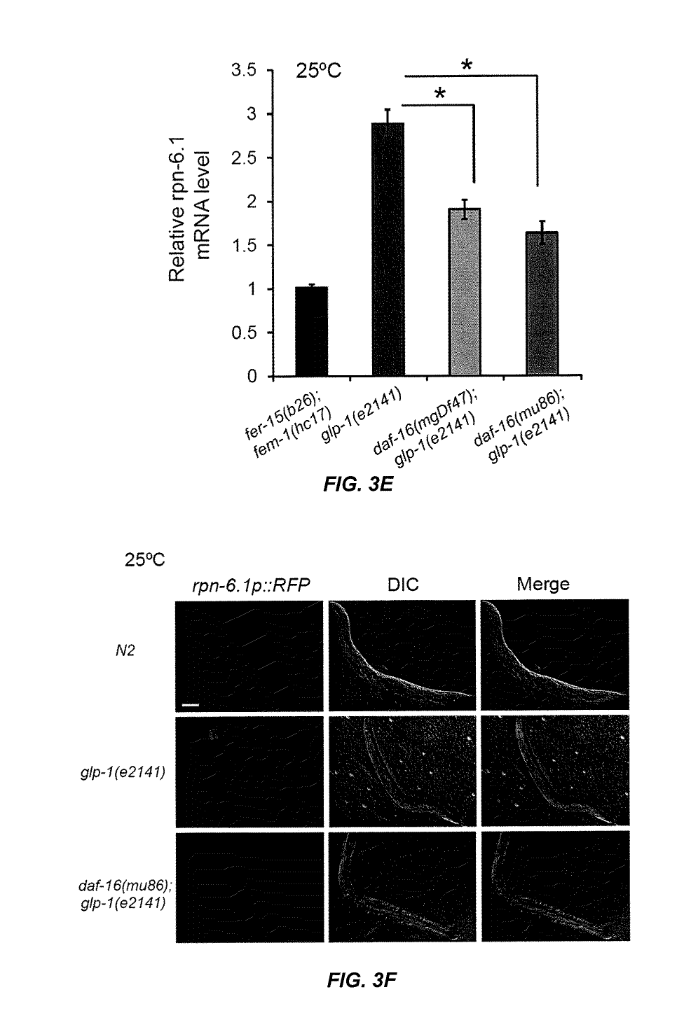

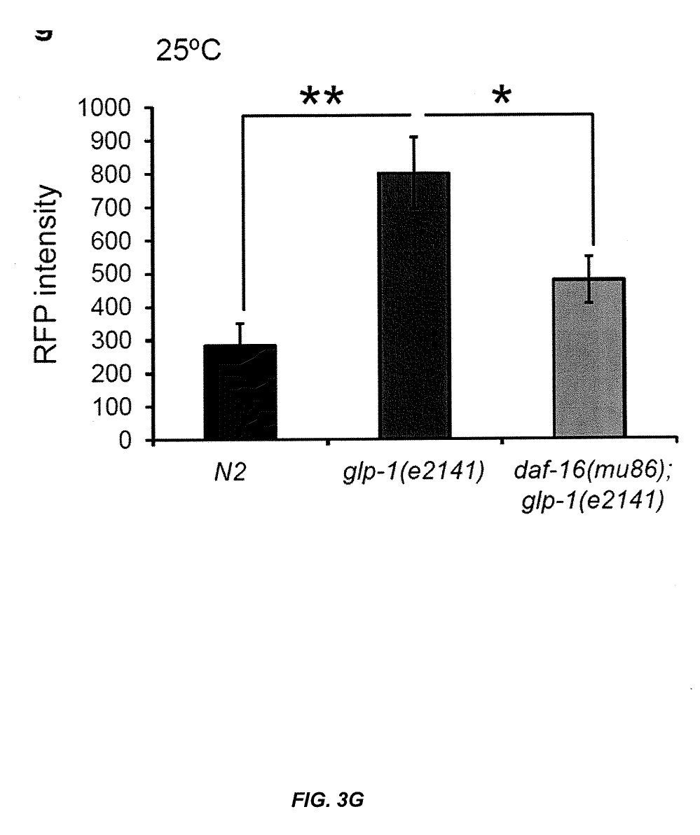

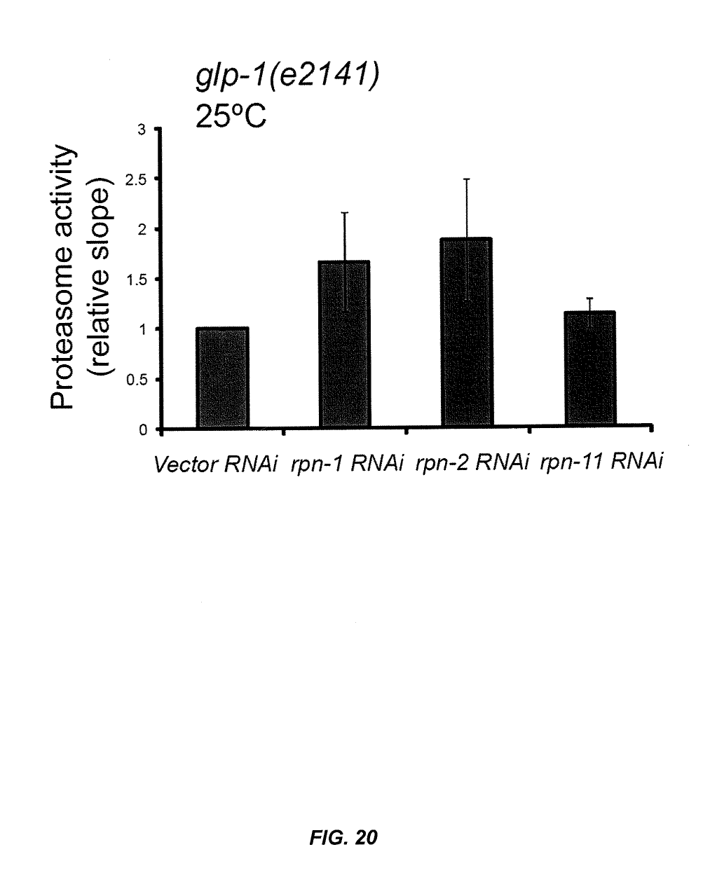

With regard to the 19S proteasome subunits, Applicants did not detect an increase of the ATPase subunits (FIG. 3a and Table 1). Notably, only one of the non-ATPase subunits was increased in glp-1 mutant animals: rpn-6.1, an essential subunit for the activity of the 26S/30S proteasome that stabilizes the otherwise weak interaction between the 20S core and the 19S cap [Pathare, G. R., et al. The proteasomal subunit Rpn6 is a molecular clamp holding the core and regulatory subcomplexes together. Proceedings of the National Academy of Sciences of the United States of America 109, 149-154 (2012); Santamaria, P. G., Finley, D., Ballesta, J. P. & Remacha, M. Rpn6p, a proteasome subunit from Saccharomyces cerevisiae, is essential for the assembly and activity of the 26 S proteasome. The Journal of biological chemistry 278, 6687-6695 (2003)] (FIG. 3a, b and Table 1). The closely related rpn-6.2 was not increased in glp-1 mutants (FIG. 3a and Table 1). rpn-6.1 had a 3-fold increase in its expression in glp-1 mutants and was by far the most increased of all subunits. Accordingly, knock-down of rpn-6.1 dramatically decreased proteasome activity in glp-1(e2141) animals (FIG. 3c) similar to loss of daf-16. In contrast, loss of other non-ATPase subunits did not affect proteasome activity of glp-1 mutants (FIG. 20). Knock-down of rpn-6.1 induced an up-regulation in the expression of the rest of the 26S proteasome subunits, likely to compensate for the reduction in proteasome activity induced by decreased levels of this critical subunit (Table 2). Moreover, over-expression of rpn-6.1 in wild-type animals was sufficient to increase proteasome activity (FIG. 3d). The increase in rpn-6.1 levels did not alter the expression of other 26S proteasome subunits (Table 3). Taken together, rpn-6.1 appears to be a key component required for activation of the proteasome machinery of the germline-lacking nematodes.

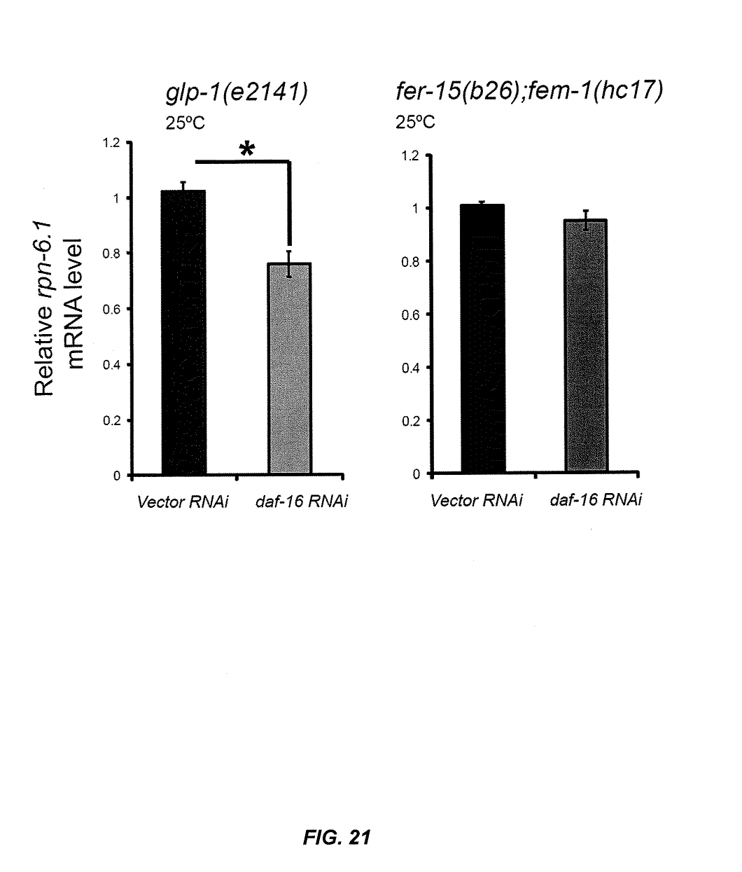

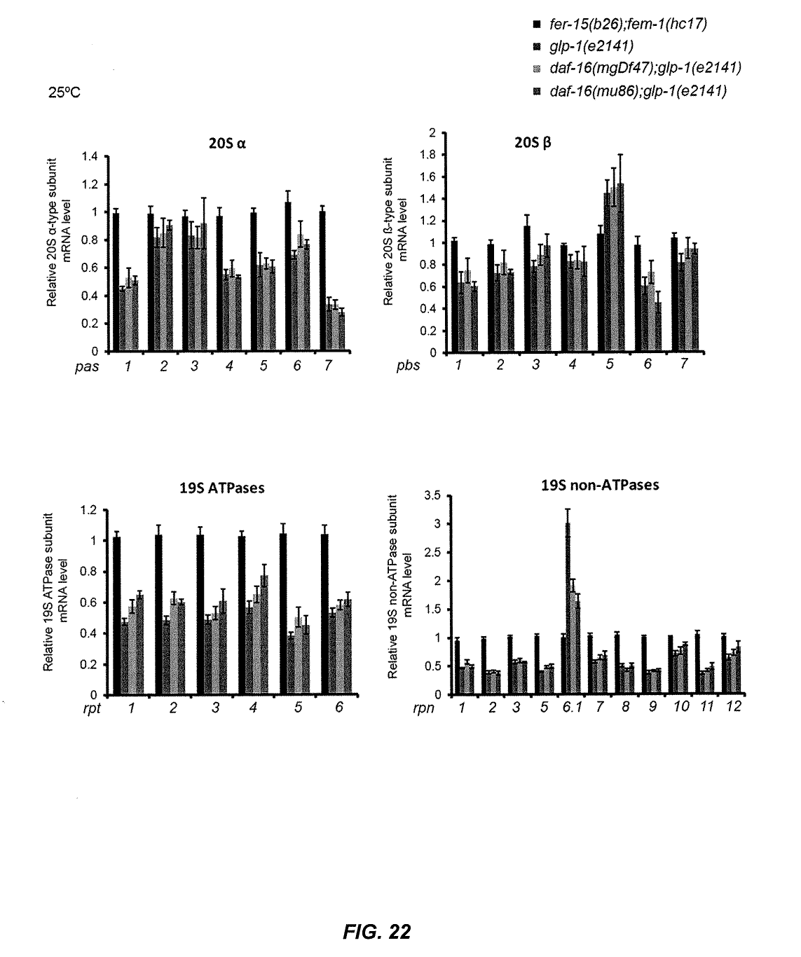

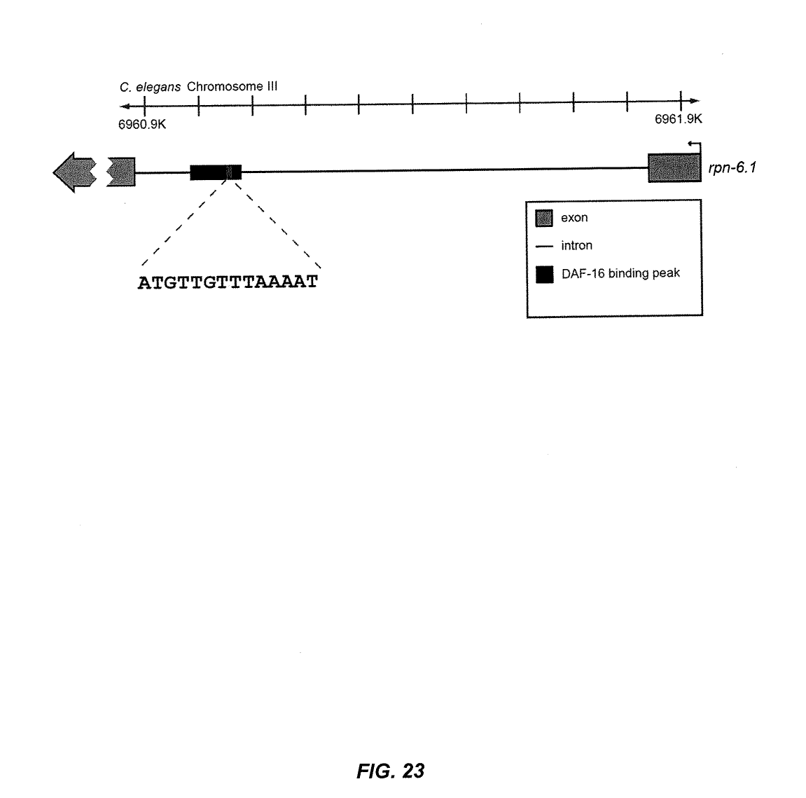

Applicants found DAF-16 necessary for the increased expression of rpn-6.1 by analyzing its mRNA levels in both daf-16;glp-1 double mutants and daf-16 RNAi-treated animals (FIG. 3e, FIG. 21, 22 and Table 4). Notably, other proteasome subunits, including pbs-5, were not decreased by loss of daf-16 in glp-1 mutant worms (FIG. 22). daf-16 RNAi did not change rpn-6.1 expression in control worms (FIG. 21). Therefore, these results display a correlation between daf-16 activity, rpn-6.1 levels and proteasome activity. Consistent with DAF-16 regulating the expression of rpn-6.1, Applicants identified a potential DAF-16 binding site [Furuyama, T., Nakazawa, T., Nakano, I. & Mori, N. Identification of the differential distribution patterns of mRNAs and consensus binding sequences for mouse DAF-16 homologues. Biochem J 349, 629-634 (2000)] within the first intron of rpn-6.1 (FIG. 23). This site is supported by a DAF-16 binding region defined by the modENCODE project [Celniker, S. E., et al. Unlocking the secrets of the genome. Nature 459, 927-930 (2009)], indicating that rpn-6.1 is likely to be a direct DAF-16 target. To further explore rpn-6.1 transcriptional regulation, Applicants generated a transcriptional reporter construct. Applicants found rpn-6.1 expressed in the pharynx and posterior intestine in control worms. Notably, rpn-6.1 expression increased dramatically in the pharynx and throughout the intestine of glp-1 mutants. In daf-16;glp-1 double mutants, Applicants found almost a 2-fold decreased expression of rpn-6.1 compared to glp-1(e2141) worms although rpn-6.1 expression is still increased compared to wild-type worms (FIG. 3f, g). These results correlated with qPCR data indicating that daf-16 mutations decrease rpn-6.1 expression in glp-1 mutants, but not to control levels (FIG. 3e, g), suggesting that a potential additional factor may be necessary for rpn-6.1 expression in addition to DAF-16.

rpn-6.1 Determines Stress Resistance

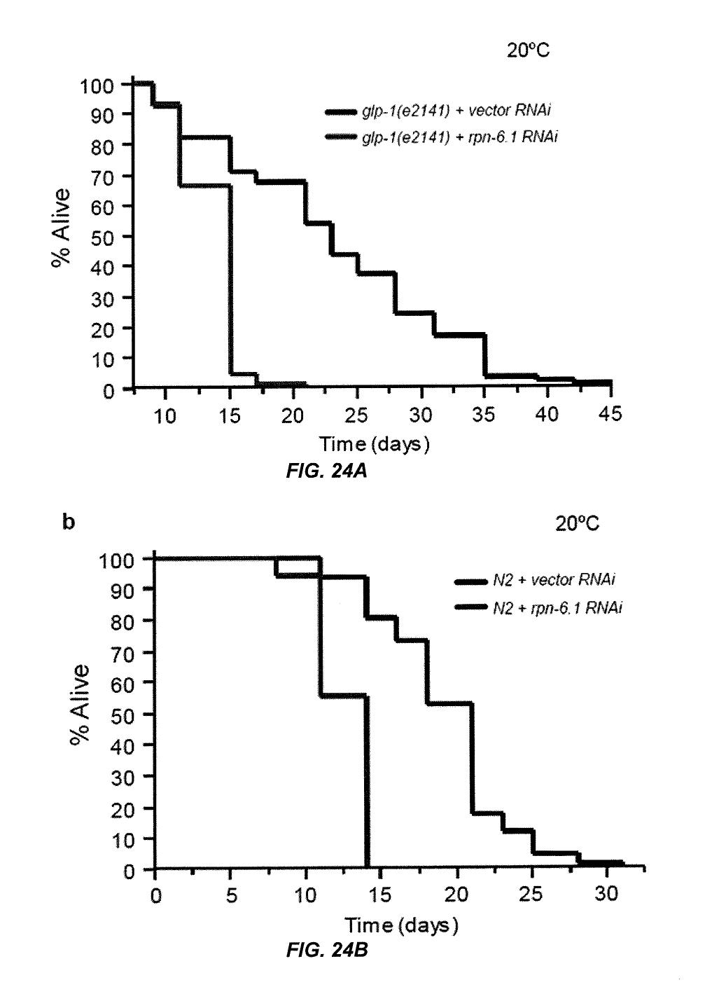

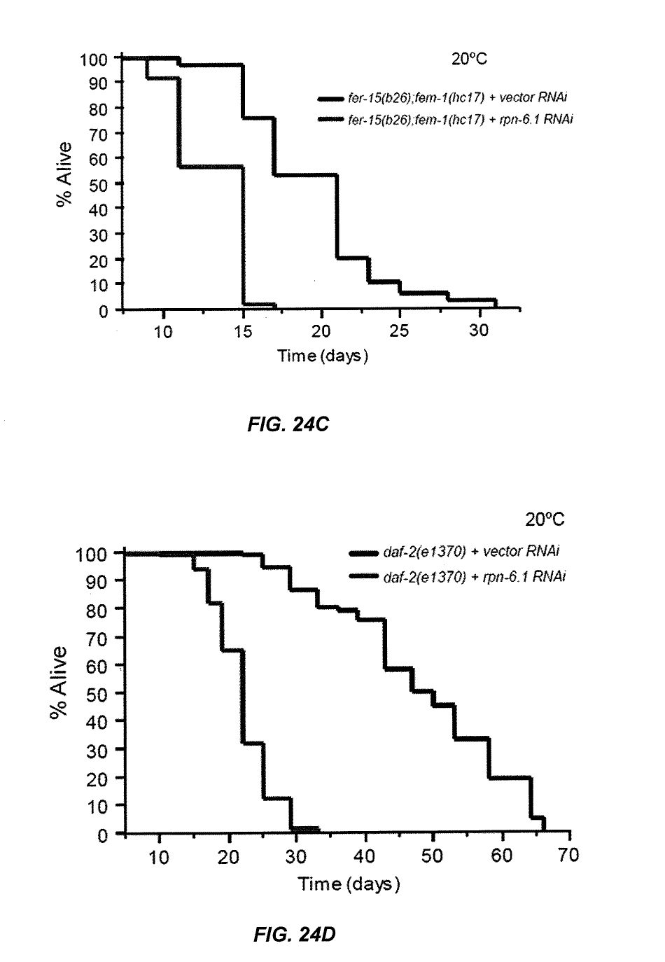

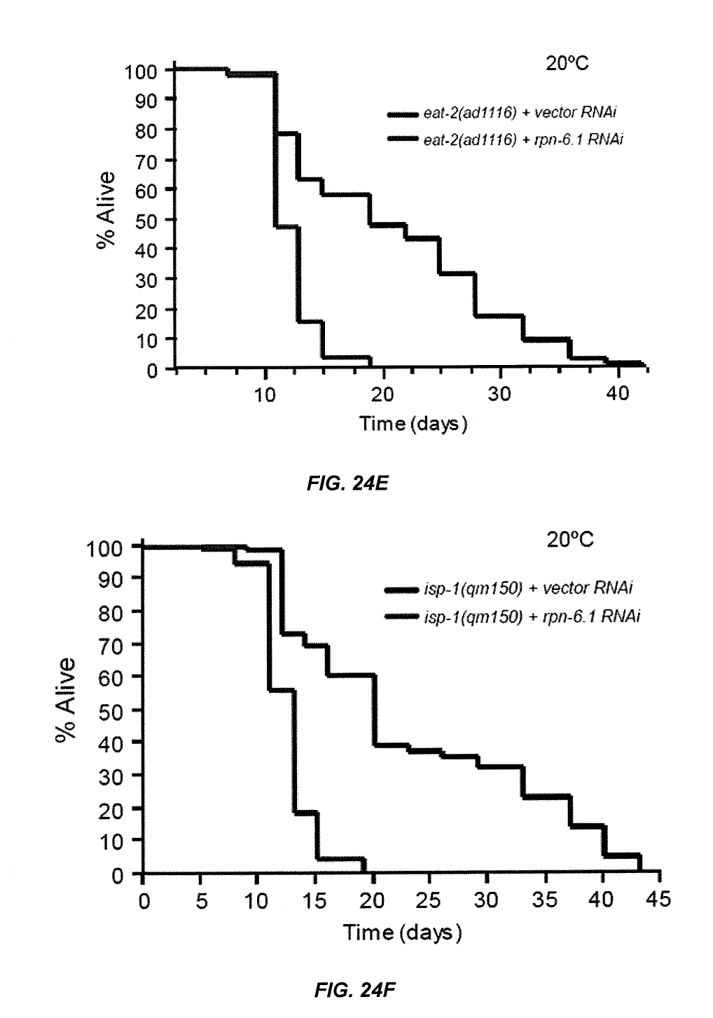

With the strong connection between daf-16, a key ageing modulator, rpn-6.1, a key proteasomal factor, increased proteasome activity in long-lived glp-1 mutant animals dependent upon both daf-16 and rpn-6.1, Applicants asked what role, if any, does rpn-6.1 play in longevity. To assess the requirement for rpn-6.1 during lifespan, Applicants conducted RNAi knock-down of this gene in C. elegans. Because proteasomal function is required during larval development [Ghazi, A., Henis-Korenblit, S. & Kenyon, C. Regulation of Caenorhabditis elegans lifespan by a proteasomal E3 ligase complex. Proceedings of the National Academy of Sciences of the United States of America 104, 5947-5952 (2007)], Applicants initiated rpn-6.1 RNAi treatment during adulthood, the time at which daf-16 is required for longevity assurance [Dillin, A., Crawford, D. K. & Kenyon, C. Timing requirements for insulin/IGF-1 signaling in C. elegans. Science 298, 830-834 (2002)]. Knock-down of rpn-6.1 substantially decreased the lifespan of glp-1 mutant animals (FIG. 24). However, loss of rpn-6.1 also decreased lifespan of both wild-type and sterile control animals (fer-15(b26); fem-1(hc17)). Besides germ-cell loss, Applicants also examined rpn-6.1 RNAi effects on other pathways that influence lifespan such as reduced IIS (daf-2(e1370) worms), reduced mitochondrial ETC (isp-1 (qm150) worms) and reduced food intake (eat-2(ad1116) worms). In all cases, knock-down of rpn-6.1 substantially decreased lifespan, confirming that this gene is essential for viability of adult animals, making lifespan analysis by rpn-6.1 loss of function difficult to interpret (FIG. 24).

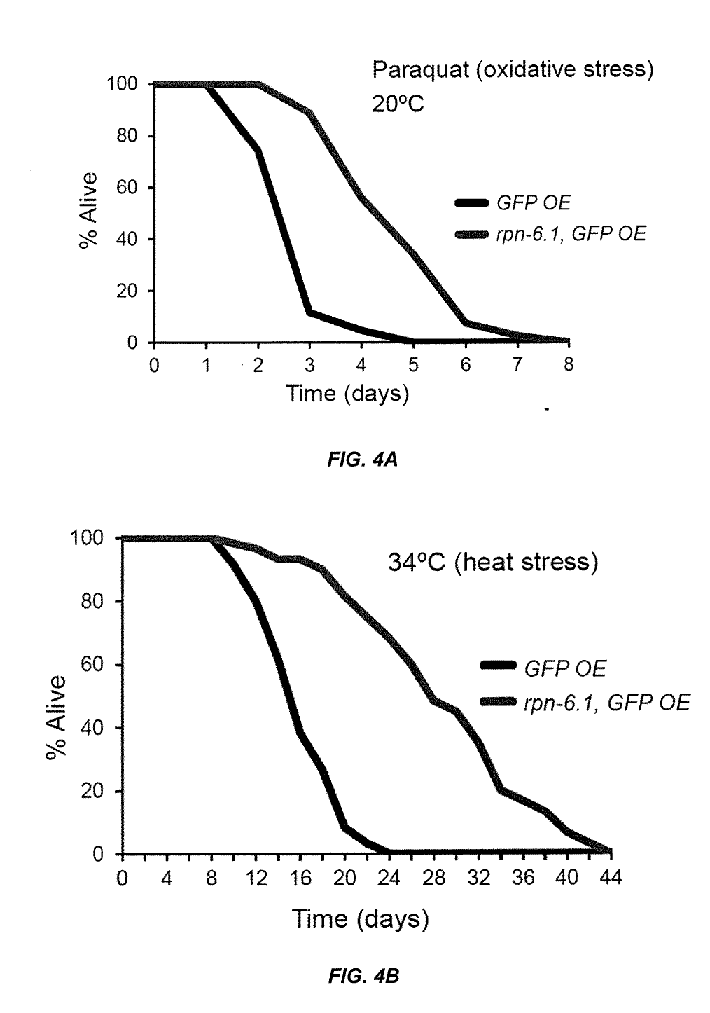

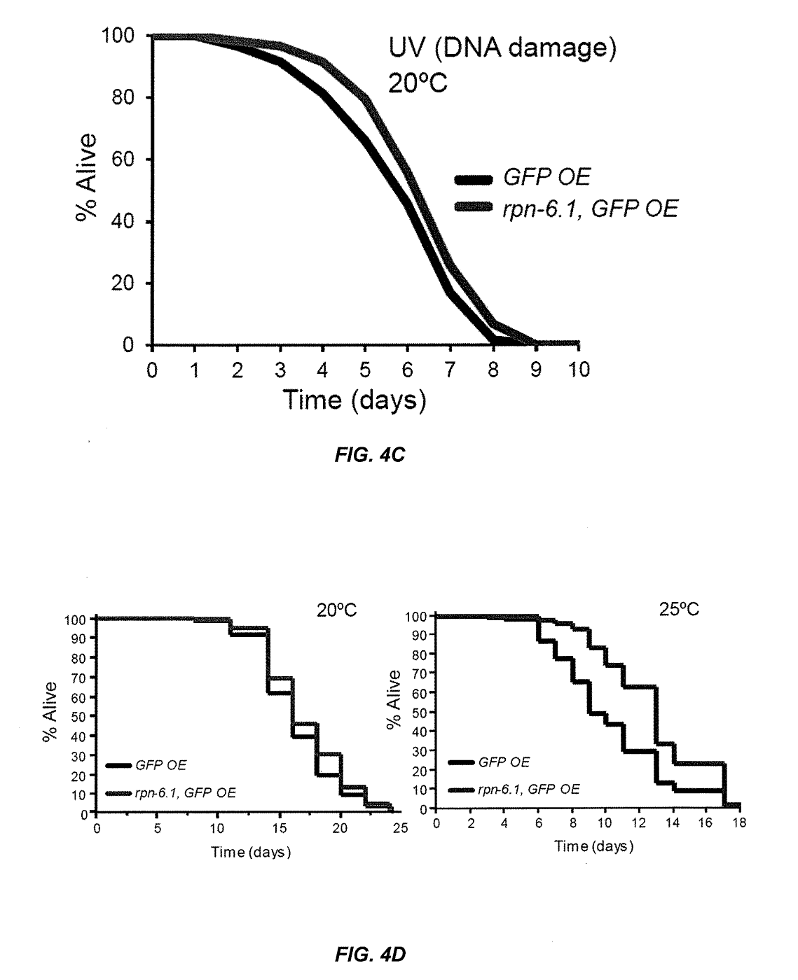

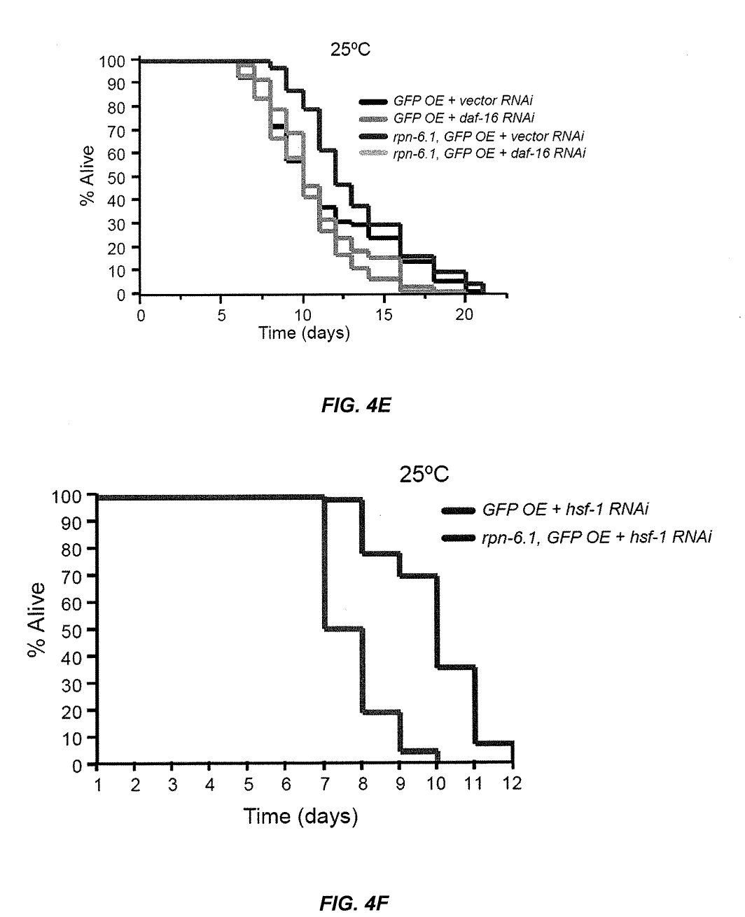

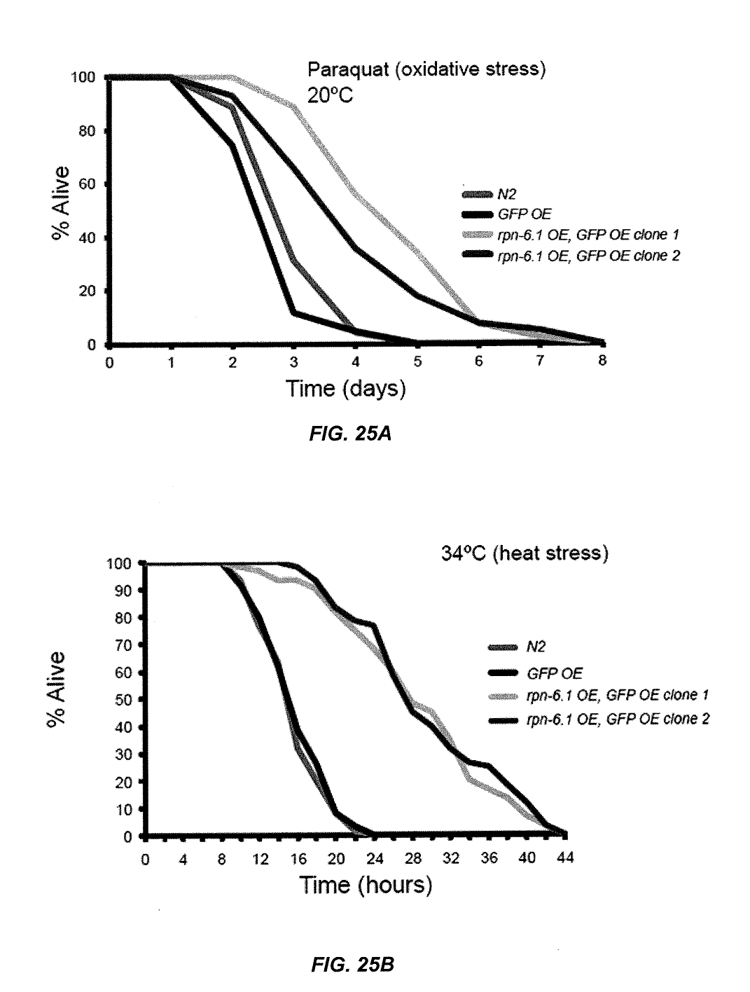

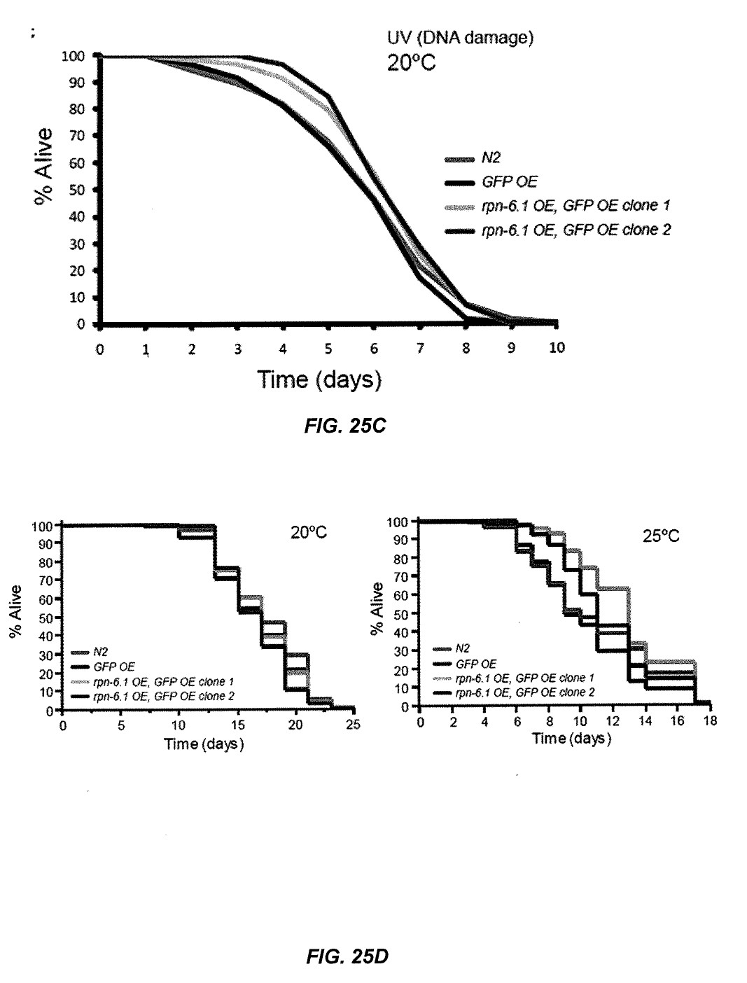

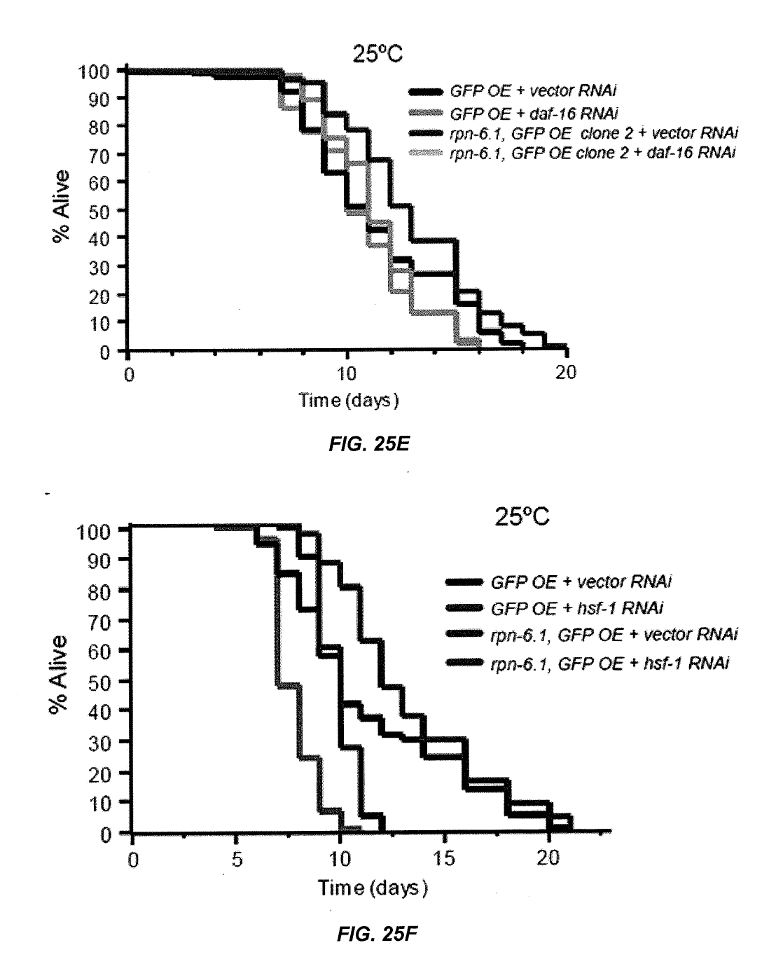

To explore whether rpn-6.1 might play a positive role in longevity Applicants tested the impact of increased rpn-6.1 levels. Applicants overexpressed rpn-6.1 in wild-type worms and conducted a series of physiological assays to measure the effects of rpn-6.1 overexpression (OE) on resistance to challenges of oxidative stress, heat-shock and ultraviolet (UV) damage, all correlated with increased longevity. rpn-6.1 (OE) animals were significantly more resistant to oxidative stress induced by growing the worms in the presence of paraquat (FIG. 4a and FIG. 25a). Under heat stress (34° C.), rpn-6.1 (OE) worms lived dramatically longer than control strains (FIG. 4b and FIG. 25b). Interestingly, increased levels of rpn-6.1 did not result in global up-regulation of all stress responses because rpn-6.1 (OE) did not protect against UV damage (FIG. 4c and FIG. 25c). Since overexpression of rpn-6.1 increased resistance to conditions that challenge the proteome, Applicants examined whether it also resulted in lifespan extension. rpn-6.1 (OE) did not extend lifespan of worms at 20° C. but did at 25° C., a temperature that results in mild heat stress (FIG. 4d and FIG. 25d). daf-16 RNAi treatment blocked the lifespan extension induced by rpn-6.1 (OE) at 25° C. (FIG. 4e and FIG. 25e). Therefore, under conditions of proteome stress, overexpression of rpn-6.1 is sufficient to promote increased survival. As a more formal test, Applicants asked whether animals with a reduced heat-shock response via hsf-1 downregulation had increased survival when rpn-6.1 was overexpressed. hsf-1 RNAi treated-rpn-6.1 (OE) worms were long-lived compared to control strains under the same treatment (FIG. 4f and FIG. 25f). This last result not only indicates that the lifespan extension induced by rpn-6.1 (OE) is hsf-1 independent, but also suggests that these worms can significantly overcome the loss of this critical transcription factor required for adult lifespan, heat-shock and proteotoxicity responses.

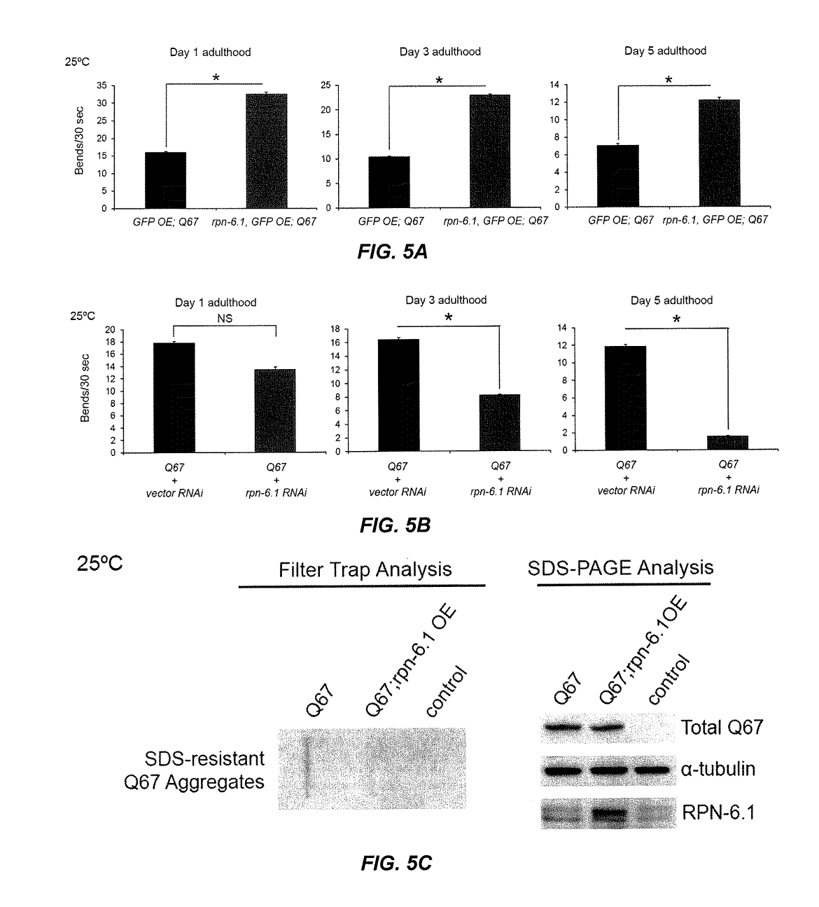

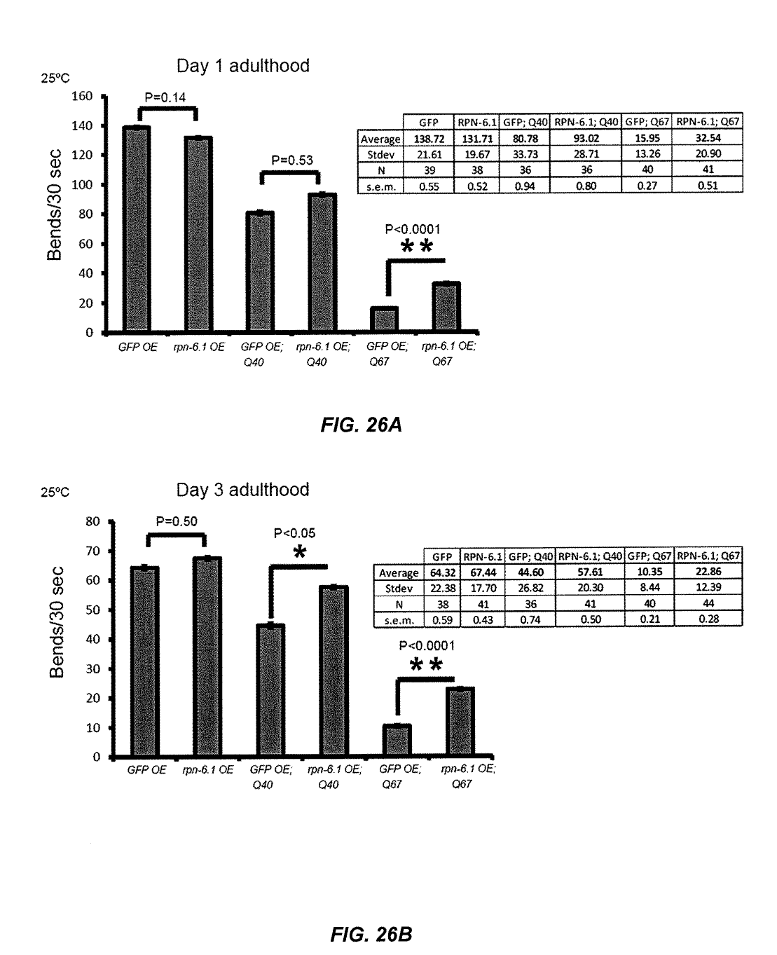

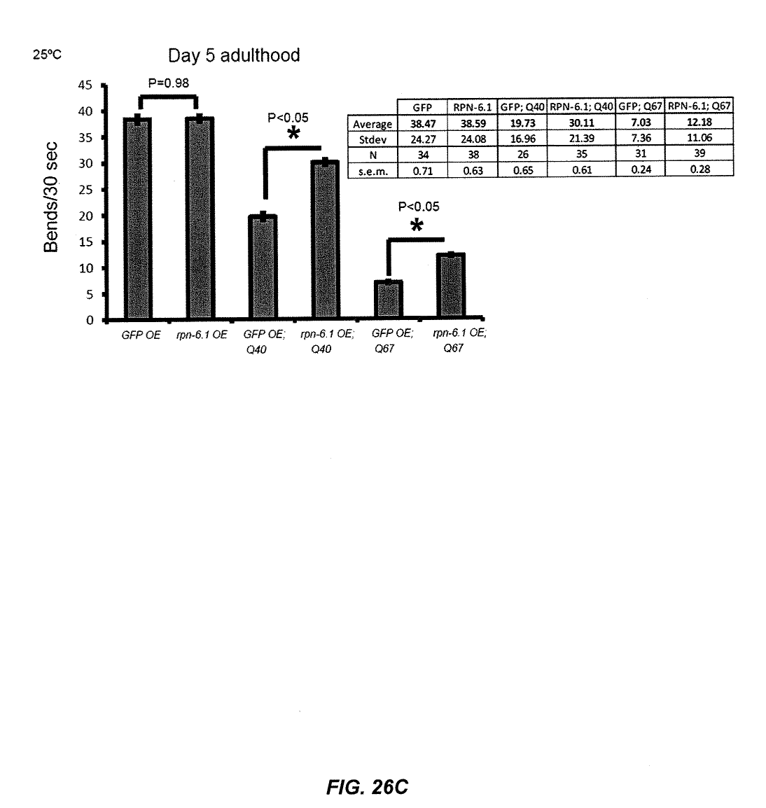

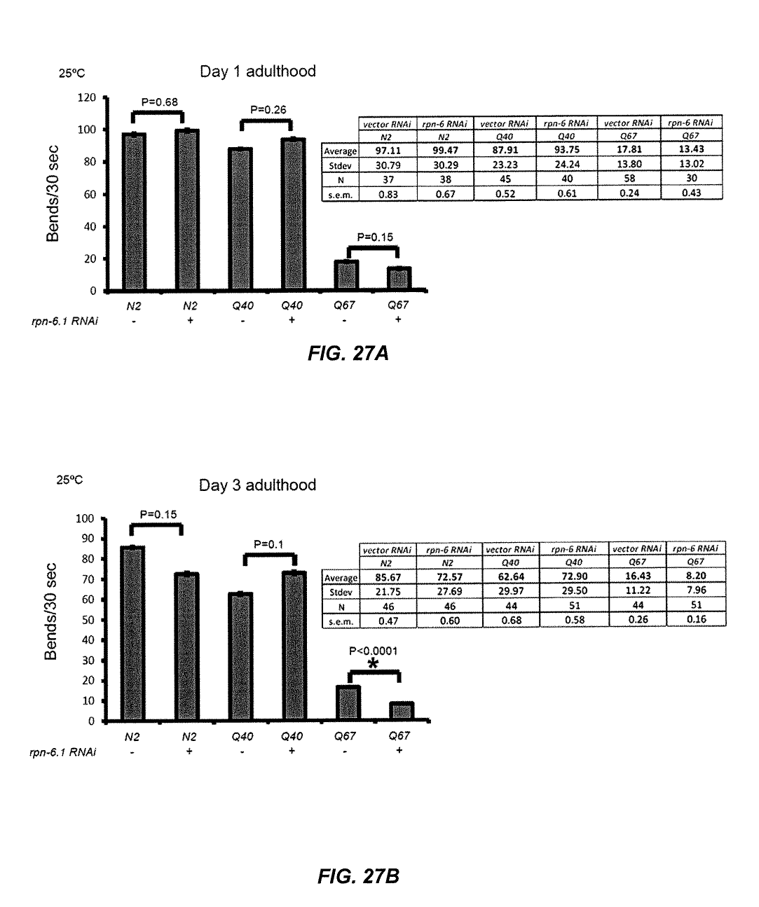

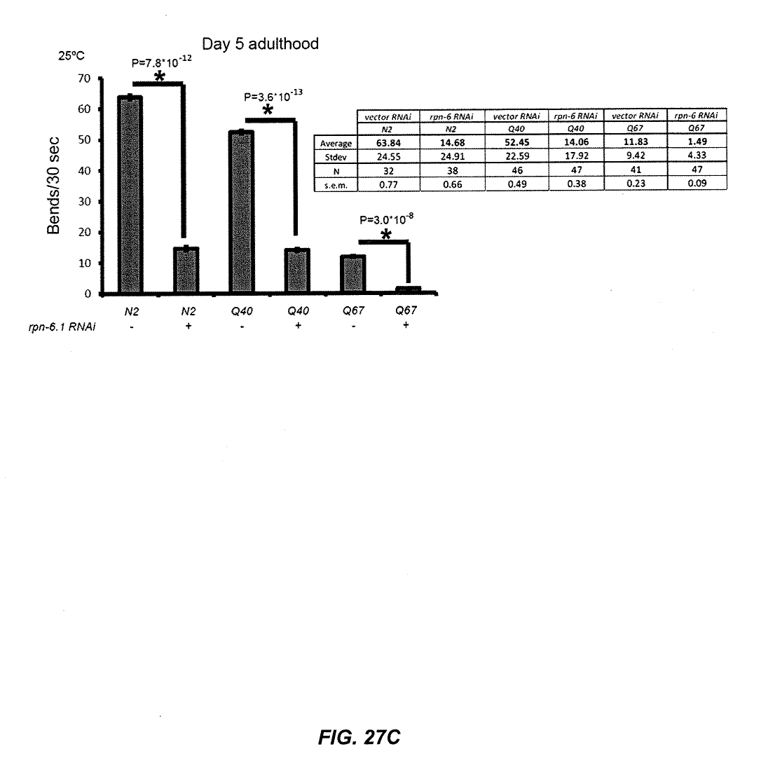

Intrigued by the protection that rpn-6.1 overexpression could confer, Applicants hypothesized that rpn-6.1 could be a potential candidate to correct protein homeostasis deficiencies underlying diseases such as Alzheimer's, Parkinson's or Huntington's disease. Since the later disease has been associated with proteasome failure [Li, X. J. & Li, S. Proteasomal dysfunction in aging and Huntington disease. Neurobiol Dis (2010)], Applicants tested whether increased levels of rpn-6.1 could have beneficial effects in a polyglutamine (polyQ) disease model. Worm motility is dramatically reduced by the aggregation of polyQ expression in neurons, with a pathogenic threshold at a length of 35-40 glutamines [Brignull, H. R., Moore, F. E., Tang, S. J. & Morimoto, R. I. Polyglutamine proteins at the pathogenic threshold display neuron-specific aggregation in a pan-neuronal Caenorhabditis elegans model. J Neurosci 26, 7597-7606 (2006)]. Notably, rpn-6.1 overexpression substantially improved motility and reduced toxicity of worms expressing polyQ40 and polyQ67 (FIG. 5a and FIG. 26). In addition, loss of rpn-6.1 worsened the motility phenotype of polyQ67 worms even at early (day 3) adulthood stages (FIG. 5b and FIG. 27). Furthermore, Applicants observed by filter trap analysis that rpn-6.1 (OE) reduced polyQ aggregate levels while polyQ67 total protein levels remained constant (FIG. 5c), suggesting that rpn-6.1 specifically reduces aggregated, but not soluble, polyQ proteins.

A growing body of evidence suggests that the protective modulation of various nodes of the proteostasis network, including the heat-shock response and autophagy, can contribute to the extended lifespan caused by the IIS [Melendez, A., et al. Autophagy genes are essential for dauer development and life-span extension in C. elegans. Science 301, 1387-1391 (2003); Morley, J. F. & Morimoto, R. I. Regulation of longevity in Caenorhabditis elegans by heat shock factor and molecular chaperones. Molecular biology of the cell 15, 657-664 (2004)], diet restriction [Hansen, M., et al. A role for autophagy in the extension of lifespan by dietary restriction in C. elegans. PLoS genetics 4, e24 (2008)], and germline-signaling pathway [Lapierre, L. R., Melendez, A. & Hansen, M. Autophagy links lipid metabolism to longevity in C. elegans. Autophagy 8 (2012)]. Applicants report here evidence for the requirement of an up-regulated proteasome activity in the extended lifespan of germline-deficient animals. Applicants' initial analysis of proteasome activity among different longevity models in the worm reveals that only glp-1 mutant and diet restricted animals share an increased proteasome activity, and Applicants hypothesize that these animals may share a strategy in which resources are actively reallocated from the germline to the soma, resulting in an enhanced protection of the proteome within somatic cells. Furthermore, Applicants find distinct differences among the proteasome activity between glp-1 and daf-2 mutant animals that is mediated by daf-16, and in part by kri-1, daf-12 and daf-9, confirming previous genetic suggestions that daf-16 activity is differentially regulated between glp-1 and daf-2 mutants. Mechanistically, in germline-deficient animals, rpn-6.1 and subsequent increases in proteasome activity appear to be direct downstream targets of DAF-16/FOXO. Applicants' results thus provide new insights into proteostasis regulation and provide a link between the longevity regulator DAF-16 and proteasome activity regulation upon rpn-6.1 expression.

Applicants further define RPN-6 as a potent factor to increase resistance to proteotoxic stress, since its up-regulation can delay the deleterious effects of strong adverse conditions. It is intriguing to speculate that one method to ensure survival of the soma maybe the direct activation of FOXO/daf-16, under limited nutrient availability or loss of the germline, resulting in increased rpn-6.1 levels and increased proteome maintenance. Recently, it has been reported that changes in the proteasome may explain why aging is a risk factor for neurodegenerative diseases such as Alzheimer's, Parkinson's and Huntington's disease [Zabel, C., et al. Proteasome and oxidative phoshorylation changes may explain why aging is a risk factor for neurodegenerative disorders. J Proteomics 73, 2230-2238 (2010)]. Therefore, RPN-6 may be a powerful candidate to correct deficiencies in disorders associated with a failure in protein homeostasis. It will be of crucial interest to explore in mammalian models if RPN-6 could indeed alleviate the associated symptoms to these disorders.

Experimental Procedures

C. elegans were cultured using standard techniques [Brenner, S. The genetics of Caenorhabditis elegans. Genetics 77, 71-94 (1974)] and fed on Escherichia coli OP50 or HT115 containing a dsRNA-expressing plasmid [Fire, A., et al. Potent and specific genetic interference by double-stranded RNA in Caenorhabditis elegans. Nature 391, 806-811 (1998)].

26S proteasome activity assays. In vitro 26S proteasome activity assays were performed as previously described [Finley, D. Recognition and processing of ubiquitin-protein conjugates by the proteasome. Annual review of biochemistry 78, 477-513 (2009)]. Worms were lysed in proteasome activity assay buffer (50 mM Tris-HCl pH 7.5, 250 mM sucrose, 5 mM MgCl₂, 0.5 mM EDTA, 2 mM ATP and 1 mM dithiothreitol) using a Precellys 24 homogenizer (Bertin technologies). Lysate was centrifuged at 10,000 g for 15 min at 4° C. 25 μg of total protein lysate was transferred to a 96-well microtiter plate (BD Falcon) and incubated with fluorogenic substrate. Fluorescence (380-nm excitation, 460-nm emission) was monitored on a microplate fluorometer (Infinite M1000, Tecan) every 5 min for 1 h at 25° C.

Motility Assay. Thrashing rate was determined as previously described [Brignull, H. R., Moore, F. E., Tang, S. J. & Morimoto, R. I. Polyglutamine proteins at the pathogenic threshold display neuron-specific aggregation in a pan-neuronal Caenorhabditis elegans model. J Neurosci 26, 7597-7606 (2006)]. Worms were transferred to a drop of M9 buffer and after 30 seconds of adaptation the number of body bends was counted for 30 seconds. A body bend was defined as change in direction of the bend at the midbody of an animal [Chai, Y., Shao, J., Miller, V. M., Williams, A. & Paulson, H. L. Live-cell imaging reveals divergent intracellular dynamics of polyglutamine disease proteins and supports a sequestration model of pathogenesis. Proceedings of the National Academy of Sciences of the United States of America 99, 9310-9315 (2002)].

Filter trap. Worm extracts were generated by glass bead disruption on ice in non-denaturing lysis buffer (50 mM Hepes pH 7.4, 150 mM NaCl, 1 mM EDTA, 0.5% Triton X100) supplemented with EDTA-free protease inhibitor cocktail (Roche). Lysate was centrifuged at 5,000 g for 5 min. 70 μg of protein extract was supplemented with SDS at a final concentration of 1% and loaded onto a cellulose acetate membrane assembled in a slot blot apparatus (BioRad). The membrane was washed with 0.1% SDS and retained Q67-GFP was assessed by immunoblotting for GFP (Roche).

A detailed description of all experimental methods including C. elegans strains, growth, imaging, lifespan analysis, stress assays and RNAi application is provided in Methods.

C. elegans strains and generation of transgenic lines. CF512 (fer-15(b26)II;fem-1(hc17)IV), CB4037 (glp-1 (e2141)III), AU147 (daf-16(mgDf47)I;glp-1(e2141)III), CF1880 (daf-16(mu86)I;glp-1(e2141)III), DA1116 (eat-2(ad1116)II) and wild-type (N2) C. elegans strains were obtained from the Caenorhabditis Genetic Center. AGD151 (eat-2(ad1116)II; fer-15(b26)II;fem-1(hc17)IV) was generated by crossing CF512 with DA1116 (eat-2(ad1116)II). CF596 (daf-2(mu150)III; fer-15(b26)II;fem-1(hc17)IV) was a gift from Cynthia Kenyon. C. elegans were handled using standard methods. [Brenner, S. The genetics of Caenorhabditis elegans. Genetics 77, 71-94 (1974)].

For generation of worm strains AGD597-AGD598 (N2, uthEx556[psur5::rpn-6.1, pmyo3::GFP] and N2, uthEx556[psur5::rpn-6.1, pmyo3::GFP]), a DNA plasmid mixture containing 75 ngμl⁻¹ pDV1 (psur5::rpn-6.1) and 20 ngμl⁻¹ pPD93_{—}97 (pmyo-3::GFP) was injected into the gonads of adult N2 hermaphrodite animals, using standard methods [Mello, C. C., Kramer, J. M., Stinchcomb, D. & Ambros, V. Efficient gene transfer in C. elegans: extrachromosomal maintenance and integration of transforming sequences. The EMBO journal 10, 3959-3970 (1991)]. GFP positive F1 progeny were selected. Individual F2 worms were isolated to establish independent lines. Control worms (AGD614) used in experiments with AGD597-AGD598 were generated by microinjecting N2 worms with 20 ngμl⁻¹ pPD93_{—}97 (pmyo-3::GFP). AGD886 (fer-15(b26)II; fem-1(hc17)IV; uthEx557[psur5::rpn-6.1, pmyo3::GFP]) was generated by crossing AGD598 to CF512. Control strain AGD885 (fer-15(b26)II;fem-1(hc17)IV; uthEx633[myo3p::GFP]) was generated by crossing AGD614 to CF512.

Both AM101 (rmIs110[pF25B3.3::Q40::YFP]) and AM716 (rmIs284[pF25B3.3::Q67::YFP]) were a gift from Richard I. Morimoto. For generation of worm strains AGD850 (rmIs110[pF25B3.3::Q40::YFP];uthEx557[psur5::rpn-6.1,pmyo3::GFP]) and AGD851 (rmIs284[pF25B3.3::Q67::YFP];uthEx557[psur5p::rpn-6.1,pmyo3::GFP]), AGD598 strain was crossed to AM101 and AM716, respectively. Control strains AGD866 (rmIs110[pF25B3.3::Q40::YFP];uthEx633[pmyo3::GFP]) and AGD867 (rmIs284[pF25B3.3::Q67::YFP]; ;uthEx633[pmyo3::GFP]) were generated by crossing AGD614 to AM101 and AM716, respectively.

For generation of worm strains AGD945-AGD946 (N2, uthEx649 [rpn-6p::tdTomato, pRF4(rol-6)] and N2, uthEx650[rpn-6p::tdTomato, pRF4(rol-6)]), a DNA plasmid mixture containing 75 ngμl⁻¹ pDV2 (rpn-6p::tdTomato) and 20 ngμl⁻¹ pRF4(rol-6) was injected into the gonads of adult N2 hermaphrodite animals. Roller phenotype positive F1 progeny were selected. Individual F2 worms were isolated to establish independent lines. AGD1047 (glp-1(e2141)III; uthEx649[rpn-6p::tdTomato, pRF4(rol-6)) was generated by crossing AGD945 to CB4037. AGD1048 (daf-16(mu86)I;glp-1 (e2141)III; uthEx649[rpn-6p::tdTomato, pRF4(rol-6)) was generated by crossing AGD945 to CF1880.

YD1(N2, xzEx1[Punc-54::Dendra2]) and YD3 (N2, xzEx3[Punc-54::UbG76V::Dendra2]) were a gift from Carina I. Holmberg. AGD1032 (glp-1(e2141)III; xzEx1[Punc-54::Dendra2]) was generated by crossing YD1 to CB4037. AGD1033 (glp-1(e2141)III; xzEx3[Punc-54::UbG76V::Dendra2]) was generated by crossing YD3 to CB4037. AGD1036 (fer-15(b26)II;fem-1(hc17)IV; xzEx1[Punc-54::Dendra2]) was generated by crossing YD1 to CF512. AGD1037 (fer-15(b26)II;fem-1(hc17)IV; xzEx3[Punc-54::UbG76V::Dendra2]) was generated by crossing YD3 to CF512.

Construction of rpn-6.1 expression construct. To construct pDV1, the rpn-6.1 C. elegans expression plasmid, pPD95.77 from the Fire Lab kit was digested with SphI and XmaI to insert 3.6 KB of the sur5 promoter. The resultant vector was then digested with KpnI and EcoRI to excise GFP and insert a multi-cloning site containing KpnI, NheI, NotI, XbaI, and EcoRI. F57B9.10.A (rpn-6.1) was PCR amplified from cDNA to include 5′ XmaI and 3′ XbaI restriction sites then cloned into the aforementioned vector. All constructs were sequence verified.

Construction of rpn-6.1 transcriptional reporter construct. To construct pDV2, pPD95.77 from the Fire Lab kit was digested to replace GFP with tdTOMATO. The promoter region and first intron of F57B9.10.A (rpn-6.1) was PCR amplified from N2 gDNA to include -363 to +1012 then cloned into the aforementioned vector using SalI and BamHI. The construct includes 46 nucleotides of exon 1. Construct was sequence verified.

RNAi constructs. RNAi-treated strains were fed E. coli (HT115) containing an empty control vector (L4440) or expressing double-stranded RNAi. daf-12, rpn-2, rpn-6.1, rpn-11 and skn-1 RNAi constructs used were taken from the Vidal RNAi library. cco-1, rpn-1, nhr-80, daf-9, hsf-1 and kri-1 RNAi constructs used were from the Ahringer RNAi library. pAD43, the daf-16 RNAi construct, was previously described [Dillin, A., Crawford, D. K. & Kenyon, C. Timing requirements for insulin/IGF-1 signaling in C. elegans. Science 298, 830-834 (2002)]. See Table 5 for further details about double-stranded RNA is used for knockdown assays.

Lifespan studies. Lifespan analyses were performed as described previously [Dillin, A., Crawford, D. K. & Kenyon, C. Timing requirements for insulin/IGF-1 signaling in C. elegans. Science 298, 830-834 (2002)]. Worms were synchronized by egg laying during 2 hours. Animals were grown at 20° C. until day 1 of adulthood. 100 animals were used per condition and scored every day or every other day. Lifespans were conducted at either 20° C. or 25° C. as stated in the figure legends. For non-integrated lines AGD597, AGD598 and AGD886, GFP positive worms were selected for lifespan studies. JMP IN 8 software was used for statistical analysis to determine means and percentiles. In all cases, P-values were calculated using the log-rank (Mantel-Cox) method.

Stress assays. For heat-shock assays, eggs were transferred to plates seeded with E. coli (OP50) bacteria and grown to day 1 of adulthood at 20° C. Worms were then transferred to fresh plates and heat shocked at 34° C. Worms were checked every hApplicants' for viability. Paraquat assays were performed as previously described [Vazquez-Manrique, R. P., et al. Reduction of Caenorhabditis elegans frataxin increases sensitivity to oxidative stress, reduces lifespan, and causes lethality in a mitochondrial complex II mutant. FASEB J 20, 172-174 (2006)]. Briefly day-1 adults were transferred to plates containing 7.5 mM paraquat and cultured at 25° C. Worms were checked every day for viability. For UV irradiation assays [Wolff, S., et al. SMK-1, an essential regulator of DAF-16-mediated longevity. Cell 124, 1039-1053 (2006)], day-5 adult worms were transferred to plates without OP50 and exposed to 1200 J/m of UV using a UV Stratalinker. Worms were transferred back to fresh plates seeded with E. coli (OP50) and scored daily for viability.

Motility Assay. Thrashing rate was determined as previously described [Brignull, H. R., Moore, F. E., Tang, S. J. & Morimoto, R. I. Polyglutamine proteins at the pathogenic threshold display neuron-specific aggregation in a pan-neuronal Caenorhabditis elegans model. J Neurosci 26, 7597-7606 (2006)]. Animals were grown at 20° C. until L4 stage and then grown at 25° C. for the rest of the experiment. Worms were fed with E. coli (OP50) bacteria. RNAi-treated strains were fed E. coli (HT115) containing an empty control vector (L4440) or expressing double-stranded RNAi of the rpn-6.1 gene. Worms were transferred at day 1, 3 or 5 of adulthood to a drop of M9 buffer and after 30 seconds of adaptation the number of body bends was counted for 30 seconds. A body bend was defined as change in direction of the bend at the midbody of an animal [Chai, Y., Shao, J., Miller, V. M., Williams, A. & Paulson, H. L. Live-cell imaging reveals divergent intracellular dynamics of polyglutamine disease proteins and supports a sequestration model of pathogenesis. Proceedings of the National Academy of Sciences of the United States of America 99, 9310-9315 (2002)].

26S proteasome fluorogenic peptidase assays. In vitro 26S proteasome activity assays were performed as previously described [Kisselev, A. F. & Goldberg, A. L. Monitoring activity and inhibition of 26S proteasomes with fluorogenic peptide substrates. Methods in enzymology 398, 364-378 (2005)]. Briefly, worms were lysed in proteasome activity assay buffer (50 mM Tris-HCl pH 7.5, 250 mM sucrose, 5 mM MgCl₂, 0.5 mM EDTA, 2 mM ATP and 1 mM dithiothreitol) using a Precellys 24 homogenizer (Bertin technologies). Lysate was centrifuged at 10,000 g for 15 min at 4° C. For each experiment, 25 μg of total protein lysate was transferred to a 96-well microtiter plate (BD Falcon) then fluorogenic substrate was added. For measuring the chymotrypsin-like activity of the proteasome either Z-Gly-Gly-Leu-AMC (Enzo) or Suc-Leu-Leu-Val-Tyr-AMC (Enzo) was used. Z-Leu-Leu-Glu-AMC (Enzo) was used to measure the caspase-like activity of the proteasome and Ac-Arg-Leu-Arg-AMC for the proteasome trypsin-like activity. Fluorescence (380-nm excitation, 460-nm emission) was monitored on a microplate fluorometer (Infinite M1000, Tecan) every 5 min for 1 h at 25° C.

Western Blot. For each strain 2000 adult worms were collected in proteasome assay activity buffer supplemented with protease inhibitors (Roche) and lysed using a Precellys 24 homogenizer. Lysate was centrifuged at 10,000 g for 15 min at 4° C. 40 μg of total protein was resolved by SDS-PAGE and transferred to nitrocellulose membrane. Western blot analysis was performed with anti-20S alpha 1-7 (Abcam), anti-Proteasome 20S C2 (Abcam), anti-Rpt6 (Enzo), anti-Rpt5 (Enzo), anti-PSMD7 (Abcam), anti-Rpn2 (Abcam), anti-PSMD11 (Novus), anti-FK1 (Enzo), GFP (Roche), anti-α-tubulin (Sigma) and anti-β-actin (Abcam).

Filter trap. Animals were grown at 20° C. until L4 stage and then grown at 25° C. for the rest of the experiment. Day 1 adult worms were collected with M9 buffer and worm pellets were frozen with liquid N2. Frozen worm pellets were thawed on ice and worm extracts were generated by glass bead disruption on ice in non-denaturing lysis buffer (50 mM Hepes pH 7.4, 150 mM NaCl, 1 mM EDTA, 0.5% Triton X100) supplemented with EDTA-free protease inhibitor cocktail (Roche). Worm and cellular debris was removed with 5000 g spin for 5 min. Approximately 70 μg of protein extract was supplemented with SDS at a final concentration of 1% and loaded onto a cellulose acetate membrane assembled in a slot blot apparatus (BioRad). The membrane was washed with 0.1% SDS and retained Q67-GFP was assessed by immunoblotting for GFP (Roche). Extracts were also analyzed by SDS-PAGE to determine protein expression levels.

Microscopy, image analysis, equipment and settings. Newly hatched larvae were grown at 25° C. until day 3 of adulthood. These young adults were mounted at room temperature (20-23° C.) on a 10% agarose pad on glass slides with 1 ul of M9, covered with cover slip. For imaging, Zeiss Axiovert microscope and AxioCam with software AxioVision Rel. 4.7 was used. Images of whole worms were acquired with 10×0.45 numerical aperture (NA) plan-apochromat objectives. Photoconversion was carried out using a 405 nm filter and an EXFO X-Cite 120Q metal halide lamp with 100% output for 60 seconds. Worms were imaged before and after photoconversion, and then were recovered on feeding plates at 20° C. After 24 hr, photoconverted worms were imaged with the same setting. Fluorescence intensities were analyzed with AxioVision Rel. 4.7.

RNA isolation and quantitative RT-PCR. Total RNA was isolated from synchronized populations of approximately 2,000 day-5 adults. Total RNA was extracted using TRIzol reagent (GIBCO). cDNA was generated using Quantitect Reverse Transcriptase kit (Qiagen). SybrGreen real-time qPCR experiments were performed with a 1:20 dilution of cDNA using an ABI Prism79000HT (Applied Biosystems) following the manufacturer's instructions. Data was analyzed with the comparative 2ΔΔCt method using the geometric mean of cdc-42, pmp-3 and Y45F10D.4 as endogenous control [Hoogewijs, D., Houthoofd, K., Matthijssens, F., Vandesompele, J. & Vanfleteren, J. R. Selection and validation of a set of reliable reference genes for quantitative sod gene expression analysis in C. elegans. BMC Mol Biol 9, 9 (2008)]. See Table 6 for details about the primers used for this assay.

### 2. Example 2

### The Role of Foxo4 in Stem Cell Function and Neurogenesis

Embryonic stem cells are able to replicate continuously in the absence of senescence and, therefore, are immortal in culture (Evans, M. J. & Kaufman, Nature 292, 154-156 (1981); Thomson, J. A., et al., Science 282, 1145-1147 (1998)). While genome stability is central for survival of stem cells; proteome stability may play an equally important role in stem cell identity and function. Additionally, with the asymmetric divisions invoked by stem cells, the passage of damaged proteins to daughter cells could potentially destroy the resulting lineage of cells. Applicants hypothesized that stem cells have an increased proteostasis ability compared to their differentiated counterparts and asked whether the proteasome activity differed among human embryonic stem cells (hESCs). Notably, hESC populations exhibit a high proteasome activity that is correlated with increased levels of the 19S proteasome subunit PSMD11/RPN-6 (Isono, E. et al., The Journal of biological chemistry 280, 6537-6547 (2005); Pathare, G. R., et al., Proceedings of the National Academy of Sciences of the United States of America 109, 149-154 (2012); Santamaria, P. G. et al., The Journal of biological chemistry 278, 6687-6695 (2003)) and a corresponding increased assembly of the 26S/30S proteasome. Ectopic expression of PSMD11 is sufficient to increase proteasome assembly and activity. FOXO4, an insulin/IGF-1 responsive transcription factor associated with long lifespan in invertebrates (Kenyon, C. et al., Nature 366, 461-464 (1993); Tatar, M., et al., Science 292, 107-110 (2001)), regulates proteasome activity by modulating the expression of PSMD11 in hESCs and is necessary for hESC differentiation into neural lineages. Applicants' results establish a novel regulation of proteostasis in hESCs that links longevity and stress resistance in invertebrates with hESC function and identity.

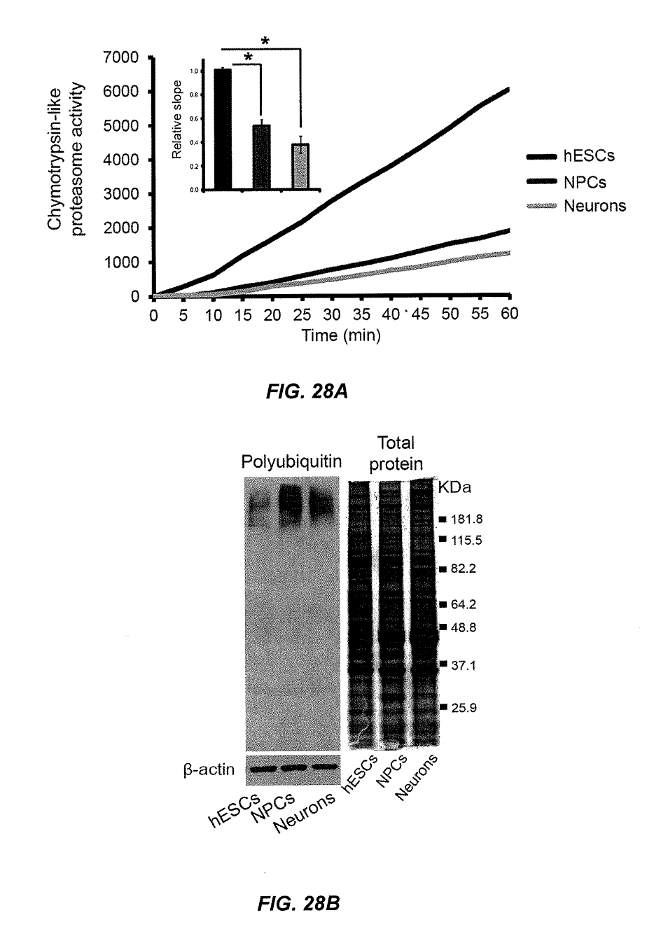

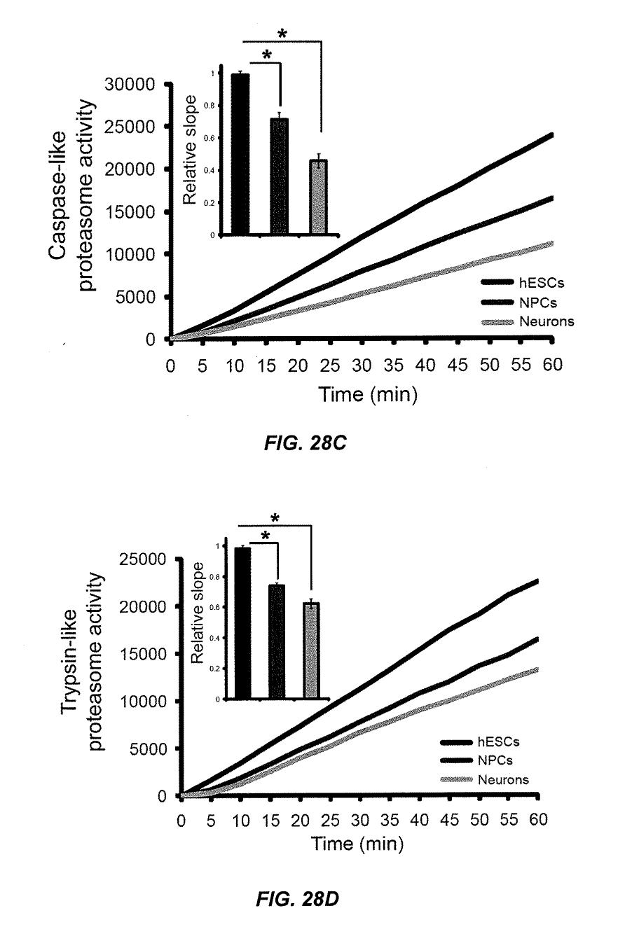

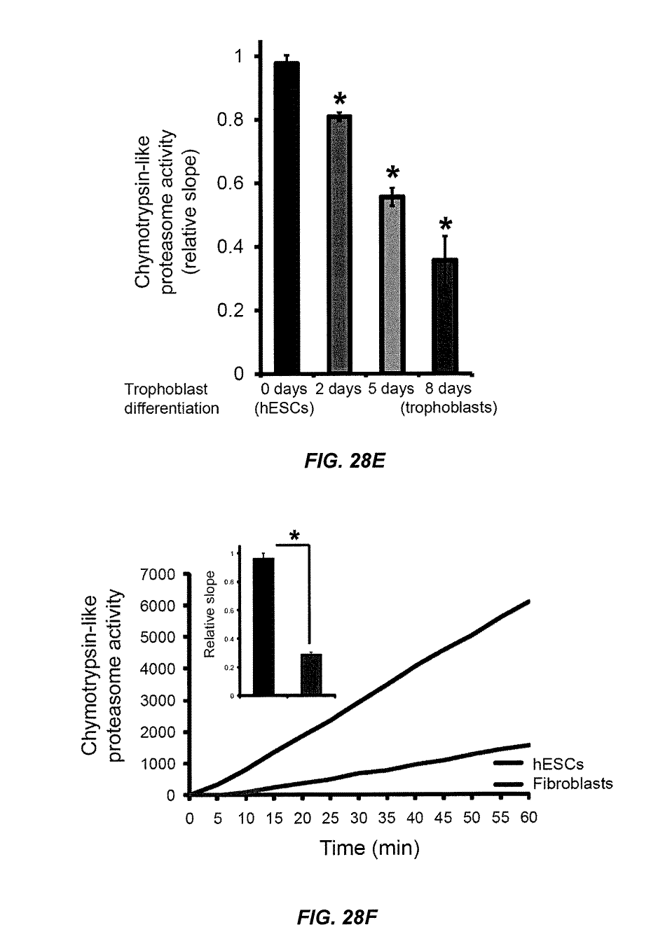

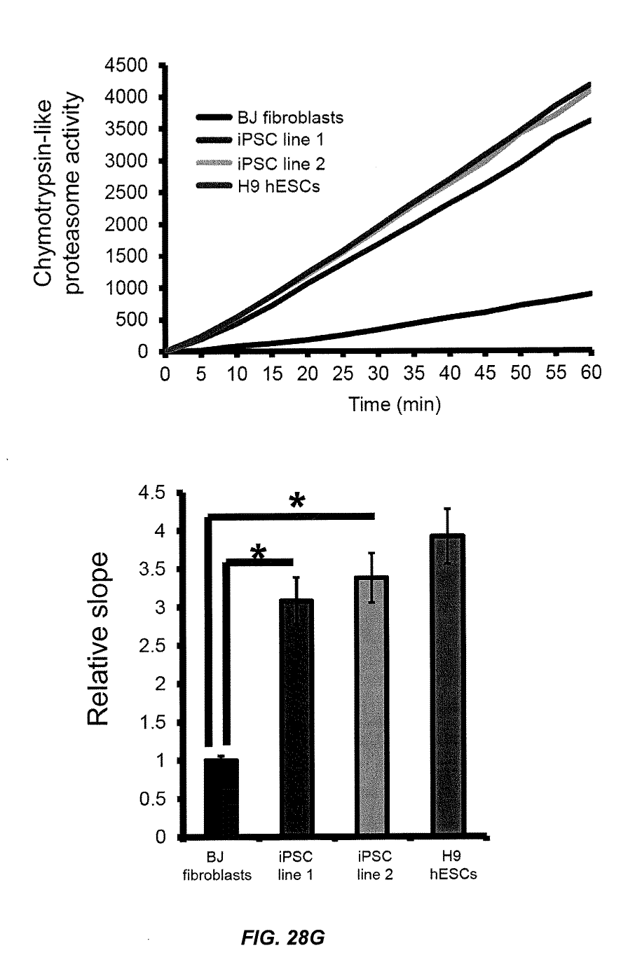

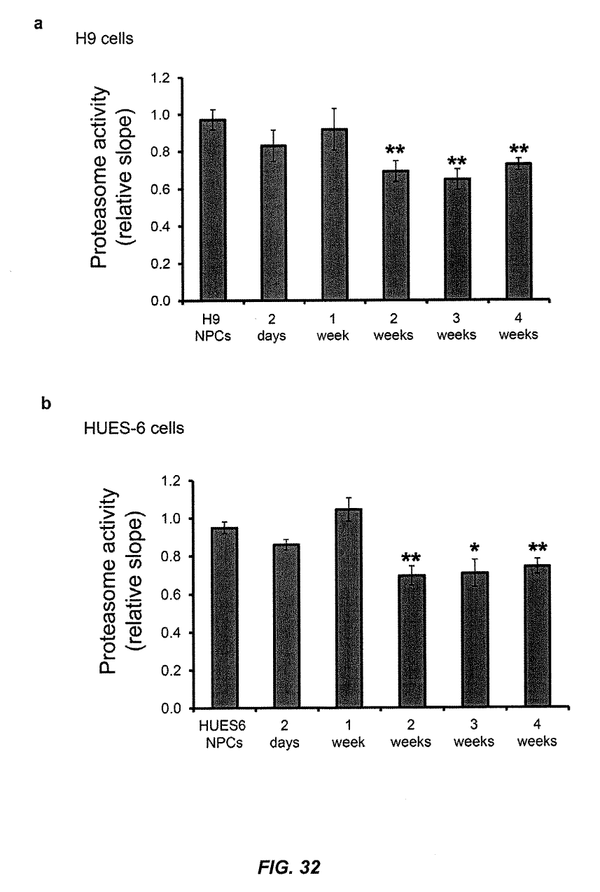

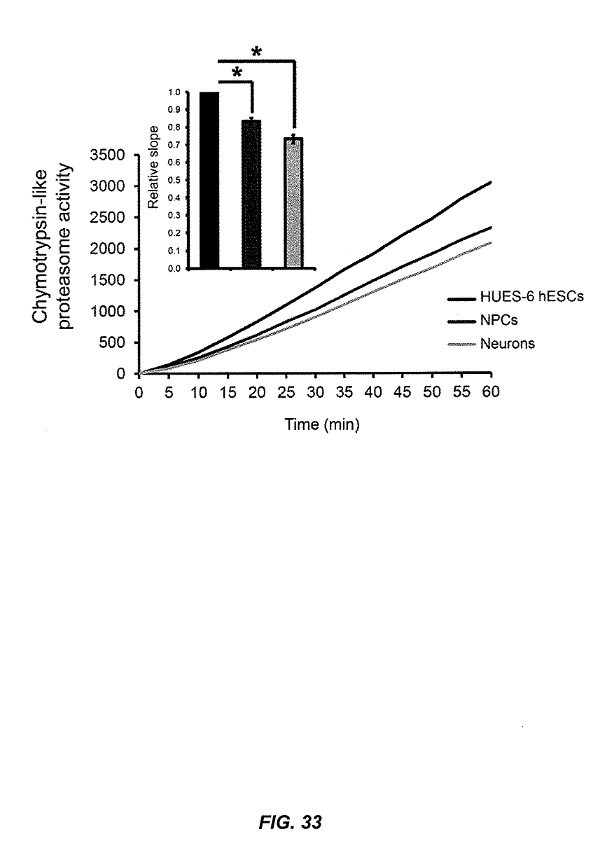

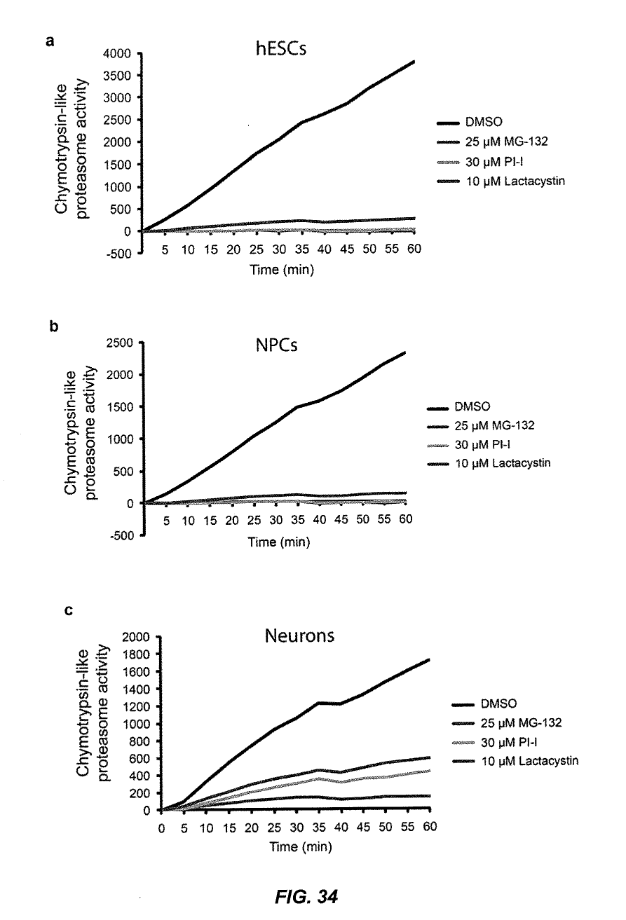

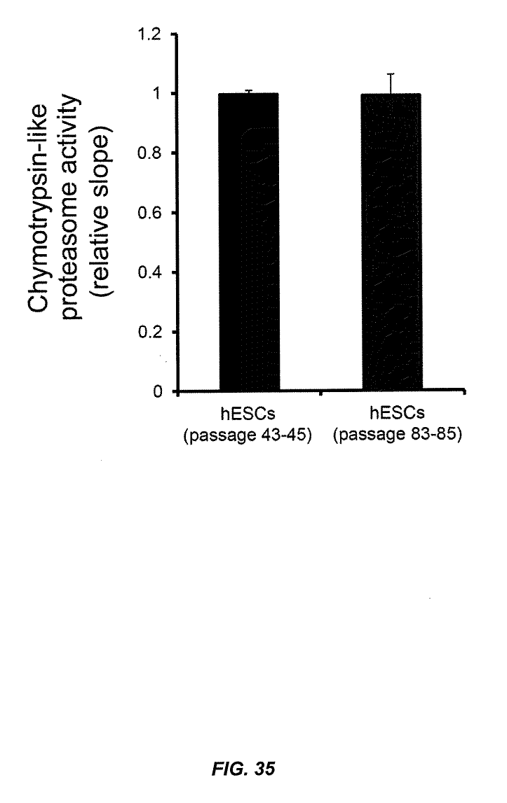

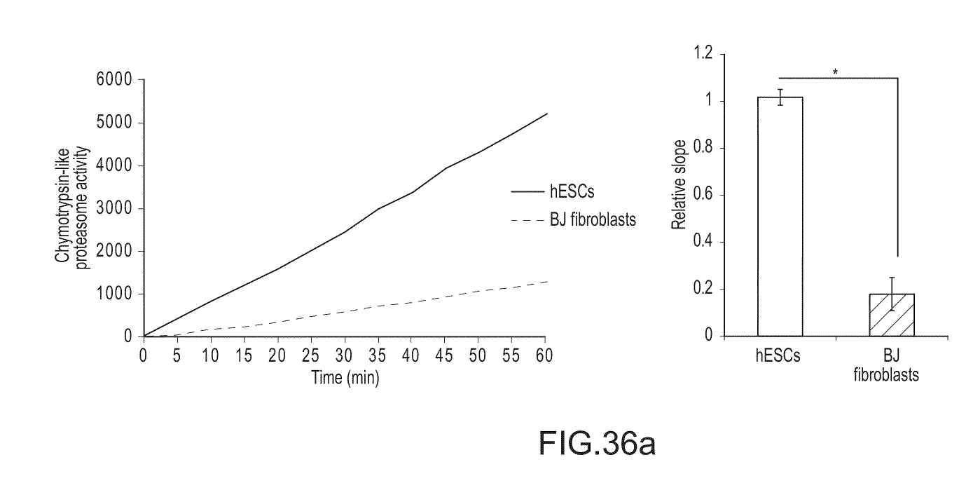

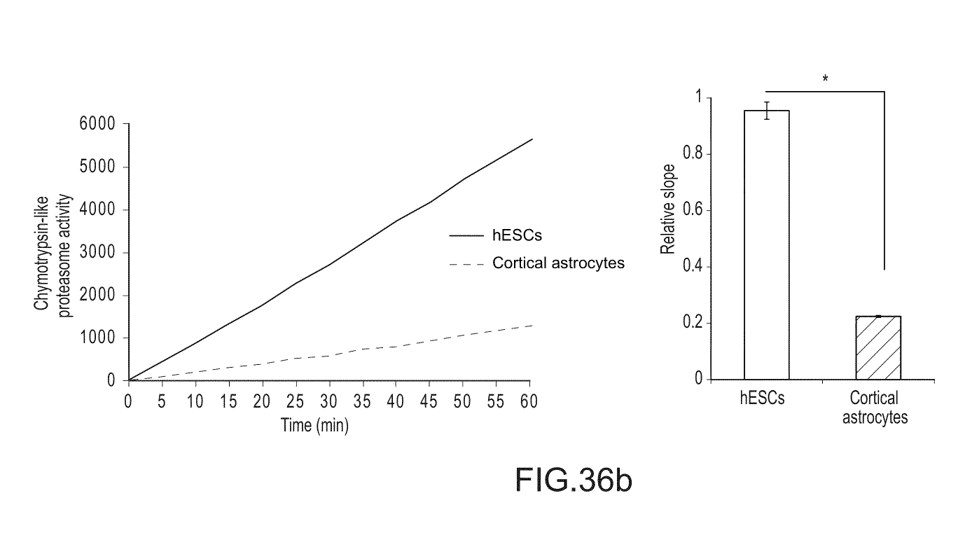

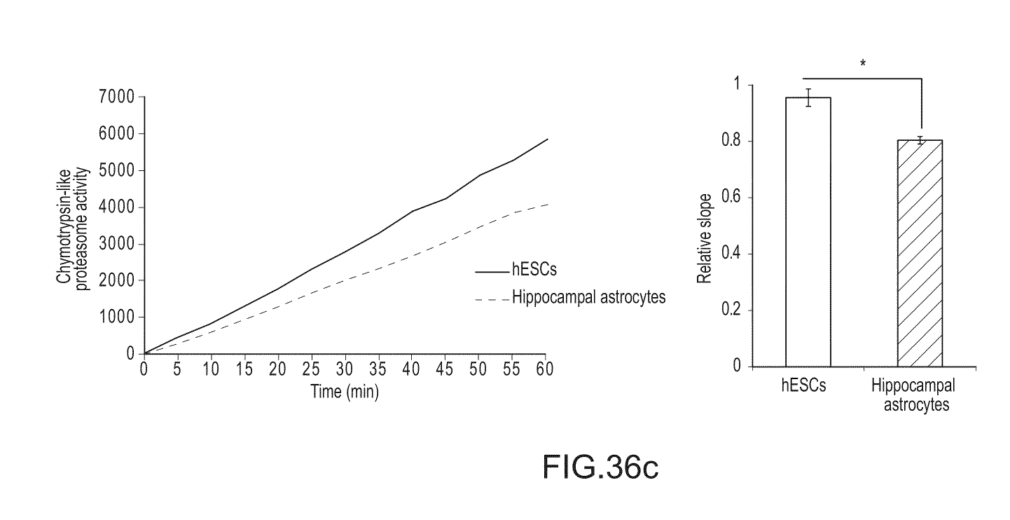

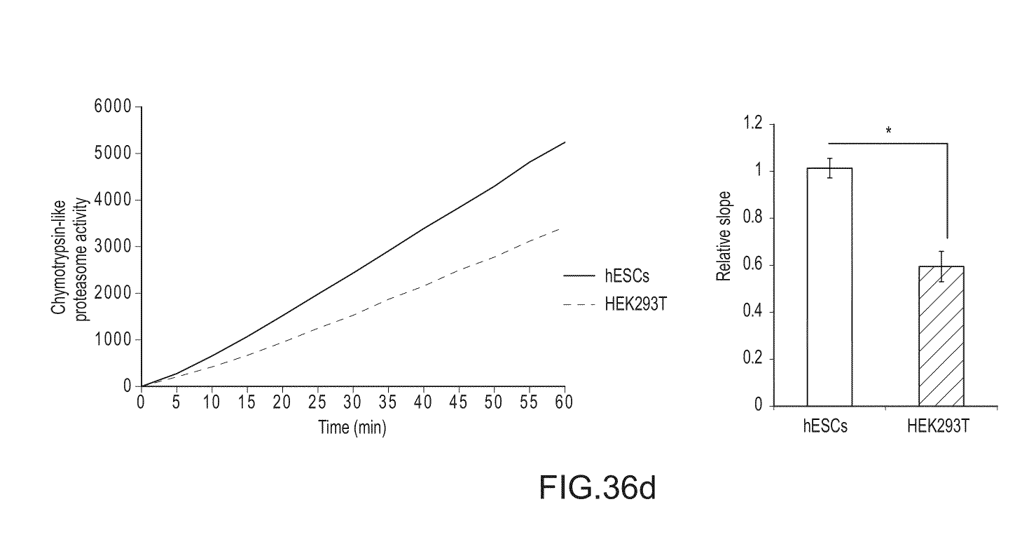

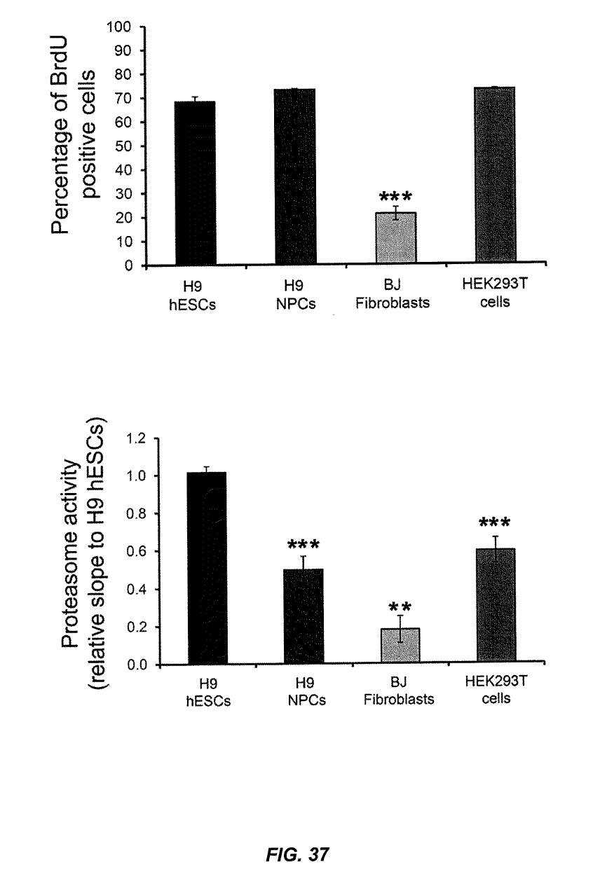

ESCs are unique among all stem cell populations examined insofar as they do not appear to undergo replicative senescence (Evans, M. J. & Kaufman, Nature 292, 154-156 (1981); Thomson, J. A., et al., Science 282, 1145-1147 (1998)). Since the ability to ensure proteostasis is critical for maintaining proper cell function (Balch, W. E. et al., Science 319, 916-919 (2008); Powers, E. T. et al., Annual review of biochemistry 78, 959-991 (2009)), hESCs could provide a novel paradigm to define proteostasis regulation and its demise in aging. To evaluate differences in the 26S/30S proteasome activity, Applicants monitored the degradation of specific fluorogenic peptide substrates (Kisselev, A. F. & Goldberg, A. L., Methods in enzymology 398, 364-378 (2005)). Applicants differentiated H9 hESCs into neural progenitors cells (NPCs), which were then further differentiated into neurons. Applicants found a dramatic decrease in the chymotrypsin-like proteasome activity when H9 hESCs were differentiated into NPCs (FIG. 28a). Moreover, when NPCs were differentiated into neurons, Applicants detected a further decrease in proteasome activity that was observable after 2 weeks during the differentiation process (FIG. 28a and FIG. 32). Consistent with enhanced proteasome activity in hESCs, Applicants found increased levels of polyubiquitinylated proteins in differentiated cells compared to hESCs (FIG. 28b). Since hESCs are known to vary in their characteristics despite unlimited capacity of self-renewal (Osafune, K., et al., Nature biotechnology 26, 313-315 (2008)), Applicants differentiated a distinct hESC line, HUES-6 cells, and found similar results (FIG. 32-33). Proteasome inhibitors blocked activity from extracts of hESCs, NPCs and neurons (FIG. 34), indicating that indeed the increased peptidase activity was due to the proteasome. In addition, the other two activities of the proteasome, the caspase-like and trypsin-like, were also increased in hESCs (FIG. 28c-d). Proteasome activity did not differ depending on the passage number (FIG. 35). The decrease in proteasome activity was not a specific phenomenon associated with the neural lineage since differentiation into either trophoblasts or fibroblasts induced a similar decrease (FIG. 28e-f). Notably, hESCs lost their high proteasome activity in a continuous progressive manner during the differentiation process (FIG. 28e). Moreover, Applicants examined other cell lines extracted from human tissues, such as astrocytes or BJ fibroblasts, or immortalized HEK293T cell and found that these cells also had lower proteasome activity compared to hESCs (FIG. 36). Applicants tested whether high proteasome activity in hESCs was associated with increased proliferation and found that hESCs and HEK293T cells had nearly identical proliferation rates, yet hESCs had higher proteasome activity (FIG. 37).

Induced pluripotent stem cells (iPSCs) can be derived from adult somatic cells by forced expression of exogenous factors that promote cell reprogramming (Takahashi, K., et al., Cell 131, 861-872 (2007); Takahashi, K. & Yamanaka, S., Cell 126, 663-676 (2006); Yu, J., et al., Science 318, 1917-1920 (2007)). iPSC lines are similar to ESCs in many aspects, such as their gene expression patterns, proteome profile and potential for differentiation (Takahashi, K. & Yamanaka, S., Cell 126, 663-676 (2006); Hanna, J. H., Saha, K. & Jaenisch, R., Cell 143, 508-525 (2010)). However, the full extent of their similarity to ESC is still being assessed (Panopoulos, A. D. et al., Cell Stem Cell 8, 347-348 (2011)). Applicants analyzed two iPSC lines carefully validated to ensure similar gene expression profile, growth characteristics and developmental potential to hESCs (Brennand, K. J., et al., Nature 473, 221-225 (2011)). Applicants discovered that these iPSC lines derived from BJ fibroblasts display increased proteasome activity similar to hESCs (FIG. 28g), indicating that proteasomal activity can indeed be reprogrammed.

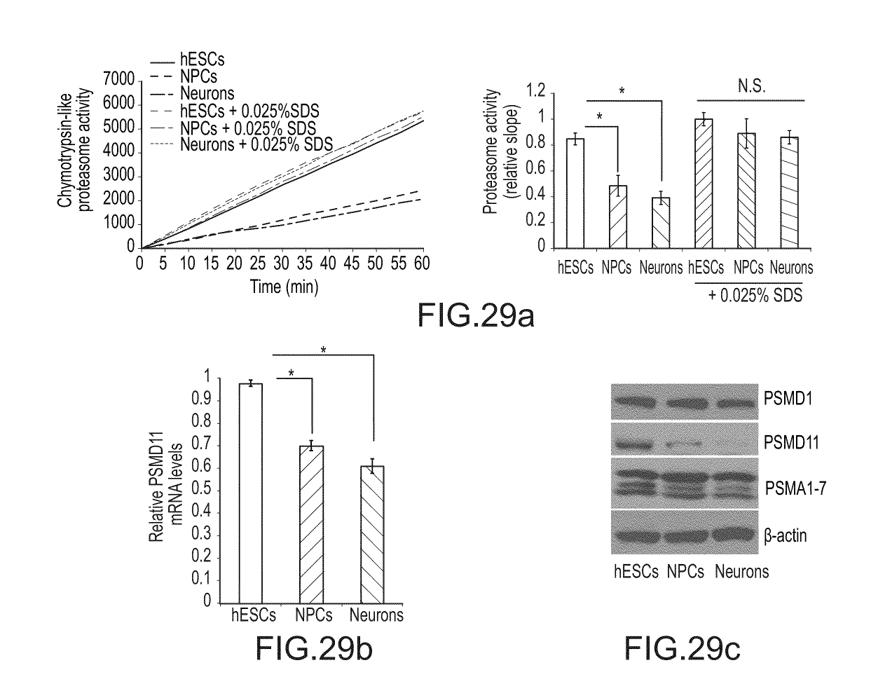

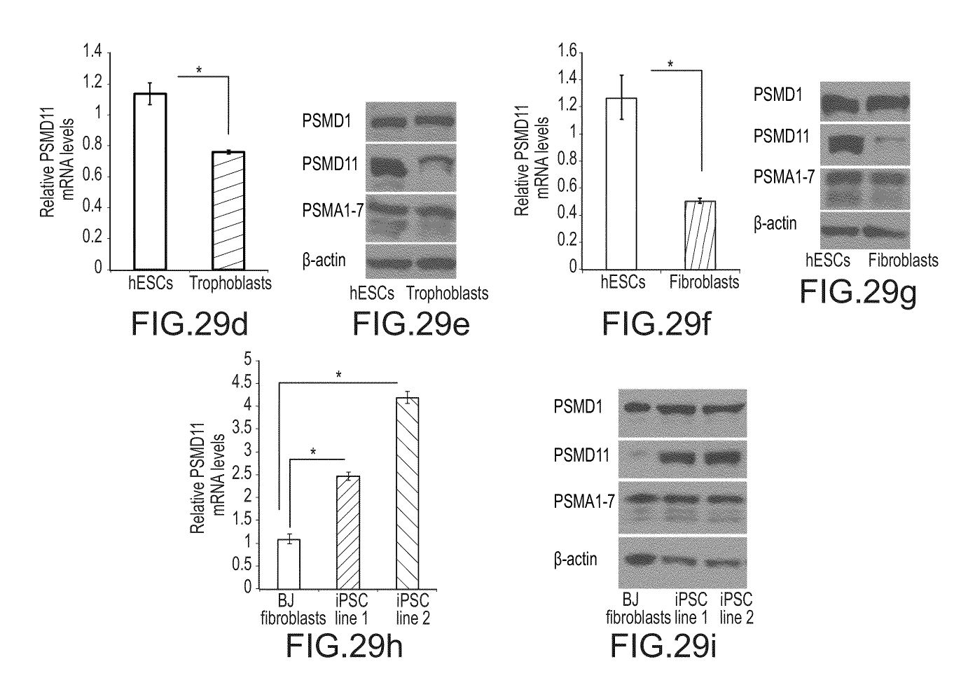

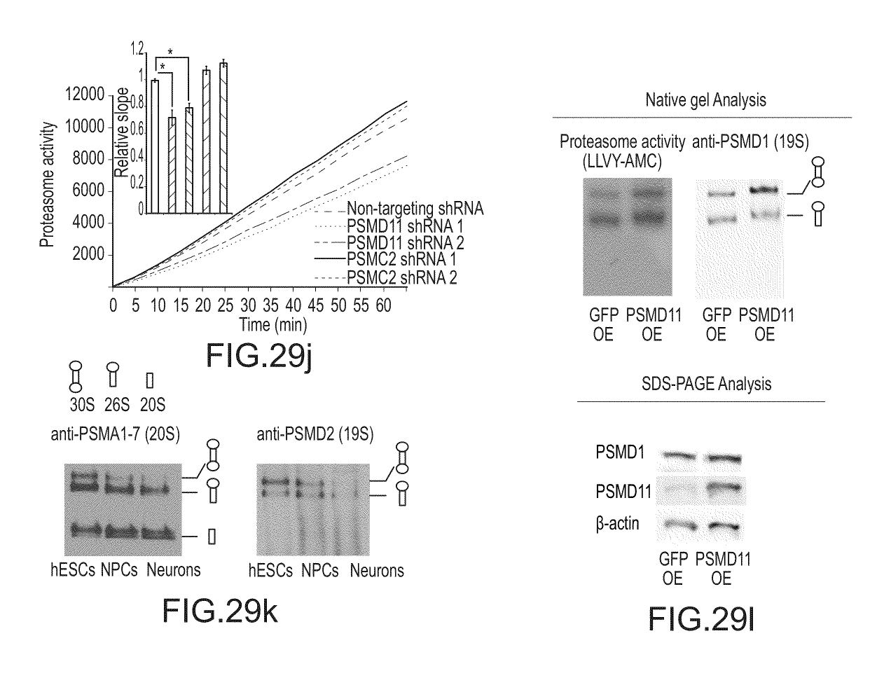

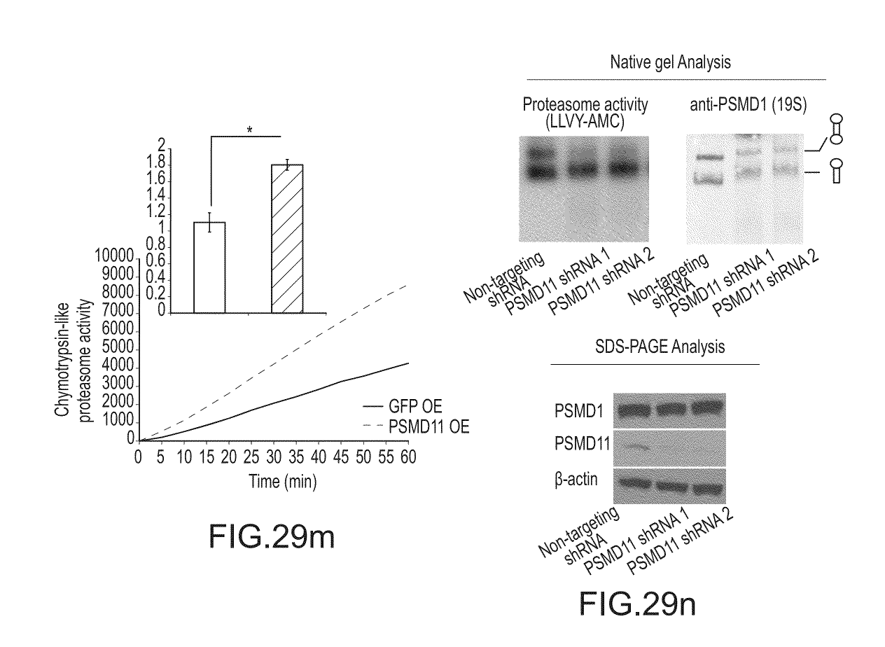

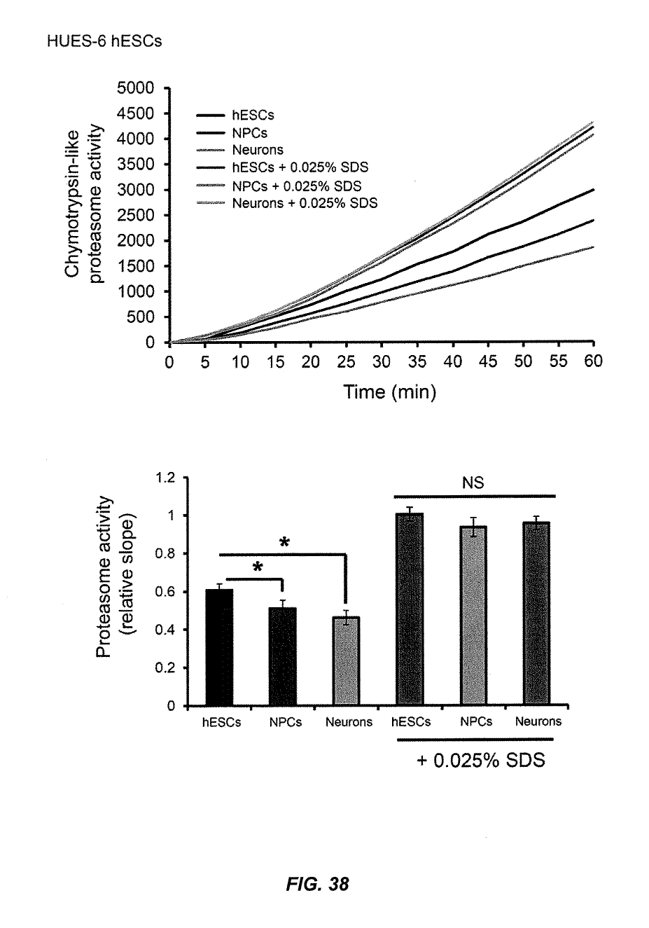

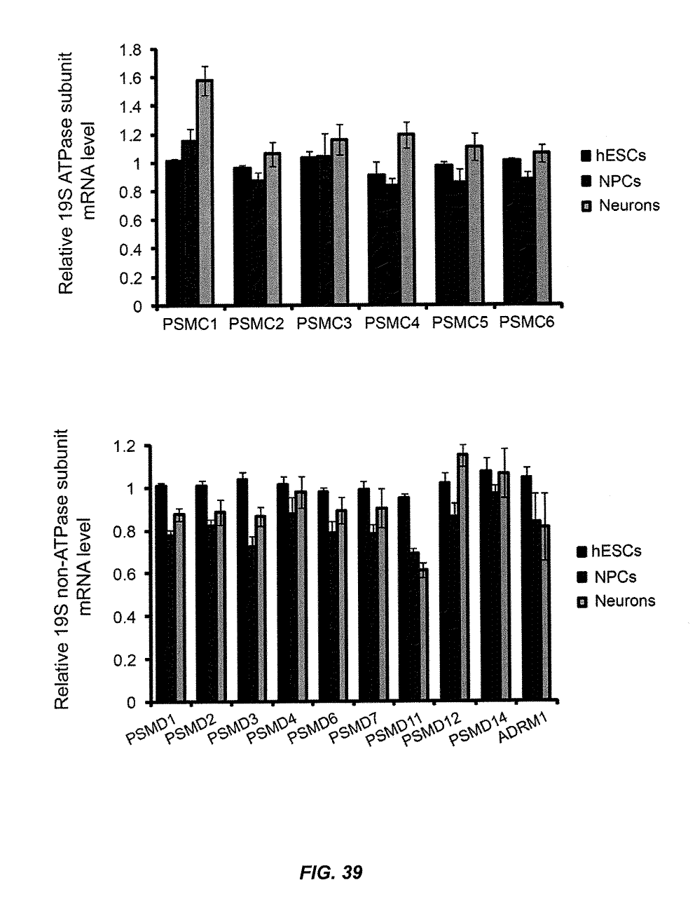

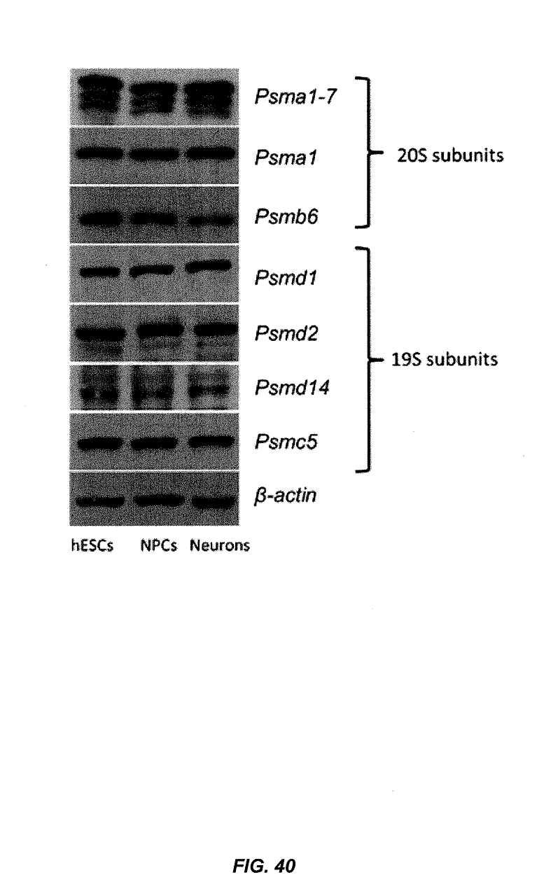

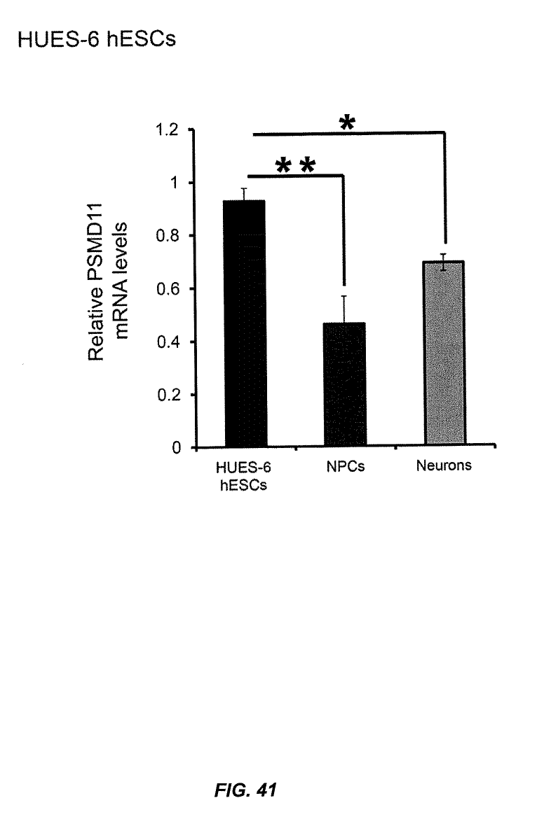

The 26S/30S proteasome consists of a 20S core structure containing the proteolytic active sites and 19S cap structures that impart regulation on the activity of the holo-complex (26S, single and 30S, double capped) (Finley, D., Annual review of biochemistry 78, 477-513 (2009)). Although 20S particles can exist in a free form, 20S particles in their most physiological form are inactive, unable to degrade denatured proteins or cleave peptides_ENREF_{—}10 (Kisselev, A. F. & Goldberg, A. L., Methods in enzymology 398, 364-378 (2005)). The 19S regulatory subunit is responsible for stimulating the 20S proteasome to degrade proteins, since ATPases of the regulatory particle open the 20S core, allowing substrates access to proteolytic active sites_ENREF_{—}19 (Kohler, A., et al., Molecular cell 7, 1143-1152 (2001)). Treatment of cell extracts with 0.025% SDS, a condition that activates 20S particles by allowing gate opening (Coux, O., Tanaka, K. & Goldberg, A. L., Annual review of biochemistry 65, 801-847 (1996)), resulted in equivalent activities among all cell types (FIG. 29a and FIG. 38). This result suggests that all cell types have an equal number of 20S particles, but hESCs have increased levels of active 26S/30S proteasomes. Applicants examined the expression of the different 19S proteasome subunits and observed that PSMD11, the human orthologue of rpn-6, was the only 19S subunit to decrease as hESCs differentiated (FIG. 29b-g and FIG. 39-41). Consistent with hESCs results, Applicants observed increased PSMD11 levels in iPSCs (FIG. 29h-i). Accordingly, decreased expression of PSMD11 in hESCs (Table 7) reduced proteasome activity (FIG. 29j), demonstrating that the increased levels of this subunit in hESCs are critical for increased proteasome activity. In contrast, knockdown of PSMC2 did not decrease proteasome activity in hESCs (FIG. 29j). PSMD11 plays a critical role in stabilizing the otherwise weak interaction between the 20S core and the 19S cap (Pathare, G. R., et al., Proceedings of the National Academy of Sciences of the United States of America 109, 149-154 (2012)), suggesting that hESCs might have more assembled proteasomes. Applicants detected more 30S particles in hESCs compared to NPCs and neurons. Additionally, as more 20S subunits are assembled into 30S particles, less free 20S is found in hESCs (FIG. 29k). Ectopic expression of PSMD11 was sufficient to increase proteasome assembly and activity in cells with relatively low proteasome activity (FIG. 29l-m). Moreover, knockdown of PSMD11 levels decreased the assembly of active proteasomes (FIG. 29n).

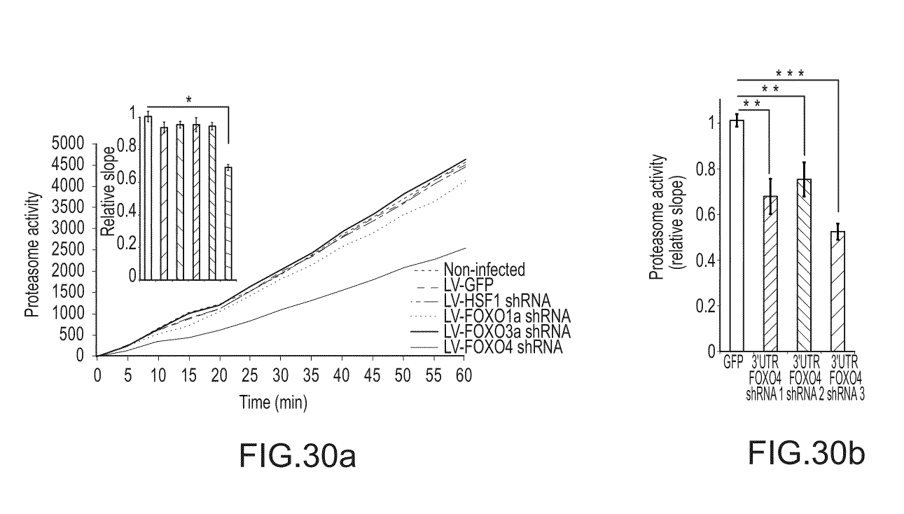

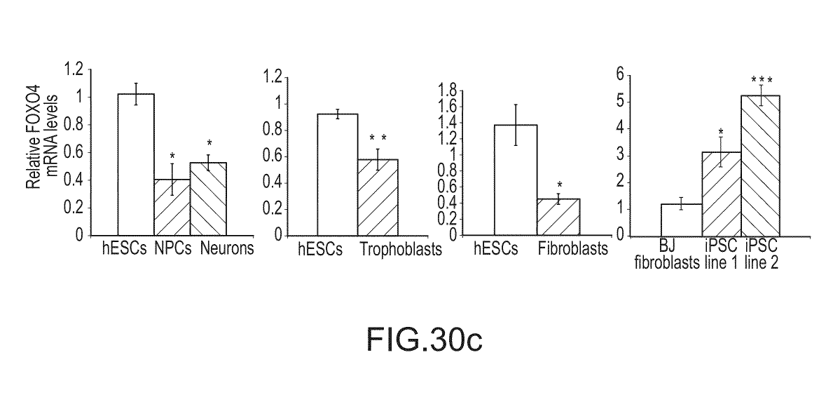

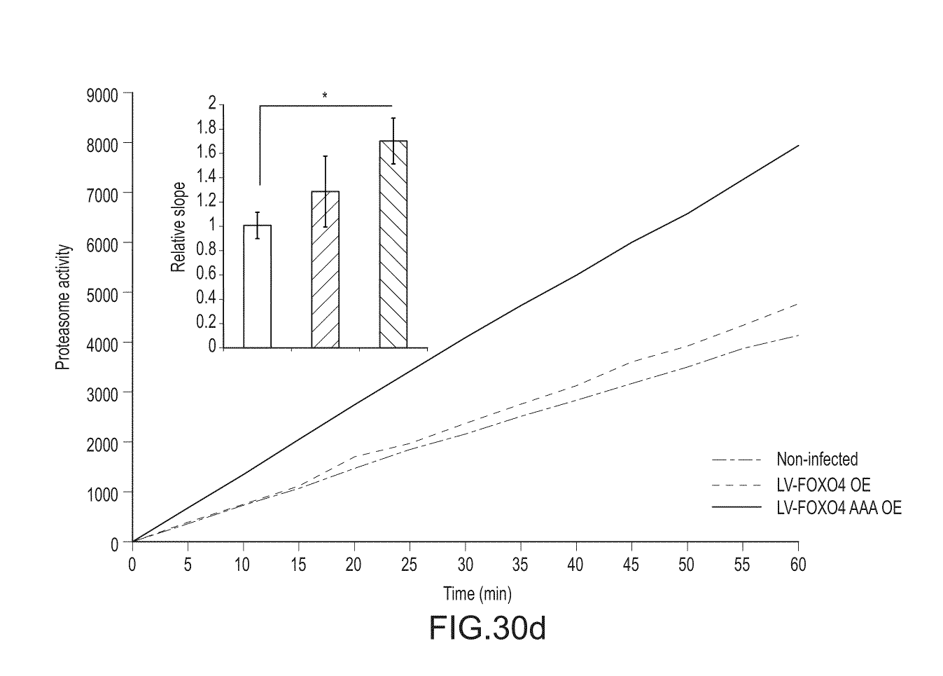

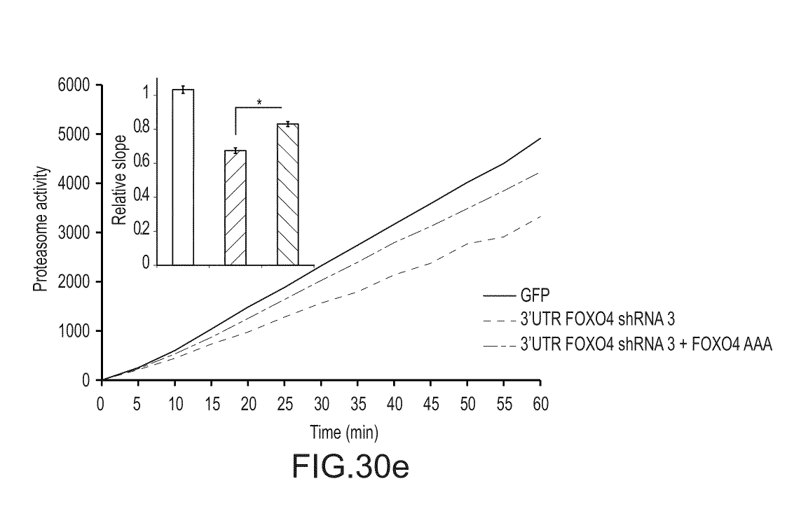

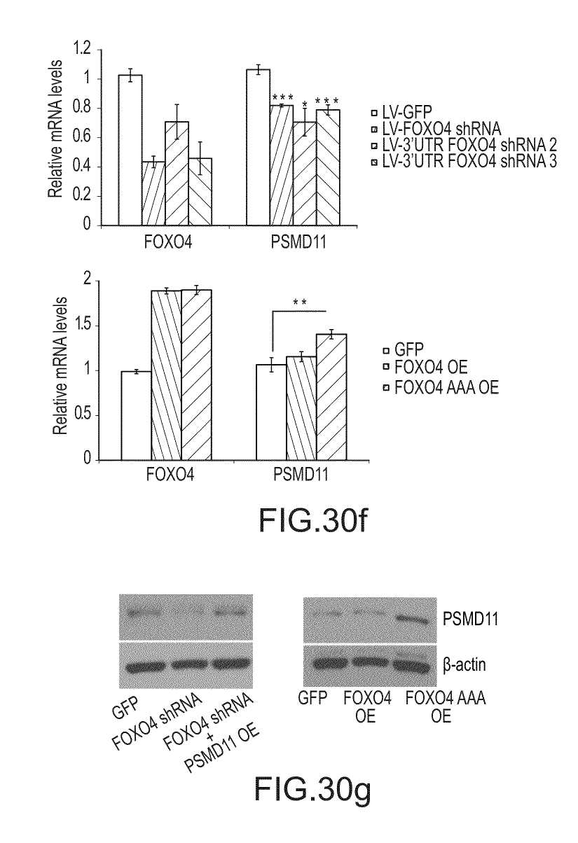

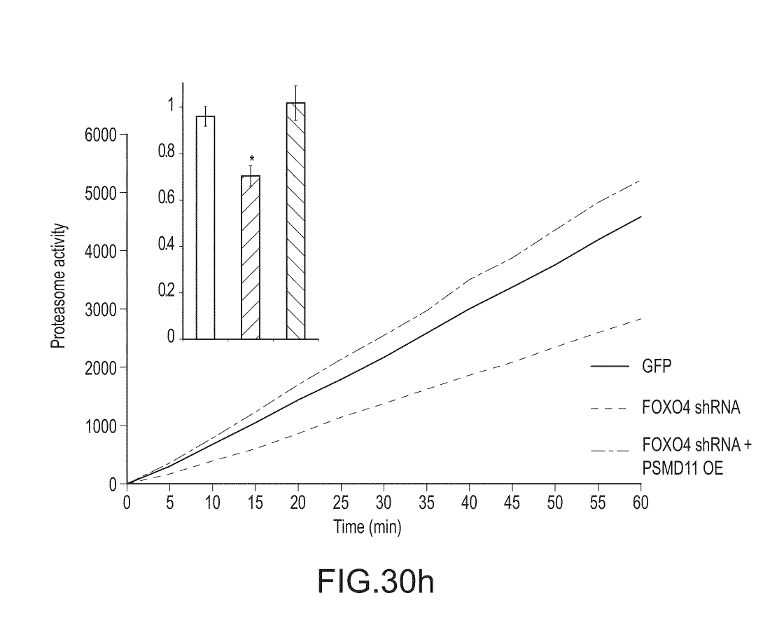

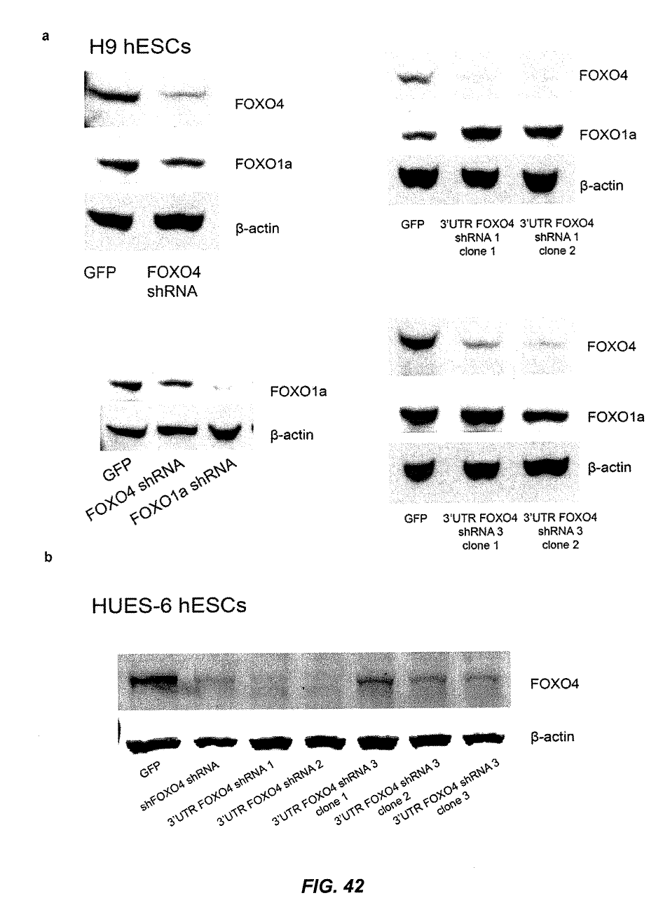

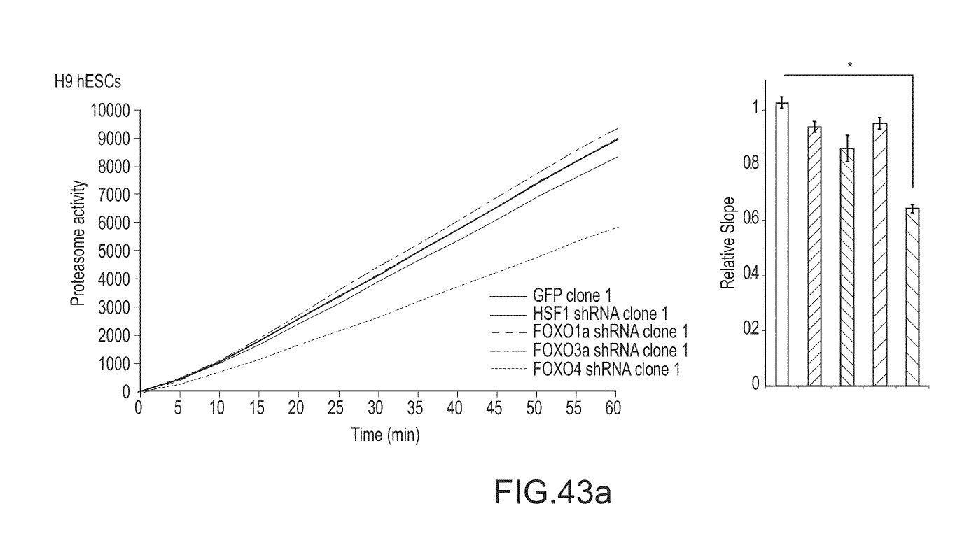

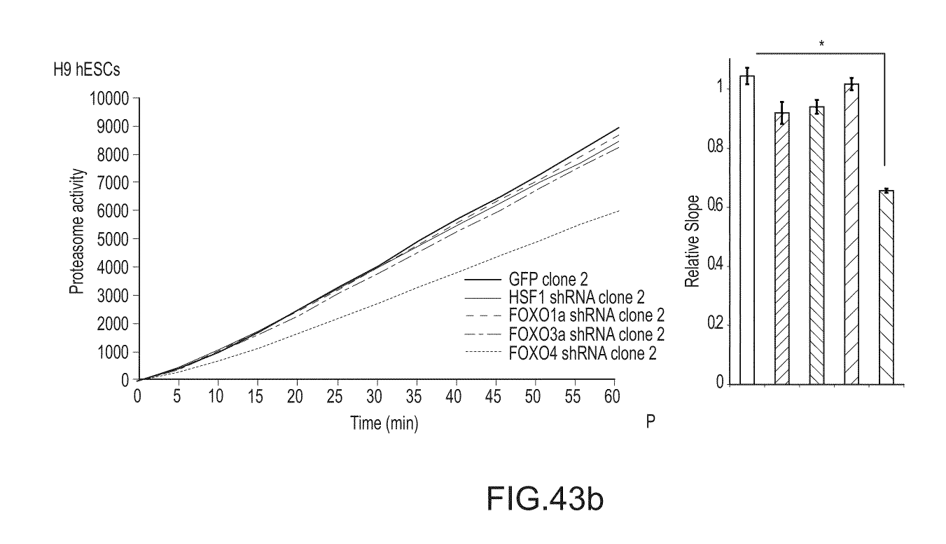

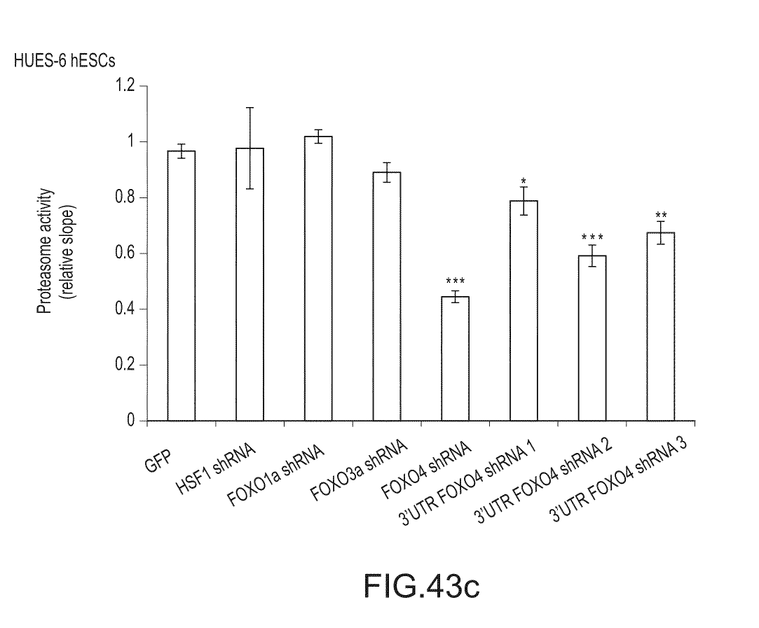

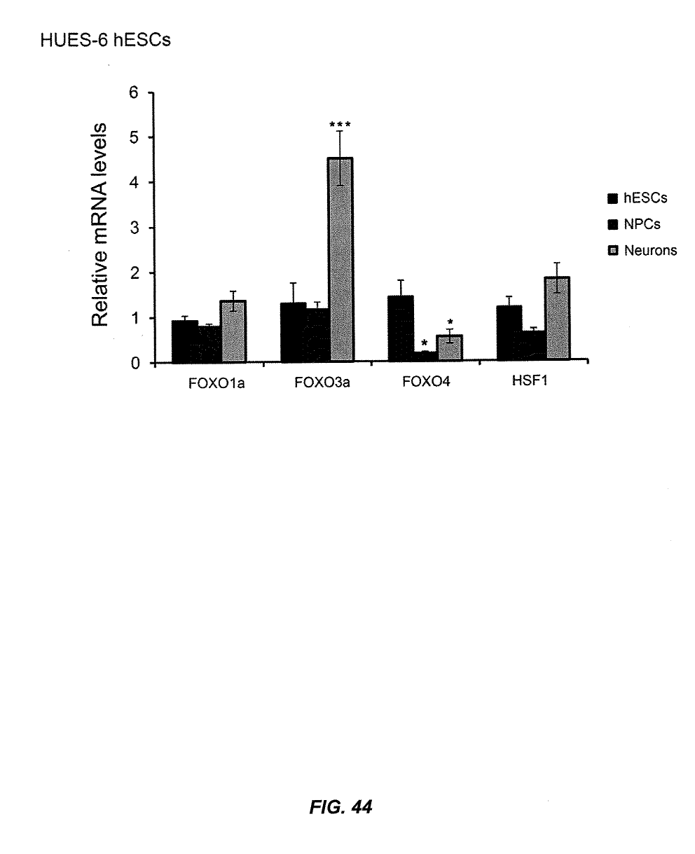

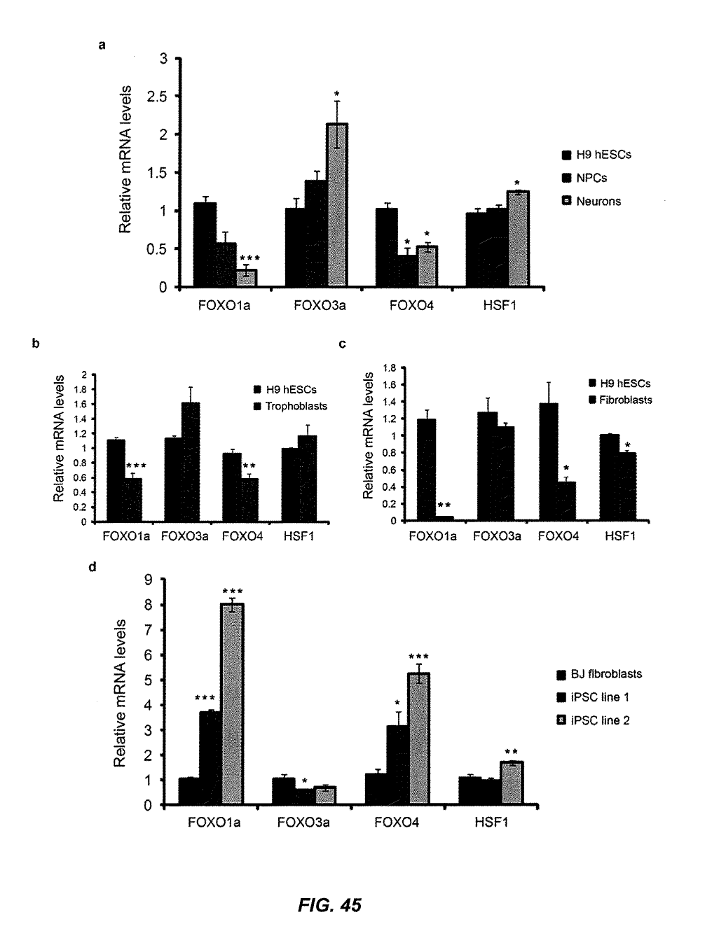

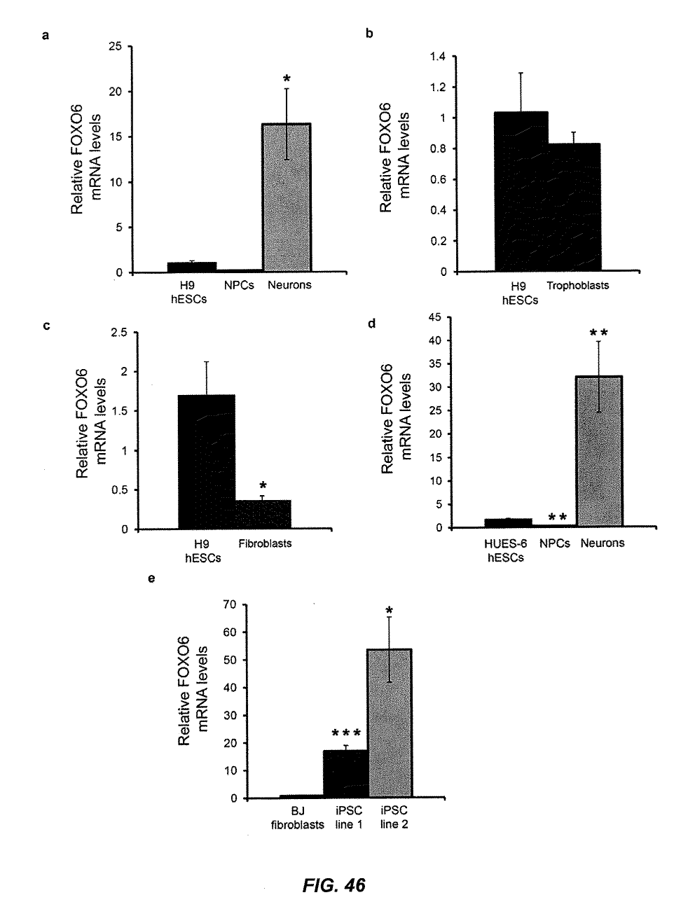

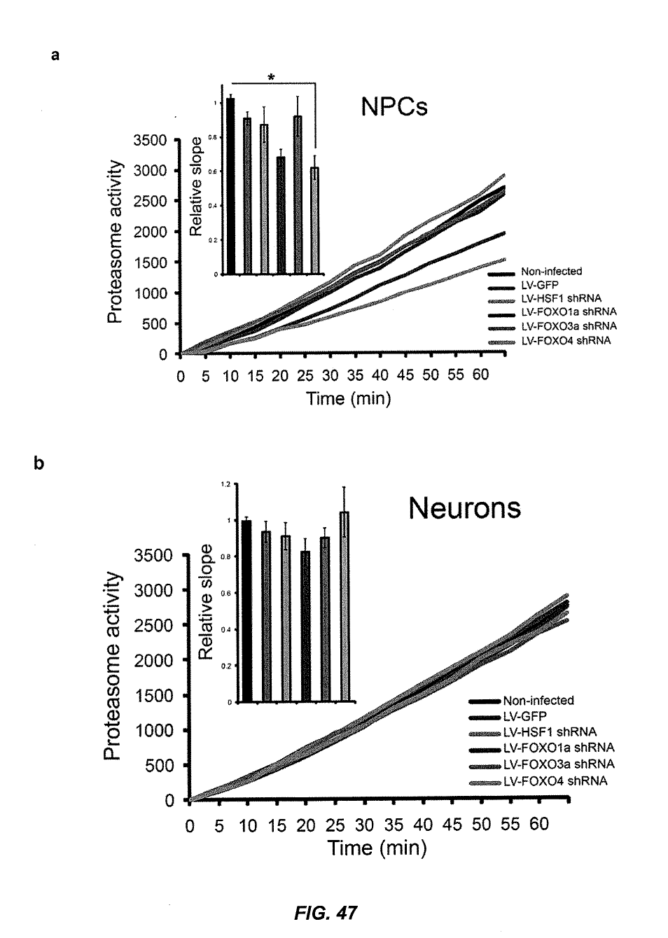

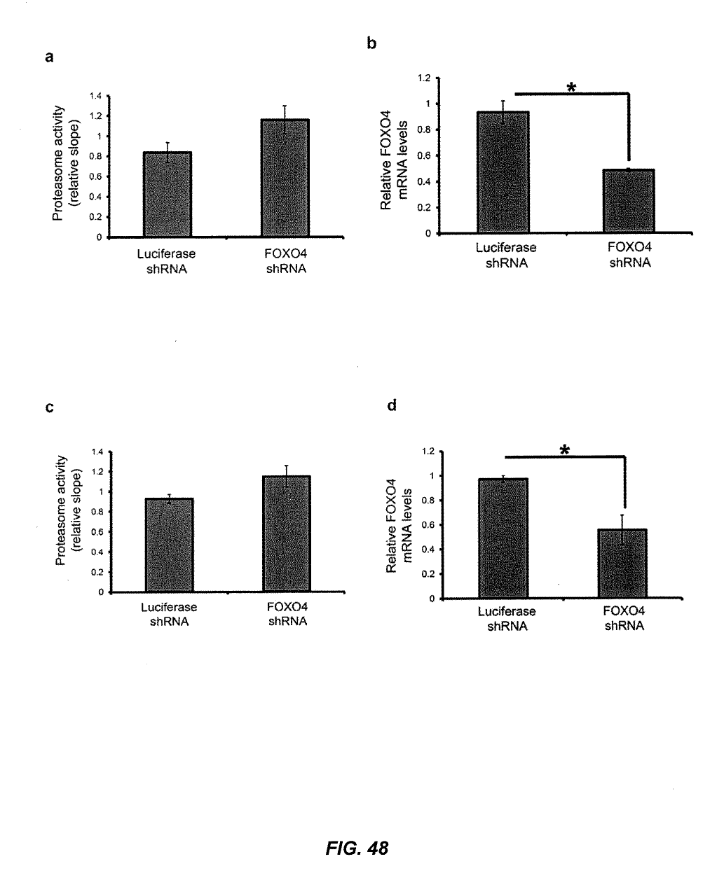

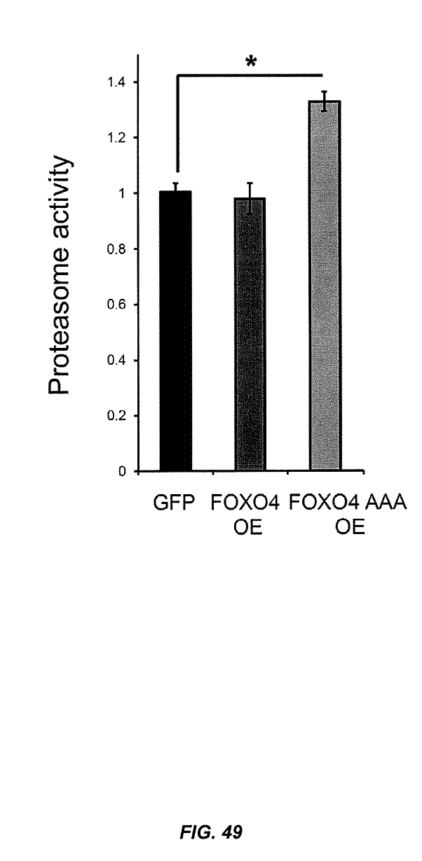

PSMD11/RPN-6 levels are increased in the long-lived C. elegans glp-1 mutant (accompanying manuscript). In this mutant, increased proteasome activity, rpn-6 expression and longevity are modulated by the DAF-16/FOXO transcription factor. To examine whether FOXO transcription factors regulate proteasome activity in hESCs, Applicants reduced expression of the closest human daf-16 orthologues: FOXO1a, FOXO3a and FOXO4 (FIG. 42 and Table 8). Strikingly, Applicants found that FOXO4 was critical to modulate proteasome activity in hESCs whereas FOXO1a and FOXO3a, as well as a HSF-1, had little or no effect on proteasome activity (FIG. 30a-b and FIG. 43). As hESCs differentiated into neural cells, trophoblasts or fibroblasts, FOXO4 had a corresponding decrease in its expression (FIG. 30c and FIG. 44-46). Accordingly, this decrease in FOXO4 expression is reprogrammed from somatic cells to iPSCs (FIG. 30c). FOXO1a had a similar expression pattern to FOXO4 in H9 but not in HUES-6 hESCs (FIG. 44-45). Furthermore, reduction of FOXO4 affected proteasome activity in the multipotent NPCs, which retain partial stem cell character, but did not affect proteasome activity in differentiated neurons (FIG. 47 and Table 9). These results raised the question whether FOXO4 regulation upon proteasome activity could be a general mechanism found in dividing cells. However, Applicants found that FOXO4 was not required for proteasome activity regulation in dividing cells such as BJ fibroblasts or HEK293T but rather appears specific to hESCs (FIG. 48). FOXO4 transcriptional activity is inhibited by phosphorylation on Thr32, Ser197, and Ser262 sites and once dephosphorylated, it translocates to the nucleus and induces target gene expression (Kops, G. J., et al., Nature 398, 630-634 (1999); Matsuzaki, H. et al., J Biochem 138, 485-491 (2005)). Expression of constitutively active FOXO4 triple alanine mutant (FOXO4 AAA), but not wild-type FOXO4, resulted in further up-regulation of proteasome activity in hESCs (FIG. 30d, FIG. 49 and Table 10a-b). In addition, ectopic expression of FOXO4 AAA in FOXO4 shRNA cells partially rescued the decreased proteasome activity of these cells (FIG. 30e and Table 10c).

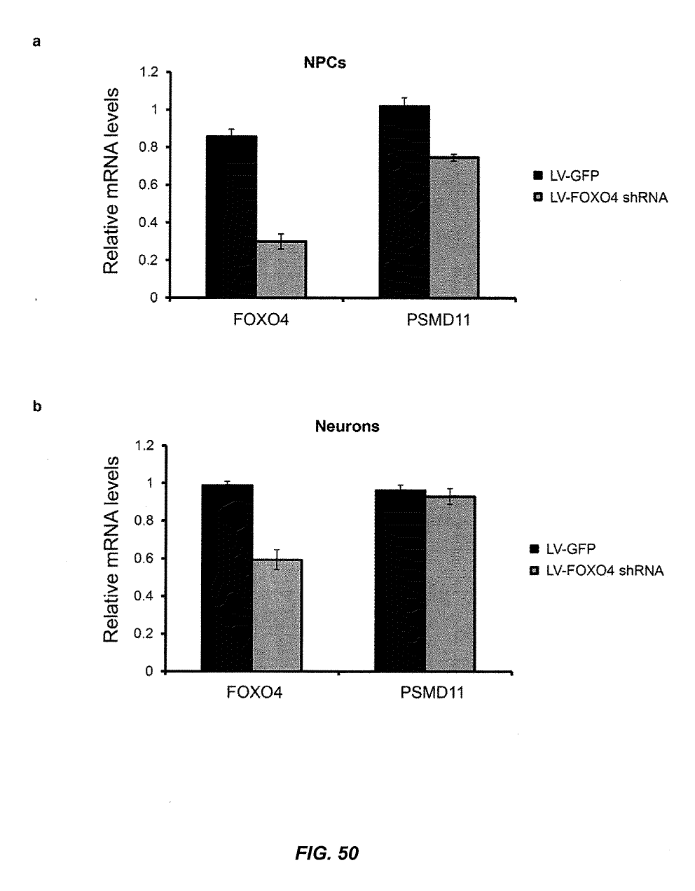

Applicants tested whether the increased proteasome activity of hESCs conferred by FOXO4 was due to PSMD11. Applicants found that loss of FOXO4 resulted in reduced expression of PSMD11 in hESCs and in the multipotent NPCs, but did not affect PSMD11 in differentiated neurons (FIG. 30f-g and FIG. 50). Knockdown of FOXO1a, FOXO3 or HSF-1 did not affect the expression of PSMD11 in any cell type tested, including hESCs (Tables 11-13). Moreover, overexpression of FOXO4 AAA increased PSMD11 levels in hESCs (FIG. 30f-g and Table 11). Ectopic expression of PSMD11 in FOXO4 shRNA hESCs rescued the decreased proteasome activity of these cells, indicating that expression of PSMD11 by FOXO4 is sufficient to regulate proteasome activity (FIG. 30g-h).

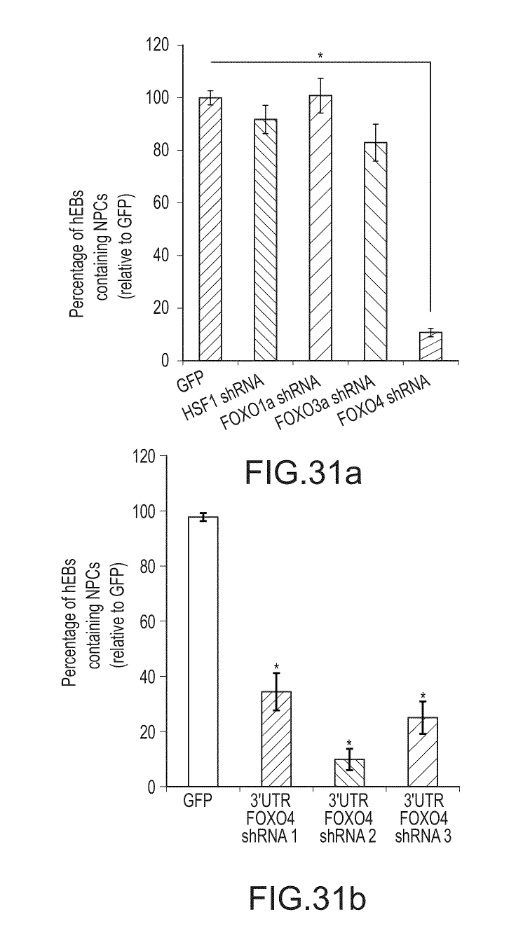

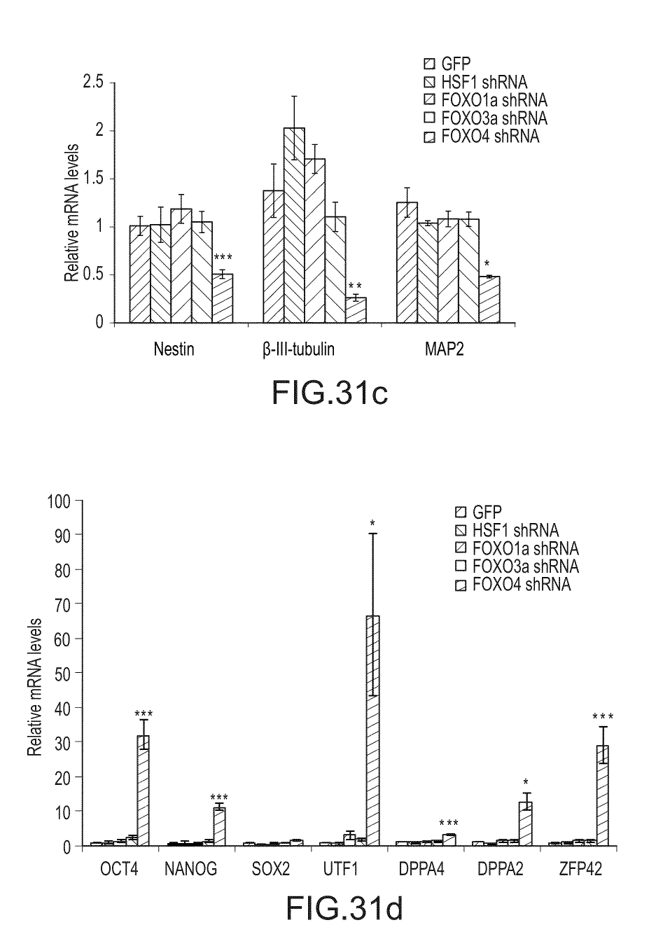

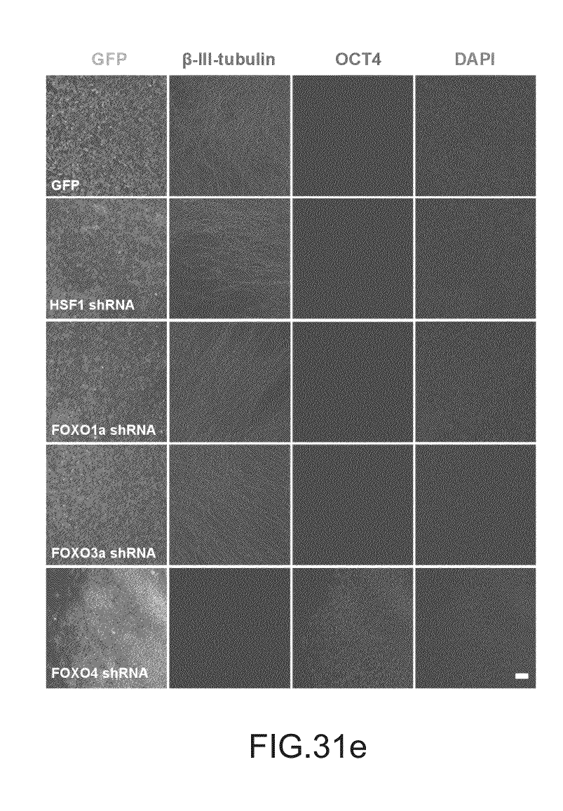

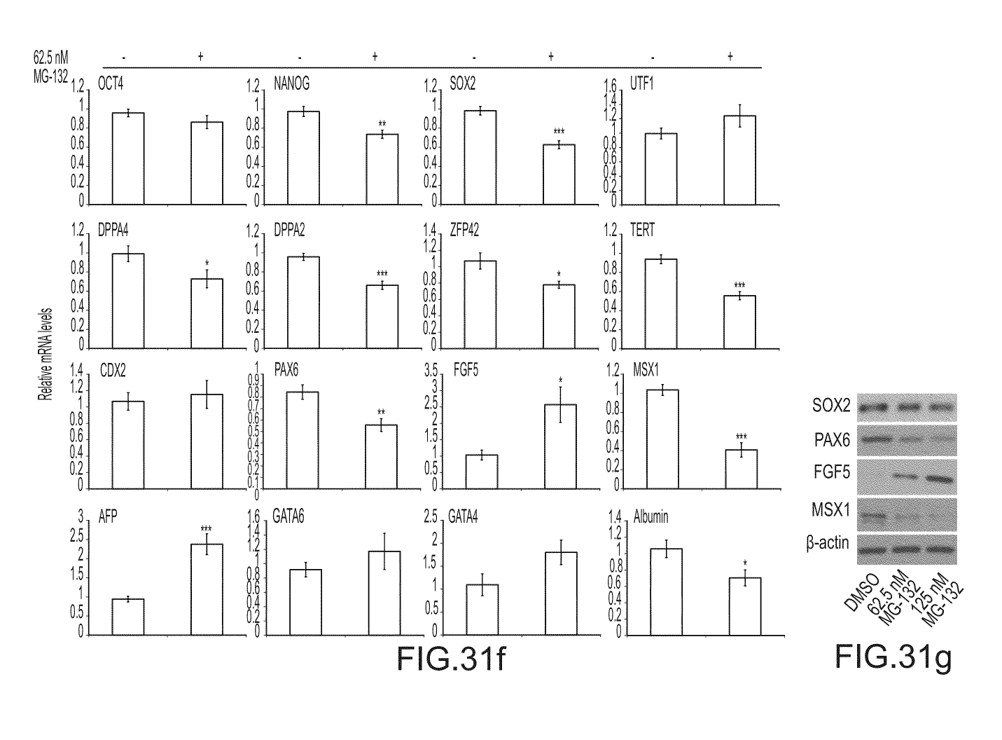

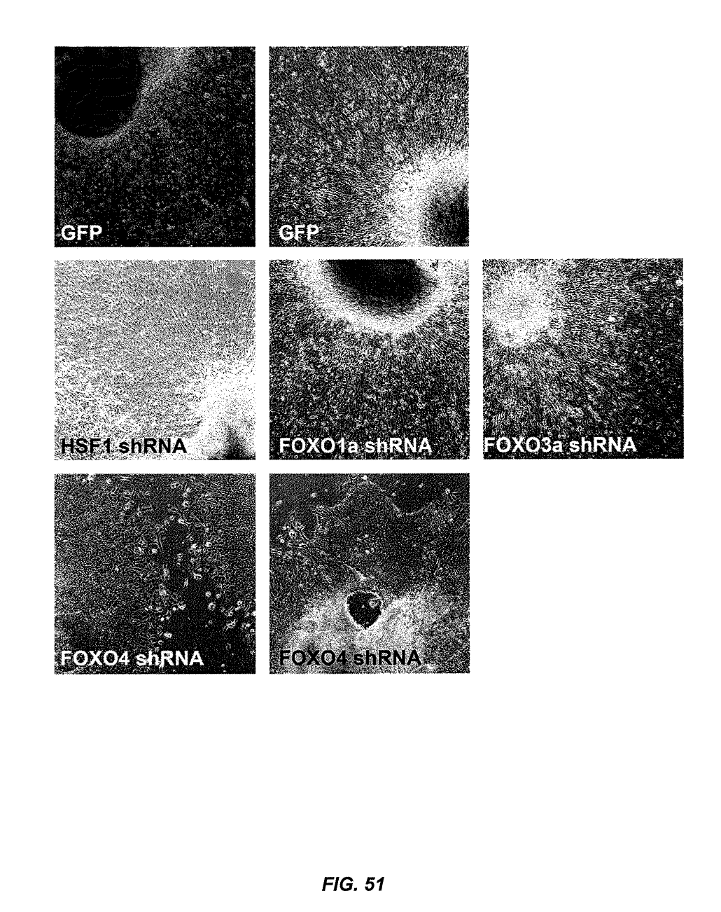

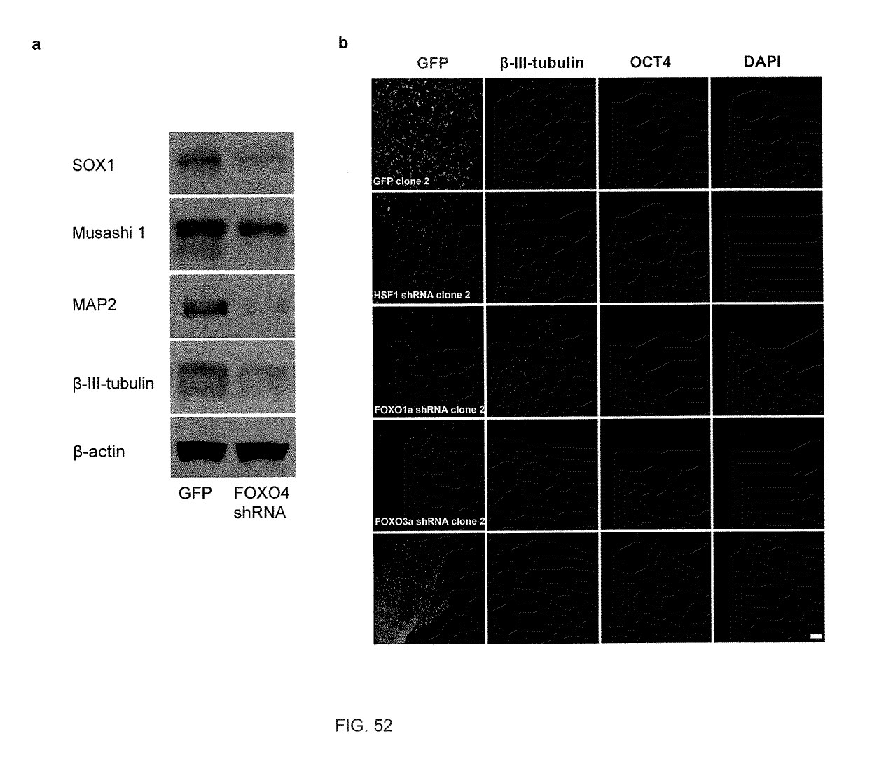

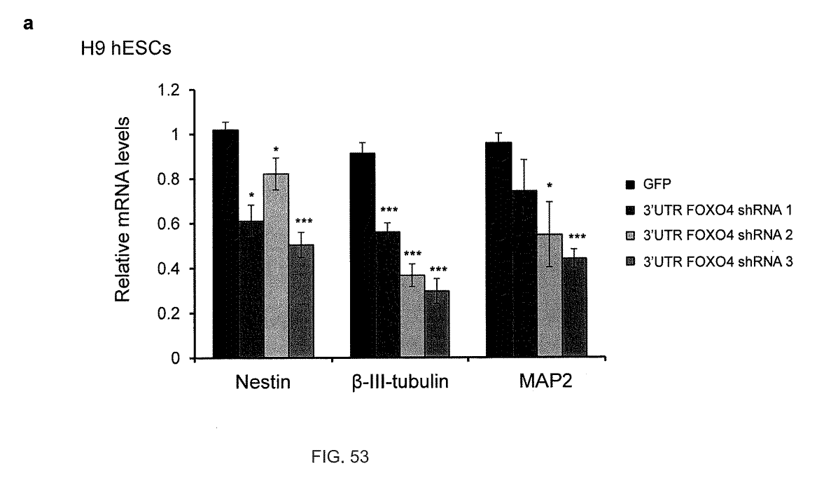

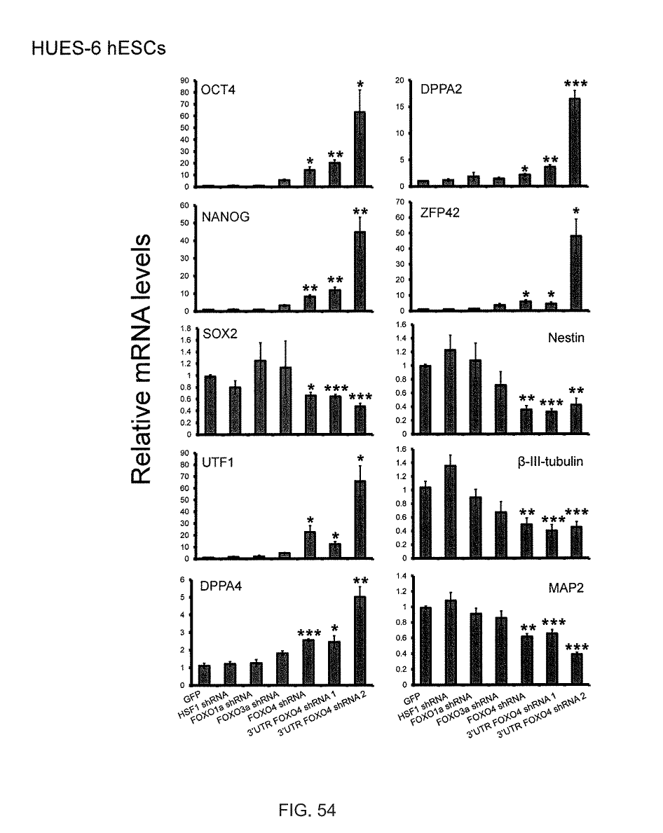

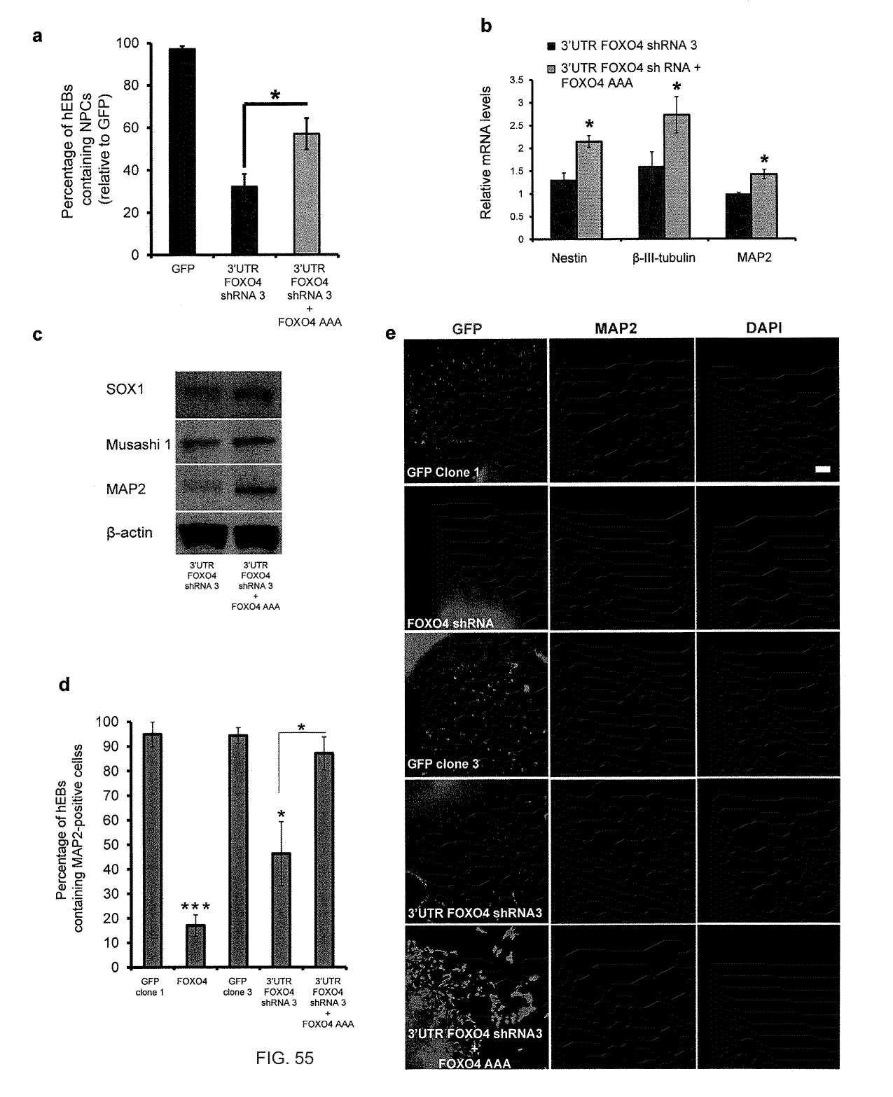

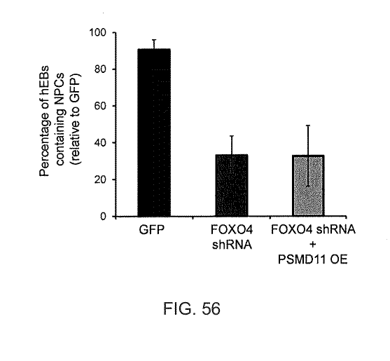

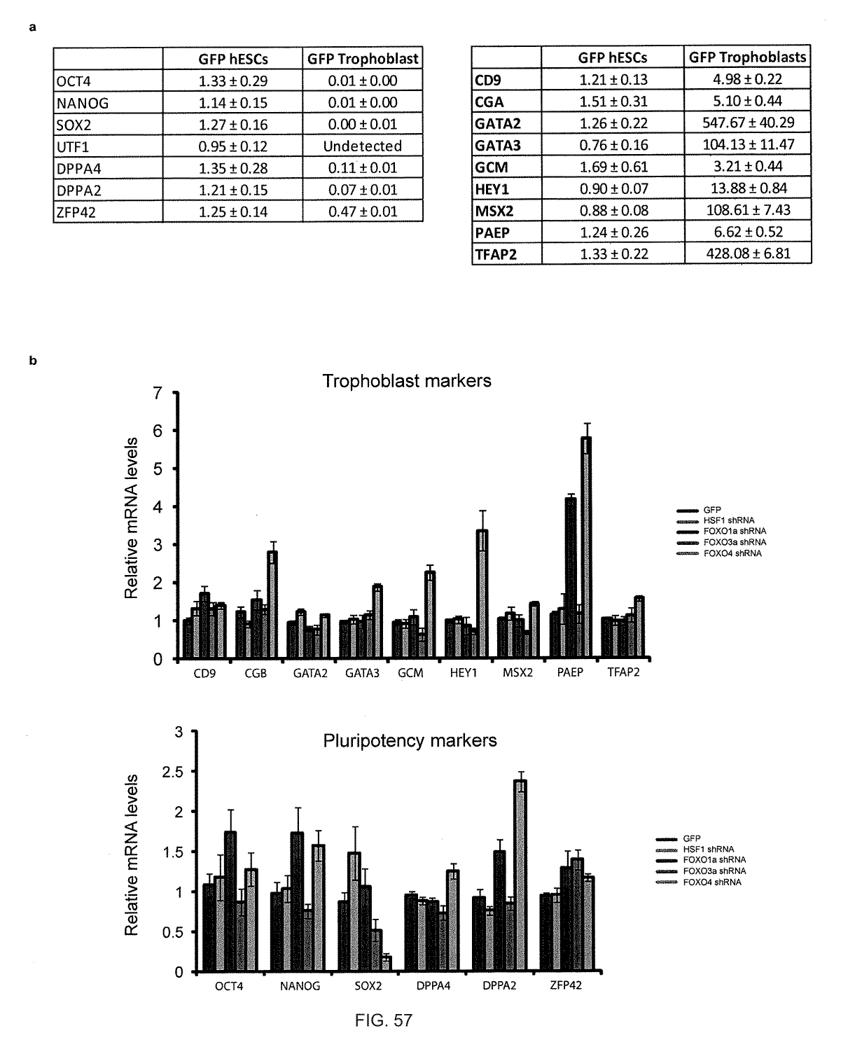

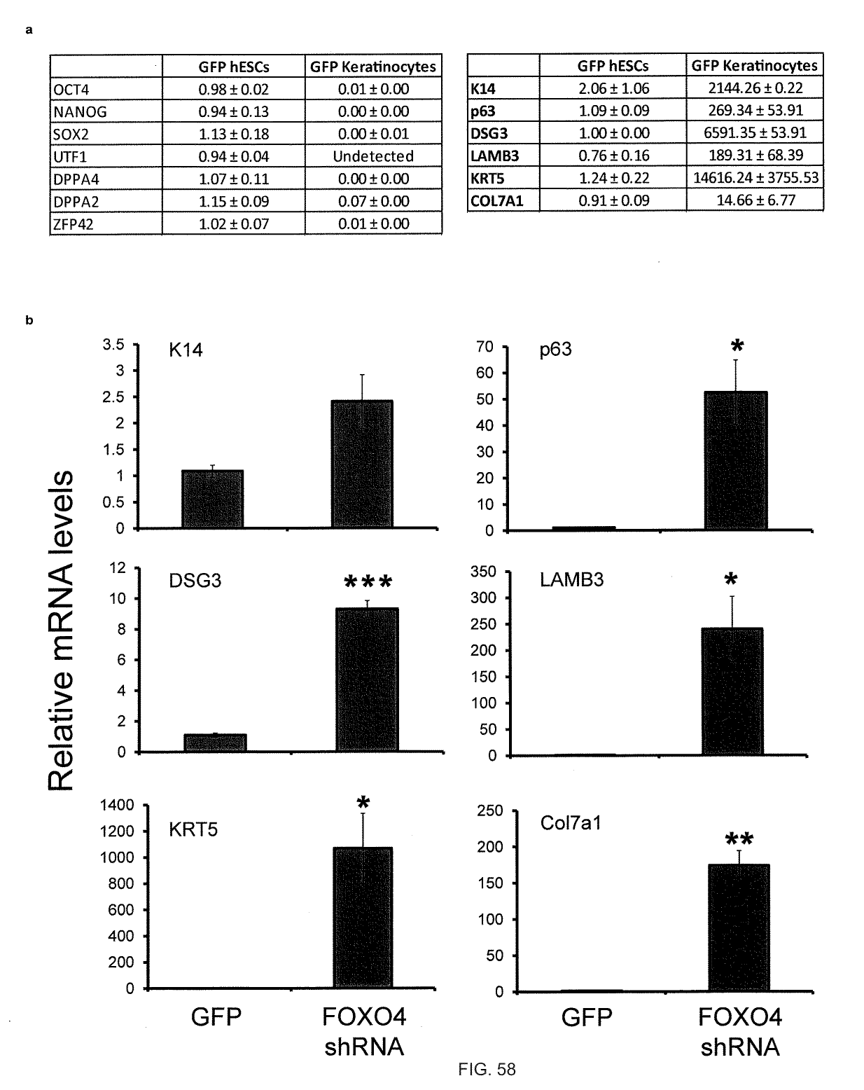

Prompted by these intriguing results, Applicants tested whether FOXO4 was required for proper function of hESCs. Applicants measured the expression levels of several markers of pluripotency in these cells prior to differentiation and found no difference at this stage (Table 14). However, when Applicants forced differentiation into neural lineage (Table 15), Applicants observed profound differences among the FOXO4 shRNA lines that were not present in the control lines: FOXO4 shRNA embryoid bodies were unable to generate rosettes and, accordingly, neural cells (FIG. 31a-b and FIG. 51). As a more direct test of molecular changes to these cells, Applicants found that the FOXO4 shRNA lines did not induce expression of NPC markers (e.g. nestin) and proteins involved in neurogenesis (β-III tubulin, MAP2) at the same extent than control lines after the differentiation treatment. Accordingly, FOXO4 shRNA cells retained pluripotency markers since most of them do not progress through differentiation into neural cells (FIG. 31c-e, FIG. 52-55 and Table 16). Moreover, ectopic expression of FOXO4 AAA ameliorated the low ability of FOXO4 shRNA hESCs to differentiate into neural cells (FIG. 55). On the contrary, ectopic expression of PSMD11 in FOXO4 shRNA hESCs was not sufficient to rescue this phenotype (FIG. 56), indicating that FOXO4 has additional target genes that are critical for neural differentiation. Applicants asked whether differentiation into other cell lineages might also be affected by reduction of FOXO4 levels. Applicants found that FOXO4 shRNA hESCs were able to properly differentiate into either trophoblasts or keratinocytes (FIG. 57-58 and Table 17). Notably, after the respective differentiation process, FOXO4 shRNA hESCs had increased levels of trophoblast or keratinocyte markers compared to control cells (FIG. 57-58 and Table 17).

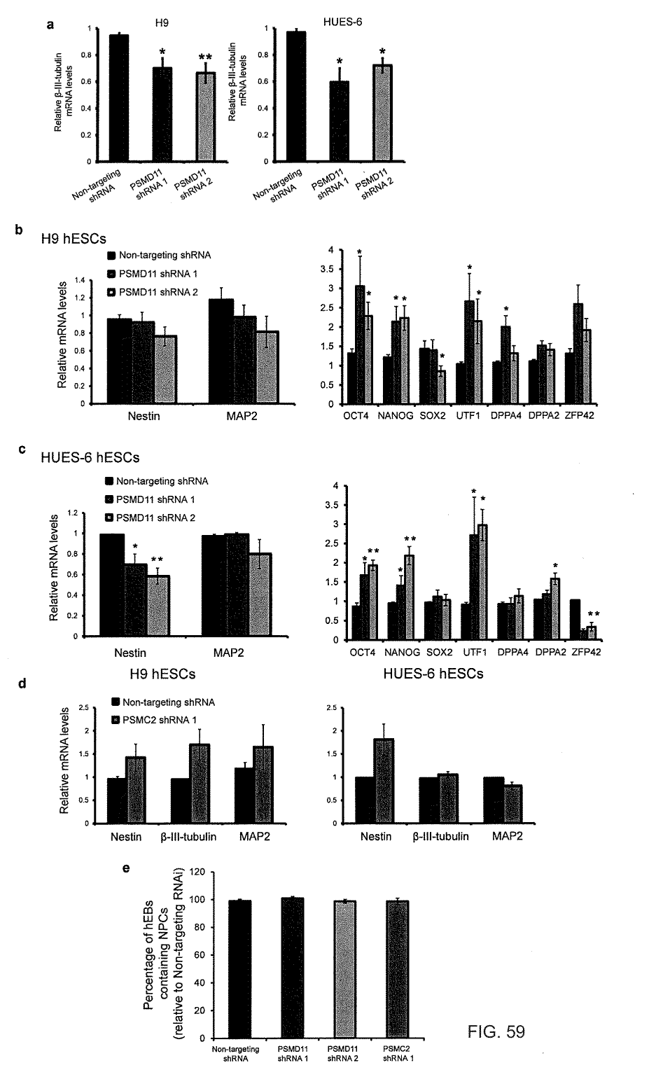

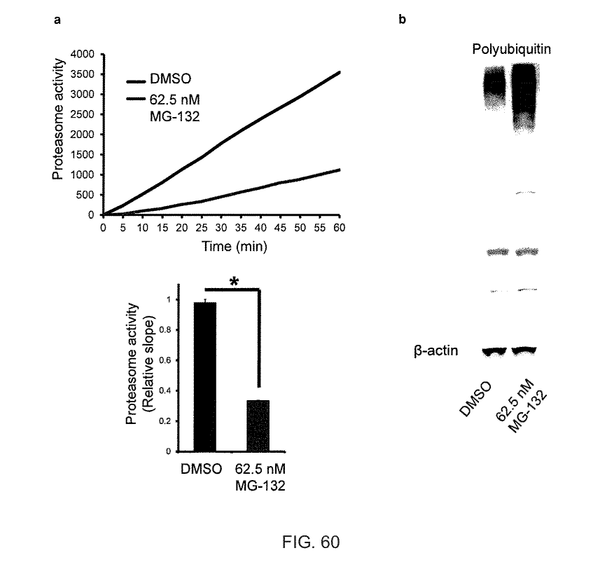

Applicants tested whether PSMD11, like FOXO4, was required for neural differentiation. Due to the important role of PSMD11 in proteasomal function, an essential structure for the cell, Applicants could not obtain stable hESCs with robust PSMD11 knockdown. However, even with weak reduction of PSMD11 Applicants observed significant decreased expression of β-III tubulin and increased pluripotency markers in PSMD11 shRNA hESCs after neural differentiation. Although reduced β-III tubulin, these cells had a similar efficiency in the generation of embryoid bodies containing rosettes and/or neuronal projections (FIG. 59). Because Applicants could not achieve robust reduction of PSMD11, Applicants induced an acute proteasome inhibition in hESCs by using MG-132 proteasome inhibitor to assess the requirement for proteasome activity in hESC function. Notably, hESCs were more sensitive to proteasome inhibition than NPCs or neurons and Applicants had to decrease MG-132 concentration almost 100 times in order to avoid cell death and detachment of hESCs (data not shown). Strikingly, low concentrations of MG-132 (62.5 nM) were sufficient to reduce proteasome activity and induce accumulation of polyubiquitinylated proteins in hESCs (FIG. 60). In the absence of differentiation treatment, Applicants already observed that proteasome inhibition resulted in decreased pluripotency markers and modified the levels of markers of the distinct germ and extraembryonic layers, while decreasing the expression of proteins involved in neurogenesis (FIG. 31f-g). Taken together, these results suggest that acute proteasome inhibition affects pluripotency of hESCs inducing differentiation towards specific cell lineages to the detriment of neurogenesis.

Collectively, Applicants' results establish increased proteasome activity as an intrinsic characteristic of hESC identity. Applicants' findings raise the intriguing question of why these cells need enhanced proteasome activity. One possibility is that hESC cannot tolerate toxic, misfolded proteins and increased proteostasis could be required to avoid hESC senescence and maintain an intact proteome either for self-renewal or the generation of an intact cell lineage. Alternatively, the high proteasome activity may be tightly linked to other cellular process, such as translation, to ensure future integrity of the proteome. In addition, Applicants' results indicate that an orthologue of DAF-16, a transcription factor that regulates both lifespan and resistance to proteotoxic stress in invertebrates, crosses evolutionary boundaries and links hESC function to invertebrate longevity modulation. It will be of particular interest to identify additional genes of the proteostasis network regulated by FOXO4 in hESCs. In conclusion, Applicants' findings may trigger new advances in understanding hESC differentiation or cell reprogramming and open new possibilities for cell therapy by modulation of either FOXO4 or the proteasome.

Experimental Procedures

hESCs culture and differentiation. Human H9 (WA09) hESC line was obtained from WiCell Research Institute. HUES-6 hESC line was obtained from the Laboratory of Douglas Melton, Harvard University. hESC lines were maintained on a mitotically inactive mouse embryonic fibroblast (MEFs) feeder layer in hES medium, DMEM/F12 (Invitrogen) supplemented with 20% Knockout Serum Replacement (Invitrogen), 1 mM L-glutamine, 0.1 mM non-essential amino acids, β-mercaptoethanol and 10 ng/ml bFGF (Joint Protein Central, S. Korea). When co-culturing hESCs with MEFs was not possible due to interference with downstream assays H9 hESCs were also maintained on Matrigel (BD Biosciences) using mTeSR1 (Stem Cell Technologies). When cultured on Matrigel, HUES-6 cells were fed on conditioned medium harvested from cultured MEFSs. hESC colonies were passaged using a solution of collagenase (1 mg/ml) or dispase (2 mg/ml) and scraping the colonies with a glass pipette. For Applicants' experimental assays, Applicants used H9 hESCs passage 40-45 and HUES-6 hESCs passage 30-35. The human iPSC lines (control BJ-iPSC lines) were derived and characterized as previously reported (Brennand, K. J., et al., Nature 473, 221-225 (2011)) and cultured similarly as described above for hESCs cells.

Neural differentiation was performed as follows. hESCs grown on inactivated MEFs were fed with N2/B27 medium (DMEM/F12-GlutaMAX (Invitrogen), 1×N2 (Invitrogen) 1× B27-RA (Invitrogen)) for two days prior to being treated with collagenase type IV (1 mg/ml in DMEM/F12) at 37° C. for ˜1 hour. Once colonies lifted off the plate, they were gently washed and then transferred to ultra-low attachment plates (Corning). Aggregates (embryoid bodies—hEBs) were allowed to form and were grown in suspension for 1 week in N2/B27 medium with medium changes as needed, roughly every other day. The hEBs were then transferred onto polyornithine (PORN)/laminin-coated plates in N2/B27 medium with 1 μg ml⁻¹ laminin (Invitrogen) where they were allowed to adhere and develop neural rosettes and projections. After 1 week, colonies were either picked for neural precursor cell (NPC) line, or scored on an Olympus SZX10 dissecting microscope for the presence of neural rosettes or projections, before being fixed or harvested for RNA. Picked colonies containing rosettes or projections are dissociated with TrypLE (Invitrogen) for 5 minutes at 37° C. and plated onto PORN/laminin coated plates in NPC medium (DMEM/F12, N2/B27-RA (Invitrogen), 1 μg/ml laminin and 20 ng/ml FGF2). The resulting monolayer culture was grown at a high density and split 1:3 every week. For Applicants' experimental assays, Applicants used NPCs passage 10-14.

For neuronal differentiation, NPCs were dissociated with TrypLE (Invitrogen) and plated into neuronal differentiation medium (DMEM/F12, N2/B27-RA (Invitrogen), 1 μg/ml laminin, 20 ng/ml BDNF (Peprotech), 20 ng/ml GDNF (Peprotech), 1 mM dibutyryl-cyclic AMP (Sigma), 200 nM ascorbic acid (Sigma)) onto PORN/laminin-coated plates. For this study, cells were differentiated in 6-well plates, with approximately 2×10⁵ cells per 6-well. Cells were differentiated for 2-3 months, with weekly feeding of neuronal differentiation medium.

Differentiation to fibroblast cells involved the formation of embryoid bodies (EBs) as described above but cultured in EB medium (IMDM base medium supplemented with 15% FBS (Atlanta Biologicals), 0.1 mM non-essential amino acids, and 1% Glutamax (Invitrogen) and maintained on ultra-low attachment plates with daily medium changes. 1 week later the floating EBs were plated on gelatin-coated plates and passaged at confluence 3 times before use. Alternatively, a non-EB method was employed involving the individualization of hESCs using Accutase 1× (Millipore) and plating the cells at a density of 25×10³ cells per square cm in EB medium containing Rock Inhibitor (Y-27632, Stemgent) at 10 μM. The cells were fed daily with straight EB medium. At confluence any areas still showing a stem cell morphology were removed by aspiration then passaged using Accutase 1×. After 3 passages the cells present with fibroblast morphology and were confirmed by PCR of lineage specific markers.

Trophoblast differentiation was performed as described previously using high levels of BMP4 (Xu, R. H., et al., Nature biotechnology 20, 1261-1264 (2002)). Keratinocyte differentiation was performed following the protocol established in (Itoh, M. et al., Proceedings of the National Academy of Sciences of the United States of America 108, 8797-8802 (2011)). BJ human fibroblasts (ATCC, CRL-2522) were cultured in DMEM (Invitrogen) supplemented with 10% FBS and 0.1 mM non-essential amino acids and passaged with trypsin. Hippocampal and Cerebellar Astrocytes are from Sciencell, Carlsbad, Calif.

Generation of lentiviral vectors. The shRNA expressing lentiviral vectors were generated by cloning the sequences described in Table 18 into the pSIH1-copGFP vector (SBI Biosystems, Mountain View, Calif.) to generate pLV-siHSF-1, pLV-siFOXO1a, pLV-siFOXO3a, pLV-siFOXO4, pLV-si3′UTR_{—}1 FOXO4, pLV-si3′UTR_{—}2 FOXO4 and pLV-si3′UTR_{—}3 FOXO4. A control shRNA vector was generated by cloning the sequence CGT GCG TTG TTA GTA CTA ATC CTA TTT designed against the sequence of luciferase (SBI Biosystems) into the same vector to generate pLV-siLuc. The GFP expressing vector was prepared from the 3rd generation self-inactivating lentivirus (Tiscornia, G., Singer, O. & Verma, I. M. Nature protocols 1, 234-240 (2006)). Lentiviruses were packaged by transient transfection in 293T cells (Tiscornia, G., Singer, O. & Verma, I. M. Nature protocols 1, 234-240 (2006)).

LV-Non targeting shRNA Control, LV-shPSMD11_{—}1 (Clone ID: TRCN0000003948), LV-shPSMD11_{—}2 (TRCN0000003950), LV-shPSMC2_{—}1(TRCN0000007181), LV-shPSMC2_{—}2 (TRCN0000007183) in pLKO. 1-puro-CMV-tGFP vector were obtained from Mission shRNA (Sigma).

FOXO4 overexpressing lentiviral constructs were generated as follows. Flag-FOXO4 construct was obtained from Addgene (plasmid 17549). PCR was performed to generate a product to be cloned into pLVX puro lentiviral plasmid (Clontech) utilizing the XhoI/SmaI sites.

Forward primer (with 5′ XhoI site for cloning):

CGC GTA CTC GAG ATG GAT CCG GGG AAT GAG AAT TCA
GCC ACA GAG GCT GCC GCG ATC ATA GAC.

Reverse primer (with 3′ SmaI site for cloning);

CCG GAA CCC GGG TCA GGG ATC TGG CTC AAA G.

To generate FOXO4 AAA (Thr 32, Ser 197, Ser 262), site-directed mutagenesis of FOXO4 wild-type was performed by using Pfu Turbo. The primers used for site-directed mutagenesis were:

T32A(FW)
GTC CCC GCT CCT GTG CTT GGC CCC TTC C
T32A(RV)
GG AAG GGG CCA AGC ACA GGA GCG GGG AC
S197A(FW)
GCA AAG CCC CCC GCC GCA GAG CCG CAG CCA TGG ATA
GCA GCA G
S197A(RV)
CTG CTG CTA TCC ATG GCT GCG GCT CTG CGG CGG GGG
GCT TTG C
S262A(FW)
GTC CAC GAA GCA GCG CAA ATG CCA GCA GTG TCA GC
S262A(RV)
GCT GAC ACT GCT GGC ATT TGC GCT GCT TCG TGG AC

PCR was performed with one set of primers at a time. DpnI was added to the PCR product for 2 hr/37° C. before transformation of DH5a bacteria. Plasmid preps were sequenced before the next mutation introduced.

PSMD11 overexpressing lentiviral construct was generated as follows. Human PSMD11 cDNA was PCR amplified and cloned into pLVX-Puro using XhoI and BamHI. Resulting constructs were transformed into One Shot Stb13 E. coli (Invitrogen). Constructs were sequence verified and thereafter transfected into packaging cells to produce high titer lentivirus.

Lentiviral infection of human stem cells. hESC colonies growing on Matrigel were incubated with mTesR1 medium containing 10 μM ROCK inhibitor (Y-27632) for one hApplicants' and individualized using Accutase 1×. 5×10⁵ cells were infected in suspension with 10 μl of concentrated lentivirus in the presence of 10 μM ROCK inhibitor. Cell suspension was centrifuged to remove virus, passed through a mesh of 40 μM to obtain individual cells and plated back on a feeder layer of fresh MEFs in hESC cell media supplemented with 10 μM ROCK inhibitor. After a few days in culture, small hES cell colonies arose. For LV-GFP and LV-shFOXOs stable lines, GFP positive colonies were selected and manually passaged onto fresh MEFs to establish new hESC cell lines. For LV-non-targeting shRNA, shPSMD11, shPSMC2, FOXO4 OE, FOXO4 AAA OE and PSMD11 OE stable lines, Applicants performed 1 μg/ml puromycin resistance selection during 3 days and then colonies were manually passaged onto fresh MEFs to establish new hESC cell lines.

Transient infection experiments were performed as follows. hESC colonies growing on Matrigel were incubated with mTesR1 medium containing 10 μM ROCK inhibitor (Y-27632) for one hApplicants' and individualized using Accutase 1×. 1×10⁵ cells were plated on Matrigel plates and incubated with mTesR1 medium containing 10 μM ROCK inhibitor (Y-27632) for one day. Cells were infected with 2 μl of concentrated lentivirus. Plates were centrifuged at 800 rpm for 1 h at 30° C. Cells were fed with fresh media the day after to remove virus. NPCs were split as described above, and infected with 2 μl of concentrated lentivirus for 1 day. Neurons were infected after 2 months of differentiation with 2 μl of concentrated lentivirus for 1 day. In all the cases, cells were collected for experimental assays after 4 days of infection.

26S proteasome fluorogenic peptidase assays. In vitro assay of 26S proteasome activities was performed as previously described (Kisselev, A. F. & Goldberg, A. L., Methods in enzymology 398, 364-378 (2005)). Cells were collected in proteasome activity assay buffer (50 mM Tris-HCl (pH 7.5), 250 mM sucrose, 5 mM MgCl₂, 0.5 mM EDTA, 2 mM ATP and 1 mM dithiothreitol) and lysed by passing 10 times through a 27 gauge needle attached to a 1 ml syringe. Lysate was centrifuged at 10,000 g for 10 min at 4° C. 15-25 μg of total protein of cell lysates were transferred to a 96-well microtiter plate (BD Falcon) and then the fluorogenic substrate was added to lysates. For measuring the chymotrypsin-like activity of the proteasome Applicants used either Z-Gly-Gly-Leu-AMC (Enzo) or Suc-Leu-Leu-Val-Tyr-AMC (Enzo). Applicants used Z-Leu-Leu-Glu-AMC (Enzo) to measure the caspase-like activity of the proteasome and Ac-Arg-Leu-Arg-AMC for the proteasome trypsin-like activity. Fluorescence (380-nm excitation, 460-nm emission) was monitored on a microplate fluorometer (Infinite M1000, Tecan) every 5 min for 1 h at 25° C. Protein concentration of the cell homogenates was determined using the BCA protein assay (Pierce).

Native gel immunoblotting of the proteasome. hESCs (H9), NPCs and neurons were collected in proteasome activity assay buffer (50 mM Tris-HCl (pH 7.6), 5 mM MgCl2, 0.5 mM EDTA, 5 mM ATP, 1 mM dithiothreitol and 10% glycerol supplemented with Roche phosphatase inhibitors) and lysed by passing 10 times through a 27 gauge needle attached to a 1 ml syringe. Lysate was centrifuged at 16,000 g for 15 min at 4° C. 15 μg of total protein was run on a 3-12% NativePAGE Bis-Tris gel (Invitrogen) in 1× NativePAGE running buffer (Invitrogen) at 4° C. for 1 hApplicants' at 150V and then increased to 200V for a further hour. Proteins were then transferred to a PVDF membrane at 25V for 1 hApplicants' in 1× NativePAGE transfer buffer (Invitrogen) in an XCell II Blot module (Invitrogen). Following transfer, the PVDF membrane was incubated for 20 min with 8% acetic acid to fix the proteins and dried. Western blot analysis was performed with anti-20S alpha 1-7 (Abcam) and anti-PSMD2 (Abcam).

HEK293T cells were run on 3.5% native gels prepared in resolving buffer (90 mM Tris base, 90 mM boric acid, 5 mM MgCl₂, 0.5 mM EDTA, 1 mM ATP) with 5 mM ATP, 1 mM dithiothreitol, and 3.5% acrylamide from a 40% stock solution of acrylamide and bisacrylamide in a 37.5:1 ratio (Bio-Rad, 161-0148). These were run at 110V for 3 hr at 4° C. Activity assays were performed by incubating the gels in activity assay buffer for 20 min at 37° C. and developed using a BioRad Gel Doc with UV illumination. Prior to transfer, the gels were incubated in transfer buffer (25 mM Tris base, 192 mM glycine) with 1% SDS for 10 min followed by a 10 min incubation in transfer buffer. The protein was transferred to PVDF at 5V for 16 h to PVDF in transfer buffer using an Idea Scientific Genie Blotter. Western blot analysis was performed with anti-PSMD1 (Abcam) and analyzed using the Odyssey system (LI-COR Biosciences). Extracts were also analyzed by SDS-PAGE to determine protein expression levels and loading control.

Western Blot. For analysis of proteasome subunits, cells were collected in proteasome activity assay buffer supplemented with protease inhibitors (Roche) and lysed by passing 10 times through a 27 gauge needle attached to a 1 ml syringe. Lysate was centrifuged at 10,000 g for 10 min at 4° C. Protein concentration of the cell homogenates was determined using the BCA protein assay (Pierce). For analysis of transcription factor and polyubiquitinylated proteins, cells were harvested from tissue culture plates by cell scraping and lysed in protein cell lysis buffer (10 mM Tris-Cl pH7.4, 10 mM EDTA, 50 mM NaCl, 50 mM NaF, 1% Triton X-100, 0.1% SDS supplemented with 2 mM sodium orthovanadate, 1 mM PMSF and Complete Mini Protease and PhosSTOP inhibitor cocktail mix) for 2 hrs at 1,000 rpm and 4° C. in a Thermomixer. Protein concentrations were determined with a standard Bradford protein assay (BioRad). 20-50 μg of total protein were separated by SDS-PAGE, transferred to nitrocellulose membranes (Whatman) and subjected to immunoblotting. Western blot analysis was performed with anti-FK1 (Enzo), anti-20S alpha 1-7 (Abcam), anti-Proteasome 20S C2 (Abcam), anti-Rpt6 (Biomol), anti-PSMD1 (Abcam), anti-PSMD2 (Abcam), anti-PSMD14 (Abcam), anti-PSMB6 (Abcam), anti-PSMD11 (Novus), anti-FoxO4 (55D4) (Cell Signaling), anti-FoxO1a (C29H4) (Cell Signaling), anti-SOX2 (D6D9) (Cell Signaling), anti-FGF5 (Abcam), anti-MSX1 (Abcam), anti-PAX6 (Abcam), anti-TIF1gamma (Abcam), anti-HERC2 (Abcam) and anti-β-actin (Abcam). The affinity of the antibody to PSMD11 has been characterized by detecting a decrease at the protein levels with shPSMD11 or an increase by ectopic expression of PSMD11. These experiments convincingly show differences in only one band and Applicants ascribe any alteration of PSMD11 to this band.

Coomassie staining Protein lysates were separated by SDS-PAGE and visualized directly in the gel by Coomassie staining (Schagger, H., Nature protocols 1, 16-22 (2006)). Gels were incubated in fixing solution (50% methanol, 10% acetic acid, 100 mM ammonium acetate) for 60 min, stained with 0.025% Coomassie dye in 10% acetic acid on a shaker overnight and destained twice in 10% acetic acid for 60 min. Gels were transferred to water and analyzed with the Odyssey imager (Li-Cor Bioscience).

Immunohistochemistry. Cells were fixed with paraformaldehyde (4% in PBS) for 15 minutes, followed by blocking and permeabilization (3% Donkey Serum, 0.1% Triton in PBS) for 30 minutes. Cells were incubated in primary antibody overnight at 4° C. (Mouse anti Oct3/4, 1:200, Santa Cruz; Rabbit anti Tuj1, 1:400, Babco/Covance; Chicken anti-GFP, 1:400, Millipore), and in secondary for 2 hours at room temperature (1:250; DyLight 649 donkey anti rabbit, DyLight 549 donkey anti mouse, DyLight 488 donkey anti chicken IgY; Jackson Immuno Research). Cells were then stained with 0.5 μml⁻¹ DAPI (4′,6-diamidino-2-phenylindole) and coverslipped with Vectashield. Images were acquired using either an Olympus IX51 fluorescent or a Bio-Rad confocal microscope.

Bromodeoxyuridine (BrdU) proliferation assay. Cells were incubated with media containing 10 mM BrdU for 40 minutes. Cells were fixed with formaldehyde 4% in PBS for 15 minutes and washed in PBS. Prior to permeabilization, cells were incubated for 1 hApplicants' in 2N HCl at room temperature followed by extensive washes in PBS. Cells were permeabilized with 0.5% Triton-X100 in PBS for 10 minutes and blocked with 5% normal donkey serum in 1% PBS-BSA for 40 min at room temperature. Rabbit anti-BrdU antibody (ABD Serotech) was diluted in 1% PBS-BSA and used for overnight incubation followed by incubation with a biotinylated anti-rabbit secondary antibody (Vector) for additional 2 hours at room temperature. Finally, cells were incubated with streptavidin-AlexaFluor 568 (Jackson Immuno Research) for 1 hour. DAPI was used to visualize nuclei at a concentration of 0.5 μg ml⁻¹ DAPI in PBS.

RNA isolation and quantitative RT-PCR. Total RNA was extracted using RNAbee (Tel-Test Inc). cDNA was created using the Quantitect Reverse Transcriptase kit (Qiagen). SybrGreen real-time qPCR experiments were performed as described in the manual using ABI Prism79000HT (Applied Biosystems) and cDNA at a 1:20 dilution. Data was analyzed with the comparative 2ΔΔCt method using β-actin and GAPDH as housekeeping genes. See Table 19 for details about the primers used for this assay.

### III. Tables

TABLE 1
26S proteasome subunit transcription levels in glp-1(e2141). Data represent the mean ± s.e.m.
of the relative expression levels to fer-15(b26); fem-1(hc17) (n = 11). Statistical comparisons
were made by Student's t-test for unpaired samples.

| | fer-15(b26); fem-1(hc17) | glp-1(e2141) | |
|---|---|---|---|
| 20S α | | | |
| pas-1 | 1.04 ± 0.08 | 0.55 ± 0.07 | DECREASED (P < 0.001) |
| pas-2 | 0.99 ± 0.03 | 1.01 ± 0.10 | NO DIFFERENCES (P = 0.88) |
| pas-3 | 0.98 ± 0.04 | 0.96 ± 0.10 | NO DIFFERENCES (P = 0.88) |
| pas-4 | 0.98 ± 0.04 | 0.66 ± 0.06 | DECREASED (P < 0.001) |
| pas-5 | 1.01 ± 0.03 | 0.78 ± 0.10 | NO DIFFERENCES (P = 0.06) |
| pas-6 | 1.06 ± 0.05 | 0.76 ± 0.04 | DECREASED (P < 0.001) |
| pas-7 | 0.99 ± 0.02 | 0.32 ± 0.02 | DECREASED (P = 1.06 * 10⁻¹³⁾ |
| 20S β | | | |
| pbs-1 | 1.02 ± 0.02 | 0.66 ± 0.08 | DECREASED (P < 0.005) |
| pbs-2 | 0.97 ± 0.03 | 0.78 ± 0.07 | DECREASED (P < 0.05) |
| pbs-3 | 1.09 ± 0.06 | 0.88 ± 0.06 | DECREASED (P < 0.05) |
| pbs-4 | 1.01 ± 0.04 | 0.90 ± 0.05 | NO DIFFERENCES (P = 0.13) |
| pbs-5 | 1.03 ± 0.05 | 1.59 ± 0.10 | INCREASED (P < 0.005) |
| pbs-6 | 0.99 ± 0.06 | 0.65 ± 0.06 | DECREASED (P < 0.001) |
| pbs-7 | 1.03 ± 0.04 | 0.91 ± 0.08 | NO DIFFERENCES (P = 0.20) |
| 19S ATPases | | | |
| rpt-1 | 1.01 ± 0.02 | 0.53 ± 0.03 | DECREASED (P = 3.93 * 10⁻⁹) |
| rpt-2 | 1.04 ± 0.04 | 0.52 ± 0.03 | DECREASED (P = 2.62 * 10⁻⁹) |
| rpt-3 | 0.99 ± 0.04 | 0.54 ± 0.03 | DECREASED (P = 9.28 * 10⁻⁸) |
| rpt-4 | 1.00 ± 0.02 | 0.64 ± 0.05 | DECREASED (P = 4.00 * 10⁻⁵) |
| rpt-5 | 1.00 ± 0.04 | 0.42 ± 0.04 | DECREASED (P = 3.25 * 10⁻¹⁰) |
| rpt-6 | 1.01 ± 0.04 | 0.60 ± 0.04 | DECREASED (P = 1.37 * 10⁻⁶) |
| 19S non-ATPases | | | |
| rpn-1 | 0.99 ± 0.03 | 0.50 ± 0.02 | DECREASED (P = 1.58 * 10⁻¹⁰) |
| rpn-2 | 1.04 ± 0.04 | 0.43 ± 0.03 | DECREASED (P = 2.34 * 10⁻⁹) |
| rpn-3 | 1.02 ± 0.02 | 0.64 ± 0.04 | DECREASED (P = 6.42 * 10⁻⁷) |
| rpn-5 | 1.02 ± 0.02 | 0.43 ± 0.02 | DECREASED (P = 1.44 * 10⁻¹⁶) |
| rpn-6.1 | 1.03 ± 0.05 | 3.00 ± 0.19 | INCREASED (P = 2.09 * 10⁻⁶) |
| rpn-6.2 | 1.01 ± 0.01 | 0.26 ± 0.00 | DECREASED (P = 4.95 * 10⁻⁶) |
| rpn-7 | 1.05 ± 0.03 | 0.62 ± 0.04 | DECREASED (P = 4.50 * 10⁻⁸) |
| rpn-8 | 1.04 ± 0.03 | 0.57 ± 0.03 | DECREASED (P = 2.40 * 10⁻⁹) |
| rpn-9 | 1.01 ± 0.01 | 0.41 ± 0.03 | DECREASED (P = 3.10 * 10⁻¹³) |
| rpn-10 | 1.02 ± 0.01 | 0.80 ± 0.05 | DECREASED (P < 0.005) |
| rpn-11 | 1.03 ± 0.04 | 0.38 ± 0.02 | DECREASED (P = 1.04 * 10⁻¹⁰) |
| rpn-12 | 1.00 ± 0.02 | 0.70 ± 0.05 | DECREASED (P < 0.001) |

TABLE 2
26S proteasome subunit transcription levels in glp-1(e2141) fed with
rpn-6.1 RNAi. Data represent the mean ± s.e.m. of the relative
expression levels to glp-1(e2141) fed with vector RNAi (glp-1 + vector
RNAi (n = 4), glp-1 + rpn-6.1 RNAi (n = 5)). Statistical
comparisons were made by Student's t-test for unpaired samples.

| | glp-1 + vector | glp-1 + rpn-6.1 | |
|---|---|---|---|
| | RNAi | RNAi | |
| 20S α | | | |
| pas-1 | 1.03 ± 0.05 | 5.33 ± 1.03 | INCREASED |
| | | | (P < 0.05) |
| pas-2 | 1.10 ± 0.08 | 4.23 ± 1.12 | INCREASED |
| | | | (P < 0.05) |
| pas-3 | 1.00 ± 0.04 | 2.32 ± 0.56 | INCREASED |
| | | | (P < 0.05) |
| pas-4 | 1.24 ± 0.23 | 7.69 ± 0.80 | INCREASED |
| | | | (P < 0.001) |
| pas-5 | 1.22 ± 0.11 | 11.67 ± 1.11 | INCREASED |
| | | | (P < 0.001) |
| pas-6 | 1.20 ± 0.21 | 8.60 ± 1.46 | INCREASED |
| | | | (P < 0.01) |
| pas-7 | 1.21 ± 0.13 | 13.74 ± 0.49 | INCREASED |
| | | | (P = 5.12 * 10⁻⁶) |
| 20S β | | | |
| pbs-1 | 0.98 ± 0.04 | 4.61 ± 0.60 | INCREASED |
| | | | (P < 0.005) |
| pbs-2 | 1.10 ± 0.11 | 7.07 ± 0.63 | INCREASED |
| | | | (P < 0.001) |
| pbs-3 | 1.25 ± 0.16 | 6.00 ± 0.75 | INCREASED |
| | | | (P < 0.005) |
| pbs-4 | 1.27 ± 0.16 | 7.77 ± 0.75 | INCREASED |
| | | | (P < 0.001) |
| pbs-5 | 1.34 ± 0.17 | 5.99 ± 1.00 | INCREASED |
| | | | (P < 0.01) |
| pbs-6 | 1.01 ± 0.01 | 5.56 ± 0.70 | INCREASED |
| | | | (P < 0.005) |
| pbs-7 | 1.22 ± 0.18 | 7.24 ± 1.01 | INCREASED |
| | | | (P < 0.005) |
| 19S ATPases | | | |
| rpt-1 | 1.12 ± 0.08 | 4.41 ± 0.91 | INCREASED |
| | | | (P < 0.05) |
| rpt-2 | 1.12 ± 0.09 | 5.49 ± 0.86 | INCREASED |
| | | | (P < 0.01) |
| rpt-3 | 1.04 ± 0.05 | 4.17 ± 0.95 | INCREASED |
| | | | (P < 0.05) |
| rpt-4 | 1.13 ± 0.09 | 4.72 ± 1.17 | INCREASED |
| | | | (P < 0.05) |
| rpt-5 | 1.02 ± 0.01 | 4.53 ± 0.95 | INCREASED |
| | | | (P < 0.05) |
| rpt-6 | 0.95 ± 0.06 | 5.93 ± 1.70 | INCREASED |
| | | | (P < 0.05) |
| 19S non-ATPases | | | |
| rpn-1 | 0.99 ± 0.04 | 3.74 ± 0.71 | INCREASED |
| | | | (P < 0.05) |
| rpn-2 | 1.06 ± 0.04 | 5.10 ± 0.46 | INCREASED |
| | | | (P < 0.01) |
| rpn-3 | 1.07 ± 0.04 | 4.75 ± 0.89 | INCREASED |
| | | | (P < 0.05) |
| rpn-5 | 1.04 ± 0.02 | 4.10 ± 0.76 | INCREASED |
| | | | (P < 0.05) |
| rpn-6.1 (5′UTR) | 1.11 ± 0.35 | 0.47 ± 0.05 | DECREASED |
| | | | (P < 0.05) |
| rpn-7 | 1.09 ± 0.05 | 3.94 ± 0.73 | INCREASED |
| | | | (P < 0.05) |
| rpn-8 | 1.13 ± 0.07 | 3.25 ± 0.59 | INCREASED |
| | | | (P < 0.05) |
| rpn-9 | 1.13 ± 0.07 | 4.80 ± 0.98 | INCREASED |
| | | | (P < 0.05) |
| rpn-10 | 1.14 ± 0.07 | 4.83 ± 0.82 | INCREASED |
| | | | (P < 0.05) |
| rpn-11 | 1.11 ± 0.07 | 8.09 ± 0.38 | INCREASED |
| | | | (P = 8.15 * 10⁻⁵) |
| rpn-12 | 1.12 ± 0.06 | 4.00 ± 0.73 | INCREASED |
| | | | (P < 0.05) |

TABLE 3
26S proteasome subunit transcription levels in rpn-6.1 OE worms. Data represent the mean ± s.e.m. of the relative
expression levels to GFP OE worms (GFP OE (n = 3), rpn-6.1, GFP OE clone 1 (n = 3), rpn-6.1, GFP OE
clone 2 (n = 3)). Statistical comparisons were made by Student's t-test for unpaired samples.

| | fer-15(b26); fem-1(hc17) | glp-1(e2141) | daf-16(mgDf47); glp-1(e2141) | daf-16(mu86); glp-1(e2141) |
|---|---|---|---|---|
| 20S α | | | | |
| pas-1 | 0.99 ± 0.03 | 0.45 ± 0.02 | 0.53 ± 0.07 (P = 0.31) | 0.51 ± 0.03 (P = 0.18) |
| pas-2 | 0.99 ± 0.05 | 0.82 ± 0.07 | 0.85 ± 0.10 (P = 0.81) | 0.91 ± 0.04 (P = 0.30) |
| pas-3 | 0.97 ± 0.04 | 0.83 ± 0.10 | 0.82 ± 0.08 (P = 0.90) | 0.92 ± 0.18 (P = 0.73) |
| pas-4 | 0.97 ± 0.06 | 0.55 ± 0.04 | 0.59 ± 0.06 (P = 0.57) | 0.53 ± 0.01 (P = 0.61) |
| pas-5 | 1.00 ± 0.03 | 0.62 ± 0.09 | 0.63 ± 0.04 (P = 0.92) | 0.61 ± 0.05 (P = 0.91) |
| pas-6 | 1.07 ± 0.08 | 0.69 ± 0.03 | 0.84 ± 0.09 (P = 0.17) | 0.77 ± 0.03 (P = 0.16) |
| pas-7 | 1.00 ± 0.04 | 0.34 ± 0.05 | 0.33 ± 0.03 (P = 0.96) | 0.28 ± 0.03 (P = 0.33) |
| 20S β | | | | |
| pbs-1 | 1.02 ± 0.03 | 0.64 ± 0.10 | 0.75 ± 0.11 (P = 0.47) | 0.61 ± 0.04 (P = 0.77) |
| pbs-2 | 0.99 ± 0.04 | 0.72 ± 0.07 | 0.82 ± 0.11 (P = 0.49) | 0.73 ± 0.02 (P = 0.93) |
| pbs-3 | 1.15 ± 0.10 | 0.78 ± 0.05 | 0.89 ± 0.09 (P = 0.36) | 0.97 ± 0.10 (P = 0.20) |
| pbs-4 | 0.97 ± 0.02 | 0.83 ± 0.06 | 0.84 ± 0.08 (P = 0.91) | 0.83 ± 0.14 (P = 0.99) |
| pbs-5 | 1.08 ± 0.07 | 1.45 ± 0.12 | 1.50 ± 0.18 (P = 0.83) | 1.54 ± 0.26 (P = 0.79) |
| pbs-6 | 0.97 ± 0.08 | 0.60 ± 0.08 | 0.73 ± 0.10 (P = 0.35) | 0.45 ± 0.10 (P = 0.28) |
| pbs-7 | 1.04 ± 0.04 | 0.81 ± 0.08 | 0.94 ± 0.09 (P = 0.32) | 0.94 ± 0.05 (P = 0.21) |
| 19S ATPases | | | | |
| rpt-1 | 1.02 ± 0.03 | 0.47 ± 0.02 | 0.60 ± 0.03 (P < 0.05) | 0.65 ± 0.03 (P < 0.005) |
| rpt-2 | 1.04 ± 0.06 | 0.48 ± 0.03 | 0.63 ± 0.04 (P < 0.05) | 0.60 ± 0.02 (P < 0.01) |
| rpt-3 | 1.04 ± 0.05 | 0.49 ± 0.03 | 0.53 ± 0.04 (P = 0.44) | 0.61 ± 0.08 (P = 0.25) |
| rpt-4 | 1.03 ± 0.03 | 0.57 ± 0.04 | 0.65 ± 0.05 (P = 0.22) | 0.77 ± 0.07 (P = 0.08) |
| rpt-5 | 1.04 ± 0.06 | 0.38 ± 0.02 | 0.50 ± 0.06 (P = 0.11) | 0.45 ± 0.06 (P = 0.37) |
| rpt-6 | 1.04 ± 0.06 | 0.53 ± 0.03 | 0.58 ± 0.03 (P = 0.26) | 0.62 ± 0.05 (P = 0.19) |
| 19S non-ATPases | | | | |
| rpn-1 | 0.95 ± 0.05 | 0.47 ± 0.01 | 0.58 ± 0.04 (P = 0.06) | 0.49 ± 0.03 (P = 0.46) |
| rpn-2 | 0.98 ± 0.03 | 0.39 ± 0.03 | 0.41 ± 0.02 (P = 0.71) | 0.37 ± 0.03 (P = 0.70) |
| rpn-3 | 1.02 ± 0.03 | 0.58 ± 0.04 | 0.60 ± 0.04 (P = 0.64) | 0.57 ± 0.01 (P = 0.85) |
| rpn-5 | 1.03 ± 0.03 | 0.40 ± 0.01 | 0.48 ± 0.02 (P = 0.06) | 0.49 ± 0.04 (P = 0.12) |
| rpn-6.1 | 1.00 ± 0.06 | 3.01 ± 0.24 | 1.91 ± 0.11 (P < 0.01) | 1.63 ± 0.13 (P < 0.01) |
| rpn-7 | 1.03 ± 0.02 | 0.58 ± 0.02 | 0.94 ± 0.04 (P = 0.17) | 0.68 ± 0.07 (P = 0.28) |
| rpn-8 | 1.04 ± 0.04 | 0.50 ± 0.03 | 0.42 ± 0.02 (P = 0.06) | 0.50 ± 0.04 (P = 0.91) |
| rpn-9 | 1.00 ± 0.02 | 0.38 ± 0.03 | 0.41 ± 0.00 (P = 0.46) | 0.42 ± 0.02 (P = 0.38) |
| rpn-10 | 1.01 ± 0.01 | 0.71 ± 0.05 | 0.76 ± 0.06 (P = 0.57) | 0.87 ± 0.03 (P = 0.57) |
| rpn-11 | 1.05 ± 0.06 | 0.37 ± 0.03 | 0.41 ± 0.03 (P = 0.26) | 0.48 ± 0.06 (P = 0.20) |
| rpn-12 | 1.01 ± 0.04 | 0.64 ± 0.05 | 0.72 ± 0.05 (P = 0.27) | 0.82 ± 0.10 (P = 0.18) |

TABLE 4
26S proteasome subunit transcription levels in daf-16; glp-1 double mutant. rpn-6.1 expression is
down-regulated in daf-16; glp-1 double mutants compared to glp-1 worms. There are no significant
differences in the levels of the rest of the proteasome subunits analyzed; with the exception of rpt-1
and rpt-2. Data represent the mean ± s.e.m. of the relative expression levels to fer-1.5(b26); fem-
1(hc17) worms (fer-15(b26); fem-1(hc17) (n = 7), glp-1(e2141) (n = 7), daf-16(mgDf47);
glp-1(e2141) (n = 7), daf-16(mu86); glp-1(e2141) (n = 4)). Statistical comparisons were made
by Student's t-test for unpaired samples (glp-1(e2141) vs daf-16(mgDf47); glp-1(e2141) and
glp-1(e2141) vs daf-16(mu86); glp-1(e2141)).

| | fer-15(b26); fem- | | daf-16(mgDf47); glp- | daf-16(mu86); glp- |
|---|---|---|---|---|
| | 1(hc17) | glp-1(e2141) | 1(e2141) | 1(e2141) |
| 20S α | | | | |
| pas-1 | 0.99 ± 0.03 | 0.45 ± 0.02 | 0.53 ± 0.07 (P = 0.31) | 0.51 ± 0.03 (P = 0.18) |
| pas-2 | 0.99 ± 0.05 | 0.82 ± 0.07 | 0.85 ± 0.10 (P = 0.81) | 0.91 ± 0.04 (P = 0.30) |
| pas-3 | 0.97 ± 0.04 | 0.83 ± 0.10 | 0.82 ± 0.08 (P = 0.90) | 0.92 ± 0.18 (P = 0.73) |
| pas-4 | 0.97 ± 0.06 | 0.55 ± 0.04 | 0.59 ± 0.06 (P = 0.57) | 0.53 ± 0.01 (P = 0.61) |
| pas-5 | 1.00 ± 0.03 | 0.62 ± 0.09 | 0.63 ± 0.04 (P = 0.92) | 0.61 ± 0.05 (P = 0.91) |
| pas-6 | 1.07 ± 0.08 | 0.69 ± 0.03 | 0.84 ± 0.09 (P = 0.17) | 0.77 ± 0.03 (P = 0.16) |
| pas-7 | 1.00 ± 0.04 | 0.34 ± 0.05 | 0.33 ± 0.03 (P = 0.96) | 0.28 ± 0.03 (P = 0.33) |
| 20S β | | | | |
| pbs-1 | 1.02 ± 0.03 | 0.64 ± 0.10 | 0.75 ± 0.11 (P = 0.47) | 0.61 ± 0.04 (P = 0.77) |
| pbs-2 | 0.99 ± 0.04 | 0.72 ± 0.07 | 0.82 ± 0.11 (P = 0.49) | 0.73 ± 0.02 (P = 0.93) |
| pbs-3 | 1.15 ± 0.10 | 0.78 ± 0.05 | 0.89 ± 0.09 (P = 0.36) | 0.97 ± 0.10 (P = 0.20) |
| pbs-4 | 0.97 ± 0.02 | 0.83 ± 0.06 | 0.84 ± 0.08 (P = 0.91) | 0.83 ± 0.14 (P = 0.99) |
| pbs-5 | 1.08 ± 0.07 | 1.45 ± 0.12 | 1.50 ± 0.18 (P = 0.83) | 1.54 ± 0.26 (P = 0.79) |
| pbs-6 | 0.97 ± 0.08 | 0.60 ± 0.08 | 0.73 ± 0.10 (P = 0.35) | 0.45 ± 0.10 (P = 0.28) |
| pbs-7 | 1.04 ± 0.04 | 0.81 ± 0.08 | 0.94 ± 0.09 (P = 0.32) | 0.94 ± 0.05 (P = 0.21) |
| 19S ATPases | | | | |
| rpt-1 | 1.02 ± 0.03 | 0.47 ± 0.02 | 0.60 ± 0.03 (P < 0.05) | 0.65 ± 0.03 |
| | | | | (P < 0.005) |
| rpt-2 | 1.04 ± 0.06 | 0.48 ± 0.03 | 0.63 ± 0.04 (P < 0.05) | 0.60 ± 0.02 (P < 0.01) |
| rpt-3 | 1.04 ± 0.05 | 0.49 ± 0.03 | 0.53 ± 0.04 (P = 0.44) | 0.61 ± 0.08 (P = 0.25) |
| rpt-4 | 1.03 ± 0.03 | 0.57 ± 0.04 | 0.65 ± 0.05 (P = 0.22) | 0.77 ± 0.07 (P = 0.08) |
| rpt-5 | 1.04 ± 0.06 | 0.38 ± 0.02 | 0.50 ± 0.06 (P = 0.11) | 0.45 ± 0.06 (P = 0.37) |
| rpt-6 | 1.04 ± 0.06 | 0.53 ± 0.03 | 0.58 ± 0.03 (P = 0.26) | 0.62 ± 0.05 (P = 0.19) |
| 19S non-ATPases | | | | |
| rpn-1 | 0.95 ± 0.05 | 0.47 ± 0.01 | 0.58 ± 0.04 (P = 0.06) | 0.49 ± 0.03 (P = 0.46) |
| rpn-2 | 0.98 ± 0.03 | 0.39 ± 0.03 | 0.41 ± 0.02 (P = 0.71) | 0.37 ± 0.03 (P = 0.70) |
| rpn-3 | 1.02 ± 0.03 | 0.58 ± 0.04 | 0.60 ± 0.04 (P = 0.64) | 0.57 ± 0.01 (P = 0.85) |
| rpn-5 | 1.03 ± 0.03 | 0.40 ± 0.01 | 0.48 ± 0.02 (P = 0.06) | 0.49 ± 0.04 (P = 0.12) |
| rpn-6.1 | 1.00 ± 0.06 | 3.01 ± 0.24 | 1.91 ± 0.11 (P < 0.01) | 1.63 ± 0.13 (P < 0.01) |
| rpn-7 | 1.03 ± 0.02 | 0.58 ± 0.02 | 0.94 ± 0.04 (P = 0.17) | 0.68 ± 0.07 (P = 0.28) |
| rpn-8 | 1.04 ± 0.04 | 0.50 ± 0.03 | 0.42 ± 0.02 (P = 0.06) | 0.50 ± 0.04 (P = 0.91) |
| rpn-9 | 1.00 ± 0.02 | 0.38 ± 0.03 | 0.41 ± 0.00 (P = 0.46) | 0.42 ± 0.02 (P = 0.38) |
| rpn-10 | 1.01 ± 0.01 | 0.71 ± 0.05 | 0.76 ± 0.06 (P = 0.57) | 0.87 ± 0.03 (P = 0.57) |
| rpn-11 | 1.05 ± 0.06 | 0.37 ± 0.03 | 0.41 ± 0.03 (P = 0.26) | 0.48 ± 0.06 (P = 0.20) |
| rpn-12 | 1.01 ± 0.04 | 0.64 ± 0.05 | 0.72 ± 0.05 (P = 0.27) | 0.82 ± 0.10 (P = 0.18) |

TABLE 5
List of double-stranded RNAis.
Vidal RNAi library

| gene | ORF ID | Chromosome | Start | Stop |
|---|---|---|---|---|
| daf-12 | F11A1.3 | X | 10644331 | 10666793 |
| rpn-2 | C23G10.4 | III | 6198841 | 6201277 |
| rpn-6.1 | F57B9.10 | III | 6961956 | 6959045 |
| rpn-11 | K07D4.3 | II | 4044330 | 4043054 |
| skn-1 | T19E7.2 | IV | 5660516 | 5651089 |

Ahringer RNAi library

| gene | ORF ID | Chromosome | Forward primer squence | Reverse primer sequence |
|---|---|---|---|---|
| cco-1 | F26E4.9 | I | CGATCGATAATTTTCTTCATTCG | GCGTTTTATTTTCACTGATGGAG |
| rpn-1 | T22D1.9 | IV | ATGGATCAGGAAGTGAACGG | CTTCTTTCATGTGCCCCATT |
| nhr-80 | H10E21.3 | III | TATCCGTCTCATCCTCCCAG | CACAAAAAGTGCCTGAGCAA |
| daf-9 | T13C5.1 | X | AATTCCCCACTGCCCTTACT | AGCCCATGGCAAAACATTAG |
| hsf-1 | Y53C10A.12 | I | TCTAGAAAATTCCGGGAAAAACT | GGTGTGCTGGAAATAGACTTTTG |
| kri-1 | ZK265.1 | I | GGGTCCAACTGTCATCGTCT | CTGGCACCAAGTCAAAATCA |

TABLE 6
List of primers used for qPCR assays.

| 20S α | Forward (5′ → 3′) | Reverse (5′ → 3′) |
|---|---|---|
| pas-1 | GGCTGATCTCAACCAGTATTACACA | GAACAAAAGAGCACATCCCAAAC |
| pas-2 | CGGCCGTAATGCAGGAATAT | AAGAAGCGATGCTCCAAACG |
| pas-3 | CGGAGAGGAAATGCCAGTTG | ACGGTCTCTTTCCTCCAATCTG |
| pas-4 | GTCGTACCACCAGGATCACAAA | TGCTGCAGCTCATCATTAACTTTT |
| pas-5 | CAACATATTGGCGTCACATTCG | TGCCCGTTCGACCAGAGT |
| pas-6 | CGAGAAATCAACTCCGGAACA | AAGTGTGTCGCGAAGAGCAA |
| pas-7 | TTTCCAAGTCGAGTACGCTCAA | TTGCCACGAATTGCAATCAT |
| 20S β | Forward (5′ → 3′) | Reverse (5′ → 3′) |
| pbs-1 | TCAGCACTGGAACCACTCTCA | TCGGTTCCGACGACAACTC |
| pbs-2 | ATTTTGGAGCGTGATTTTAAGGTT | GGCGCGTTGGACAAGCT |
| pbs-3 | GCTCCACGCGATTTCGTT | GCGCCAGAAGTTTTCACAAAC |
| pbs-4 | GGGCAACAGCCGTACTTGTT | CGATCCATAATGGCATAGCAGAA |
| pbs-5 | CTGCAATTTGTGCCACATCAC | TCACGTCCATTGGTGGAAGA |
| pbs-6 | GATATGAGCGTCCGGAACTCA | ACGGAACGAATCCTTCATCAA |
| pbs-7 | CTCTACGCCAAACGTTGCAA | ACTCCGGCGACAACAAGTG |
| 19S ATPases | Forward (5′ → 3′) | Reverse (5′ → 3′) |
| rpt-1 | TGGAAACATCAAGGTGCTTATGG | CTCATGAGAGCGGGATCGA |
| rpt-2 | CCTGACGCCGCTAGCAAA | GCAACTTCAGACGGCATCTG |
| rpt-3 | TGGAGAAGGACCACGAATGG | GATGGGCTGTTTTCCTTTGC |
| rpt-4 | GTCAAGTTGTCCGACGGATTC | TGGCAAACATTCCAGCTTCTG |
| rpt-5 | GAAGATGAATGTCAACAAGGATGTAAA | TGCATTGTGCTCCGTTGAAG |
| rpt-6 | CCGAAGAATCCGATGAGAAAAC | CACTTTTTGCTGCGCATCA |
| 19S non-ATPases | Forward (5′ → 3′) | Reverse (5′ → 3′) |
| rpn-1 | CGGAAAGCCAAAGACAATCAC | GAGATATTCATCGTTCGCCAACT |
| rpn-2 | TGACATTGTTGAACAGATGGAGATC | TGCGGCTGCGTTTGAA |
| rpn-3 | ATACATTGTGGCGAAGGCTATTG | TGTACCGAGGTCCATCACGAA |
| rpn-5 | GGAGAGCACAACATGCGTATGA | CAGCGAGACGTTCGAAAGTG |
| rpn-6.1 | AATATTGGAAAAGCACCTGAAATGT | TTTGATGTGGAAGTGAAGTCATTGT |
| rpn-6.2 | AACTTGGCGAAGGCAAAGAC | AAGCAAACGCCGAATTGGT |
| rpn-7 | TCATTCAGTTGGCCGCTCTT | TGTGGCGATAGATAGCGATCAA |
| rpn-8 | TCAGGAAGTTCACGATGATGGA | TCTGAAGGCACATGCTCGAA |
| rpn-9 | GGGTGCAGCCAAGAGTTTTAGA | GGAGTTGACATCGTTCCTCCAT |
| rpn-10 | AGTACTATGATTTGTGTCGACAATTCG | GGAGCCGAGTTGGTTGGAA |
| rpn-11 | ACGTTTTCGCTATGCCACAGT | TGGATCGACCGCTTCGA |
| rpn-12 | CAAAGGAGCCAAAAGATCTTGTC | CACTGAGAACCTTCGTCAACTCA |
| rpn-6.1 (5′ UTR) | TTGAAGTTTTGACATCCTCGAA | TTATTAGTGTCTTCTCGTGAACCTCG |
| Housekeeping genes | Forward (5′ → 3′) | Reverse (5′ → 3′) |
| cdc-42 | CTGCTGGACAGGAAGATTACG | CTCGGACATTCTCGAATGAAG |
| pmp-3 | GTTCCCGTGTTCATCACTCAT | ACACCGTCGAGAAGCTGTAGA |
| Y45F10D.4 | GTCGCTTCAAATCAGTTCAGC | GTTCTTGTCAAGTGATCCGACA |

TABLE 7
PSMD11 and PSMC2 knockdown efficiencies in hESCs. a,
Data represent the mean ± s.e.m. of the relative expression
levels to non-targeting shRNA H9 hESCs (n = 3). b, Data
represent the mean ± s.e.m. of the relative expression
levels to non-targeting shRNA HUES-6 hESCs (n = 4).
a H9 hESCs

| | Non-targeting | PSMD11 | PSMD11 |
|---|---|---|---|
| | RNAi | shRNA 1 | shRNA 2 |
| PSMD11 | 1.01 ± 0.11 | 0.62 ± 0.02 | 0.69 ± 0.03 |
| PSMD1 | 1.00 ± 0.03 | 0.84 ± 0.06 | 0.99 ± 0.01 |
| | Non-targeting | PSMC2 | PSMC2 |
| | RNAi | shRNA 1 | shRNA 2 |
| PSMC2 | 1.00 ± 0.12 | 0.43 ± 0.07 | 0.36 ± 0.05 |
| PSMD1 | 0.95 ± 0.04 | 0.77 ± 0.11 | 0.78 ± 0.07 |

b HUES-6 hESCs

| | Non-targeting | PSMD11 | PSMD11 |
|---|---|---|---|
| | RNAi | shRNA 1 | shRNA 2 |
| PSMD11 | 0.99 ± 0.01 | 0.70 ± 0.12 | 0.74 ± 0.05 |
| PSMD1 | 0.98 ± 0.02 | 1.03 ± 0.10 | 0.93 ± 0.26 |

| | Non-targeting | PSMC2 |
|---|---|---|
| | RNAi | shRNA 1 |
| PSMC2 | 0.95 ± 0.05 | 0.66 ± 0.06 |
| PSMD1 | 0.98 ± 0.02 | 0.94 ± 0.12 |

TABLE 8
Knockdown efficiencies in hESCs. a, Knockdown efficiencies in transiently infected H9 hESCs. Data represent the
mean ± s.e.m. of the relative expression levels to LV-GFP cells (Non-infected (n = 11), LV-GFP (n = 16), LV-HSF1
shRNA (n = 10), LV-FOXO1a shRNA (n = 12), LV-FOXO3a shRNA (n = 10) and LV-FOXO4 shRNA (n = 16)). b,
Knockdown efficiencies in stably infected H9 hESCs. Data represent the mean ± s.e.m. of the relative expression
levels to GFP clone 1 H9 cells (GFP clone 1 (n = 28), HSF1 shRNA clone 1 (n = 15), FOXO1a shRNA clone 1 (n = 18),
FOXO3a shRNA clone 1 (n = 24), FOXO4 shRNA clone 1 (n = 25), GFP clone 2 (n = 23), HSF1 shRNA clone 2
(n = 13), FOXO1a shRNA clone 2 (n = 17), FOXO3a shRNA clone 2 (n = 15) and FOXO4 shRNA clone 2 (n = 17)). c,
Knockdown efficiencies in stably infected H9 hESCs. Data represent the mean ± s.e.m. of the relative expression
levels to GFP clone 3 H9 cells (GFP clone 3 (n = 6), 3′UTR FOXO4 shRNA 1 (n = 7), 3′UTR FOXO4 shRNA 2
(n = 5), 3′UTR FOXO4 shRNA 3 (n = 6)). d, Knockdown efficiencies in stably infected HUES-6 hESCs. Data
represent the mean ± s.e.m. of the relative expression levels to GFP HUES-6 cells (GFP (n = 17), HSF1 shRNA
(n = 6), FOXO1a shRNA (n = 9), FOXO3a shRNA (n = 4), FOXO4 shRNA (n = 7), 3′UTR FOXO4 shRNA
1 (n = 11), 3′UTR FOXO4 shRNA 2 (n = 10), 3′UTR FOXO4 shRNA 3 (n = 6)).
a Transient transfected H9 hESCs

| | | | | LV-FOXO1a | LV-FOXO3a | LV-FOXO4 |
|---|---|---|---|---|---|---|
| | Non-infected | LV-GFP | LV-HSF1 shRNA | shRNA | shRNA | shRNA |
| HSF1 | 1.14 ± 0.09 | 1.03 ± 0.05 | 0.68 ± 0.02 | 1.10 ± 0.07 | 1.37 ± 0.18 | 0.98 ± 0.06 |
| FOXO1a | 0.98 ± 0.03 | 1.08 ± 0.05 | 0.93 ± 0.12 | 0.69 ± 0.01 | 1.08 ± 0.11 | 0.98 ± 0.07 |
| FOXO3a | 1.36 ± 0.17 | 1.01 ± 0.08 | 1.20 ± 0.19 | 1.02 ± 0.10 | 0.59 ± 0.06 | 0.84 ± 0.06 |
| FOXO4 | 0.92 ± 0.06 | 1.04 ± 0.04 | 1.00 ± 0.08 | 1.46 ± 0.11 | 1.03 ± 0.10 | 0.43 ± 0.04 |

b Stable transfected H9 hESCs

| | HSF1 shRNA | FOXO1a | FOXO3a | FOXO4 shRNA | |
|---|---|---|---|---|---|
| GFP clone 1 | clone 1 | shRNA clone 1 | shRNA clone 1 | clone 1 | |
| HSF1 | 1.08 ± 0.06 | 0.59 ± 0.04 | 0.96 ± 0.04 | 1.01 ± 0.08 | 0.90 ± 0.08 |
| FOXO1a | 0.92 ± 0.03 | 0.81 ± 0.04 | 0.49 ± 0.07 | 0.95 ± 0.07 | 1.04 ± 0.10 |
| FOXO3a | 1.06 ± 0.03 | 0.91 ± 0.09 | 0.80 ± 0.06 | 0.76 ± 0.04 | 0.77 ± 0.06 |
| FOXO4 | 1.06 ± 0.06 | 1.06 ± 0.11 | 1.00 ± 0.06 | 0.98 ± 0.09 | 0.46 ± 0.04 |
| | | HSF1 shRNA | FOXO1a | FOXO3a | FOXO4 shRNA |
| | GFP clone 2 | clone 2 | shRNA clone 2 | shRNA clone 2 | clone 2 |
| HSF1 | 1.09 ± 0.11 | 0.64 ± 0.03 | 0.97 ± 0.09 | 1.02 ± 0.09 | 0.90 ± 0.15 |
| FOXO1a | 0.95 ± 0.06 | 0.76 ± 0.05 | 0.51 ± 0.07 | 0.78 ± 0.07 | 1.04 ± 0.17 |
| FOXO3a | 1.02 ± 0.05 | 0.99 ± 0.13 | 0.81 ± 0.11 | 0.65 ± 0.10 | 0.77 ± 0.11 |
| FOXO4 | 1.08 ± 0.11 | 1.17 ± 0.10 | 1.02 ± 0.10 | 0.81 ± 0.11 | 0.46 ± 0.08 |

c Stable transfected H9 hESCs

| GFP clone 3 | 3′UTR FOXO4 | shRNA 1 | 3′UTR FOXO4 shRNA 2 | 3′UTR FOXO4 shRNA 3 |
|---|---|---|---|---|
| FOXO1a | 0.99 ± 0.01 | 1.06 ± 0.06 | 1.09 ± 0.14 | 1.16 ± 0.14 |
| FOXO3a | 1.12 ± 0.12 | 1.14 ± 0.14 | 1.40 ± 0.22 | 1.68 ± 0.24 |
| FOXO4 | 0.97 ± 0.04 | 0.23 ± 0.04 | 0.48 ± 0.06 | 0.37 ± 0.03 |

d Stable transfected HUES-6 hESCs

| | | | | | | 3′UTR | 3′UTR | 3′UTR |
|---|---|---|---|---|---|---|---|---|
| | | HSF1 | FOXO1a | FOXO3a | FOXO4 | FOXO4 | FOXO4 | FOXO4 |
| | GFP | shRNA | shRNA | shRNA | shRNA | shRNA 1 | shRNA 2 | shRNA 3 |
| HSF1 | 1.19 ± 0.16 | 0.52 ± 0.08 | 1.13 ± 0.24 | 1.14 ± 0.13 | 0.85 ± 0.03 | 1.10 ± 0.18 | 1.88 ± 0.43 | 0.85 ± 0.13 |
| FOXO1a | 0.99 ± 0.03 | 1.06 ± 0.07 | 0.71 ± 0.04 | 0.88 ± 0.19 | 0.64 ± 0.06 | 0.76 ± 0.08 | 0.63 ± 0.06 | 0.99 ± 0.19 |
| FOXO3a | 1.00 ± 0.03 | 0.95 ± 0.16 | 0.93 ± 0.13 | 0.77 ± 0.04 | 1.82 ± 0.30 | 1.06 ± 0.15 | 0.70 ± 0.10 | 0.86 ± 0.08 |
| FOXO4 | 1.03 ± 0.04 | 1.01 ± 0.14 | 1.11 ± 0.19 | 0.89 ± 0.25 | 0.34 ± 0.03 | 0.08 ± 0.02 | 0.14 ± 0.01 | 0.62 ± 0.11 |

TABLE 9
Knockdown efficiencies in transiently infected NPCs and neurons. a, Data represent the mean ± s.e.m. of the relative
expression levels to LV-GFP NPCs (Non-infected (n = 4), LV-GFP (n = 6), LV-HSF1 shRNA (n = 5), LV-FOXO1a
shRNA (n = 5), LV-FOXO3a shRNA (n = 5) and LV-FOXO4 shRNA (n = 5)). b, Data represent the mean ± s.e.m.
of the relative expression levels to LV-GFP neurons (Non-infected (n = 3), LV-GFP (n = 7), LV-HSF1 shRNA (n = 5),
LV-FOXO1a shRNA (n = 5), LV-FOXO3a shRNA (n = 5) and LV-FOXO4 shRNA (n = 5)).

| | | | | | |
|---|---|---|---|---|---|
| Non-infected | LV-GFP | LV-HSF1 shRNA | LV-FOXO1a shRNA | LV-FOXO3a shRNA | LV-FOXO4 shRNA |

a. Transient transfected NPCs.

| | | | | | | |
|---|---|---|---|---|---|---|
| HSF1 | 1.23 ± 0.15 | 0.98 ± 0.04 | 0.68 ± 0.05 | 1.24 ± 0.08 | 1.01 ± 0.12 | 0.62 ± 0.23 |
| FOXO1a | 1.22 ± 0.22 | 1.10 ± 0.18 | 0.98 ± 0.06 | 0.65 ± 0.03 | 0.91 ± 0.10 | 1.01 ± 0.18 |
| FOXO3a | 1.31 ± 0.21 | 1.19 ± 0.27 | 0.93 ± 0.10 | 1.08 ± 0.14 | 0.61 ± 0.06 | 0.65 ± 0.08 |
| FOXO4 | 1.25 ± 0.14 | 0.91 ± 0.07 | 0.90 ± 0.05 | 1.00 ± 0.10 | 0.72 ± 0.14 | 0.30 ± 0.06 |

b. Transient transfected Neurons.

| | | | | | | |
|---|---|---|---|---|---|---|
| HSF1 | 0.86 ± 0.01 | 0.99 ± 0.02 | 0.69 ± 0.05 | 0.93 ± 0.03 | 0.77 ± 0.19 | 0.85 ± 0.15 |
| FOXO1a | 1.10 ± 0.38 | 1.00 ± 0.03 | 1.04 ± 0.07 | 0.78 ± 0.02 | 0.98 ± 0.06 | 0.95 ± 0.13 |
| FOXO3a | 1.06 ± 0.02 | 0.94 ± 0.10 | 1.03 ± 0.06 | 0.99 ± 0.11 | 0.69 ± 0.04 | 0.65 ± 0.20 |
| FOXO4 | 0.86 ± 0.16 | 0.99 ± 0.04 | 0.87 ± 0.07 | 0.86 ± 0.04 | 1.11 ± 0.07 | 0.59 ± 0.11 |

TABLE 10
FOXO4 overexpression levels in H9 hESCs. a, Data represent the
mean ± s.e.m. of the relative expression levels to non-infected H9
hESCs (Non-infected (n = 4), LV-FOXO4 OE (n = 4), LV-FOXO4
AAA OE (n = 4)). b, Data represent the mean ± s.e.m. of the
relative expression levels to GFP H9 hESCs (GFP (n = 10), FOXO4
OE (n = 8), FOXO4 AAA OE (n = 6)). b, Data represent the
mean ± s.e.m. of the relative expression levels to GFP H9 hESCs
(GFP (n = 4), 3′UTR_3 (n = 4), 3′UTR_3 + FOXO4
AAA OE (n = 4)).
a. Transient transfected H9 hESCs.

| | Non-infected | LV-FOXO4 OE | LV-FOXO4 AAA OE |
|---|---|---|---|
| HSF1 | 1.01 ± 0.02 | 1.13 ± 0.01 | 1.12 ± 0.06 |
| FOXO1a | 1.01 ± 0.02 | 0.91 ± 0.01 | 0.95 ± 0.05 |
| FOXO3a | 0.99 ± 0.01 | 0.97 ± 0.01 | 0.93 ± 0.05 |
| FOXO4 | 1.02 ± 0.01 | 3.23 ± 0.04 | 2.16 ± 0.11 |

b. FOXO4 OE stable H9 hESCs.

| GFP | FOXO4 OE | FOXO4 AAA OE | |
|---|---|---|---|
| FOXO1a | 1.19 ± 0.13 | 1.32 ± 0.18 | 1.45 ± 0.11 |
| FOXO3a | 0.92 ± 0.11 | 0.99 ± 0.13 | 1.31 ± 0.19 |
| FOXO4 | 1.05 ± 0.04 | 2.81 ± 0.44 | 2.51 ± 0.28 |

c. Ectopic expression of FOXO4 AAA in FOXO4 KD H9 hESCs.

| | | | 3′UTR FOXO4 |
|---|---|---|---|
| | | 3′UTR FOXO4 | shRNA 3 + FOXO4 |
| | GFP | shRNA 3 | AAA OE |
| FOXO4 | 0.95 ± 0.04 | 0.59 ± 0.05 | 1.05 ± 0.09 |

TABLE 11
26S proteasome subunit transcription levels. a, 26S proteasome subunit transcription levels in transient FOXO KD H9
hESCs. Data represent the mean ± s.e.m. of the relative expression levels to LV-GFP hESCs (Non-infected (n = 5), LV-GFP
(n = 10), LV-HSF1 shRNA (n = 5), LV-FOXO1a shRNA (n = 4), LV-FOXO3a shRNA (n = 4) and LV-FOXO4 shRNA
(n = 5)). b, 26S proteasome subunit transcription levels in transient FOXO4 KD H9 hESCs. Data represent the mean ± s.e.m.
of the relative expression levels to LV-GFP hESCs (LV-GFP (n = 5), LV-3′UTR FOXO4 shRNA 2 (n = 4), LV-3′UTR FOXO4
shRNA 3 (n = 5)). c, 26S proteasome subunit transcription levels in stable FOXO4 OE H9 hESCs. Data represent the mean ± s.e.m.
of the relative expression levels to GFP hESCs ((GFP (n = 7), FOXO4 OE (n = 8), FOXO4 AAA OE (n = 7)).
a

| | WT | LV-GFP | LV-HSF1 shRNA | LV-FOXO1a shRNA | LV-FOXO3a shRNA | LV-FOXO4 shRNA |
|---|---|---|---|---|---|---|
| 20S α | | | | | | |
| PSMA1 | 1.06 ± 0.06 | 1.07 ± 0.08 | 0.97 ± 0.03 | 0.88 ± 0.05 | 0.91 ± 0.11 | 0.88 ± 0.03 |
| PSMA2 | 0.97 ± 0.04 | 0.98 ± 0.07 | 1.01 ± 0.08 | 0.98 ± 0.07 | 0.99 ± 0.17 | 0.93 ± 0.08 |
| PSMA3 | 1.04 ± 0.05 | 1.06 ± 0.06 | 0.95 ± 0.08 | 0.93 ± 0.05 | 0.87 ± 0.07 | 0.96 ± 0.03 |
| PSMA4 | 1.07 ± 0.04 | 1.05 ± 0.08 | 0.99 ± 0.07 | 0.83 ± 0.04 | 0.87 ± 0.04 | 0.91 ± 0.09 |
| PSMA5 | 0.86 ± 0.06 | 1.07 ± 0.15 | 0.85 ± 0.02 | 0.84 ± 0.05 | 0.93 ± 0.05 | 0.96 ± 0.12 |
| PSMA6 | 1.13 ± 0.03 | 1.03 ± 0.08 | 1.00 ± 0.08 | 0.91 ± 0.05 | 0.97 ± 0.11 | 0.92 ± 0.04 |
| PSMA7 | 1.02 ± 0.09 | 1.09 ± 0.07 | 0.96 ± 0.07 | 0.93 ± 0.05 | 1.01 ± 0.08 | 0.99 ± 0.08 |
| 20S β | | | | | | |
| PSMB1 | 0.93 ± 0.07 | 1.13 ± 0.12 | 0.93 ± 0.05 | 0.97 ± 0.05 | 0.91 ± 0.02 | 1.13 ± 0.10 |
| PSMB2 | 1.01 ± 0.02 | 1.07 ± 0.08 | 0.94 ± 0.06 | 0.90 ± 0.04 | 0.94 ± 0.03 | 0.95 ± 0.08 |
| PSMB3 | 1.02 ± 0.05 | 1.16 ± 0.14 | 0.89 ± 0.02 | 1.04 ± 0.09 | 1.01 ± 0.06 | 1.10 ± 0.15 |
| PSMB4 | 0.93 ± 0.02 | 1.07 ± 0.09 | 0.90 ± 0.06 | 1.02 ± 0.02 | 1.01 ± 0.03 | 0.97 ± 0.07 |
| PSMB5 | 1.15 ± 0.08 | 1.06 ± 0.09 | 0.97 ± 0.06 | 1.14 ± 0.08 | 1.08 ± 0.13 | 0.96 ± 0.04 |
| PSMB6 | 0.98 ± 0.09 | 1.13 ± 0.12 | 0.91 ± 0.06 | 0.98 ± 0.04 | 1.07 ± 0.10 | 1.00 ± 0.10 |
| PSMB7 | 1.01 ± 0.03 | 1.08 ± 0.06 | 0.96 ± 0.02 | 0.98 ± 0.03 | 1.06 ± 0.05 | 0.99 ± 0.05 |
| 19S ATPases | | | | | | |
| PSMC1 | 0.81 ± 0.04 | 1.18 ± 0.13 | 0.85 ± 0.10 | 0.98 ± 0.04 | 0.93 ± 0.06 | 0.99 ± 0.08 |
| PSMC2 | 0.95 ± 0.09 | 1.08 ± 0.09 | 0.90 ± 0.09 | 1.00 ± 0.08 | 1.10 ± 0.12 | 0.59 ± 0.03 |
| PSMC3 | 1.04 ± 0.09 | 1.14 ± 0.09 | 0.87 ± 0.08 | 1.16 ± 0.08 | 1.19 ± 0.09 | 1.13 ± 0.08 |
| PSMC4 | 1.00 ± 0.10 | 1.21 ± 0.14 | 1.00 ± 0.13 | 1.22 ± 0.12 | 1.19 ± 0.15 | 1.13 ± 0.10 |
| PSMC5 | 0.95 ± 0.09 | 1.21 ± 0.11 | 0.94 ± 0.12 | 1.13 ± 0.10 | 1.15 ± 0.09 | 1.26 ± 0.19 |
| PSMC6 | 0.94 ± 0.06 | 1.15 ± 0.11 | 0.88 ± 0.07 | 0.94 ± 0.05 | 0.87 ± 0.06 | 0.91 ± 0.06 |
| 19S non-ATPases | | | | | | |
| PSMD1 | 0.87 ± 0.05 | 1.14 ± 0.12 | 0.91 ± 0.05 | 0.95 ± 0.02 | 1.10 ± 0.05 | 1.03 ± 0.06 |
| PSMD2 | 1.05 ± 0.04 | 1.12 ± 0.07 | 0.98 ± 0.03 | 0.95 ± 0.04 | 0.98 ± 0.04 | 1.15 ± 0.03 |
| PSMD3 | 0.97 ± 0.04 | 1.15 ± 0.09 | 0.87 ± 0.10 | 0.91 ± 0.05 | 1.04 ± 0.11 | 0.95 ± 0.07 |
| PSMD4 | 1.03 ± 0.04 | 1.17 ± 0.12 | 0.98 ± 0.12 | 0.97 ± 0.05 | 1.05 ± 0.05 | 1.04 ± 0.07 |
| PSMD6 | 1.06 ± 0.04 | 1.20 ± 0.11 | 1.00 ± 0.09 | 1.03 ± 0.10 | 0.93 ± 0.13 | 0.95 ± 0.06 |
| PSMD7 | 0.93 ± 0.08 | 1.20 ± 0.16 | 0.88 ± 0.08 | 1.15 ± 0.10 | 1.21 ± 0.07 | 1.23 ± 0.15 |
| PSMD11 | 0.96 ± 0.03 | 1.08 ± 0.07 | 0.98 ± 0.05 | 1.06 ± 0.10 | 1.12 ± 0.12 | 0.82 ± 0.01 |
| PSMD12 | 0.90 ± 0.06 | 1.15 ± 0.11 | 0.84 ± 0.06 | 0.99 ± 0.05 | 1.12 ± 0.09 | 1.10 ± 0.10 |
| PSMD14 | 0.94 ± 0.07 | 1.13 ± 0.07 | 0.94 ± 0.09 | 1.01 ± 0.03 | 1.12 ± 0.06 | 1.05 ± 0.07 |
| ADRM1 | 1.02 ± 0.09 | 1.13 ± 0.08 | 0.85 ± 0.04 | 1.07 ± 0.04 | 1.06 ± 0.20 | 1.16 ± 0.04 |

b

| | | LV-3′UTR FOXO4 | LV-3′UTR FOXO4 |
|---|---|---|---|
| | LV-GFP | shRNA 2 | shRNA 3 |
| PSMD1 | 1.08 ± 0.04 | 0.90 ± 0.03 | 0.91 ± 0.04 |
| PSMD11 | 1.07 ± 0.03 | 0.71 ± 0.09 | 0.79 ± 0.03 |
| PSMC2 | 1.06 ± 0.03 | 1.12 ± 0.26 | 0.93 ± 0.06 |

c

| | LV-GFP | LV-FOXO4 OE | LV-FOXO4 AAA OE |
|---|---|---|---|
| PSMD1 | 1.15 ± 0.16 | 1.19 ± 0.05 | 1.16 ± 0.05 |
| PSMD11 | 1.07 ± 0.02 | 1.16 ± 0.03 | 1.41 ± 0.05 |
| PSMC2 | 1.09 ± 0.08 | 1.00 ± 0.06 | 1.02 ± 0.05 |

TABLE 12
26S proteasome subunit transcription levels in transient FOXO KD NPCs. Data represent the mean ± s.e.m. of the
relative expression levels to LV-GFP cells (Non-infected (n = 4), LV-GFP (n = 6), LV-HSF1 shRNA (n = 3),
LV-FOXO1a shRNA (n = 3), LV-FOXO3a shRNA (n = 4) and LV-FOXO4 shRNA (n = 4)).

| | WT | LV-GFP | LV-HSF1 shRNA | LV-FOXO1a shRNA | LV-FOXO3a shRNA | LV-FOXO4 shRNA |
|---|---|---|---|---|---|---|
| 20S α | | | | | | |
| PSMA1 | 0.90 ± 0.04 | 1.01 ± 0.04 | 1.02 ± 0.01 | 0.99 ± 0.07 | 1.84 ± 0.20 | 1.17 ± 0.10 |
| PSMA2 | 0.83 ± 0.07 | 0.99 ± 0.09 | 1.24 ± 0.33 | 0.89 ± 0.07 | 1.82 ± 0.09 | 1.19 ± 0.15 |
| PSMA3 | 0.80 ± 0.04 | 1.00 ± 0.05 | 0.92 ± 0.06 | 1.03 ± 0.08 | 1.85 ± 0.10 | 1.05 ± 0.18 |
| PSMA4 | 0.90 ± 0.07 | 1.02 ± 0.05 | 1.07 ± 0.03 | 0.85 ± 0.06 | 1.88 ± 0.07 | 0.98 ± 0.10 |
| PSMA5 | 0.73 ± 0.06 | 0.98 ± 0.02 | 0.92 ± 0.04 | 0.94 ± 0.04 | 1.98 ± 0.19 | 1.11 ± 0.10 |
| PSMA6 | 0.88 ± 0.03 | 0.97 ± 0.02 | 0.98 ± 0.07 | 1.00 ± 0.08 | 1.00 ± 0.35 | 1.27 ± 0.17 |
| PSMA7 | 0.81 ± 0.06 | 1.01 ± 0.04 | 0.90 ± 0.04 | 1.02 ± 0.05 | 2.12 ± 0.08 | 1.14 ± 0.17 |
| 20S β | | | | | | |
| PSMB1 | 0.97 ± 0.07 | 1.05 ± 0.05 | 1.11 ± 0.08 | 1.29 ± 0.08 | 1.60 ± 0.06 | 1.54 ± 0.17 |
| PSMB2 | 0.92 ± 0.04 | 1.03 ± 0.02 | 0.93 ± 0.04 | 0.98 ± 0.12 | 2.03 ± 0.12 | 1.03 ± 0.19 |
| PSMB3 | 0.73 ± 0.10 | 1.05 ± 0.03 | 0.90 ± 0.06 | 0.90 ± 0.04 | 1.99 ± 0.08 | 0.94 ± 0.12 |
| PSMB4 | 0.81 ± 0.10 | 0.99 ± 0.01 | 0.79 ± 0.05 | 0.88 ± 0.07 | 1.97 ± 0.07 | 0.77 ± 0.02 |
| PSMB5 | 0.84 ± 0.10 | 1.05 ± 0.06 | 1.18 ± 0.10 | 0.77 ± 0.01 | 2.57 ± 0.18 | 0.90 ± 0.12 |
| PSMB6 | 0.81 ± 0.06 | 0.94 ± 0.04 | 0.98 ± 0.02 | 0.92 ± 0.03 | 2.09 ± 0.25 | 0.89 ± 0.21 |
| PSMB7 | 0.77 ± 0.05 | 0.99 ± 0.00 | 0.90 ± 0.06 | 0.80 ± 0.03 | 1.71 ± 0.02 | 0.85 ± 0.05 |
| 19S ATPases | | | | | | |
| PSMC1 | 0.88 ± 0.05 | 0.97 ± 0.06 | 0.99 ± 0.07 | 1.04 ± 0.07 | 1.41 ± 0.08 | 1.00 ± 0.11 |
| PSMC2 | 0.82 ± 0.05 | 0.92 ± 0.08 | 0.94 ± 0.12 | 0.91 ± 0.07 | 1.45 ± 0.09 | 0.30 ± 0.07 |
| PSMC3 | 1.00 ± 0.01 | 0.99 ± 0.09 | 1.09 ± 0.07 | 0.75 ± 0.07 | 1.09 ± 0.08 | 0.65 ± 0.06 |
| PSMC4 | 1.07 ± 0.09 | 1.00 ± 0.08 | 1.11 ± 0.02 | 1.12 ± 0.04 | 1.20 ± 0.20 | 1.06 ± 0.09 |
| PSMC5 | 0.90 ± 0.04 | 1.02 ± 0.08 | 1.05 ± 0.05 | 0.84 ± 0.08 | 1.06 ± 0.07 | 0.78 ± 0.07 |
| PSMC6 | 0.93 ± 0.03 | 0.95 ± 0.06 | 1.07 ± 0.05 | 1.14 ± 0.09 | 1.31 ± 0.11 | 0.74 ± 0.08 |
| 19S non-ATPases | | | | | | |
| PSMD1 | 1.09 ± 0.08 | 0.94 ± 0.09 | 1.01 ± 0.13 | 1.21 ± 0.20 | 1.20 ± 0.09 | 0.76 ± 0.14 |
| PSMD2 | 1.07 ± 0.09 | 0.94 ± 0.05 | 1.01 ± 0.04 | 0.98 ± 0.01 | 1.18 ± 0.12 | 0.62 ± 0.04 |
| PSMD3 | 0.99 ± 0.05 | 0.95 ± 0.07 | 0.94 ± 0.01 | 0.99 ± 0.02 | 1.08 ± 0.16 | 0.66 ± 0.04 |
| PSMD4 | 0.91 ± 0.03 | 0.96 ± 0.08 | 1.05 ± 0.07 | 1.00 ± 0.11 | 1.24 ± 0.06 | 0.73 ± 0.09 |
| PSMD6 | 0.88 ± 0.06 | 0.97 ± 0.06 | 1.09 ± 0.03 | 0.98 ± 0.03 | 1.36 ± 0.08 | 0.84 ± 0.09 |
| PSMD7 | 1.11 ± 0.11 | 0.93 ± 0.07 | 1.01 ± 0.04 | 1.03 ± 0.05 | 1.23 ± 0.06 | 0.88 ± 0.06 |
| PSMD11 | 0.87 ± 0.04 | 1.02 ± 0.08 | 0.91 ± 0.05 | 0.97 ± 0.06 | 1.26 ± 0.20 | 0.75 ± 0.03 |
| PSMD12 | 1.02 ± 0.06 | 0.92 ± 0.06 | 0.98 ± 0.06 | 1.22 ± 0.20 | 1.18 ± 0.03 | 0.80 ± 0.08 |
| PSMD14 | 0.73 ± 0.12 | 0.96 ± 0.04 | 1.06 ± 0.09 | 1.23 ± 0.09 | 1.44 ± 0.10 | 0.77 ± 0.05 |
| ADRM1 | 0.91 ± 0.05 | 0.97 ± 0.04 | 1.14 ± 0.12 | 0.93 ± 0.15 | 1.30 ± 0.14 | 0.90 ± 0.14 |

TABLE 13
26S proteasome subunit transcription levels in FOXO KD neurons. Data represent the mean ± s.e.m. of the relative
expression levels to LV-GFP cells (Non-infected (n = 3), LV-GFP (n = 4), LV-HSF1 shRNA (n = 3), LV-FOXO1a
shRNA (n = 3), LV-FOXO3a shRNA (n = 3) and LV-FOXO4 shRNA (n = 3)).

| | WT | LV-GFP | LV-HSF1 shRNA | LV-FOXO1a shRNA | LV-FOXO3a shRNA | LV-FOXO4 shRNA |
|---|---|---|---|---|---|---|
| 20S α | | | | | | |
| PSMA1 | 1.21 ± 0.10 | 1.08 ± 0.11 | 1.18 ± 0.09 | 1.37 ± 0.14 | 1.20 ± 0.03 | 1.23 ± 0.08 |
| PSMA2 | 0.97 ± 0.04 | 0.98 ± 0.04 | 1.03 ± 0.11 | 1.29 ± 0.03 | 0.95 ± 0.20 | 0.96 ± 0.06 |
| PSMA3 | 1.24 ± 0.10 | 1.08 ± 0.13 | 1.17 ± 0.11 | 1.34 ± 0.12 | 1.19 ± 0.06 | 1.06 ± 0.09 |
| PSMA4 | 1.12 ± 0.02 | 1.00 ± 0.07 | 1.08 ± 0.08 | 1.20 ± 0.00 | 0.96 ± 0.03 | 1.02 ± 0.06 |
| PSMA5 | 1.12 ± 0.02 | 0.99 ± 0.03 | 1.02 ± 0.09 | 1.33 ± 0.12 | 1.04 ± 0.05 | 1.05 ± 0.04 |
| PSMA6 | 0.95 ± 0.05 | 1.03 ± 0.01 | 1.02 ± 0.02 | 1.40 ± 0.10 | 1.15 ± 0.05 | 1.00 ± 0.01 |
| PSMA7 | 0.97 ± 0.01 | 1.05 ± 0.04 | 1.15 ± 0.07 | 1.30 ± 0.02 | 1.13 ± 0.03 | 0.96 ± 0.03 |
| 20S β | | | | | | |
| PSMB1 | 0.90 ± 0.04 | 1.04 ± 0.04 | 1.14 ± 0.02 | 1.18 ± 0.05 | 0.97 ± 0.10 | 0.89 ± 0.02 |
| PSMB2 | 0.85 ± 0.02 | 0.95 ± 0.03 | 0.80 ± 0.09 | 1.01 ± 0.05 | 0.84 ± 0.04 | 0.92 ± 0.06 |
| PSMB3 | 0.97 ± 0.02 | 1.07 ± 0.07 | 1.19 ± 0.05 | 1.26 ± 0.10 | 0.94 ± 0.11 | 1.09 ± 0.00 |
| PSMB4 | 0.97 ± 0.04 | 1.03 ± 0.01 | 1.01 ± 0.02 | 1.26 ± 0.06 | 1.08 ± 0.05 | 0.99 ± 0.04 |
| PSMB5 | 0.99 ± 0.02 | 1.05 ± 0.06 | 1.13 ± 0.06 | 1.26 ± 0.01 | 1.12 ± 0.11 | 1.00 ± 0.09 |
| PSMB6 | 1.16 ± 0.04 | 1.09 ± 0.08 | 1.29 ± 0.04 | 1.38 ± 0.13 | 1.16 ± 0.03 | 1.12 ± 0.11 |
| PSMB7 | 0.89 ± 0.08 | 0.98 ± 0.02 | 0.88 ± 0.01 | 1.18 ± 0.08 | 1.04 ± 0.14 | 0.93 ± 0.08 |
| 19S ATPases | | | | | | |
| PSMC1 | 0.95 ± 0.11 | 0.95 ± 0.05 | 1.10 ± 0.09 | 0.94 ± 0.01 | 1.01 ± 0.09 | 0.90 ± 0.05 |
| PSMC2 | 0.93 ± 0.19 | 1.11 ± 0.12 | 0.97 ± 0.17 | 1.11 ± 0.16 | 1.21 ± 0.26 | 0.90 ± 0.13 |
| PSMC3 | 0.97 ± 0.04 | 0.99 ± 0.02 | 1.08 ± 0.02 | 0.96 ± 0.05 | 1.01 ± 0.09 | 0.99 ± 0.04 |
| PSMC4 | 0.93 ± 0.09 | 0.91 ± 0.06 | 1.14 ± 0.08 | 0.97 ± 0.06 | 1.08 ± 0.15 | 1.00 ± 0.07 |
| PSMC5 | 1.06 ± 0.06 | 0.94 ± 0.04 | 1.07 ± 0.10 | 1.07 ± 0.00 | 1.04 ± 0.10 | 0.91 ± 0.07 |
| PSMC6 | 0.91 ± 0.04 | 0.97 ± 0.05 | 1.01 ± 0.05 | 0.98 ± 0.02 | 0.99 ± 0.13 | 0.98 ± 0.04 |
| 19S non-ATPases | | | | | | |
| PSMD1 | 0.98 ± 0.01 | 1.02 ± 0.08 | 1.06 ± 0.03 | 0.90 ± 0.01 | 0.90 ± 0.03 | 1.03 ± 0.09 |
| PSMD2 | 0.84 ± 0.01 | 0.99 ± 0.03 | 0.94 ± 0.02 | 0.99 ± 0.00 | 1.03 ± 0.15 | 0.99 ± 0.05 |
| PSMD3 | 0.91 ± 0.08 | 1.00 ± 0.07 | 1.12 ± 0.06 | 1.00 ± 0.01 | 1.11 ± 0.23 | 1.03 ± 0.11 |
| PSMD4 | 0.94 ± 0.01 | 0.98 ± 0.02 | 0.99 ± 0.05 | 0.96 ± 0.02 | 0.95 ± 0.16 | 1.04 ± 0.08 |
| PSMD6 | 0.88 ± 0.11 | 0.94 ± 0.06 | 0.96 ± 0.12 | 0.88 ± 0.03 | 0.96 ± 0.14 | 0.93 ± 0.09 |
| PSMD7 | 0.93 ± 0.05 | 0.95 ± 0.07 | 1.10 ± 0.01 | 1.04 ± 0.03 | 0.95 ± 0.10 | 1.03 ± 0.01 |
| PSMD11 | 0.86 ± 0.09 | 0.96 ± 0.05 | 1.00 ± 0.05 | 1.09 ± 0.09 | 1.12 ± 0.11 | 0.93 ± 0.06 |
| PSMD12 | 0.85 ± 0.03 | 0.92 ± 0.05 | 1.23 ± 0.10 | 1.21 ± 0.02 | 1.13 ± 0.17 | 1.05 ± 0.12 |
| PSMD14 | 1.04 ± 0.08 | 0.97 ± 0.04 | 1.09 ± 0.03 | 0.98 ± 0.02 | 1.02 ± 0.09 | 0.98 ± 0.02 |
| ADRM1 | 0.92 ± 0.03 | 0.95 ± 0.04 | 0.90 ± 0.11 | 0.93 ± 0.02 | 0.94 ± 0.25 | 1.04 ± 0.12 |

TABLE 14
Pluripotency marker levels in shFOXO4 hESCs. a, Data represent the mean ± s.e.m. of the relative expression levels to
GFP clone 1 H9 cells (GFP clone 1 (n = 11), HSF1 shRNA clone 1 (n = 6), FOXO1a shRNA clone 1 (n = 6), FOXO3a shRNA
clone 1 (n = 17), FOXO4 shRNA clone 1 (n = 14). b, Data represent the mean ± s.e.m. of the relative expression levels
to GFP clone 3 H9 cells (GFP clone 3 (n = 10), 3′UTR FOXO4 shRNA 1(n = 7), 3′UTR FOXO4 shRNA 2(n = 7),
3′UTR FOXO4 shRNA 3(n = 8)). c, Data represent the mean ± s.e.m. of the relative expression levels to GFP HUES-6 cells
(GFP (n = 9), HSF1 shRNA (n = 3), FOXO1a shRNA (n = 4), FOXO4 shRNA (n = 4), 3′UTR FOXO4 shRNA 1(n = 4),
3′UTR FOXO4 shRNA 2(n = 4), 3′UTR FOXO4 shRNA 3(n = 7)).
a H9 hESCs

| | | HSF1 shRNA | FOXO1a shRNA | FOXO3a shRNA | FOXO4 shRNA |
|---|---|---|---|---|---|
| | GFP clone 1 | clone 1 | clone 1 | clone 1 | clone 1 |
| 0CT4 | 0.98 ± 0.06 | 0.97 ± 0.10 | 0.84 ± 0.04 | 1.39 ± 0.12 | 1.52 ± 0.20 |
| NANOG | 0.89 ± 0.08 | 0.74 ± 0.21 | 1.69 ± 0.30 | 1.75 ± 0.13 | 1.63 ± 0.25 |
| UTF1 | 1.04 ± 0.06 | 0.65 ± 0.15 | 1.56 ± 0.36 | 1.49 ± 0.16 | 0.98 ± 0.10 |
| DPPA4 | 1.02 ± 0.03 | 1.26 ± 0.24 | 1.46 ± 0.29 | 1.34 ± 0.08 | 1.17 ± 0.13 |
| DPPA2 | 0.93 ± 0.03 | 0.65 ± 0.06 | 1.42 ± 0.37 | 1.95 ± 0.17 | 1.99 ± 0.13 |
| ZFP42 | 1.07 ± 0.06 | 1.02 ± 0.11 | 1.28 ± 0.19 | 1.16 ± 0.06 | 1.26 ± 0.10 |
| SOX2 | 0.98 ± 0.04 | 1.01 ± 0.17 | 0.75 ± 0.04 | 0.95 ± 0.08 | 0.43 ± 0.03 |

b H9 hESCs

| | | 3′UTR FOXO4 | 3′UTR FOXO4 | 3′UTR FOXO4 |
|---|---|---|---|---|
| | GFP clone 3 | shRNA 1 | shRNA 2 | shRNA 3 |
| 0CT4 | 0.90 ± 0.06 | 0.94 ± 0.07 | 1.05 ± 0.14 | 0.93 ± 0.08 |
| NANOG | 0.91 ± 0.05 | 1.08 ± 0.17 | 1.01 ± 0.10 | 1.00 ± 0.06 |
| UTF1 | 0.98 ± 0.05 | 1.45 ± 0.25 | 1.30 ± 0.25 | 1.62 ± 0.18 |
| DPPA4 | 0.97 ± 0.02 | 1.03 ± 0.08 | 0.97 ± 0.05 | 0.93 ± 0.07 |
| DPPA2 | 1.06 ± 0.09 | 1.36 ± 0.15 | 1.31 ± 0.23 | 1.07 ± 0.05 |
| ZFP42 | 0.89 ± 0.04 | 0.99 ± 0.10 | 1.12 ± 0.22 | 0.92 ± 0.06 |
| SOX2 | 0.99 ± 0.04 | 1.05 ± 0.09 | 0.99 ± 0.13 | 0.88 ± 0.05 |

c HUES-6 hESCs

| | | HSF1 | | | 3′UTR FOXO4 | 3′UTR FOXO4 | 3′UTR FOXO4 |
|---|---|---|---|---|---|---|---|
| | GFP clone 1 | shRNA | FOXO1a shRNA | FOXO4 shRNA | shRNA 1 | shRNA 2 | shRNA 3 |
| 0CT4 | 1.03 ± 0.11 | 0.96 ± 0.14 | 1.10 ± 0.15 | 0.62 ± 0.06 | 0.80 ± 0.09 | 1.65 ± 0.35 | 0.83 ± 0.09 |
| NANOG | 1.06 ± 0.17 | 1.18 ± 0.33 | 1.38 ± 0.28 | 1.03 ± 0.18 | 0.62 ± 0.29 | 1.62 ± 0.45 | 0.81 ± 0.19 |
| UTF1 | 1.06 ± 0.20 | 0.52 ± 0.12 | 1.90 ± 0.73 | 3.05 ± 0.39 | 1.62 ± 0.80 | 5.75 ± 1.10 | 0.66 ± 0.16 |
| DPPA4 | 1.02 ± 0.15 | 1.04 ± 0.23 | 1.76 ± 0.57 | 0.65 ± 0.04 | 1.15 ± 0.53 | 1.81 ± 0.44 | 1.05 ± 0.24 |
| DPPA2 | 0.99 ± 0.08 | 0.69 ± 0.03 | 1.42 ± 0.42 | 1.36 ± 0.51 | 0.80 ± 0.24 | 2.75 ± 0.44 | 0.96 ± 0.17 |
| ZFP42 | 1.27 ± 0.20 | 0.69 ± 0.28 | 1.90 ± 0.80 | 1.23 ± 0.09 | 0.73 ± 0.25 | 4.27 ± 1.33 | 1.10 ± 0.19 |
| SOX2 | 0.95 ± 0.05 | 1.06 ± 0.12 | 1.35 ± 0.16 | 0.69 ± 0.03 | 1.05 ± 0.18 | 0.87 ± 0.14 | 1.15 ± 0.11 |

TABLE 15
Neural differentiation of hESCs. After the differentiation process into
the neural lineage, cells show a dramatic decrease in pluripotency
markers and increased expression of NPC and neurogenesis markers.
Data represent the mean ± s.e.m.of the relative expression levels to
GFP overexpressing (OE) stable H9 hESCs (GFP OE stable H9
cells (n = 4), GFP OE NPCs (n = 6)).

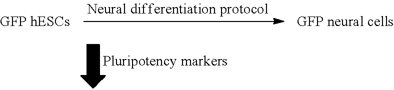

| | GFP hESCs | GFP neural cells |
|---|---|---|
| OCT4 | 1.13 ± 0.13 | 0.01 ± 0.00 |
| NANOG | 0.96 ± 0.04 | 0.05 ± 0.00 |
| SOX2 | 1.04 ± 0.04 | 0.84 ± 0.08 |
| UTF1 | 1.03 ± 0.03 | 0.10 ± 0.03 |
| DPPA4 | 1.11 ± 0.11 | 0.15 ± 0.02 |
| DPPA2 | 1.04 ± 0.03 | 0.12 ± 0.01 |
| ZFP42 | 1.00 ± 0.01 | 0.03 ± 0.00 |

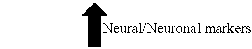

| | GFP H9 | GFP neural cells |
|---|---|---|
| Nestin | 1.04 ± 0.04 | 9.79 ± 0.93 |
| β-III-tubulin | 1.03 ± 0.03 | 41.35 ± 11.07 |
| MAP2 | 1.03 ± 0.04 | 44.60 ± 5.42 |

TABLE 16
FOXO4 is essential for hESCs differentiation into neural cells. After culturing in neural differentiation
media, FOXO4 shRNA hESCSs show decreased expression in neural markers and maintain
increased expression of pluripotency markers compared to the other cells. Graph (relative expression
to GFP clone 1 cells) represents the mean ± s.e.m. (n = 7).

| | | HSF1 shRNA | FOXO1a shRNA | FOXO3a shRNA | FOXO4 shRNA |
|---|---|---|---|---|---|
| | GFP clone 1 | clone 1 | clone 1 | clone 1 | clone 1 |
| HSF1 | 1.24 ± 0.12 | 0.63 ± 0.03 | 1.02 ± 0.07 | 1.07 ± 0.08 | 1.07 ± 0.09 |
| FOXO1a | 0.93 ± 0.06 | 0.78 ± 0.05 | 0.39 ± 0.05 | 0.78 ± 0.06 | 0.78 ± 0.08 |
| FOXO3a | 1.15 ± 0.05 | 0.86 ± 0.10 | 0.77 ± 0.10 | 0.52 ± 0.04 | 0.74 ± 0.09 |
| FOXO4 | 1.22 ± 0.13 | 1.19 ± 0.12 | 1.00 ± 0.07 | 0.71 ± 0.05 | 0.44 ± 0.03 |
| 0CT4 | 0.99 ± 0.04 | 1.05 ± 0.31 | 1.50 ± 0.37 | 2.58 ± 0.75 | 32.16 ± 4.28 |
| NANOG | 1.09 ± 0.10 | 1.03 ± 0.32 | 0.96 ± 0.16 | 1.55 ± 0.37 | 11.45 ± 0.91 |
| SOX2 | 1.13 ± 0.12 | 0.64 ± 0.05 | 0.79 ± 0.23 | 1.05 ± 0.12 | 1.75 ± 0.22 |
| UTF1 | 1.16 ± 0.07 | 0.86 ± 0.36 | 3.15 ± 1.33 | 1.98 ± 0.56 | 66.97 ± 23.39 |
| DPPA4 | 1.14 ± 0.16 | 0.94 ± 0.15 | 1.32 ± 0.19 | 1.44 ± 0.17 | 3.36 ± 0.27 |
| DPPA2 | 1.19 ± 0.12 | 0.63 ± 0.19 | 1.62 ± 0.42 | 1.52 ± 0.34 | 12.84 ± 2.52 |
| ZFP42 | 0.89 ± 0.05 | 1.02 ± 0.31 | 1.63 ± 0.43 | 1.50 ± 0.26 | 29.15 ± 5.36 |
| Nestin | 1.01 ± 0.10 | 1.02 ± 0.18 | 1.19 ± 0.15 | 1.05 ± 0.11 | 0.51 ± 0.05 |
| B-III tubulin | 1.38 ± 0.28 | 2.03 ± 0.33 | 1.71 ± 0.15 | 1.10 ± 0.15 | 0.26 ± 0.04 |
| MAP2 | 1.25 ± 0.15 | 1.04 ± 0.03 | 1.08 ± 0.08 | 1.08 ± 0.07 | 0.48 ± 0.01 |
| | | HSF1 shRNA | FOXO1a shRNA | FOXO3a shRNA | FOXO4 shRNA |
| | GFP clone 2 | clone 2 | clone 2 | clone 2 | clone2 |
| HSF1 | 1.21 ± 0.14 | 0.59 ± 0.01 | 1.14 ± 0.08 | 1.11 ± 0.09 | 1.06 ± 0.11 |
| FOXO1a | 0.98 ± 0.02 | 0.74 ± 0.07 | 0.40 ± 0.06 | 0.80 ± 0.08 | 0.84 ± 0.08 |
| FOXO3a | 1.06 ± 0.05 | 0.90 ± 0.13 | 0.85 ± 0.10 | 0.61 ± 0.03 | 0.78 ± 0.10 |
| FOXO4 | 1.13 ± 0.07 | 1.16 ± 0.13 | 1.00 ± 0.02 | 0.73 ± 0.06 | 0.40 ± 0.04 |
| 0CT4 | 0.99 ± 0.01 | 0.68 ± 0.17 | 0.57 ± 0.14 | 1.31 ± 0.44 | 31.7 ± 5.84 |
| NANOG | 0.94 ± 0.06 | 0.67 ± 0.19 | 0.77 ± 0.21 | 1.01 ± 0.28 | 11.57 ± 1.25 |
| SOX2 | 1.01 ± 0.02 | 0.63 ± 0.07 | 0.52 ± 0.06 | 1.10 ± 0.21 | 1.71 ± 0.23 |
| UTF1 | 1.10 ± 0.10 | 0.52 ± 0.09 | 0.81 ± 0.15 | 1.46 ± 0.44 | 78.08 ± 30.8 |
| DPPA4 | 0.94 ± 0.06 | 0.79 ± 0.10 | 1.11 ± 0.31 | 1.36 ± 0.28 | 3.14 ± 0.30 |
| DPPA2 | 1.17 ± 0.17 | 0.74 ± 0.25 | 0.66 ± 0.34 | 1.55 ± 0.48 | 13.6 ± 3.27 |
| ZFP42 | 0.94 ± 0.06 | 0.89 ± 0.29 | 0.69 ± 0.20 | 1.17 ± 0.24 | 30.75 ± 6.79 |
| Nestin | 0.92 ± 0.06 | 0.90 ± 0.16 | 1.27 ± 0.32 | 1.01 ± 0.10 | 0.50 ± 0.07 |
| B-III tubulin | 1.06 ± 0.04 | 1.72 ± 0.23 | 1.24 ± 0.13 | 1.10 ± 0.24 | 0.21 ± 0.04 |
| MAP2 | 1.00 ± 0.01 | 1.02 ± 0.02 | 0.93 ± 0.02 | 1.10 ± 0.14 | 0.47 ± 0.01 |

TABLE 17
Trophoblast differentiation of stable FOXO4 shRNA hESCs. Data (relative expression
to GFP cells clone 1) represent the mean ± s.e.m. (n = 8).

| | | HSF1 shRNA | FOXO1a shRNA | FOXO3a shRNA | FOXO4 shRNA |
|---|---|---|---|---|---|
| | GFP clone 1 | clone 1 | clone 1 | clone 1 | clone 1 |
| 0CT4 | 1.09 ± 0.15 | 1.18 ± 0.28 | 1.74 ± 0.29 | 0.87 ± 0.16 | 1.28 ± 0.20 |
| NANOG | 0.97 ± 0.15 | 1.04 ± 0.17 | 1.73 ± 0.32 | 0.76 ± 0.09 | 1.58 ± 0.19 |
| SOX2 | 0.89 ± 0.13 | 1.48 ± 0.33 | 1.06 ± 0.24 | 0.52 ± 0.14 | 0.18 ± 0.05 |
| DPPA4 | 0.96 ± 0.04 | 0.89 ± 0.05 | 0.87 ± 0.05 | 0.73 ± 0.09 | 1.25 ± 0.09 |
| DPPA2 | 0.92 ± 0.11 | 0.76 ± 0.05 | 1.49 ± 0.16 | 0.85 ± 0.09 | 2.37 ± 0.13 |
| ZFP42 | 0.94 ± 0.08 | 0.95 ± 0.09 | 1.28 ± 0.22 | 1.39 ± 0.13 | 1.17 ± 0.05 |
| CD9 | 1.00 ± 0.14 | 1.32 ± 0.17 | 1.72 ± 0.21 | 1.31 ± 0.17 | 1.40 ± 0.10 |
| CGB | 1.23 ± 0.05 | 0.91 ± 0.09 | 1.55 ± 0.26 | 1.29 ± 0.12 | 2.80 ± 0.29 |
| GATA2 | 0.94 ± 0.04 | 1.23 ± 0.09 | 0.78 ± 0.07 | 0.76 ± 0.12 | 1.14 ± 0.04 |
| GATA3 | 0.96 ± 0.07 | 1.03 ± 0.12 | 0.98 ± 0.17 | 1.15 ± 0.12 | 1.88 ± 0.10 |
| GCM | 0.94 ± 0.04 | 0.92 ± 0.11 | 1.09 ± 0.20 | 0.63 ± 0.17 | 2.27 ± 0.20 |
| HEY1 | 1.00 ± 0.04 | 1.02 ± 0.10 | 0.85 ± 0.22 | 0.72 ± 0.09 | 3.35 ± 0.53 |
| MSX2 | 1.03 ± 0.09 | 1.17 ± 0.16 | 0.99 ± 0.15 | 0.67 ± 0.03 | 1.42 ± 0.07 |
| PAEP | 1.14 ± 0.04 | 1.27 ± 0.40 | 4.16 ± 0.61 | 1.16 ± 0.25 | 5.77 ± 0.41 |
| TFAP2 | 1.02 ± 0.04 | 0.98 ± 0.13 | 0.97 ± 0.12 | 1.12 ± 0.18 | 1.56 ± 0.08 |
| | | HSF1 shRNA | FOXO1a shRNA | FOXO3a shRNA | FOXO4 shRNA |
| | GFP clone 2 | clone 2 | clone 2 | clone 2 | clone2 |
| 0CT4 | 1.00 ± 0.02 | 0.94 ± 0.11 | 1.43 ± 0.14 | 1.10 ± 0.28 | 1.06 ± 0.18 |
| NANOG | 1.06 ± 0.17 | 1.04 ± 0.18 | 1.51 ± 0.34 | 1.71 ± 0.11 | 1.23 ± 0.03 |
| SOX2 | 1.04 ± 0.13 | 1.99 ± 0.46 | 1.42 ± 0.29 | 0.43 ± 0.25 | 0.17 ± 0.09 |
| DPPA4 | 1.00 ± 0.04 | 0.82 ± 0.06 | 0.84 ± 0.04 | 0.83 ± 0.11 | 1.09 ± 0.09 |
| DPPA2 | 1.01 ± 0.07 | 0.84 ± 0.05 | 1.32 ± 0.22 | 0.98 ± 0.09 | 2.39 ± 0.05 |
| ZFP42 | 1.00 ± 0.03 | 0.82 ± 0.15 | 1.62 ± 0.31 | 1.57 ± 0.20 | 1.15 ± 0.07 |
| CD9 | 1.02 ± 0.10 | 1.54 ± 0.15 | 1.81 ± 0.16 | 1.29 ± 0.19 | 1.50 ± 0.17 |
| CGB | 1.01 ± 0.07 | 0.82 ± 0.06 | 1.10 ± 0.13 | 0.82 ± 0.26 | 3.60 ± 0.25 |
| GATA2 | 1.01 ± 0.05 | 1.18 ± 0.11 | 0.87 ± 0.02 | 0.76 ± 0.15 | 1.13 ± 0.06 |
| GATA3 | 1.01 ± 0.02 | 1.04 ± 0.25 | 1.02 ± 0.24 | 1.06 ± 0.17 | 1.71 ± 0.12 |
| GCM | 1.01 ± 0.08 | 1.02 ± 0.22 | 0.74 ± 0.16 | 0.77 ± 0.20 | 1.96 ± 0.18 |
| HEY1 | 1.01 ± 0.02 | 1.17 ± 0.11 | 0.36 ± 0.03 | 0.82 ± 0.09 | 2.82 ± 0.19 |
| MSX2 | 1.01 ± 0.06 | 1.43 ± 0.24 | 0.68 ± 0.08 | 0.65 ± 0.06 | 1.48 ± 0.08 |
| PAEP | 1.01 ± 0.10 | 1.56 ± 0.45 | 3.50 ± 0.80 | 0.70 ± 0.17 | 6.57 ± 0.99 |
| TFAP2 | 1.01 ± 0.04 | 0.88 ± 0.16 | 1.00 ± 0.16 | 0.91 ± 0.14 | 1.44 ± 0.08 |

TABLE 18
Sequences cloned to generate lentiviral vectors.

| Gene | Sequence |
|---|---|
| HSF1 | CAC ATT CCA TGC CCA AGT A |
| FOXO1a | GCG CTT AGA CTG TGA CAT G |
| FOXO3a | AAG GAT AAG GGC GAC AGC AA |
| FOXO4 | AGA AGC CGA TAT GTG GAC C |
| FOXO4 (3′UTR_1) | CACTTAGGCTTTGTAGCAAGA |
| FOXO4 (3′UTR_2) | GCGTGTTCATATCTACTCTTT |
| FOXO4 (3′UTR_3) | TGATAGTGACATGATACAAAC |

TABLE 19
List of primers used for qPCR assays.
HOUSEKEEPING

| Gene | Forward (5′ → 3′) | Reverse (5′ → 3′) |
|---|---|---|
| ACTB | CTGGCACCCAGCACAATG | CCGATCCACACGGAGTACTTG |
| GAPDH | GCACCGTCAAGGCTGAGAAC | GGATCTCGCTCCTGGAAGATG |

FOXOs/HSF-1

| Gene | Description | Forward (5′ → 3′) | Reverse (5′ → 3′) |
|---|---|---|---|
| HSF1 | heat shock | AACATGTATGGCTTCCGGAAA | TGTCGTCTCTCTCTGGCTTGAC |
| | transcription factor 1 | | |
| FOXO1a | forkhead box O1 | TCATGTCAACCTATGGCAG | CATGGTGCTTACCGTGTG |
| FOXO3a | forkhead box O3 | TGCCGGCTGGAAGAACTC | CCGCATGAATCGACTATGCA |
| FOXO4 | forkhead box O4 | TGGTCCGTACTGTACCCTACTTCA | GGCGGATCGAGTTCTTCCAT |

Proteasome subunits

| 20S α | Forward (5′ → 3′) | Reverse (5′ → 3′) |
|---|---|---|
| PSMA1 | GCCTGTGTCTCGTCTTGTATCTCTAA | TCCGGCCATATCGTTGTGTT |
| PSMA2 | TGAATATGCTTTGGCTGCTGTAG | CCACACCATTTGCAGCTTTAATT |
| PSMA3 | GAATGACGGTGCGCAACTCT | CAGCCCCAATAACCGTATGAA |
| PSMA4 | ACTATATTTTCTCCAGAAGGTCGCTTA | CAAACAGGTGCCTGCATGTC |
| PSMA5 | CCAGAGTGGAGACACAGAACCA | GGGTCACACTCTCCACTGTCATT |
| PSMA6 | CCCGAGGGTCGGCTCTA | GATGTAAGGCCACCCTGGTTAA |
| PSMA7 | GCCAGTCTGAAGCAGCGTTAT | TGAGGGCAGAGATGCCAAAC |
| 20S β | Forward (5′ → 3′) | Reverse (5′ → 3′) |
| PSMB1 | AGGCGCTTCTTTCCATACTATGTT | GCCCCCTTTCCTTCTTCATC |
| PSMB2 | GATGCGAAATGGATATGAATTGTC | AGGTTTCGGCGTGTGAAGTT |
| PSMB3 | CTGTGGACCGGGATGCA | GATTTTGTCCTTCTCGATGATGTG |
| PSMB4 | ACATGCTTGGTGTAGCCTATGAAG | AGAGGCTGAGCCAAGTATGCA |
| PSMB5 | CAGAAGAGCCAGGAATCGAAA | TCCATGGCGGAACTTGAAG |
| PSMB6 | CAAGGAAGAGTGTCTGCAATTCA | CCGCTCCATGGCCAAA |
| PSMB7 | CAGCCAATCGGATGCTGAA | AGGGCTGCACCAATGTAACC |
| 19S ATPases | Forward (5′ → 3′) | Reverse (5′ → 3′) |
| PSMC1 | AGACCAGGCCGCATTGAC | TGCGCTTCTTCGTCTTTTCA |
| PSMC2 | TTGGCTGCAGATAAGCAGACA | TCTTTGTACACCTGGCAACCTGTA |
| PSMC3 | CACACGGCTGCTGGACAGT | CTTGGAGCTCATGGGTGACTCT |
| PSMC4 | GGCCCGGCCAGATAAGATT | CAACATTCCACTCTCCTGACAGAT |
| PSMC5 | CGAGAACGGCGAGTCCAT | TTCTGCATGACCTTGGCTACTG |
| PSMC6 | CCAGAGTTATTTCAGCGTGTAGGA | CGTACCTGGTGGTCCATATAACAA |
| 19S non-ATPases | Forward (5′ → 3′) | Reverse (5′ → 3′) |
| PSMD1 | GCTTCTGTGCCTGGATCCA | TCCATCGAGTCACTGTCTTTCTCT |
| PSMD2 | GCGTGAGTGCCTCAAGTATCG | CCAGATGCCTGACATACTCATGA |
| PSMD3 | GAGCAGGCCAACAACAATGA | GCTTTGATTCGCCCTGTGTAG |
| PSMD4 | CACTATGGTGTGTGTGGACAACAG | TGCAGCCTGGTGGGTAAGA |
| PSMD6 | TGACAAAGAGGGAGCTCTGACA | GACCCAGGGCCACAGTTTT |
| PSMD7 | GAAGCTGAGGAAGTTGGAGTTGA | TGCCCACCGTCGTGTCTT |
| 20S β | Forward (5′ → 3′) | Reverse (5′ → 3′) |
| PSMD11 | GCCATCTACTGCCCCCCTAA | ATGGATAATACCCGACTGCATGT |
| PSMD12 | GACTCGTACTGCTTCCGATATGG | TCATAGCACATCTTCACTACTGCAACT |
| ADRM1 | GGGTCCAAGCGGCTTTTC | GCTCCTCATCCTGGTCTGTCTT |
| PSMD14 | TGAACAGCTGGCAATAAAGAATG | AGTACATCCACATGTTCCTCCAAA |

Pluripotency markers

| Gene | Description | Forward (5′ → 3′) | Reverse (5′ → 3′) |
|---|---|---|---|
| 0CT4 | POU class 5 homeobox 1 | GGAGGAAGCTGACAACAATGAAA | GGCCTGCACGAGGGTTT |
| NANOG | Nanog homeobox | AAATCTAAGAGGTGGCAGAAAAACA | GCCTTCTGCGTCACACCATT |
| SOX2 | SRY (sex determining region Y)- | TGCGAGCGCTGCACAT | TCATGAGCGTCTTGGTTTTCC |
| | box 2 | | |
| UTF1 | undifferentiated embryonic cell | CGCCGCTACAAGTTCCTTAAA | GGATCTGCTCGTCGAAGGG |
| | transcription factor 1 | | |
| DPPA4 | developmental pluripotency | CTGGTGCCAACAATTGAAGCT | AGGCACACAGGCGCTTATATG |
| | associated 4 | | |
| DPPA2 | developmental pluripotency | GTACTAATGGCAAGAAAATCGAAGTTT | GCCGTTGTTCAGGGTAAGCA |
| | associated 2 | | |
| ZFP42 | zinc finger protein 42 homolog | CCTGCAGGCGGAAATAGAAC | GCACACATAGCCATCACATAAGG |
| | (mouse) | | |
| TERT | telomerase reverse transcriptase | CATTTTTCCTGCGCGTCAT | GCGACATCCCTGCGTTCT |

Markers for the germ and extraembryonic layers

| Gene | Description | Forward (5′ → 3′) | Reverse (5′ → 3′) |
|---|---|---|---|
| CDX2 | caudal type homeobox 2 | CTCGGCAGCCAAGTGAAAA | GGTCCGTGTACACCACTCGAT |
| PAX6 | paired box 6 | CATACCAAGCGTGTCATCAATAAAC | TGCGCCCATCTGTTGCT |
| FGF5 | fibroblast growth factor 5 | ACGAGGAGTTTTCAGCAACAAAT | TTGGCACTTGCATGGAGTTTT |
| MSX1 | msh homeobox 1 | CTCCGCAAACACAAGACGAAC | CACATGGGCCGTGTAGAGTC |
| AFP | alpha-fetoprotein | GAGGGAGCGGCTGACATTATT | ACCAGGGTTTACTGGAGTCATTTC |
| GATA6 | GATA binding protein 6 | AGCGCGTGCCTTCATCA | GTGGTAGTTGTGGTGTGACAGTTG |
| GATA4 | GATA binding protein 4 | TCCGTGTCCCAGACGTTCTC | GAGAGGACAGGGTGGATGGA |
| ALB | albumin | TGAGGTTGCTCATCGGTTTAAA | GCAATCAACACCAAGGCTTTG |

Neuronal markers

| Gene | Description | Forward (5′ → 3′) | Reverse (5′ → 3′) |
|---|---|---|---|
| NES | nestin | TGAAGGGCAATCACAACAGG | TGACCCCAACATGACCTCTG |
| TUBB3 | tubulin, beta 3 | GGCCAAGTTCTGGGAAGTCA | CGAGTCGCCCACGTAGTTG |
| MAP2 | microtubule-associated protein | AAAGAAGCTCAACATAAAGACCAGACT | GTGGAGAAGGAGGCAGATTAGC |
| | 2 | | |

Trophoblast markers

| Gene | Description | Forward (5′ → 3′) | Reverse (5′ → 3′) |
|---|---|---|---|
| CD9 | CD9 molecule | TGGGCATCGGCATTGC | ACAGCACAAGATCATACTGAAGATCA |
| CGB | chorionic | CAGGGGACGCACCAAGG | CAGCACGCGGGTCATGG |
| | gonadotropin, | | |
| | beta | | |
| | polypeptide | | |
| GATA2 | GATA binding | CCTCTACTACAAGCTGCACAATGTTAA | CCGAGTCTGGATCCCTTCCT |
| | protein 2 | | |
| GATA3 | GATA binding | TTCACAATATTAACAGACCCCTGACT | GATTTGCTAGACATTTTTCGGTTTC |
| | protein 3 | | |
| GCM1 | glial cells | GAAGCAGCAGCGGAAACG | GGCAAGGGATGAGCTTCAGA |
| | missing | | |
| | homolog 1 | | |
| | (Drosophila) | | |
| HEY1 | hairy/enhancer- | CGGCAGGAGGGAAAGGTTAC | TCCCAAACTCCGATAGTCCATAG |
| | of-split related | | |
| | with YRPW | | |
| | motif 1 | | |
| MSX2 | msh homeobox | CGCCGCCAAGACATATGAG | GCTTCCGATTGGTCTTGTGTTT |
| | 2 | | |
| PAEP | progestagen- | TGAGAAGAAGGTCCTTGGAGAGA | CGCCACCGTATAGTTGATCTTG |
| | associated | | |
| | endometrial | | |
| | protein | | |
| TFAP2 | transcription | GCAAGTGACAAGAAAAAACATGCT | AGCAGGTCGGTGAACTCTTTG |
| | factor AP-2 | | |
| | alpha | | |

Keratinocyte markers

| Gene | Description | Forward (5′ → 3′) | Reverse (5′ → 3′) |
|---|---|---|---|
| K14 | keratin 14 | GCCTGCTGAGATCAAAGACTACAGT | GTGGCTGTGAGAATCTTGTTCCT |
| p63 | tumor protein p63 | CCACCTCCGTATCCCACAGA | GACATGATGAACAGCCCAACCT |
| DSG3 | desmoglein 3 | GAAGTCCGTACTTTGACCAATTCTC | CCACTCACAACCAGACGATAGC |
| LAMB3 | laminin, beta 3 | AGGATGAAAGACATGGAGTTGGA | CATTGATGTGGTCACGGATCTG |
| KRT5 | keratin 5 | TGAGAGCCGAGATTGACAATGT | TCCGCAATGGCGTTCTG |
| Col7a1 | collagen, type VII, alpha | ACCCGACCTCCGGATGA | ATTGGCTGCTTGGCTCAGA |
| | 1 | | |

Fibroblast markers

| Gene | Description | Forward (5′ → 3′) | Reverse (5′ → 3′) |
|---|---|---|---|
| VIM | vimentin | TCTGCCTCTTCCAAACTTTTCC | AACCAGAGGGAGTGAATCCAGAT |
| S100A4 | fibroblast-specific | GGTGACAAGTTCAAGCTCAACAA | CATCTGTCCTTTTCCCCAAGAA |
| | protein-1 | | |
| COL6A2 | collagen, type VI, | CTCTCCTCCGTCTTCCTGTG | GTGACCTGATGCAGCAAAGA |
| | alpha 2 | | |
| COL1A1 | collagen, type I, alpha | AAGAGGAAGGCCAAGTCGAG | CACACGTCTCGGTCATGGTA |
| | 1 | | |
| P4H1 | prolyl 4-hydroxylase, | CTTCCAGGAGTGAAACACAAATCTT | GCCACTTTGCCCAACTCAAA |
| | alpha polypeptide I | | |
| FAP | fibroblast activation | AGCGACTACGCCAAGTACTATGC | CATCATGAAGGGTGGAAATGG |
| | protein, alpha | | |
| THY-1 | Thy-1 cell surface | TCTCCTCCCAGAACGTCACAGT | TGAGCCAGCAGGCTGATG |
| | antigen | | |
| ACTA2 | actin, alpha 2, smooth | CACCATCGGAAATGAACGTTT | GACTCCATCCCGATGAAGGA |
| | muscle | | |
| DES | desmin | CCGACACCAGATCCAGTCCTA | GGGAATCGTTAGTGCCCTTCA |

What is claimed is:

1. A method of modulating a proteasome activity in a cell comprising:
modulating an rpn-6.1 protein activity or an rpn-6.1 protein level in said cell thereby modulating said proteasome activity.

2. The method of claim 1, wherein modulating said rpn-6.1 protein activity or said rpn-6.1 protein level further comprises increasing said rpn-6.1 protein activity or said rpn-6.1 protein level, thereby increasing said proteasome activity.

3. The method of claim 1, wherein modulating said rpn-6.1 protein activity or said rpn-6.1 protein level further comprises decreasing said rpn-6.1 protein activity or said rpn-6.1 protein level, thereby decreasing said proteasome activity.

4. The method of claim 1, wherein said modulating said rpn-6.1 protein protein level comprises introducing to said cell a nucleic acid encoding an rpn-6.1 polypeptide.

5. The method of claim 1, wherein said modulating said rpn-6.1 protein activity comprises administering an rpn-6.1 antagonist or agonist to said cell, thereby modulating said proteasome activity.

6. A method as in claim 1, 2, 3, 4, or 5, wherein said cell forms an organism.

7. A method of increasing cell survival of a cell suffering from proteotoxic stress comprising:
increasing an rpn-6.1 protein activity or an rpn-6.1 protein level in a cell thereby increasing cell survival of said cell suffering from proteotoxic stress.

8. The method of claim 7, wherein said proteotoxic stress is oxidative stress.

9. The method of claim 7, wherein said increasing said rpn-6.1 protein level comprises introducing to said cell a nucleic acid encoding an rpn-6.1 polypeptide.

10. The method of claim 7, wherein said increasing said rpn-6.1 protein activity comprises administering an rpn-6.1 agonist to said cell, thereby increasing said rpn-6.1 protein activity.

11. The method of claim 7, wherein said increasing said rpn-6.1 protein activity or said rpn-6.1 protein level comprises increasing stress tolerance in said cell.

12. A method of treating a protein-misfolding disease in a subject in need thereof comprising:
administering to said subject a therapeutically effective amount of a rpn-6.1 modulator.

13. The method of claim 12, wherein said rpn-6.1 modulator increases an rpn-6.1 protein activity or an rpn-6.1 protein level.

14. The method of claim 12, wherein said protein misfolding-disease is a neurodegenerative disease.

15. The method of claim 14, wherein said neurodegenerative disease is Huntington's disease, Alzheimer's disease, or Parkinson's disease.

16. A method of increasing neurogenesis in a cell comprising increasing a Foxo4 protein activity or a Foxo4 protein level in said cell.

17. The method of claim 16, wherein said increasing said Foxo4 protein activity or said Foxo4 protein level further comprises increasing a PSMD 11 protein activity or a PSMD11 protein level.

18. The method of claim 17, wherein said increasing said PSMD 11 protein activity or said PSMD11 protein level further comprises increasing the proteasome activity of said cell.

19. The method as in claim 16, 17, or 18, wherein said cell forms an organism.

20. A method of preparing an induced pluripotent stem cell comprising:
modulating a Foxo4 protein activity or a Foxo4 protein level in a non-pluripotent cell thereby forming a modulated non-pluripotent cell; and
allowing said modulated non-pluripotent cell to divide thereby forming said induced pluripotent stem cell.

21. The method of claim 20, wherein said modulating comprises increasing a Foxo4 protein activity or a Foxo4 protein level in said non-pluripotent cell.
